(19) United States
(12) Patent Application Publication
(10) Pub. No.: US 2003/0229037 A1
(43) Pub. Date: Dec. 11, 2003

(54) Novel cationic amphiphiles

(76) Inventors: Ulrich Massing, Merzhausen (DE); Thomas Fichert, Krailling (DE)

Correspondence Address: BRINKS HOFER GILSON & LIONE, P.O. BOX 10395, CHICAGO, IL, 60611, US

(21) Appl. No.: 10/182,449

(22) PCT Filed: Feb. 5, 2001
(86) PCT No.: PCT/US01/40020

(30) Foreign Application Priority Data
Feb. 7, 2000 (US) 60180600

(51) Int. Cl. A61K 48/00; A61K 38/17; A61K 31/56; A61K 9/127; C12N 15/88; C07J 9/00

(52) U.S. Cl. 514/44; 424/450; 514/12; 435/458; 552/544; 514/171

(57) ABSTRACT

A cationic amphiphile for facilitating transport of a biologically active molecule into a cell has the structure A-F-D, in which A is a lipid anchor, D is a head group, and F is a spacer group having the structure described herein. A method for facilitating transport of a biologically active molecule into a cell comprises preparing a lipid mixture comprising a cationic amphiphile having structure A-F-D, preparing a lipoplex by contacting the lipid mixture with a biologically active molecule; and contacting the lipoplex with a cell, thereby facilitating transport of the biologically active molecule into the cell.

### BACKGROUND

The present invention relates to novel cationic amphiphiles for delivery of biologically active molecules into cells (i.e., transfection).

Rapid advances in molecular biology lead to a continual improvement in the understanding of the genetic origins of physiological processes. Of particular interest in this context is the comprehensive research into the genetic basis of disease, because this is the decisive prerequisite for treating diseases with genetic etiologies using gene therapy [Mulligan, 1993]. Gene therapy is defined as the introduction of exogenous genetic material into cells that results in a therapeutic benefit for the patient [Morgan and Anderson, 1993]. In addition to diseases that are not due to genetic defects, such as AIDS, diseases that are caused by congenital defects or defects that are acquired during an individual's life are especially suitable for gene therapy [Friedmann, 1997]. As a result, many studies on the treatment of cystic fibrosis (also called mucoviscidosis, an example of a congenital genetic defect) have been described [Crystal, 1995]. In cystic fibrosis, a chloride ion channel of lung epithelial cells is defectively expressed. The introduction of an intact gene into the affected cells has led to initial clinical success [Welsh and Zabner, 1999; Knowles et al., 1995]. Carcinoses based on acquired genetic defects also represent a promising target for gene therapy [Blaese, 1997]. Various strategies have been described in this context for the specific destruction of malignant cells and cells that the host immune system no longer recognizes as malignant.

Before gene therapy can be introduced as a clinical concept with broad application, an effective, reliable technique must be developed for the selective introduction of therapeutic genes into defective cells (transfection). Additionally, the required DNA vehicles must be available in large quantities, they must be reproducible, and their process of manufacture must be reliable [Deshmukh and Huang, 1997]. The development of new transfection methods and the improvement of existing methods have therefore taken on considerable importance in the fields of biology, chemistry, and medicine in recent years.

A number of new cationic lipids have been synthesized in recent years, the transfection rates of which are not yet as high as the rates that can be achieved using viral transfection. Only a few systematic studies into the relationships between the structure and effect of varied, cationic lipids have been carried out to date. These studies are limited mainly to variations in apolar hydrocarbon chains [Wang et al., 1998] and are less concerned with the question of the effect of systematic structural variations of the spacer [Ren and Leu, 1999] and head group [Cooper et al., 1998; Huang et al., 1998] of cationic lipids on the transfection result.

Thus, in accord with presently preferred embodiments of the invention, new transfection lipids with systematic variations in the spacer and head group, and new synthesis strategies for preparing simple cationic and polycationic lipids are provided.

### SUMMARY

Briefly stated, in a composition of matter aspect, the present invention is directed to a cationic amphiphile having the structure A-F-D, wherein:

A is a lipid anchor;

F is a spacer group having the structure

O—C(O)-G¹-[C(R¹)(R²)]ₘ-G²-{C(O)-E-[C(R³)(R⁴)]ₙ}ₚ; and

D is a head group; and wherein:

G¹ and G² are the same or different, and are independently either oxygen or a bond;

R¹, R², R³ and R⁴ are the same or different, and are independently selected from the group consisting of hydrogen and alkyl radicals;

m, n and p are the same or different, and are independently either 0, 1, 2, 3, 4, 5, or 6; and

E is oxygen or N(R⁵), wherein R⁵ is hydrogen or an alkyl radical, provided that E does not contain nitrogen when D is N(CH₃)₂ and when A is cholesterol, and when R⁵ is hydrogen, and when both G¹ and G² are bonds, and when each of R¹, R², R³ and R⁴ is hydrogen, and when both m and n are 2, and when p is 1.

In a method aspect, the present invention is directed to providing a method for facilitating transport of a biologically active molecule into a cell, which includes preparing a liposomal dispersion comprising a cationic amphiphile having the structure A-F-D, wherein:

A is a lipid anchor;

F is a spacer group having the structure

O—C(O)-G¹-[C(R¹)(R²)]ₘ-G²-{C(O)-E-[C(R³)(R⁴)]ₙ}ₚ; and

D is a head group; and wherein:

G¹ and G² are the same or different, and are independently either oxygen or a bond;

R¹, R², R³ and R⁴ are the same or different, and are independently selected from the group consisting of hydrogen and alkyl radicals;

m, n and p are the same or different, and are independently either 0, 1, 2, 3, 4, 5, or 6; and

E is oxygen or N(R⁵), wherein R⁵ is hydgrogen or an alkyl radical, provided that E does not contain nitrogen when D is N(CH₃)₂ and when A is cholesterol, and when R⁵ is hydrogen, and when both G¹ and G² are bonds, and when each of R¹, R², R³ and R⁴ is hydrogen, and when both m and n are 2, and when p is 1.

The method also includes preparing a lipoplex by contacting the liposomal dispersion with a biologically active molecule, and contacting the lipoplex with a cell, thereby facilitating transport of a biologically active molecule into the cell.

The scope of the present invention is defined solely by the appended claims, and is not affected to any degree by the statements within this summary.

### BRIEF DESCRIPTION OF THE DRAWINGS

FIG. 1 shows a diagram of the “sandwich-like” structure of lipids.

FIG. 2 shows an electron microscopic image of endocytosis of gold-labelled lipoplexes.

FIG. 3 shows the proposed mechanism of passage of lipoplexes into the cell and the subsequent release of DNA from then endosomes (cationic and anionic/zwitterionic lipids are filled-in and non-filled-in circles, respectively).

FIG. 4 shows an overview of structural variations in synthesized simple cationic lipids.

FIG. 5 shows an overview of structural variations in synthesized bicationic lipids.

FIG. 6 shows an overview of structural variations in synthesized tricationic lipids.

FIG. 7 shows examples of typical transfection diagrams.

FIG. 8 shows transfection diagrams of the acetyl and carbonate derivatives with a tertiary amino group.

FIG. 9 shows transfection diagrams of lipids 10 and 11 with a permethylated amino group.

FIG. 10 shows transfection diagrams of lipids 12 and 13 having an additional 2-hydroxy ethyl group.

FIG. 11 shows transfection diagrams of lipids 6-9 having succinyl spacers.

FIG. 12 shows transfection diagrams of bicationic lipids with spacer variations.

FIG. 13 shows transfection diagrams of bicationic lipids with head group variations.

FIG. 14 shows transfection diagrams of carbonate and succinyl derivatives with two lipid anchors.

FIG. 15 shows transfection diagrams of tricationic lipids varied in the spacer.

FIG. 16 shows transfection diagrams of lipids that were varied systematically in the head group.

FIG. 17 shows a comparison of the maximum transfection efficiencies of lipids varied in the head group (most effective lipid/DNA ratio in each case).

FIG. 18 shows a transfection diagram of the DMG lipid 110 having a tertiary amino group.

FIG. 19 shows a transfection diagram of DMG lipid 111 having a permethylated amino group.

FIG. 20 shows a transfection diagram of DMG lipid 113 having a bicationic head group.

FIG. 21 shows a transfection diagram of DMG lipid 115 having a tricationic head group.

FIG. 22 shows transfection efficiencies of the most effective lipids having the same spacers and head groups and containing one (10, 13, 6, 8), two (57, 58, 59, 60), or three (104, 98, 99, 100) amino group(s).

FIG. 23 shows a transfection diagram of lipid 104.

FIG. 24 shows an exampe of calculating lipid/DNA ratios.

### DETAILED DESCRIPTION OF PREFERRED EMBODIMENTS

Various transfection techniques are in use today; they include the classic “physical” methods such as electroporation [Bertling et al., 1987], microinjection [Capecchi, 1980], and the particle bombardment of cells [Klein et al., 1987]. “Chemical” methods are also used frequently, such as calcium phosphate precipitation [Chen et al., 1993] and DEAE-dextran precipitation [Keown et al., 1990]. The known techniques cannot be used systematically, that is, in in vivo gene therapy applications (e.g., via injection into the bloodstream). Transfection techniques using viral and non-viral synthetic vectors can be performed systematically, however.

In viral transfection, viral genes in a virus are replaced with therapeutic genes. Adenoviruses (DNA viruses), retroviruses (RNA viruses), and adeno-associated viruses [Crystal, 1995] naturally deliver DNA/RNA into cells with a high level of efficiency. This advantage is offset by a few critical disadvantages, however: activation of the host immune system, the minimal but persistent risk of infection, and potential insertion mutagenesis (possibility of inducing cancer) if the therapeutic gene is inserted in unfavorable sites in the host genome of the cell [Gao and Huang, 1995; Reifers and Kreuzer, 1995]. These problems have not been solved to date. A disadvantage of viral transfection is the size limitation of the usable therapeutic gene [Behr, 1993 and 1994]. It is still uncertain as to how the requirements for the production of viral vectors in large quantities and elaborate quality assurance procedures can be fulfilled [Deshmukh and Huang, 1997].

To circumvent the disadvantages of viral transfection, research has focused on transfection using positively charged polymers such as polylysines [Ferkol et al., 1996], and on lipid-mediated transfection (lipofection). In initial experiments, cells were transfected with neutral or negatively charged liposomes containing DNA [Wong et al., 1980]. Low liposomal delivery efficiencies and low transfection efficiencies using neutral/negatively charged lipids finally led to the successful use of synthetic cationic lipids for gene transfer in 1987 [Felgner et al., 1987].

Despite the fact that gene transfer is less effective than viruses and the occasional occurrence of lipid cytotoxicities, lipofection is currently favored over other techniques for use in in vivo or ex vivo gene therapy for the following reasons: in comparison with viral transfection, lipofection offers advantages in that the size of the therapeutic gene to be inserted is not restricted, and it does not involve immunogenicity or risk of infection. Additionally, cationic lipids can be manufactured in large quantities with relatively little effort.

The structure of cationic lipids can be broken down into three structural elements: a lipophilic lipid anchor comprising two long alkyl chains or cholesterol, a spacer, and a polar, positively charged head group consisting of one or more quaternized or protonatable amino groups.

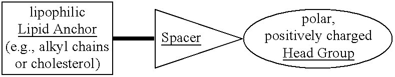

The mechanism of cationic lipid-mediated transfection is complex and only partially understood in detail at this time. In order to describe the processes in detail, lipofection is broken down into three individual steps [Miller, 1998]: the formation of positively charged lipid/DNA complexes, the passage of the complexes into the cell, and the passage of DNA into the nucleus. While it is not the intention of the applicant to be bound to any particular theory, nor to affect in any measure the scope of the appended claims, the following discussion on the mechanism of transfection is proffered solely for the purpose of illustration and explanation.

Formation of Lipid/DNA Complexes (Lipoplexes)

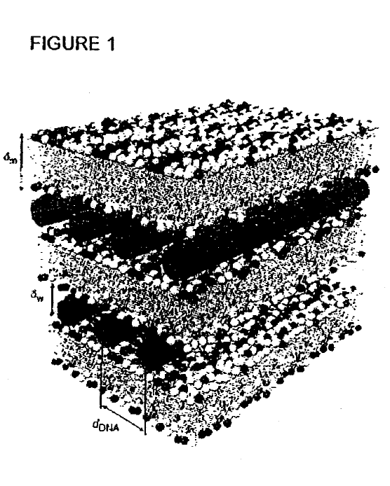

Lipoplexes with a positive excess charge are typically used in transfection because they apparently interact better with the negatively charged surface of cells, and because cells can take them up better [Zabner et al., 1995]. To form positively charged lipoplexes, liposomes are formed from cationic lipids and then added in excess to the DNA to be introduced into the cells. In this process, ionic interactions enable the lipids to bind via their positively charged head groups to the backbone of the DNA from negatively charged phosphate groups. A decisive factor for the shape and structure of the resultant lipoplexes and, therefore, the success of transfection, is the proportion of lipid/DNA [Sternberg et al., 1994; Eastman et al., 1997]. Mixing experiments have revealed that lipid/DNA ratios of >1 result in positively charged lipoplexes in which the DNA is present in highly condensed form. This was verified using electron microscopic images of complexes taken of different proportions [Gershon et al., 1993]. The strong condensation of DNA also explains why it is protected in the lipoplexes before it is broken down by nucleases [Bhattacharya and Mandal, 1998]. X-ray diffraction studies of lipid/DNA complexes were used to refine the structural models based on electron microscopy [Yoshikawa et al., 1996; Lasic et al., 1997; Gustafsson et al., 1995]. They also provided evidence that lipoplexes have regular structures (FIG. 1) [Rädler et al., 1997].

FIG. 1 illustrates the molecular construction of a lipoplex. This model is discussed, as well as others that have not been investigated as thoroughly [Dan, 1998]. The lipoplex shown consists of lamellar layers, whereby DNA layers are surrounded by lipid bilayers like a sandwich, producing a regular grid. Cryoelectron microscopic investigations of lipoplexes revealed similar results [Battersby et al., 1998].

Passage of Lipid/DNA Complexes into the Cell

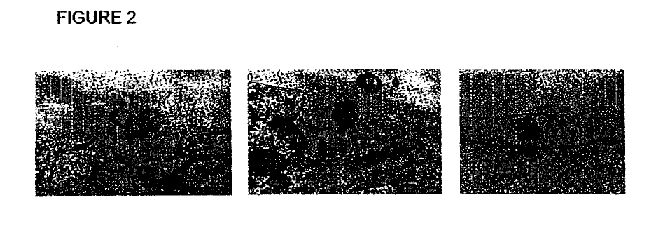

Due to their positive charge, the lipoplexes added to the cells interact with the negatively charged external cell membrane. In contrast to earlier speculations that lipoplexes pass into the cell by fusing with the cell membrane [Felgner et al., 1987], it appears certain today that passage into the cell takes place primarily via endocytosis [Wrobel and Collins, 1995]. This has been demonstrated using various cells by taking electron microscopic images of the passage of gold-labelled lipoplexes into the cell (FIG. 2) [Zabner et al., 1995].

After passage into the cell via endocytosis, the lipoplexes are located in the endosomes which apparently do not fuse with lysosomes. This process probably results in the rapid breakdown of the DNA. Rather, a considerable number of endocytotic vesicles accumulate in the vicinity of the nucleus after a few hours. Investigations carried out using fluorescence-labelled complexes show that lipoplexes can be detected in the cytosol in almost every cell that has been treated [Escriou et al., 1998].

Passage of DNA into the Nucleus

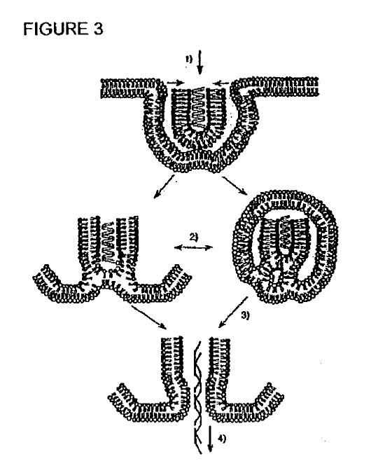

Direct insertion of lipoplexes into the nucleus does not induce expression of the corresponding proteins [Zabner et al., 1995]. Apparently the DNA—when it is complexed with cationic lipids—cannot be detected by the transcription apparatus of the cell. It appears that the DNA is not released by the lipoplexes in the nucleus. The DNA must therefore break free of the protective lipid envelope in the cytosol before it can pass into the nucleus. An interesting model of the mechanism of DNA release from lipoplexes that is based on the results of fusion experiments using cationic and anionic liposomes is shown in FIG. 3 [Xu and Szoka, 1996].

After endocytosis of the lipoplexes in the endosomes, interactions take place between the positively charged and negatively charged lipids in the endosomal membrane. In this process, anionic lipids diffuse into the lipoplex, form close lipid pairs with the cationic lipids, thereby neutralizing the positive charge. This weakens the interaction of the cationic lipids with the DNA. The DNA is released from the lipoplexes in the cytosol and can enter the nucleus.

In addition to cholesterol [Crook et al., 1998], DOPE, a naturally occurring, zwitterionic phospholipid is added to the cationic lipids to prepare the liposomes [Smith et al., 1993]. In the mechanism of DNA release shown above, the function of DOPE as a helper lipid that increases efficiency could be demonstrated by the fact that it supports the necessary membrane perturbation processes by means of its fusogenic properties [Litzinger and Huang, 1992; Farhood et al., 1995]. The passage of DNA into the nucleus is an ineffective step in transfection procedures using lipoplexes. This is due to the fact that almost every cell contains lipoplexes in the cytosol, but the desired genetic product is expressed by only a fraction of the cells [Zabner et al., 1995]. This could be caused by the DNA being released ineffectively from the lipoplexes and/or the free DNA being broken down before it reaches the nucleus.

The preceding discussion of the mechanism of transfection was provided solely by way of illustration and explanation, and is not intended to limit the scope of the appended claims.

Classification of Cationic Lipids

Since Felgner et al. used cationic lipids for transferring DNA into cells for the first time in 1987, a number of new cationic lipids have been synthesized and investigated for their transfection properties. Starting with DOTMA [Felgner et al., 1987], the first cationic lipid used systematically for transfection purposes, the chemical structure was further developed in a variety of ways [Miller, 1998; Gao and Huang, 1995; Deshmukh and Huang, 1997].

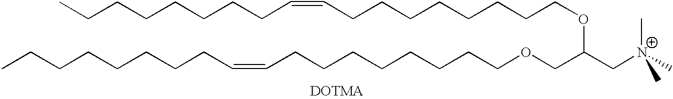

All cationic lipids can be classified as either simple cationic or polycationic lipids based on the number of charges per lipid.

Simple Cationic Lipids

All compounds in this group contain head groups that carry a tertiary or quaternary amino group. While tertiary amino groups are basically in equilibrium with the unprotonated and, therefore, uncharged form under physiological conditions (pH; ˜7.4), quaternary amino groups carry a permanent positive charge. Permethylated amino functions as with DOTMA (above) and DOTAP [Leventis and Silvius, 1990] have been described, as well as quaternizations via introduction of an additional hydroxyethyl group as in DORI [Bennett et al., 1995; Felgner et al., 1994].

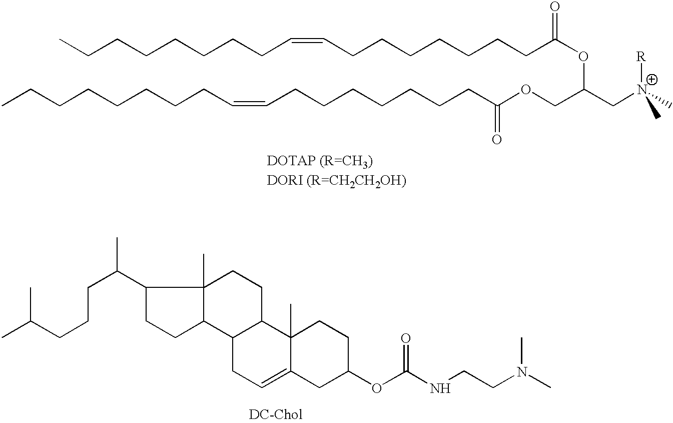

Introducing a hydroxylethyl group increases the polarity of the positively-charged head group that interacts with the DNA. This has a direct effect on the transfection properties of a lipid.

Unsaturated or saturated hydrocarbon chains are used as lipophilic lipid anchors. Although C₁₋₈-hydrocarbon chains (oleoyl or oleyl unit) are only used in unsaturated compounds, structural variations with C₁₄, C₁₆, or C₁₈-hydrocarbon chains in saturated compounds are known [Felgner et al., 1994]. The lipophilic units are linked with a parent structure (usually glycerol) via ether (e.g., DOTMA) or ester bridges (e.g., DOTMA). Ester bridges are often used to create the linkage in order to avoid cytotoxicity, because ether bridges are more difficult to break down biologically than ester bridges [Obika et al., 1997 and 1999]. Substances that are easy to decompose and are therefore often used as spacers are carbamate units (e.g., DC-Chol), amide units [Geall and Blagbrough, 1998; Okayama et al., 1997], and phosphate esters [Solodin et al., 1996]. A direct correlation between toxicity and the type of bond has never been definitively demonstrated due to the variety of possible causes of toxic side-effects.

The cholesterol unit was first used to synthesize DC-Chol [Gao and Huang, 1991]. This is a lipid that had already been tested in clinical trials [Caplen et al., 1995]. When it is not possible to form stable lipid bilayers (i.e., liposomes) using a single lipid, then it may be necessary to combine the lipid with one or more helper lipids. For example, cholesterol derivatives are typically used in combination with the helper lipid DOPE to perform transfection [overview: Miller, 1998]. This explains the favorable effect of this zwitterionic phospholipid that does not interact with DNA (above).

Polycationic Lipids

Polycationic lipids have head groups that contain more than one quaternary or protonatable, primary, secondary, or tertiary amino function. Many of these compounds have head groups that are derived from naturally occurring polyamines. The examples shown below carry the spermine (DOGS [Behr et al., 1989]) or spermidine unit (SpdC [Guy-Caffey et al., 1995]), respectively. In these examples, the distance between the amino groups is three or four methylene groups, respectively.

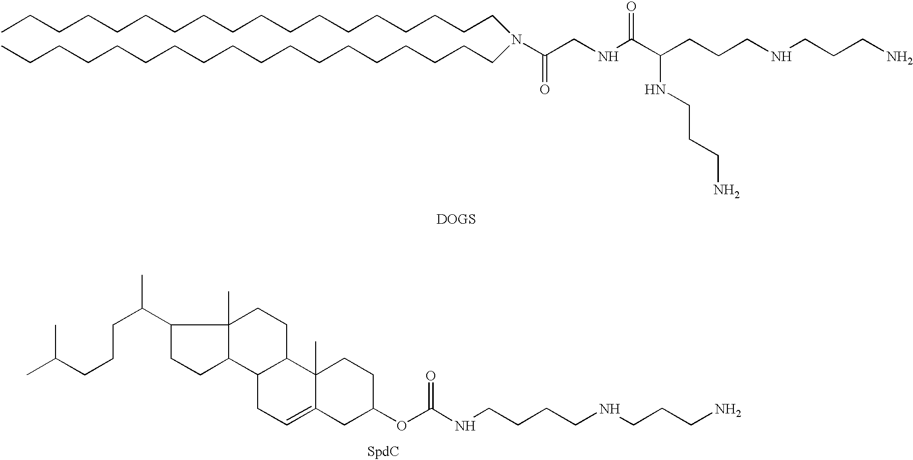

Such “natural” structures should be minimally toxic due to their ability to be broken down biologically. Additionally, these lipids should be able to bind with this very compact lipoplex due to the natural ability of polyamines to bind well with DNA. This correlates with improved transfection efficiency. Different linkages of these head groups with the lipid components resulted in linear (SpdC) or T-shaped (DOGS) arrangements of polycationic lipids. In lipids where cholesterol is used as the lipid anchor, the T arrangement resulted in more enhanced transfection rates in initial studies than that of the analog lipids having a linear structure [Lee et al., 1996]. Using bi-chained lipids, however, the linear arrangement yielded higher transfection rates [Byk et al., 1998].

Using synthetic strategies in accord with the practice of the present invention, lipids having the following structural features are provided:

1. Cationic head groups are used that contain one, two, or three amino group(s) as potentially positive charge carriers. However, it is within the scope of the present invention to provide lipids having head groups containing more than three amino groups (i.e., polyamine head groups). Amino groups that can be suitably included within the head groups are primary amines, secondary amines, tertiary amines and quaternary amines. Preferably, secondary amines, tertiary amines, and quaternary amines contained in the head groups are alkylated with at least one radical selected from the group consisting of methyl, ethyl, propyl, isopropyl, hydroxymethyl, hydroxyethyl, hydroxypropyl, glycerol and mannitol. In head groups containing two, three or more amino groups, the number of methylene groups between the amino groups is variable (e.g., x and y). Suitable head groups include but are not limited to spermine and spermidine.

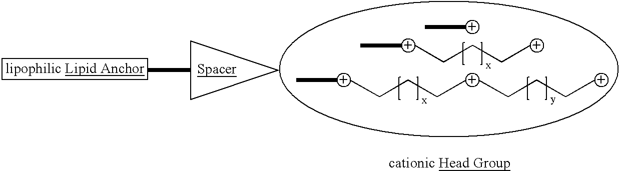

Preferred embodiments of the present invention are directed to cationic amphiphiles having the structure A-F-D, wherein:

A is a lipid anchor;

F is a spacer group having the structure

O—C(O)-G¹-[C(R¹)(R²)]ₘ-G²-{C(O)-E-[C(R³)(R⁴)]ₙ}ₚ; and

D is a head group; and wherein:

G¹ and G² are the same or different, and are independently either oxygen or a bond;

R¹, R², R³ and R⁴ are the same or different, and are independently selected from the group consisting of hydrogen and alkyl radicals;

m, n and p are the same or different, and are independently either 0, 1, 2, 3, 4, 5, or 6; and

E is oxygen or N(R⁵), wherein R⁵ is hydrogen or an alkyl radical. Preferably, E does not contain nitrogen when D is N(CH₃)₂. More preferably, E does not contain nitrogen when D is N(CH₃)₂ and when A is cholesterol. Still more preferably E does not contain nitrogen when D is N(CH₃)₂, A is cholesterol, and R⁵ is hydrogen. Even still more preferably, E does not contain nitrogen when D is N(CH₃)₂, A is cholesterol, R⁵ is hydrogen and both G¹ and G² are bonds. Still more preferably, E does not contain nitrogen when D is N(CH₃)₂, A is cholesterol, R⁵ is hydrogen, both G¹ and G² are bonds, and each of R¹, R², R³ and R⁴ is hydrogen. Still more preferably, E does not contain nitrogen when D is N(CH₃)₂, A is cholesterol, R⁵ is hydrogen, both G¹ and G² are bonds, each of R¹, R², R³ and R⁴ is hydrogen, and both m and n are 2. Most preferably E does not contain nitrogen when D is N(CH₃)₂, A is cholesterol, R⁵ is hydrogen, both G¹ and G² are bonds, each of R¹, R², R³ and R⁴ is hydrogen, both m and n are 2, and p is 1.

2. Spacers are used that are varied systematically in terms of polarity and length.

3. Cholesterol is used as the lipid anchor

All synthesized lipids were investigated in standardized cell experiments and the effect of the individual structural variations on the transfection properties of lipids was determined.

Synthesis Procedures

The ensuing description of the synthesis procedures includes a) a brief description of the process used to select the lipid anchors, spacers, and head groups used to prepare cationic lipids, b) the procedures used to synthesize lipids with simple cationic, bicationic, and tricationic head groups and cholesterol as the lipid anchor, and c) the procedures used to synthesize cationic lipids with 1-(2,3-di-tetradecyloxy)-propanol as the lipid anchor.

Selecting the Structures for Head Group, Spacer, and Lipid Anchor

The goal is to synthesize lipids with simple cationic, bicationic, and tricationic head groups. The simple cationic lipids were varied systematically by means of the rate of substitution of the amino group (tertiary or quaternary) and the structure of the substituents (methyl or hydroxyethyl group). The bicationic and tricationic head groups will be varied systematically in terms of the length of the hydrocarbon chains between the amino groups (2 to 6 methylene groups). The bicationic and tricationic head groups will be linked with the lipid components in a linear arrangement.

The spacers to use will vary in terms of their polarity and length.

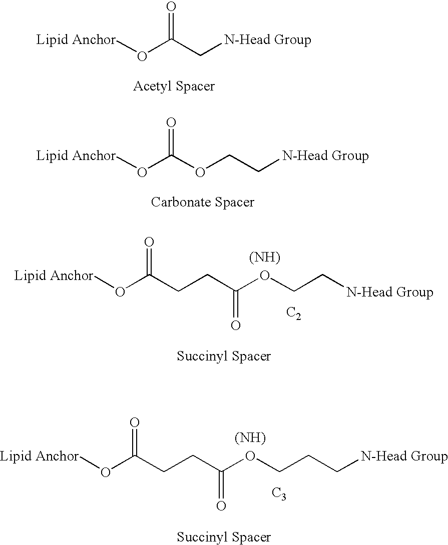

The very short acetyl and carbonate spacers were used as relatively apolar spacers. They were extended with a short alkyl chain of 2 methylene groups via an ester bond. Succinyl units were used as the more polar spacers that were extended with either an alkyl chain having either 2 or 3 methylene groups via an ester or amide bond. While it is not the intention of the applicant to be bound to any particular theory, nor to affect in any measure the scope of the appended claims, it is presently believed that the succinyl spacers are more polar than acetyl and carbonate spacers due to the presence of an additional oxo group which increases the number of opportunities for forming hydrogen bridge bonds. Within the group of succinyl spacers, the spacers linked via amide bonds are more polar than the homologous ester derivatives. This is also due to the additional hydrogen bridge bonds. The ester and amide bonds should be hydrolyzable by the enzymes in the cell (esterases and amidases), making the cationic lipids less cytotoxic than compounds with ether bonds (Gao and Huang, 1993]. Lipids should not accumulate within the cell. Simple cationic lipids with acetyl spacers [Aberle et al., 1998] and succinyl spacers [Takeuchi et al., 1996] have been described in the literature. The overall structures of these spacers are basically not comparable to those of the compounds synthesized in this study, however.

Preferably, the lipid anchor is selected from the group consisting of steroids and and lipophilic lipids comprising two long alkyl chains. Suitable steroids include but are not limited to bile acids, cholesterol and related derivatives, vitamin D, certain insect molting hormones, certain sex hormones, corticoid hormones, certain antibiotics, and derivatives of all of the above wherein additional rings are added or are deleted from the basic structure. Preferred steroids include cholesterol, ergosterol B1, ergosterol B2, ergosterol B3, androsterone, cholic acid, desoxycholic acid, chenodesoxycholic acid, and lithocholic acid. Suitable lipophilic lipids comprising two long alkyl chains are preferably ones wherein the alkyl chains have at least eight contiguous methylene units. More preferably, the length of these alkyl chains is between eight and twenty-four carbon atoms. The alkyl chains may be saturated, unsaturated, straight, branched, or any combination thereof, as is well known in the art. More preferably, the lipid anchor is selected from the group consisting of cholesterol, diacylglycerol, dierucylglycerol, and 1-(2,3-di-tetradecyloxy)-propanol (DMG). Most preferably, the lipid anchor is cholesterol.

All compounds from the three groups of simple cationic, bicationic, and tricationic lipids should have the same lipid anchor so the lipids can be compared with each other. Since effective cholesterol derivatives with cationic and polycationic head groups have already been described [Miller, 1998], and the cholesterol parent structure remains chemically stable even under the various synthesis conditions, cholesterol was used as the lipid anchor. To basically show that bi-chained lipid anchors can also be used in the synthesis strategies developed in this study, individual model compounds were also synthesized using 1-(2,3-di-tetradecyloxy)-propanol (DMG) as the lipid anchor. DMG is already known as a lipid anchor for a number of lipids such as DMRIE [Felgner et al., 1994].

Synthesis of Simple Cationic Lipids

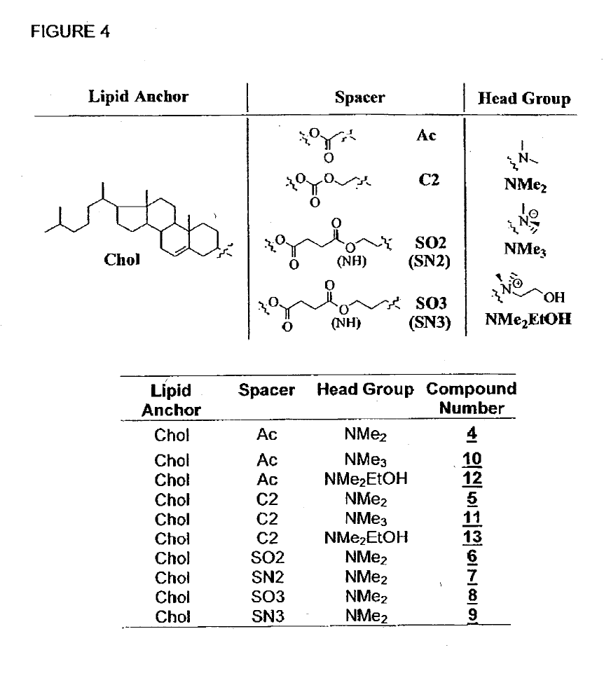

The synthesis procedures that led to simple cationic lipids with the lipid anchors, spacers, and cationic head groups shown in FIG. 4 are described below.

The compounds were prepared using a successive synthesis strategy that consisted of three steps:

1. Link cholesterol, the lipid anchor, with the various spacers

2. Link the simple cationic head groups with the various lipid components

3. Possible derivatization of the head groups

Synthesis of Lipid Components

Cholesterylchlorformiate, a lipid component, is commercially available and was not manufactured. Basically, it can be obtained by reacting cholesterol with phosgene, however. The lipid component chloroacetic acid cholesterylester (1) was prepared via esterification of cholesterol with a slight excess of chloroacetic acid chloride in dichloromethane without DMAP. Since a simple purification procedure via recrystallization from acetone resulted in yields of just 60%, a purification procedure using cyclohexane/ethylacetate (2:1) in column chromatography was preferred. It resulted in a higher yield (97%).

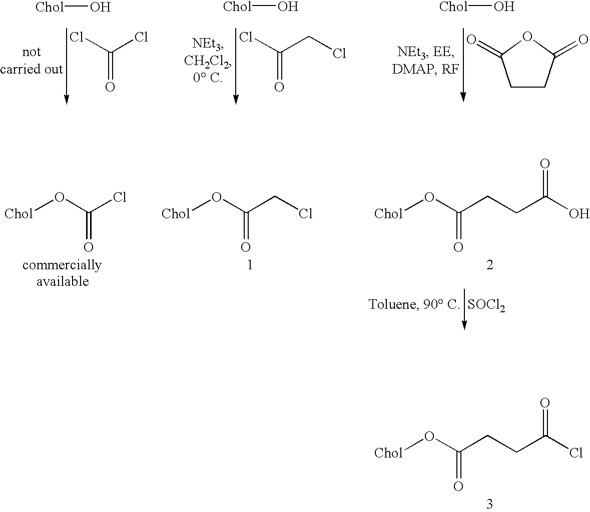

Cholesterylhemisuccinoylchloride (3) was obtained in a two-step reaction [Kley et al., 1998]: cholesterylhemisuccinate (2) was first manufactured via esterification of cholesterol with succinic acid anhydride with DMAP catalysis. The acid 2 that was obtained in a yield of 89% was converted to the corresponding acid chloride 3 in toluene with a 2.5-fold excess of thionylchloride. After the toluene and excess thionylchloride were removed in a vacuum, cholesterylhemisuccinoylchloride (3) remained as a solid. Assuming that this reaction proceeded with a quantitative conversion, dried toluene was added to make a 0.5 M stock solution. This solution was stable against hydrolysis for months when stored at 5° C. The advantage of the stock solution was the fact that the subsequent acylation reactions are especially easy to carry out by adding the acid chloride stock solution in drops to the corresponding alcohol or amine components.

Synthesis of Lipids with Tertiary Amino Groups

The lipid components were linked with the head group by means of an alkylation reaction (acetyl spacer) or an acylation reaction (carbonate or succinyl spacer), depending on the lipid component used.

Lipids with a Tertiary Amino Group and Acetyl Spacers

The lipid component chloroacetic acid cholesterylester (1) and the amino function which serves as the cationic head group were linked via an alkylation reaction. In contrast to haloalkanes, in the homologous series of which the chlorine function is replaced most poorly and the iodine function is replaced most easily with nucleophiles, the lipid component 1 proved to be a very good alkylation reagent. The strong inductive effect of the ester function makes the methylene group which is adjacent to the chlorine function very electron-deficient (positive nature increased considerably), and the chlorine function should be easy to substitute with good nucleophiles such as amines under very mild conditions (room temperature).

To prepare the simple cationic lipid N-cholesteryloxycarbonylmethyl-N,N-dimethylamine (4), 1 was converted with an ethanolic dimethylamine solution under refrigeration in toluene. After just a few hours, no adduct could be detected (inspected via thin-layer chromatography). Two new compounds had been formed, however: the desired product 4, as well as a by-product. This by-product was finally identified as cholesterol using thin-layer chromatography and then ¹H-NMR spectroscopy.

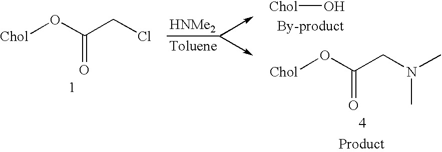

Apparently the desired alkylation reaction as well as a consecutive or competitive reaction took place despite the refrigeration which led to the production of cholesterol as the by-product by means of ester cleavage. When the cholesterol was removed via column chromatography, 4 was obtained in a yield of 53%.

Lipids with a Tertiary Amino Group and Carbonate and Succinyl Spacers

The head group was linked with the respective lipid component containing the carbonate or succinyl unit as a spacer using an acylation reaction. Lipid components with spacer units containing an activated acid (acid chloride) were used in this process. Cholesterylchlorformiate, which is commercially available, and cholesterylhemisuccinoylchloride (3) were used. To link the head group with the lipid component via formation of an ester or amide bond, the head groups had to be equipped with an additional hydroxy or primary amino function (bifunctional amines).

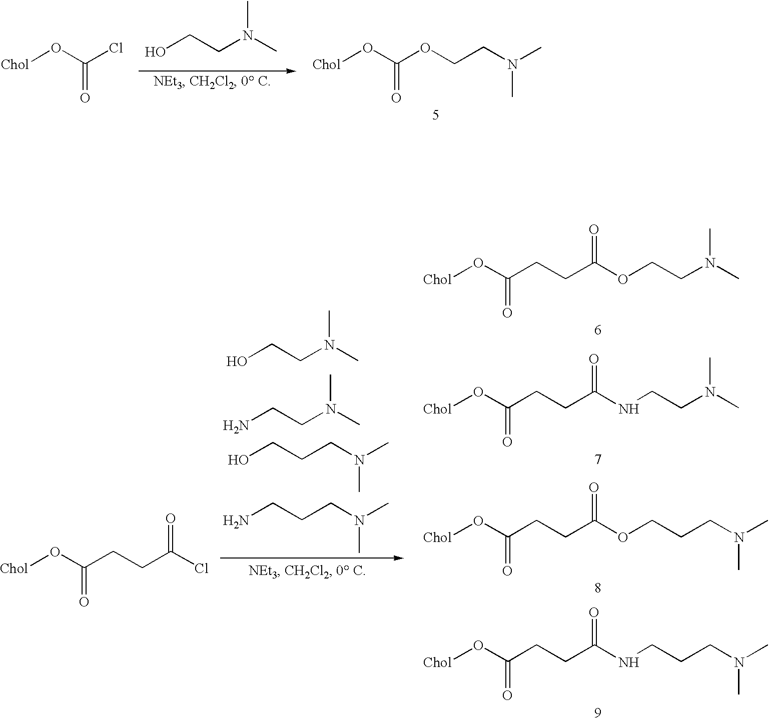

2-(dimethylamino)-ethanol was used to prepare N-(2-cholesteryloxycarbonyloxy-ethyl)-N,N-dimethylamine (5). This led to a yield of 80% after column chromatography. The preparation was carried out analogously to the synthesis of DC-Chol, a structurally homologous cationic lipid, in which the 2-(dimethylamino)-ethylamine was linked with cholesterylchlorformiate with the formation of an amide bond [Gao and Huang, 1991].

The structure of the cationic lipids that contain the succinyl spacers was varied broadly by using various bifunctional amines. The lipids (N=(cholesteryl-hemisuccinoyloxy-2-ethyl)-N,N-dimethylamine (6), N-(cholesterylhemisuccinoylamino-2-ethyl)-N,N-dimethylamine (7), N-(cholesterylhemi-succinoyloxy-3-propyl)-N,N-dimethyl-amine (8), and N-(cholesterylhemisuccinoylamino-3-propyl)-N,N-dimethylamine (9) differ in the following ways: in addition to linking the head group with the lipid component via an amide or ester bond, the number of methylene groups (2 or 3) between the succinyl unit and the tertiary amino function were varied. The lipids 6 and 8 linked via an ester bond were obtained easily via recrystallization from acetonitrile with a yield of 62% and 42% (per thin-layer chromatography: quantitative conversion). In contrast, purification of the lipids 7 and 9, which were linked via an amide bond, via recrystallization was not successful, because cholesterol was produced quantitatively when warmed in acetonitrile (monitored using thin-layer chromatography). While it is not the intention of the applicant to be bound to any particular theory, nor to affect in any measure the scope of the appended claims, it is presently believed that the corresponding cyclic succinic acid imide derivative is formed from the desired product with heating, with cleavage of cholesterol. Both 7 and 9 were therefore purified via column chromatography using a mixture of ethyl acetate and methanol (yields: 52% (7) and 64% (9)).

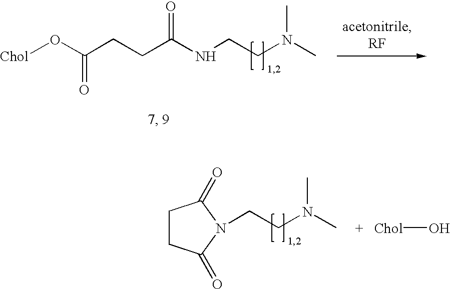

Lipids with a Quaternary Amino Group

Model compounds with a quaternary amino group were prepared in addition to lipids with a tertiary amino group. If a positive charge is first produced via protonation of the tertiary amino group, the quaternary amino group is a permanent positive charge. Starting with the tertiary acetyl and carbonate derivative (4 and 5), quaternizations were carried out by introducing an additional methyl group or a 2-hydroxyethyl group.

Quaternization via Introduction of a Methyl Group

The tertiary amino groups of lipids 4 and 5 were quaternized in acetone using dimethyl sulphate at room temperature. The use of methyl iodide in a mixture of chloroform and DMSO to produce quaternization is also described in the literature (synthesis of DOTAP, [Leventius and Silvius, 1990]). Dimethyl sulphate was preferred over methyl iodide in the conversions described here, because it is the less volatile of the two highly toxic methylating agents, and the product is not light-sensitive due to the presence of the mesylate counterion.

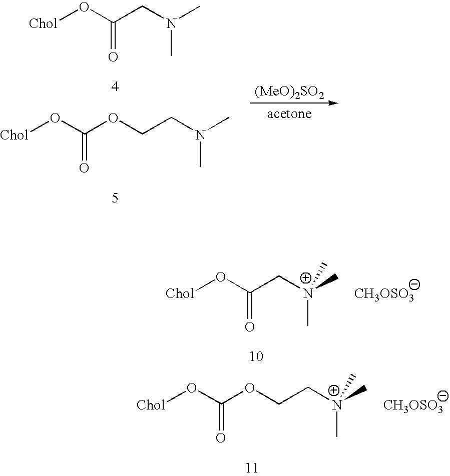

In the quaternization with dimethyl sulphate, acetone had an advantage over other solvents such as tetrahydrofuran and ethyl acetate in which quantitative conversions were also observed in that the respective adducts were soluble in acetone, while the quaternary products formed disappeared after just a few minutes. Rewashing with a small quantity of acetone was enough to separate excess dimethyl sulphate. After drying, lipids 10 and 11 were obtained in very good yields (72% and 88%) with no impurities.

Quaternization via Introduction of a 2-Hydroxyethyl Group

In addition to quaternization via introduction of a methyl group, a quaternary acetyl and carbonate derivative will also be produced, the amino groups of which carry an additional 2-hydroxyethyl group. In preliminary experiments an attempt was made to introduce the 2-hydroxyethyl group in analogous fashion to the preparation of the cationic lipid DORI [Felgner et al., 1994] by alkylating the tertiary amino functions of 4 and 5 with 2-bromoethanol. The yields were poor: a conversion with a satisfactory yield did not take place in acetone, acetonitrile, or in mixtures of various alcohols (isopropanol, ethanol) (monitored using thin-layer chromatography: maximum 30%). The conversion did not increase at higher temperatures, either. The maximum temperature selected was 50° C. to minimize the risk of breaking down the adduct/product. Due to these difficulties, the 2-iodoethanol alkylation reagent was tested, even though it is very light-sensitive.

The preparations of N-cholesteryloxycarbonylmethyl-N,N-dimethyl-N-hydroxyethyl-ammonium iodide (12) and N-(2-cholesteryloxycarbonyloxy-ethyl)-N,N-dimethyl-N-hydroxyethyl-ammonium iodide (13) via aklylation of 4 and 5 with 2-iodoethanol were quantitative after inspection via thin-layer chromatography. The reactions were carried out in acetone and the subsequent purification step was very simple: the homologous permethylated products and lipids 12 and 13 precipitated out of the acetone. Acetone was then used in a washing step to completely remove the excess 2-iodoethanol.

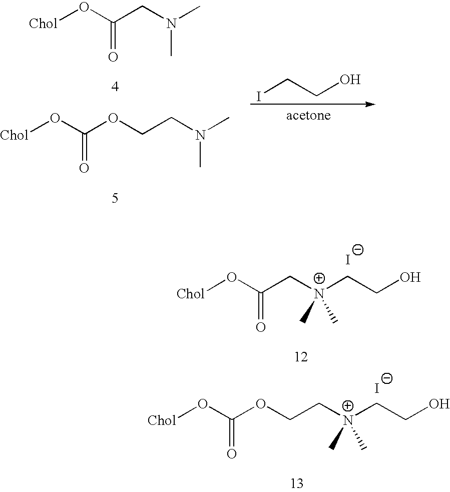

Due to the presence of the counterion iodide, the quaternary products were light-sensitive and were stored at 0° C. in the dark.

Synthesis of Bicationic Lipids

Two different synthesis strategies for preparing bicationic lipids were investigated: according to one strategy, the lipids were synthesized linearly starting with a lipid anchor. According to the other, a convergent synthesis strategy was investigated in which the bicationic head group and the lipid component were synthesized separately from each other and then linked together.

Linear Synthesis Strategy for Bicationic Lipids

To investigate the possibility of linearly synthesizing bicationic lipids, the hexadecyl unit, which is commercially available, was used as the lipid component. The distance between the two amino groups that were to be successively linked with the lipid anchor should be 2 to 6 methylene groups wide. The first synthesis strategy is illustrated below:

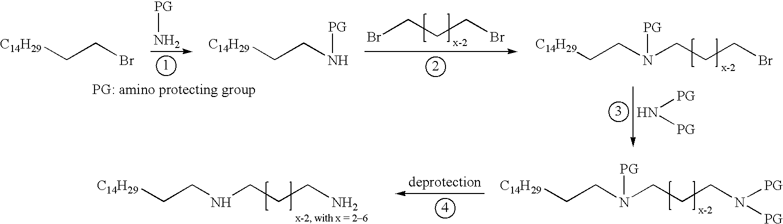

Starting with hexadecyl bromide, the bromo function would be substituted with a protected amino group in the first reaction step 1. This initial amino group would then be alkylated with various α,β-dibromoalkanes (alkylation reaction 2), whereby commercially available α,β-dibromoalkane would be used. A clear excess of the alkylation reagent would be used to convert just one of the two bromo functions. The remaining terminal bromo function of the alkylation product can then be substituted with another protected amino group (3), whereby the required second amino group is introduced. The protective groups are cleaved in the final step (4).

The controlled monoalkylation of amines represents an important prerequisite for the realization of the synthesis strategy described. Amines are characterized by the fact that their monoalkylation is difficult to control without using appropriate protective groups [Hendrickson and Bergeron, 1973]. When converting ammonia with an alkylhalogenide, for instance, one obtains a mixture of amines that have been alkylated and peralkylated one-fold, two-fold, and three-fold, because the reactivitity (alkalinity) increases as the degree of alkylation increases. Amino protective groups must be used for the planned synthesis so that controlled monoalkylations can be carried out. These protective groups should be inert to the alkylation conditions, and it should be possible to perform quantitative cleavage under mild conditions.

Various types of amino protective groups are described in the literature [Kocienski, 1994; Greene and Wuts, 1991]. Protective groups that protect the amines using an acylation reaction as amides are used to synthesize secondary amines [Fichert and Massing, 1998; Fukuyama et al., 1995] and polyamines [Fiedler and Hesse, 1993; Ganem, 1982]. Examples of this include the tosyl protective group [Kiedrowski and Dörwald, 1988] and the trifluoracetyl protective group [Nordlander et al., 1978]. Additionally, protective groups are used that protect amines using an alkylation reaction such as the benzyl protective group [Niitsu and Samejima, 1986] or the allyl protective group [Garro-Helion et al., 1993]. Amines that are protected via conversion to an amide derivative lose their alkaline character. For this reason, amides do not alkylate with very strong bases such as NaH until deprotonation is complete. Since this can lead to undesired secondary reactions under certain conditions (e.g., elimination) [Fichert, 1996], amino protective groups should be used that are introduced with an alkylation reaction. Amines protected in this manner basically retain their alkaline character and monoalkylation can therefore be carried out under milder conditions. Additionally, these protective groups protect the amino groups from an undesired multiple alkylation by taking up a great deal of space (steric hindrance). They also protect amines by exercising an electron attraction on the amino group, thereby reducing the alkalinity or reactivity so that only monoalkylation can be carried out. The benzyl and allyl protective groups were investigated to realize the planned synthesis strategy (above).

The Benzyl Protective Group

The desired bicationic model compounds contain a terminal, primary amino group and a secondary amino group. Procedures for synthesizing secondary amines starting with benzyl-protected, primary amines are described in the literature: in a two-stage synthesis procedure, monoalkylation of a benzyl-protected, primary amino group takes place first. The benzyl protective group is then cleaved under hydrogenolytic conditions (Pd-C, H₂) with the release of the secondary amino function [Bergeron, 1986].

To test the suitability of the benzyl protective group for the planned synthesis of bicationic lipids, 3 eq. benzylamine were monoalkylated with 1 eq. hexadecylbromide (THF, K₂CO₃, 70%). An attempt was then made to convert the resultant N-benzyl-N-hexadecyl-amine with 1,4-dibromobutane (as an example of a bifunctional alkylation reagent from the group of various α,ω)-dibromoalkanes). To prevent an undesired substitution of both halogen functions, a three-fold excess of bifunctional alkylation reagent was used, as it was in all similar, subsequent conversions. The desired product was not obtained in the conversion, just a by-product that was not characterized further. Since the reflux conditions required for a conversion (boiling point of toluene: 110° C.) may have been responsible for the formation of the by-product, the alkylation reagent 1,4-dimesyloxybutane was tested. This alkylation reagent carries mesyl leaving groups, which are much more reactive than the bromo function and should therefore react with the adduct as desired at lower temperatures. Unfortunately this approach resulted in the quantitative production of a cyclic by-product at a temperature of just 50° C. that was identified as the pyrrolidine derivative illustrated below:

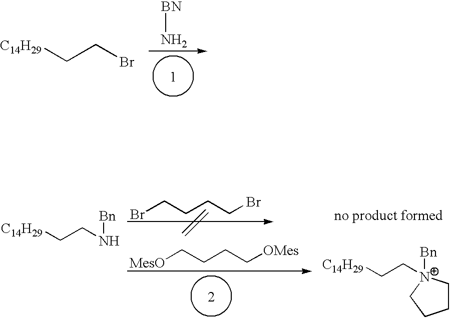

The alkylation of N-benzyl-N-hexadecyl-amine with 1,3-dibromo-propane (C₃ components), unlike C₄ components, is possible in the form desired [Fichert, 1996]. The formation of a 4-ring was not expected due to the high ring strain. The formation of a 6-ring as would be produced if N-benzyl-N-hexadecyl-amine were reacted with 1,5-dibromo-pentane (C₃ components) would be highly likely in any case. To avoid ring formation, 1,4-dimesyloxy-bu-2-yne was tested as the alkylation reagent. It contains a C—C triple bond and, as a linear compound, makes ring formation impossible. After successful alkylation, the second mesyl group was substituted with a benzyl-protected amine. In the subsequent cleavage of the benzyl groups via hydrogenolysis, the triple bond was also reduced to a single bond.

The alkylation of benzyl-protected hexadecylamine with 1,4-dimesyloxy-but-2-yne did not proceed as hoped. Although cyclization was not observed, multiple alkylation did occur, forming the quaternary product, which was even less desired by a factor of about 10.

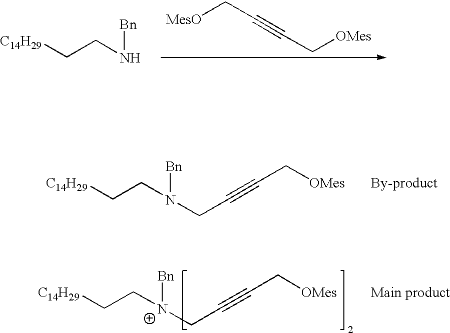

Since the multiple alkylation is probably due to the high reactivity of 1,4-dimesyloxy-but-2-yne, 1,4-dichlor-but-2-yne, which is less reactive, was used. As a matter of fact, this approach resulted in the quantitative preparation (monitored using thin-layer chromatography) of the corresponding product with a yield of 62% after purification via column chromatography.

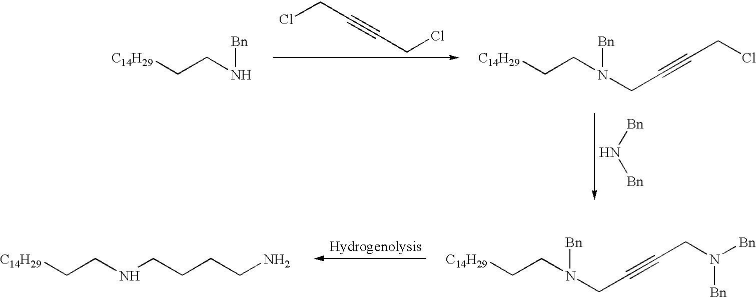

The second terminal amino function was then introduced via substitution of the terminal chlorine function with dibenzylamine (65% yield).

The hydrogenolytic cleavage of the benzyl protective groups, on the other hand, was not without problems: in the initial attempt to remove the protection, a product with a yield of 71% was formed after purification via column chromatography (per thin-layer chromatography: quantitative). Using ¹H-NMR spectroscopy, this product was identified as the compound that was still carrying a benzyl group on the terminal amino group. The triple bond had been successfully hydrogenated into a single bond. Extending the reaction time (2 days) and adding more catalyst (Pd-C) until a molar catalyst/adduct ratio of 1:3 (common: 1:10) was reached did not completely remove the protection. The remaining benzyl group was finally removed by carrying out another hydrogenation process and purifying the monobenzylated product in-between (using column chromatography).

The fact that it is more difficult to remove protection from primary benzyl-protected amines than secondary benzyl-protected amines has been described in the literature [Velluz et al., 1954; Erhardt, 1983]. This does not completely explain the problems encountered when trying to remove protection completely, because the second attempt to remove protection was successful, with a good yield of 93%. While it is not the intention of the applicant to be bound to any particular theory, nor to affect in any measure the scope of the appended claims, it is presently believed that the by-products acted as catalyst poisons in the first attempt to eliminate protection and prevented complete debenzylation.

The Allyl Protective Group

Since it took a long time to remove protection from the primary amino function, it was decided to test the suitability of the allyl protective group to prepare polyamines.

To cleave the allyl protective group, the allyl group (as a stabilized allyl cation) is converted to a different nucleophile in a palladium-catalyzed reaction. Both H₂O [Benz, 1984] and N,N′-dimethylbarbituric acid (NDMBA) [Garro-Helion et al., 1993] were used as “allyl acceptors”. The successive synthesis strategy using the allyl protective group is illustrated below:

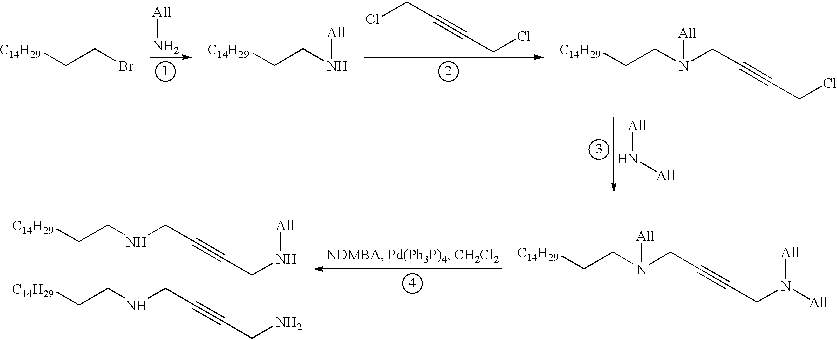

N-allyl-N-hexadecyl-amine was successfully prepared via alkylation 1 (acetonitrile, K₂CO₃) of 3 eq. allylamine with 1 eq. hexadecyl bromide. At a reaction temperature of 50° C. (allyl amine has a boiling point of 530 C), the yield after purification via column chromatography was 94%. Due to the risk of ring formation, 1,4-dichlor-but-2-yne was used as the bifunctional alkylation reagent 2. In this reaction, a product yield of just 26% but a considerable portion of polar product (probably bialkylated adduct) was found under reflux conditions (acetonitrile). To suppress the secondary reaction in favor of the monoalkylation, the reaction was carried out at 40° C. This increased the yield to about 35%. No further optimization steps were carried out.

The subsequent substitution of the chlorine function with diallyl amine 3 proceeded smoothly, with a yield of 74% after purification via column chromatography. To remove the protection from the amino groups, an attempt was made to convert the allyl group to a barbituric acid derivative using palladium catalysis 4 [Goulaouic-Dubois et al., 1995]. Unfortunately the method, which was described in the literature as being very effective, was unsatisfactory in terms of the yields obtained. The yield of product with all protection removed was only 25%, and a by-product (50% yield) was obtained after column chromatography that was identified via ¹H-NMR spectroscopy as a compound with protection only partially removed. The subsequent hydrogenation of the triple bond into a single bond—which, based on experience, is non-problematic—was not carried out, but it is a necessary, additional and final step in the synthesis process.

In the linear synthesis strategy developed, every single bicationic lipid must be synthesized step-by-step. This approach in particular requires a considerable amount of effort for the synthesis procedure if a great number of lipids are to be synthesized using various lipid anchors, spacers, and head groups.

Convergent Synthesis Strategies for Bicationic Lipids

In comparison with a linear synthesis strategy, it should be possible to synthesize a great number of systematically varied compounds using a convergent synthesis strategy that requires even less effort. The convergent synthesis strategy to be developed should fulfill the following requirements:

1. α,ω-diamino-alkane like the ones shown below, which are commercially available, should be used to synthesize the head groups. Alkylation, which is problematic, is circumvented with α,ω)-dibromo alkanes and α,ω-dimesyloxy-alkanes (see above).

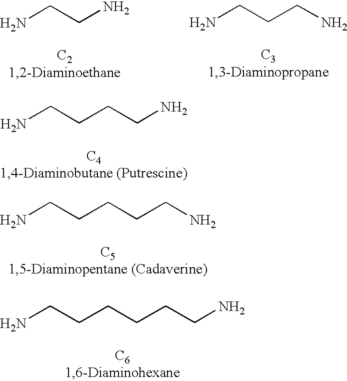

2. The terminal amino group should carry an additional alkyl group. The head group then contains only secondary amino groups from which protection is easier to remove than primary amino groups when the benzyl protective group is used.

3. A model structure (hexadecyl chain, see above) will not be used as the lipid anchor in this synthesis strategy, but rather cholesterol directly.

4. The target compounds should have the spacer structures that are varied in terms of polarity and length, the selection of which is described above.

The planned convergent synthesis can be carried out as follows (R=alkyl group):

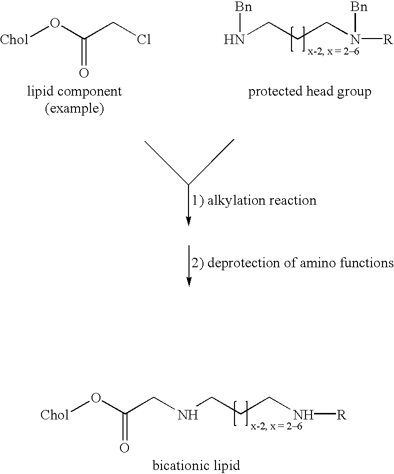

In this planned synthesis procedure, the head group and lipid components should be linked with each other in an alkylation reaction. This strategy differs basically from linkage via an acylation reaction that is described frequently in the literature [Blagbrough and Geall, 1998]. Due to the conversion of an amine to an amide, the acylation of an amino group leads to a reduction in the number of potentially positively charged amino groups.

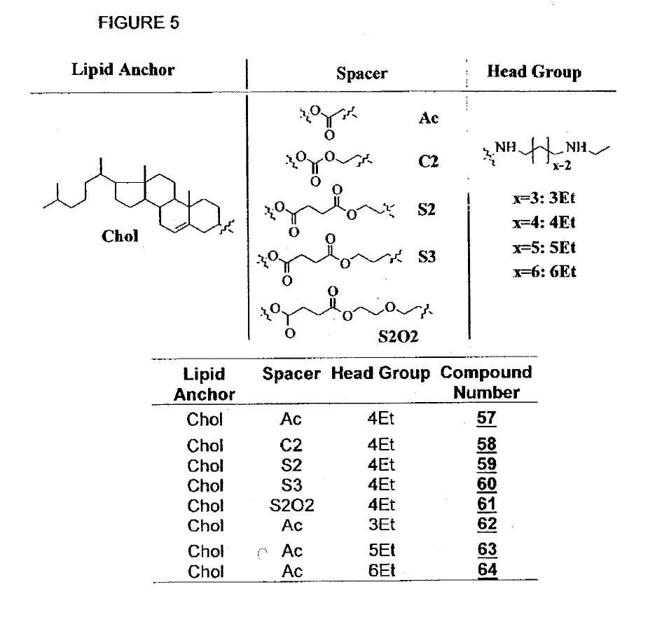

In the planned linkage, the lipid component that carries a corresponding leaving group, alkylates the terminal, primary benzyl-protected amino group (NH function) of the head group. In this process, the second secondary, benzyl-protected amino group should be protected from alkylation, because the benzyl group—which takes up a lot of space—does not allow alkylation to take place and form the quaternary amino group under the alkylation conditions used. The target compounds should be obtained in a final step by removing the benzyl protective groups. The bicationic lipids shown in FIG. 5 were obtained using the synthesis strategy described here.

Preparation of Lipid Components

The goal was to synthesize lipid components with the acetyl spacer, the carbonate spacer, and various succinyl spacers. All lipid components must carry a suitable leaving group to couple with the protected bicationic head groups. While the synthesis of chloroacetic acid cholesterylester (1) was previously described as a suitable lipid component with an acetyl spacer, the synthesis of lipid components with the carbonate and succinyl spacers will be described below.

Preparation of 2-bromoethyl-cholesterylcarbonate (14), 2-bromoethyl-cholesterylsuccinate (15), 3-bromopropyl-cholesterylsuccinate (16), N-(2-bromoethyl)-cholesterylsuccinylamide (17), and N-(3-bromopropyl)-cholesterylsuccinylamide (18)

14 was prepared by esterification of 2-bromoethanol with cholesterylchlorformiate in dichloromethane. Although the product was easy to recrystallize from acetone, with a yield of 70%, it still contained polar impurities that could be detected (monitored using thin-layer chromatography). Clean product was finally obtained with a yield of 60% after column chromatography.

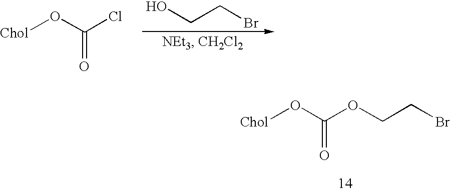

To prepare lipid components 15 and 16, cholesterylhemisuccinoylchloride (3, as a stock solution in toluene) was esterified with 2-bromoethanol and 3-bromo-propanol in dichloromethane. After purification via column chromatography, satisfactory yields of the products (63% of 15 and 72% of 16) were obtained.

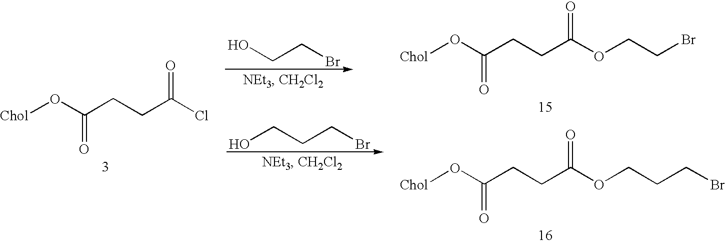

Conversions of 3 with the amines 2-bromo-ethylamine and 3-bromopropylamine in place of the corresponding alcohols were also carried out. The resultant lipid components 17 and 18 therefore do not contain an ester bond but rather an amide bond as the spacer.

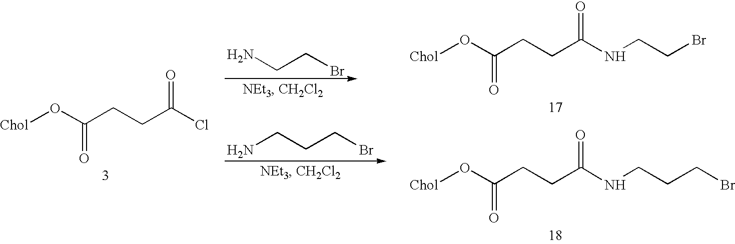

An attempt was made to purify both amide derivatives via recrystallation out of methanol. Compound 18 was obtained in a yield of only 50%, although, after inspection via thin-layer chromatography, a 100% conversion was observed with both formulations. The purification conditions were not optimized. Satisfactory yields of compound 17 could not be obtained via recrystallization. When warmed in methanol, considerable quantities of cholesterol (monitored using thin-layer chromatography) formed. Pure product in a yield of 35% was obtained only after purification via column chromatography using dichloromethane/ethylacetate mixtures.

Preparation of Cholesteryl-(2-(2-mesyloxyethyloxy)-ethyl)-succinate (20)

20 was prepared in two reaction steps starting with cholesterylhemisuccinoylchloride (3). In the initial step, diethylene glycol was converted to the monoester using a 10-fold excess quantity with 3. Excess diethylene glycol was separated via simple extraction of the organic phase (dichloromethane) against water. After inspection via thin-layer chromatography, only a very small quantity of diester was produced as a by-product.

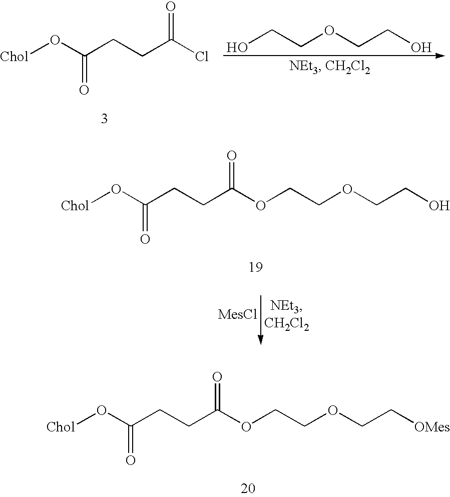

The hydroxy function was then converted to the reactive mesyl ester with mesyl chloride in dichloromethane. After inspection via thin-layer chromatography, a quantitative conversion was observed and the only thing left to do was to remove ammonium salts of the base triethylamine that had formed and excess mesyl chloride. Purification of 20 was therefore very easy and entailed extraction of the dichloromethane phase against 2 N HCl (yield after drying in a vacuum: 95%).

Synthesis of the Protected Bicationic Head Groups

Benzyl-protected, bicationic head groups were prepared in four synthesis steps. An ethyl unit was used as the additional alkyl group for the terminal amino group that was to be introduced in a monoalkylation step that is easy to monitor.

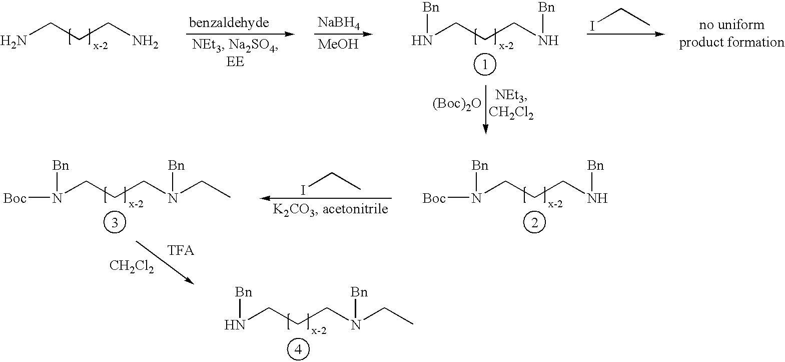

| x = | 1 | 2 | 3 | 4 |
|---|---|---|---|---|
| 2 | 21 | 26 | 31 | 36 |
| 3 | 22 | 27 | 32 | 37 |
| 4 | 23 | 28 | 33 | 38 |
| 5 | 24 | 29 | 34 | 39 |
| 6 | 25 | 30 | 35 | 40 |

Starting with various commercially available α,ω-diamino-alkanes that vary in terms of the number of methylene groups located between the two amino groups (from 2 to 6), one benzyl protective group was introduced per amino function in the initial step (compound group 1). In this process, the diamines were first converted with benzaldehyde to form the corresponding diimines and the converted into the amine compounds via reduction with sodium borohydride [Samejima et al., 1984]. Since a great deal of foam can be produced in this reduction step at room temperature due to the formation of hydrogen, the reaction temperature was maintained at 0° C. The slower reaction speed at this temperature was offset by a longer reaction time. Excess sodium borohydride was separated via filtration through silica gel when the reaction was complete. This prevented the formation of gas (hydrogen gas) when the product was loaded into the column, which would have greated compromised the quality of separation. The slimy by-products/residues produced in the reduction process were also filtered off in the filtration step. They would have compromised the flow of solvent through the column. Due to mixed fractions in the purification via column chromatography that were discarded, the yields of 1 were between 50 and 70%.

It was demonstrated in preliminary experiments that the final compounds 4 cannot be prepared via monoalkylation of N,N′-dibenzyl-α,ω-diamino-alkanes (1) with ethyl iodide. Products that had been alkylated multi-fold were detected. This can be explained by the high reactivity of ethyl iodide. One of the two amino functions should therefore first be protected by means of an additional amino protective group that can be removed as the benzyl protective group under other conditions (“orthogonal protective group”) so that the non-reacted amino function can then be alkylated with ethyl iodide. The additionally introduced protective group would be removed again in a final step.

The Boc protective group (tert-butyloxycarbonyl protective group) was used as the additional orthogonal amino protective group. The advantage of the Boc protective group is the good yields obtained during introduction of the amino group and removal of its protection.

To prepare the compound group 2, in which just one amino group carries the additional Boc protective group, a solution of Boc anhydride in dichloromethane was added dropwise to 1 very slowly under refrigeration. Using this process, 1 was added in a 1.5-fold excess, because an equimolar quantity of 1 led primarily to the formation of a by-product in which both amino groups were blocked. After the by-product and excess diamine adduct (1) were removed via column chromatographic separation, the compounds 2 were obtained in yields of 60-70% (based on the Boc anhydride used).

Ethyl iodide was then used to introduce the ethyl group not only because it is a stronger alkylation reagent compared with ethyl bromide (which could also be used in principle) but because it also has a higher boiling point (71° C.) (ethyl bromide: 38° C.). The alkylations to the compounds 3 could then be carried out at 60° C. in acetonitrile with good yields (64 to 86%).

The Boc protective group was then removed completely using trifluoroacetic acid. Simple purification via extraction of the dichloromethane phase against 1 N NaOH quantitatively yielded (except for 31: 74%) the purified products of compound group 4.

Coupling of Lipid Components and Protected Head Group

The benzyl-protected, bicationic head groups were successfully prepared via alkylation of the protected, primary amino group with the lipid components carrying the bromo, chlorine, or mesylate function.

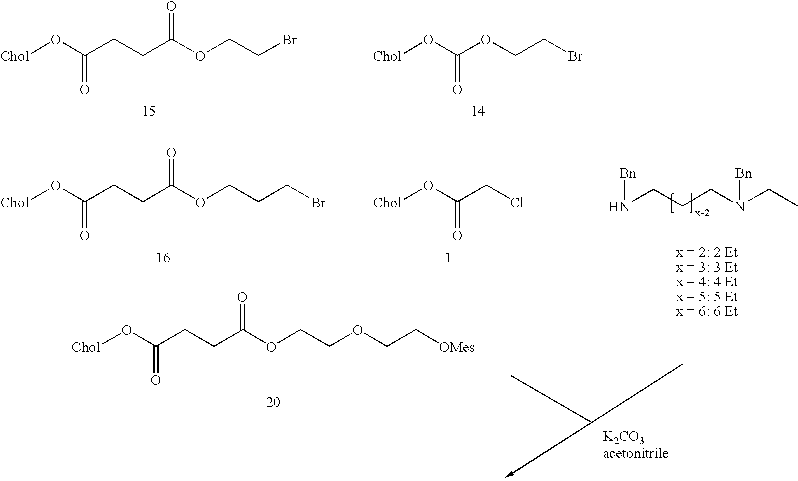

| Lipid Component | Head Group | Product |
|---|---|---|
| 1 | 4 Et | 41 |
| 14 | 4 Et | 42 |
| 15 | 4 Et | 43 |
| 16 | 4 Et | 44 |
| 20 | 4 Et | 45 |
| 1 | 2 Et | 46 |
| 1 | 3 Et | 47 |
| 1 | 5 Et | 48 |
| 1 | 6 Et | 49 |

The lipid anchors that carry the bromo and mesylate functions proved to be very reactive alkylation reagents, as expected (monitored using thin-layer chromatography: quantitative reactions). The yields after purification via column chromatography were 50 to 68%. Even alkylations with the lipid anchor that carries a chlorine function as the leaving group (1) led to good yields (59 to 75% after purification). The strong electron attraction exercised by the ester function apparently offsets disadvantages caused by the moderately good chlorine leaving group.

K₂CO₃ was used as the base for the alkylations described in this study [Hidai et al., 1999], but other bases, e.g., KF celite [Lochner et al., 1998] are described as well. All alkylations were carried out in a mixture of acetonitrile and toluene (8:1). Toluene had to be added in order to completely dissolve the very apolar lipid components with acetyl spacers and carbonate spacers, which accelerated the reaction time.

Attempts to Alkylate the Protected Head Group with Lipid Components 17 and 18

Alkylation of the benzyl-protected head group 37 with lipid components 17 and 18 (which are derived from lipid components 15 and 16 via substitution of the ester bond with an amide bond) was unsuccessful. Interestingly, conversion of the benzyl-protected head group was not observed in the reaction in acetonitrile/toluene with reflux. Rather, the lipid components reacted quantitatively to a by-product which was identified as cholesterol.

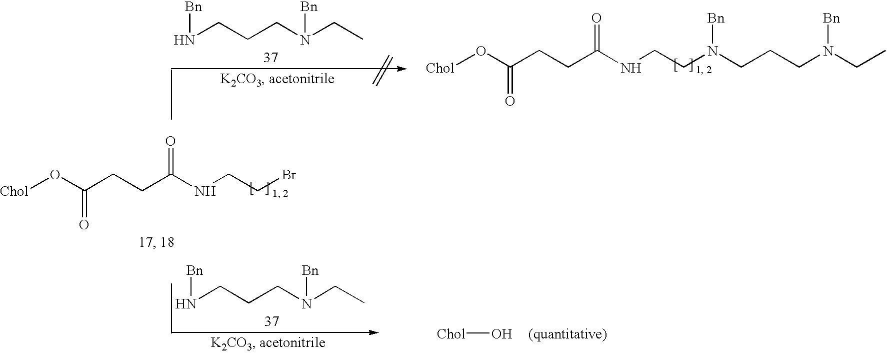

The desired conversion was not achieved at lower temperatures, either. While it is not the intention of the applicant to be bound to any particular theory, nor to affect in any measure the scope of the appended claims, it is presently believed that a possible reason for the formation of cholesterol is a nucleophilic attack on the cholesterol-succinyl ester bond by the amide nitrogen, which would lead to an energetically more favorable, cyclic succinic acid imide.

Debenzylation of Amino Groups and Preparation of Final Compounds

Initial experiences with the removal of protection from benzyl-protected amino functions were collected in preliminary studies of the selection of suitable amino protective groups for polyamine synthesis. Building on the initial experiences collected in this case, a few optimizations were developed to remove protection from bicationic lipids via hydrogenolysis. These optimizations will be presented here. All debenzylations were carried out using gaseous hydrogen (atmospheric pressure) in a reaction catalyzed by palladium (adduct/Pd: 10:1). Other hydrogen sources such as formic acid [Jacobi et al., 1984] or cyclohexene [Overman et al., 1983] as alternatives to hydrogen gas are also basically described.

The respective adduct was dissolved in as little solvent as possible to debenzylate the amino groups. An optimally concentrated adduct solution could then be presented to the reactive hydrogen gas while stirring vigorously in a hydrogen atmosphere. This resulted in an accelerated reaction of the adducts. If debenzylations are carried out frequently with palladium as the catalyst in highly polar, protic solvents such as ethanol or methanol, a dichloromethane/methanol mixture (2:1) would have to be used to remove protection from the bicationic lipids because of solubility (cholesterol as the lipid anchor). Adducts as well as products dissolved in this solvent mixture. This is important because the surface of the catalyst is therefore blocked for further reactions by insoluble products/adducts, and the catalyst is therefore inactivated.

Since the final compounds should be obtained as salts of acetic acid, acetic acid was also added to the protection-removal formulation. An accelerated reaction was observed in some cases after acetic acid was added. This is due to the elevated proton concentration. Before the catalyst was added, the formulation was stirred for 30 minutes with one spatula tip of activated charcoal to bind any catalyst poisons that might be present.

The following bicationic lipids were obtained via hydrogenolysis:

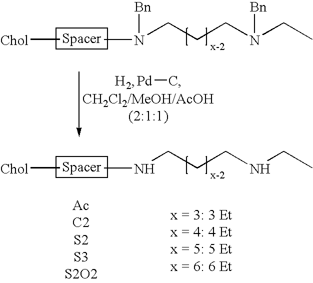

| Spacer | Head Group | Product |
|---|---|---|
| Ac | 4 Et | 57 |
| C2 | 4 Et | 58 |
| S2 | 4 Et | 59 |
| S3 | 4 Et | 60 |
| S2O2 | 4 Et | 61 |
| Ac | 3 Et | 62 |
| Ac | 5 Et | 63 |
| Ac | 6 Et | 64 |

The C—C double bond contained in the cholesterol parent structure is stable under the hydrogenolysis conditions used. This was demonstrated using ¹H-NMR spectroscopy in all final compounds synthesized as part of this study, in conformance with the literature [e.g., Cooper et al., 1998].

All bicationic lipids were obtained as salts of acetic acid. The advantage of this was that the final compounds occur as solids, which makes it easier to weigh out the compounds for transfection experiments, for instance. The final compounds purified under alkaline conditions occurred as slime. To completely convert all bicationic lipids to the solid state, the procedure for precipitating the final compounds was optimized and carried out as follows.

The products obtained via column chromatography were dissolved in a small amount of dichloromethane. After addition of an equivalent quantity of an acetone/diisopropylether mixture, the product precipitated out quickly. Dichloromethane (lowest boiling point) and then acetone and diisopropylether were removed in a vacuum using a rotary evaporator at room temperature. In this process, the products precipitated out as solids due to their insolubility in acetone/diisopropyl ether mixtures. The final compounds were dried in a high vacuum.

Compound 46 could not be hydrogenolyzed. In this compound, the distance between the amino functions is two methylene groups.

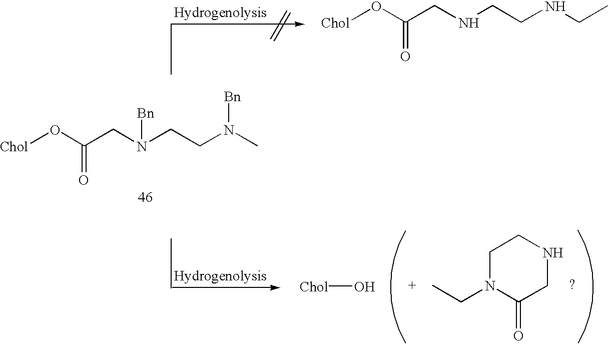

The desired final compound was not obtained in this case. Rather, cholesterol was obtained as the main product. While it is not the intention of the applicant to be bound to any particular theory, nor to affect in any measure the scope of the appended claims, it is presently believed that this is most likely due to ester cleavage after debenzylation. In addition to cholesterol, another potential product is a 6-ring compound (a lactam derivative). Due to the favorable ring tension (6-ring) and gain in energy, the ring formation would be a preferred subsequent product after removal of protection from the terminal amino group.

Preparation of Bicationic Lipids with Two Lipid Anchors

The synthesis strategy and preparation of bicationic lipids with two lipid anchors is described below:

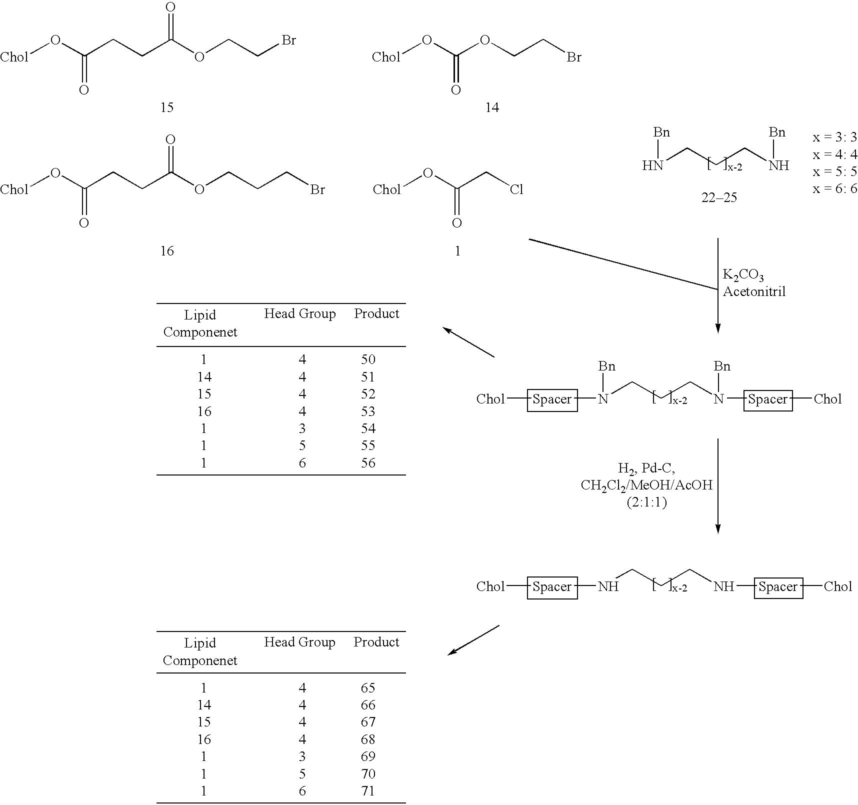

N,N′-dibenyl-α,ω-diaminoalkanes, which were systematically varied in terms of the distance between the amino groups, were used as the starting point to link two lipid components with just one bicationic head group. They were obtained as intermediate products in the synthesis of protected bicationic head groups (see above). The two benzyl-protected amino groups of the head groups (22 through 25) were alkylated with a 2.6-fold excess of the respective lipid components in acetonitrile (reflux) and with K₂CO₃ as the base. A quantitative reaction was observed in all reactions (monitored using thin-layer chromatography). Purification via column chromatography, on the other hand, led to somewhat unsatisfactory yields of protected compounds that were between 45% and 76%. Apparently a few compounds exhibited a high affinity to silica gel, because it was not possible to quantitatively elute the products.

The hydrogenolysis conditions used to prepare bicationic lipids with a lipid anchor (H₂, Pd-C) were used to debenzylate the protected compounds into bicationic target compounds (65 to 71). It took just 1-2 hours for the reaction to be completed (monitored using thin-layer chromatography), which is much faster than the time required to remove protection from the lipids with a lipid anchor (up to 10 hours in some cases). To completely precipitate the products as salts of acetic acids, the products were precipitated out of acetone/diisopropyl ether/dichloromethane mixtures by slowly removing dichloromethane. After the solvents were completely removed in a vacuum, the yields were between 49% and 92%.

Synthesis of Tricationic Lipids

A preferred feature of the synthesis strategy is that the number of methylene groups between the amino groups in the tricationic lipids can be shaped as necessary independently of each other. The synthesis is carried out in convergent fashion in order to minimize the amount of effort required.

The protected tricationic head groups prepared separately will be linked with the lipid components used to prepare the bicationic lipids via alkylation.

The various N,N′-dibenzyl-α,ω)-diamino-alkanes are interesting starting compounds in terms of preparing the protected head groups. amino function for coupling to

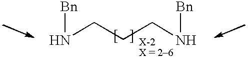

amino function for introducing lipid components via alkylation the third amino function

One of the two benzyl-protected amino groups of the N,N′-dibenzyl-α,ω-diamino-alkanes will be used for coupling with the various lipid components via alkylation. The other amino group will be used to introduce the third amino group via alkylation with an alkylation reagent that contains a primary amino group. This third amino group has to carry a protective group that reliably rules out alkylation of this amino group. This should prevent secondary reactions in the alkylation of the benzyl-protected, primary amino group with the lipid components.

In addition to the Boc protective group [Pak and Hesse, 1998], the Z protective group [Blagbrough et al., 1996] is one of the protective groups used most often for amino groups. Both protect amino groups as carbamate. The Z protective group was selected for use for the third, primary amino group of the tricationic head group because it can be cleaved under the same hydrogenolytic conditions as the benzyl protective group. After the lipid is broken down, the two different amino protective groups will be removed in a single protection-removal step, leaving the final compounds. The synthesis strategy resulting from these considerations is illustrated below using an acetyl spacer as an example:

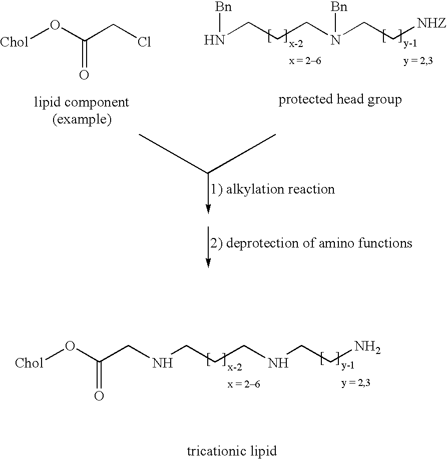

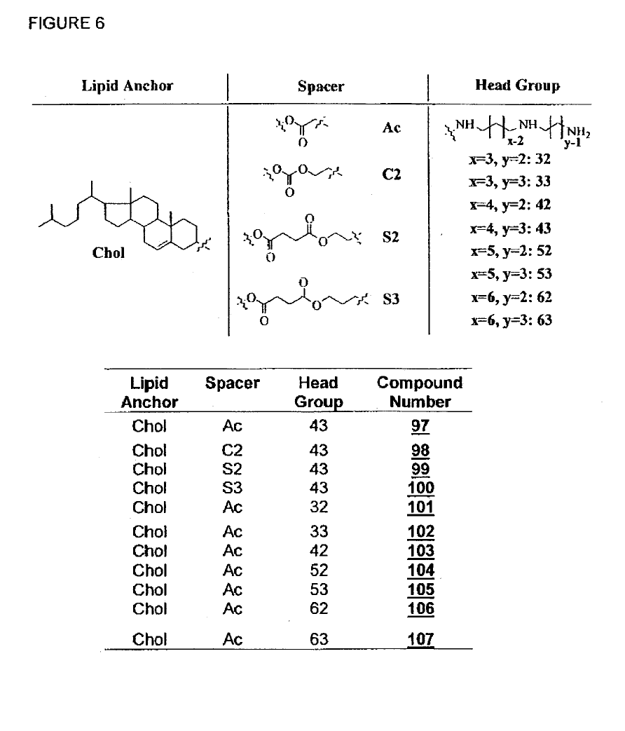

The tricationic lipids shown in FIG. 6 were prepared using the synthesis strategy developed, the individual steps of which will be described in greater detail below.

Synthesis of Tricationic Head Groups

The protected tricationic head groups were successfully prepared using the synthesis sequence described below:

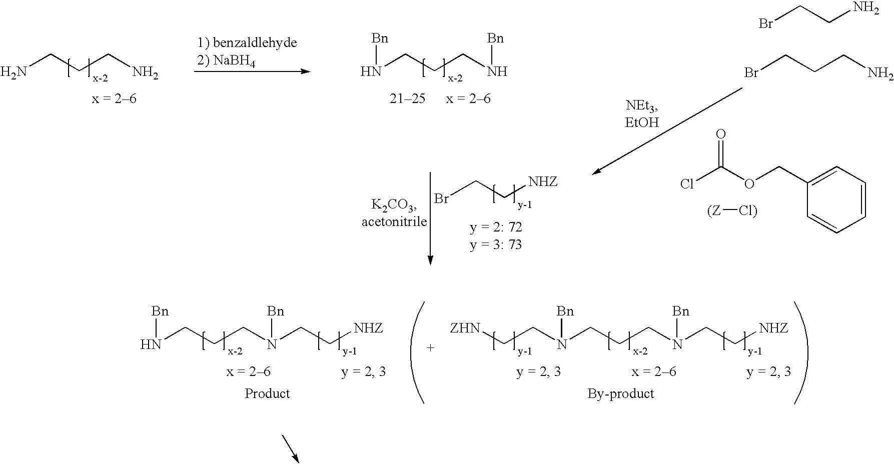

| x = | y = | Product |
|---|---|---|
| 2 | 2 | 74 |
| 2 | 3 | 75 |
| 3 | 2 | 76 |
| 3 | 3 | 77 |
| 4 | 2 | 78 |
| 4 | 3 | 79 |
| 5 | 2 | 80 |
| 5 | 3 | 81 |
| 6 | 2 | 82 |
| 6 | 3 | 83 |

N-Z-2-bromomethylamine (72) and N-Z-3-bromopropylamine (73) were prepared via conversion of 2-bromomethylamine and 3-bromopropylamine, respectively, in ethanol with a 1.5-fold excess of benzyl chlorformiate with triethylamine as the base [Khan and Robins, 1985]. The very polar adducts (the amines were used as salts of hydrobromic acid) were easily soluble in ethanol. With large batches in particular, care had to be taken to add the benzyl chlorformiate (solution in toluene) very slowly in drops to an ice-cooled solution of adduct and triethylamine in ethanol in order to keep the reaction temperature low. It is known that amines, due to their higher nucleophilicity, react much more quickly than alcohols in a strongly exothermic reaction with Z-chloride. If addition was accelerated and the reaction temperature was therefore higher, lower yields were obtained. This may be due to a secondary reaction in which ethanol, the solvent, reacts with Z-chloride. Under well-controlled reaction conditions (0 degrees Celsius), the yields were 71% (73) and 87% (72), respectively, after purification via column chromatography.

To prepare the protected tricationic head groups 74 to 83, an equimolar quantity of the respective N,N′-dibenzyl-α,ω-diamino-alkane (21 through 25) were converted with 72 and 73, respectively, in acetonitrile with K₂CO₃ as the base with reflux. A considerable quantity of by-products were formed that were identified via ¹H-NMR spectroscopy as bialkylated adducts. To reduce the formation of by-products, an 1.5-fold excess of N,N′-dibenzyl-α,ω-diamino-alkanes were used and the conversion was optimized as follows: three equivalents of diamine components (21 through 25) were added to acetonitrile with K₂CO₃ as the base. One equivalent of 72 and 73, respectively, was then added to acetonitrile very slowy in drops and dissolved under reflux conditions. A further equivalent of alkylation reagent was added in drops only after the first equivalent had been completed converted (monitored using thin-layer chromatography). Based on this approach, the yield of monoalkylated product (74 to 83) was increased from 40% to 64% (based on the quantity of alkylation reagent used) after purification via column chromatography.

Coupling of Lipid Components and Protected Head Group

Lipid components 1, 14, 15, and 16 were used to prepare the protected tricationic lipids 84 through 96:

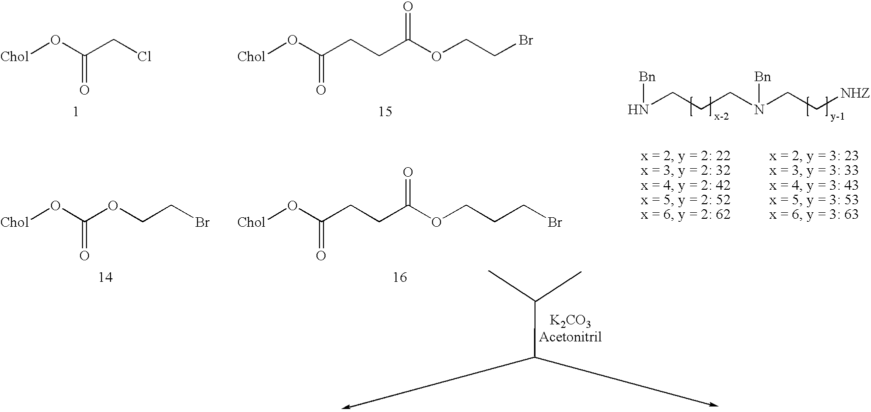

| Lipids with Spacer Variations | | | Lipids with Head Group Variations | | |
|---|---|---|---|---|---|
| Lipid | Head | | Lipid | Head | |
| Component | Group | Product | Component | Group | Product |
| 1 | 43 | 84 | 1 | 22 | 88 |
| 14 | 43 | 85 | 1 | 23 | 89 |
| 15 | 43 | 86 | 1 | 32 | 90 |
| 16 | 43 | 87 | 1 | 33 | 91 |
| | | | 1 | 42 | 92 |
| | | | 1 | 43 | 84 |
| | | | 1 | 52 | 93 |
| | | | 1 | 53 | 94 |
| | | | 1 | 62 | 95 |
| | | | 1 | 63 | 96 |

Since the amount of effort spent on synthesis to prepare the protected tricationic head groups was greater than that used to prepare the various lipid components, a 1.8-fold excess (compared to the head group) of lipid component was used for the coupling. Under the alkylation conditions (acetonitrile, K₂CO₃, reflux), the conversion of the head group used was quantitative after just a few hours (monitored using thin-layer chromatography). The excess quantity of lipid components was removed via column chromatography and the yields obtained were between 60% and 80%.

Removing Protection from Amino Groups and Preparation of Final Compounds

In the hydrogenolytic removal of protection, it was demonstrated that both amino protective groups, the Z protective group and the benzyl protective group, could be removed in one reaction step. The yields fluctuated between 43% and 78% in the protection-removal process.

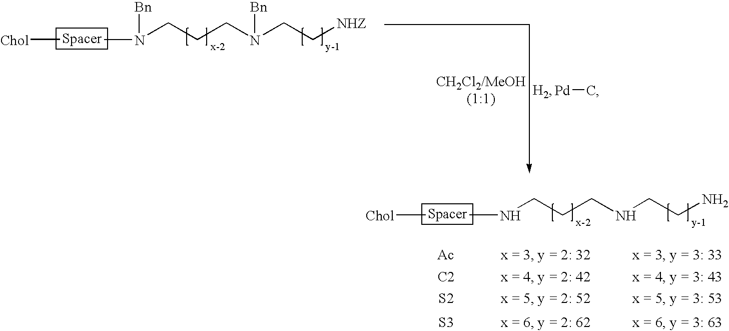

| Spacer | Head Group | Compoun Number |
|---|---|---|
| Ac | 43 | 97 |
| C2 | 43 | 98 |
| S2 | 43 | 99 |
| S3 | 43 | 100 |
| Ac | 32 | 101 |
| Ac | 33 | 102 |
| Ac | 42 | 103 |
| Ac | 52 | 104 |
| Ac | 53 | 105 |
| Ac | 62 | 106 |
| Ac | 63 | 107 |

The first step was to carry out the hydrogenolytic debenzylations in an acetic acidic environment to prepare the tricationic lipids analogous to the bicationic lipids. Purification of the products as salts of acetic acid via column chromatography was problematic, however. Apparently there were pronounced interactions between the protonated, positively-charged lipids and the acidic and rather negatively-charged silica gel, which led to considerable problems during elution of the products. The quantity of product eluted was very small even when highly polar, hydrous solvent mixtures were used. In addition, the separation quality was unsatisfactory.

For this reason, the removal of protection was carried out without addition of acetic acid. The final compounds were therefore not obtained in protonated form at first. Subsequent purification of the products via column chromatography was carried out with an alkaline solvent mixture. The product was successively eluted quantitatively without formation of mixed fractions because ammonia was added to the chloroform/methanol mixtures used for elution. In order to still obtain the final compounds as salts of acetic acid, however, the products were dried thoroughly in a high vacuum to completely remove the ammonia. The product was dissolved in a 1:1 mixture of dichloromethane and acetone. After acetic acid was added, a large portion of the products usually precipitated out as salts of acetic acid. Residues of ammonia would also precipitate out as salt after the addition of acid, which is why the products had to be dried thoroughly after column chromatography. Complete precipitation of the products was achieved by slowly removing the halogenated solvent (dichloromethane boils at 40° C. and acetone boils at 56° C), because the product salts are not soluble in acetone. The final compounds were obtained as solids, which made it easier to weigh out very small quantities for the transfection experiments in particular.

Problems were encountered in the process of removing protection from the protected tricationic lipids 88 and 89. In addition to the acetyl spacer, these two lipids share as a common structural element a distance of two methylene groups between the first (adjacent to the spacer) and the middle amino group.

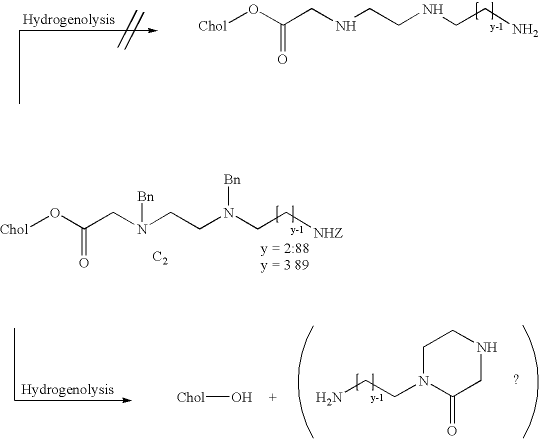

Cholesterol was obtained instead of the desired products (monitored using thin-layer chromatography). The same phenomenon was observed earlier in the experiment to debenzylate one of the bicationic lipids (46) having a related structure. While it is not the intention of the applicant to be bound to any particular theory, nor to affect in any measure the scope of the appended claims, it is presently believed that the cause in this case too is ester cleavage after denbenzylation. The possible formation of a 6-ring compound (lactam) was another by-product in addition to cholesterol. This would be a preferred subsequent product due to the favorable ring tension (6-ring) and gain in entropy. The exact structure of the cyclic by-product was not investigated.

Synthesis of the DMG Derivatives

The syntheses of simple cationic, bicationic, and tricationic lipids with an acetyl spacer that contain 1-(2,3-di-tetradecyloxy)-propanol instead of cholesterol as the lipid anchor will now be described. With the successful preparation of cationic DMG derivatives, it was demonstrated that the synthesis strategy developed not only allows the spacer and head group unit to be varied, but that the lipid anchor can basically be varied as well.

Preparation of Lipid Components

Synthesis of the lipid anchor 1-(2,3-di-tetradecyloxy)-propanol (108): 1-(2,3-di-tetradecyloxy)-propanol was synthesized in 4 steps starting with 1,2-O-isopropyliden-glycerin, which is commercially available [Eibl and Woolley, 1986]:

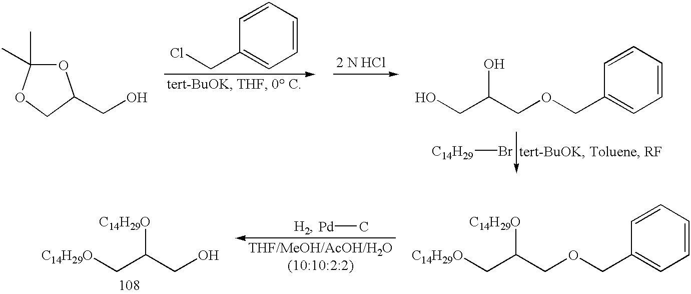

In the initial step, the free hydroxy function of 1,2-O-isopropyliden-glycerin was converted to the benzyl ether using benzyl chloride and tert-BuOK in tetrahydrofuran. The isopropyliden group, which is stable under alkaline conditions, protects the other two hydroxy functions during this process. Adduct could no longer be detected after two hours (monitored using thin-layer chromatography). The acid-labile isopropyliden protective group could therefore be removed by adding 2 N hydrochloric acid very slowly. Before the two de-protected hydroxy functions could be etherified in a third step, the 1-benzyl glycerol ether had to be roughly purified via extraction. The two hydroxy functions were then converted to the corresponding ether using tetradecyl bromide and tert-BuOK as the base. In order to use tetradecyl bromide, which is highly apolar in solution, toluene was used as an apolar solvent. The product was first purified via column chromatography (to remove the excess tetradecyl bromide), then the benzyl ether was cleaved via catalytic hydrogenolysis (Pd-C, H₂). The product 1-(2,3-di-tetradecyloxy)-propanol (108) was obtained in a yield of 69% (over 4 synthesis steps) after purification via column chromatography.

Synthesis of the lipid component chloroacetic acid-1-(2,3-di-tetradecyloxy)-propylester (109): Lipid component (109) was prepared via esterification of 108 with chloroacetic acid anhydride with triethylamine as the base:

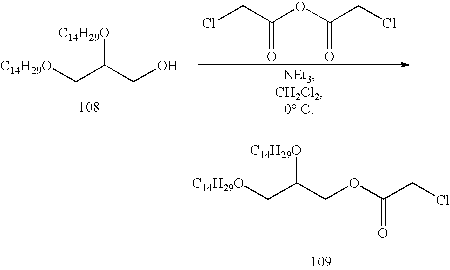

Conversion was successful. The yield after purification via column chromatography was 96%.

Preparation of Simple Cationic DMG Derivatives

To prepare the simple cationic DMG derivatives 110 and 111, lipid component 109 was reacted with an ethanolic dimethylamine solution in an alkylation reaction under refrigeration in toluene.

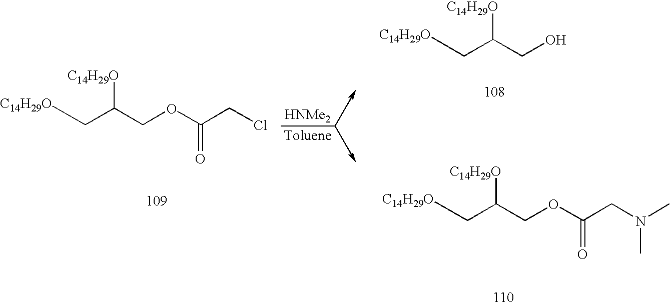

Adduct could no longer be detected after a short time (2 hours). In addition to the desired DMG lipid 110 (32%), however, another product was formed as well. It was identified via thin-layer chromatography as 1-(2,3-di-tetradecyloxy)-propanol (108). Apparently ester cleavage took place during the reaction and 108 was released.

Lipid 111, which carries a quaternary amino group, was carried out via alkylation of 110 with dimethyl sulphate at room temperature:

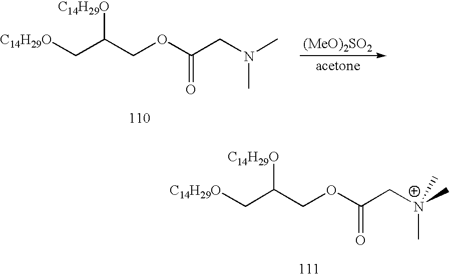

The conversion was carried out in acetone, out of which the formed product precipitated after a brief period. It was purified via rewashing with a small quantity of acetone. Unfortunately an even larger quantity of 111 remained dissolved in the mother liquor. This explains the low yield of 40%. Purification via column chromatography would lead to higher yields, but was not carried out due to the extra effort required.

Preparation of a Bicationic DMG Derivative

The convergent synthesis strategy previously developed was used to prepare a DMG lipid with a bicationic head group. The benzyl-protected, bicationic head group 38—which has a distance of 4 methylene groups between the amino groups—was used to couple with the DMG lipid component 109. Lipid 112 was obtained in a yield of 66% using the alkylation conditions (K₂CO₃, acetonitrile and reflux) described previously. Subsequent removal of the benzyl protective groups via catalytic hydrogenation (Pd—C, H₂) also proceeded smoothly and led to the desired bicationic lipid 113 with a yield of 66% after purification via column chromatography.

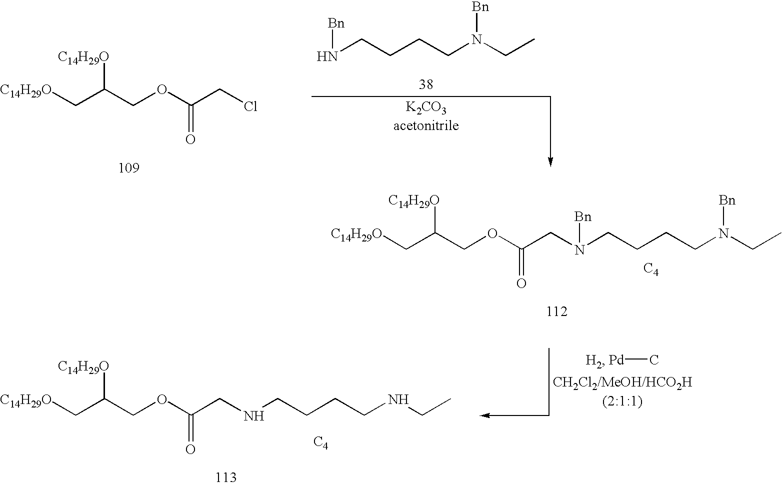

In contrast to the bicationic cholesterol derivatives, formic acid was added to the protection removal formulation instead of acetic acid. As a result, lipid 113 was obtained as a salt of formic acid.

Preparation of a Tricationic DMG Derivative

Preparation of the tricationic DMG lipid is illustrated in below:

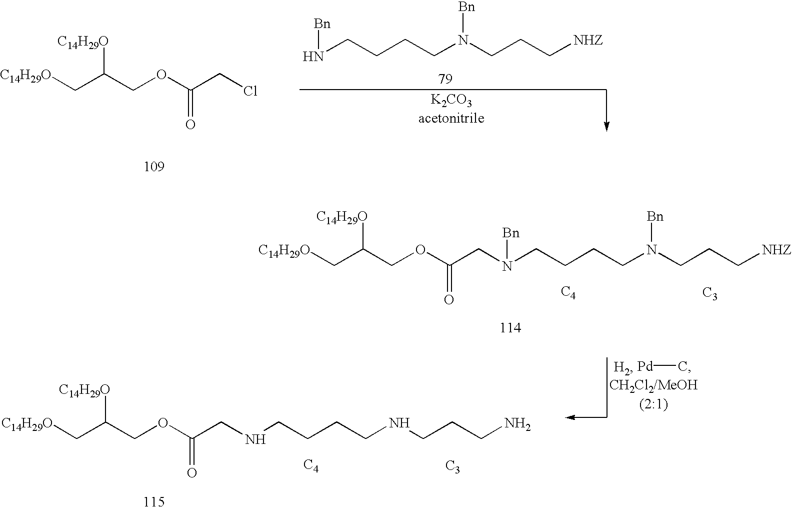

Using the convergent synthesis strategy described previously, the protected bicationic head group 79 (spermidine) was linked with the DMG lipid component 109 in an alkylation reaction to form compound 114 (yield: 72%). The two amino protective groups, the benzyl and the Z protective group, were then removed in a protection removal step via catalytic hydrogenation (Pd—C, H₂). The target compound 115 was first purified under basic conditions via column chromatography in a procedure analogous to that described previously. After the lipid was redissolved in a mixture of dichloromethane and acetone, acetic acid was added and dichloromethane was removed slowly in a vacuum. The compound was then precipitated out as a salt of acetic acid and carefully dried in a high vacuum (yield: 52%).

Transfection Results

Transfection experiments with presently preferred embodiments of the present invention will now be described and the details of the individual transfection steps discussed.

Individual Steps of a Transfection Experiment

A transfection experiment with cationic lipids can be broken down into four different individual steps. An important prerequisite for the success of a transfection experiment is the successful preparation of liposomes from the lipids to be tested (Step 1). DNA dissolved in buffer is then added to these liposomes, forming lipid/DNA complexes called lipoplexes (Step 2). The lipoplexes are then added to the cells (Step 3). The lipoplexes mediate the uptake of the DNA in the cell. Finally, the success of a transfection is quantified by determining the quantity of gene product forms and any toxicity that may occur is quantified by measuring the quantity of total protein (Step 4).

Liposome Preparation

When it is not possible to form stable lipid bilayers (i.e., liposomes) using a single lipid, then it may be necessary to combine the lipid with one or more helper lipids. As used herein, “lipid mixture” will be understood to refer both to individual cationic amphiphiles used by themselves and cationic amphiphiles used in combination with one or more helper lipids. Furthermore, as used herein, “liposome” will be understood to refer to lipid mixtures in the form of lipid bilayers.

Liposomes prepared in accordance with the present invention may contain other auxilirary/helper lipids in addition to the cationic lipids. Suitable helper lipids include but are not limited to neutral or acidic phospholipids including phosphatidylcholines, lyso-phosphatidylcholine, dioleoyl phosphatidylcholine (i.e., DOPC), phosphatidyl ethanolamines, lyso-phosphatidylethanolamines, diphytanoylphosphatidylethanolamine, dioleoylphosphatidyl-ethanolamine (i.e., DOPE), and cholesterol. Preferably, the helper lipid is selected from the group consisting of DOPE, lecithins and cholesterol. More preferably, the helper lipid is DOPE.

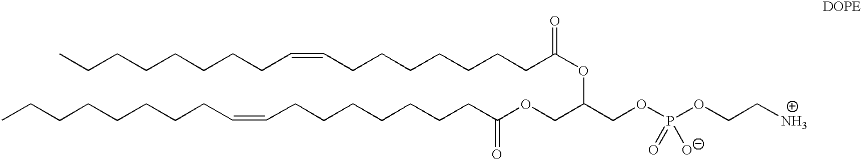

The lipid complexes of the invention may also contain negatively charged lipids as well as cationic lipids so long as the net charge of the complexes formed is positive. Negatively charged lipids of the invention are those comprising at least one lipid species having a net negative charge at or near physiological pH or combinations of these. Suitable negatively charged lipid species comprise phosphatidyl glycerol and phosphatidic acid or a similar phospholipid analog.

Preferably, the cationic amphiphile and helper lipid are present in a molar mixing ratio of from about five to one to about one to five. More preferably, the cationic amphiphile and helper lipid are present in a molar mixing ratio of from about two to one to about one to two. Still more preferably, the molar mixing ratio of cationic amphiphile to helper lipid is about 1:1. To prepare liposomes, the cationic lipid to be tested and a helper lipid were dissolved in an organic solvent (e.g., chloroform/methanol mixtures).

Amphiphilic lipids that carry a polar or charged head group are especially soluble in chloroform/methanol mixtures. Due to their low boiling points, these solvents are removed very quickly in a nitrogen stream, with formation of a thin lipid film with a large surface. The nitrogen used also protects the lipids from oxidation via ambient oxygen in this process. It was demonstrated that the lipids in this lipid film are chemically stable for months when stored at −20° C. This was verified by performing thin-layer chromatography of the lipids after storage.

The lipid films can be hydrated by adding buffer, which also gives rise to multilamellar vesicles (MLV). The MLV are then converted into small unilamellar vesicles (SUV) via ultrasonic treatment. [Moog, 1999; Lasic, 1994 and 1995]. The duration of exposure to ultrasound required until a homogenous population of SUV formed depended on the strength of the ultrasound bath. When a common commercial ultrasound bath was used, the process lasted 15-20 minutes. When a high performance ultrasound bath from Bandelin was used (which is also used to homogenize tissue samples) the same results were obtained after just 2 minutes. All liposomes were characterized by measuring their size distribution using photon correlation spectroscopy. In this process, the sizes of liposomes found were preferably between about 20 and about 200 nm, more preferably between about 50 and about 150 nanometers.

DOPE, the Helper Lipid

According to studies described in the literature, cationic lipids with cholesterol as the lipid anchor do not form stable liposomes directly, but rather in a mixture with bi-chained helper lipids [Deshmukh and Huang, 1997]. For this reason, cationic lipids that contain cholesterol as the lipid anchor are normally used for transfection as a mixture with DOPE, which occurs naturally in bi-chained form [Miller, 1998]. A number of preliminary studies using the cationic lipids described in this dissertation confirmed these results: without admixing DOPE to cationic lipids with cholesterol as the lipid anchor, no liposomes could be formed and, as a result, no measurable transfection results were obtained. The bi-chained DMG derivatives were able to form liposomes without DOPE. For reasons of comparability of the transfection results, all lipids were used with DOPE in a mixing ratio of preferably 1:1 for transfection. This mixing ratio is described often as being very effective [Miller, 1998; Felgner et al., 1994].

Formation of Lipoplexes

A reporter plasmid pCMXluc containing 8,600 base pairs (provided by R. Schüle) that codes for the enzyme luciferase was used to manufacture the lipoplexes. The composition of the lipoplexes and especially the proportion of lipid/DNA [Weibel et al., 1995; Felgner et al., 1994] are critical factors for obtaining a high level of protein expression. The number of negative charges of the plasmid used (one negatively-charged phosphate group per base) and the number of positive charges caused by adding cationic liposomes were used to calculate the proportion of lipid/DNA. For polycationic lipids, the number of all amino functions contained in the head group was made equal to the number of positive charges per lipid. Eight different lipoplexes with charge ratios from 1:1 to 15:1 were manufactured for each cationic lipid. In this process, each lipoplex received the same quantity of DNA but different quantities of lipid. An overview of the model calculation of various proportions of lipid/DNA is presented below.

After the plasmid (dissolved in buffer) was added to the liposomes, a pipette was used to carefully mix the formulation. It was then allowed to stand at room temperature for 60 minutes to allow the lipoplexes to form. The ripening conditions used here to prepare lipoplexes are based on findings described earlier [Yang and Huang, 1998] and also represent the result of a systematic optimization of lipoplex ripening conditions that were determined using a simple cationic lipid and a tricationic lipid, and which were used here to prepare all lipoplexes [Regelin, 2000].

Cell Experiments

The COS-7 cell line (kidney cells from the green meerkat, fibroblast-like cells) which is often used in transfection studies [You et al., 1999; Yu et al., 1999] was used for the cell experiments. This cell line is known to be easy to transfect and grows adherently. It was therefore expected that transfection data could be matched up with all synthesized cationic lipids, and that structure/effectiveness relationships could therefore be identified. The cell experiments were carried out in 96-well microtiter plates with 5,000 cells per well. Cell density has a considerable influence on the success of a transfection experiment when COS-7 cells are used [Deshmukh and Huang, 1997]: on the one hand, cells that are sowed too densely are harder to transfect than less densely sowed cells due to their reduced tendency to divide. While it is not the intention of the applicant to be bound to any particular theory, nor to affect in any measure the scope of the appended claims, it is presently believed that the explanation lies in that plasmids pass more easily into the nucleus of dividing cells [Mortimer et al., 1999], because the cell membrane becomes full of holes or is dissolved during cell division. On the other hand, transfections of cells that have a very low cell density are often associated with elevated cytotoxicity, because more lipoplexes are now available per cell. A moderate cell density of 40-50% confluence, at which a sufficient level of cell division is observed, was selected for the transfection experiments to be carried out as part of this work. After various lipoplexes were added to the cells and a short centrifugation step was carried out (better interaction with the cells), the medium was replaced with fresh medium after four hours [Gao and Huang, 1991]. This was done to eliminate any disadvantages to cell growth caused by diluting the medium with the lipoplex solution. All experiments were carried out in triplicate determinations and the results were indicated in means with standard deviations.

Cationic amphiphiles embodying features of the present invention can be employed in admixture with conventional excipients (i.e., pharmaceutically acceptable organic or inorganic carrier substances suitable for oral, parenteral, inhalation or topical application which do not deleteriously react with the active compositions). Suitable pharmaceutically acceptable carriers include but are not limited to water, salt solutions, buffer solutions, protein solutions (e.g., albumen), carbohydrates such as sugars or sugar alcohols (e.g., dextrose, sucrose, lactose, trehalose, maltose, galactose, and mannitol).

Cationic amphiphiles embodying features of the present invention can be used to facilitate delivery into cells of a variety of biologically active, molecules including but not limited to: polynucleotides such as DNA, RNA and synthetic congeners therof; polynucleotides such as genomic DNA, cDNA, and mRNA that encode for therapeutically useful proteins as are known in the art; ribosomal RNA; antisense polynucleotides, whether RNA or DNA, that are useful to inactivate transcription products of genes and which are useful, for example, as therapies to regulate the growth of malignant cells; missense polyncletides; nonsense polynucleotides; ribozymes; proteins; biogically active polypeptides; small molecular weight drugs such as antibiotics or hormones.

In accord with the practice of presently preferred embodiments of the present invention, a method for treating patients suffering from cancer is provided wherein the biologically active molecule delievered into cells is an anti-tumor agent, and the cells into which the biologically active molecule is delivered are tumor cells and/or tumor related cells (e.g., tumor vasculature cells).

Also in accord with the practice of presently preferred embodiments of the present invention, a method for treating patients suffering from inflammatory disease is provided wherein the biologically active molecule delievered into cells is an anti-inflammatory agent, and the cells into which the biologically active molecule is delivered are involved in the inflammatory process. While it is not the intention of the applicant to be bound to any particular theory, nor to affect in any measure the scope of the appended claims, it is presently believed that the mechanism of such anti-inflammatory agency would probably involve inhibition of the negatively charged lipid responsible for PKC activation.

Determination of Transfection Data and Graphic Illustration

48 hours after the lipoplexes were added, the medium was removed and the cells were washed, because the medium would interfere with the tests to determine the transfection data. The cells were then lysed in order to make a homogeneous cell protein solution. The lysate of every single well was distributed and the total quantity of protein and the luciferase activity were determined in independent assays.

The total protein quantity was determined using the BCA test with bovine serum albumin as the standard and indicated in the unit μg/well. The protein quantity measured correlates with the number of adherent, living cells still present after transfection and can therefore be used as a measure of toxicity of the respective lipoplexes [Gao and Huang, 1991]. Comparisons of the protein quantities measured with results from cytotoxicity assays that were performed [Regelin, 2000] verify this relationship. The measured values were used in the calculation of transfection efficiency (see below).

If the reporter plasmid (pCMXluc) that codes for the enzyme luciferase is successfully transfected into the cells, luciferase is expressed, which can then be detected in the cell lysate using the scheme illustrated below:

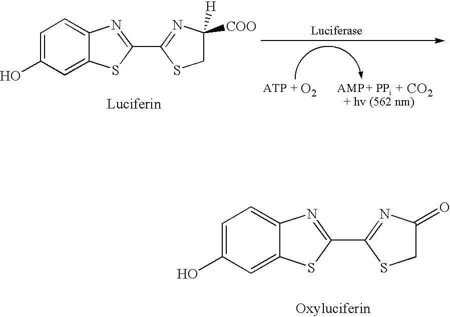

Light emitted as a result of the luciferase reaction can be quantified at a wavelength of 562 nm by adding ATP and luciferin to the lysate. The value measured for the luciferase activity is indicated in relative light units per well (RLU/well). According to the information commonly presented in the literature, the value for the luciferase activity obtained for each lipoplex is based on the respective total quantity of protein. The transfection efficiencies, which are independent of the number of transfected cells, are obtained using the following formula:

$$\text{transfection efficiency} = \frac{\text{luciferase activity}}{\text{protein qty}} [ \frac{RLU}{µg} ]$$

Cell growth and, therefore, the transfection efficiencies obtained, are subject to natural fluctuations. Transfection experiments carried out under identical conditions using lipoplexes that were manufactured under identical conditions can yield different absolute values on different days. The results can also vary considerably from microtiter plate to microtiter plate (on the same day). In order to be able to compare the transfection efficiencies of all synthesized cationic lipids with each other, however, the transfection efficiency of an external standard lipid was also determined in each microtiter plate. The simple cationic transfection lipid DOTAP (which has been described in many studies as effective) was used for this purpose. This means that a few cells in each microtiter plate were transfected with DOTAP (proportion of lipid/DNA: 2.5:1) instead of the lipids to be tested, and the DOTAP transfection efficiencies obtained were defined as 100%. The transfection efficiencies of all other cationic lipids were then based on this value. The “relative transfection efficiencies” were then calculated by forming the quotient:

$$\text{relative transfection efficiency [\%]} = 100 \times \frac{tr \cdot eff \cdot lipid [ RLU \text{/} µg protein ]}{tr \cdot eff \cdot DOTAP [ RLU \text{/} µg protein ]}$$

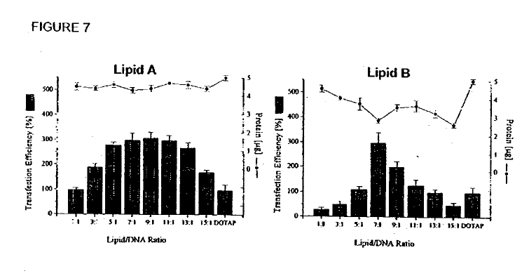

All data on the relative transfection efficiencies (simply called “transfection efficiency”) and total protein quantities as a measure of the cytotoxicity of cationic lipids were summarized in a transfection diagram (FIG. 7).

Each transfection diagram represents the transfection results from 8 different types of lipoplexes of a new lipid with a 1:1 to 15:1 proportion of lipid/DNA, and of lipoplexes of DOTAP, the standard lipid (proportion: 2.5:1) (x-axis). The calculated transfection efficiencies are shown as bars (with the corresponding scale on the right y-axis), and the protein values are shown as dots (with the scale on the right y-axis). The individual points are connected to make the diagram easier to understand.

To facilitate understanding, the discussion of the transfection results of various lipids are often broken down into three sections: a comparison of the transfection efficiencies, the transfection profile, and the cytotoxicities.

Comparison of Transfection Efficiencies:

When considering the transfection efficiencies of the two examples (Lipid A and Lipid B), it becomes clear that the transfection efficiencies obtained, at approx. 300%, far exceed the transfection efficiency of DOTAP. The lipids exhibit their maximum transfection efficiency with a lipid/DNA ratio of 7:1 and 9:1, respectively.

Comparison of Transfection Profiles:

The different transfection profiles of lipids A and B make it clear that various lipid/DNA ratios that are systematically varied across a broad range must be investigated to find the best transfection result: slight variations of the ratio in some cases, such as changing from 5:1 to 7:1 for lipid B in particular, lead to much different transfection efficiencies, for instance. Lipid A, on the other hand, reaches a plateau. In other words, similarly high transfection efficiencies are achieved over a broad range of systematically varied lipid/DNA ratios. A transfection profile that is typical for the lipid can therefore be assigned to both lipid A and lipid B.

Comparison of Cytotoxicities:

The cytotoxicities of the two lipids are much different: lipoplexes from lipid A do not exhibit elevated cytotoxicities in any of the 8 different ratios, although, considering the transfection efficiency, a great deal of lipid must have been taken up in the cells. Lipoplexes that contain lipid B exhibit clearly elevated levels of cytotoxicity. The very efficient lipoplexes (lipid/DNA ratio of 7:1) and complexes with a high percentage of lipid (lipid/DNA ratio of 15:1) lead to increased cell damage.

Transfection Results of Simple Cationic Lipids

The spacers of the simple cationic lipids are different in terms of their polarity as well as their length. The various spacers can be broken down into 3 groups based on their chemical structure that vary in terms of their polarity. The order of priority of the polarity was determined based on the R_{f} values (thin-layer chromatography) of the lipid components varied in the spacer.

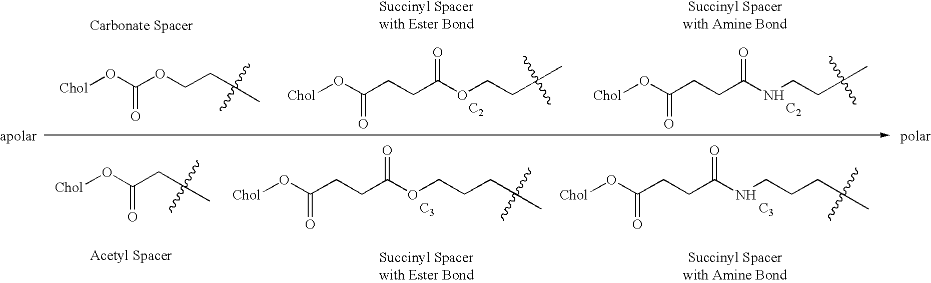

The carbonate and acetyl spacers represent the apolar spacers in this order of priority (Group 1) while the succinyl spacers are much more polar due to the second ester bond (Group 2) and the second amide bond (Group 3).

Lipids with Apolar Acetyl and Carbonate Spacers

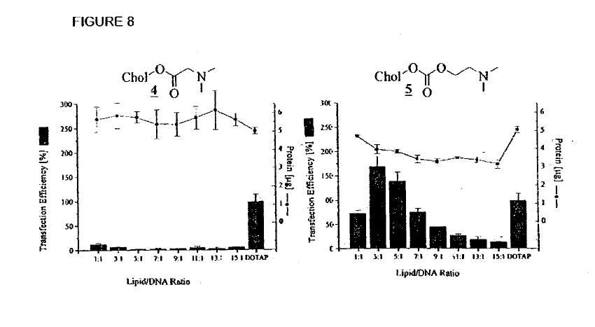

Acetyl and Carbonate Derivatives with Tertiary Amino Groups: Although the two lipids have different spacers (acetyl and carbonate spacers), they both carry a tertiary amino group. Tertiary amino groups should be presented in protonated form under the conditions used (physiological pH of 7.4), and the lipids with such a head group should, as a result, should have a positive charge, which is important for an interaction with the negatively charged DNA during formation of the lipoplex. Transfection experiments with these two lipids led to different results, as illustrated in FIG. 8.

The carbonate derivative 5 had a transfection efficiency of up to 169% with a lipid/DNA ratio of from 3:1 to 5:1, making it much more effective than DOTAP. Deviations from this optimal ratio resulted in a significant decrease in efficiency. The acetyl derivative 4, on the other hand, did not result in a single transfection in any of the systematically varied lipid/DNA ratios. Additionally, the cytotoxicity caused by the lipid was highly dependent on the lipid/DNA ratio of the lipoplexes used: an increasing portion of lipid 5 used for the complexation of DNA correlated with an increasing cytotoxicity. No cytotoxicity was observed with lipid 4. Apparently no lipoplexes had entered the cells (no reporter gene activity was observed).

Under the selected pH conditions of 7.4, both lipids also exhibited considerable biophysical differences: while liposomes with a typical diameter of 55 nm could be manufactured easily from the carbonate derivative 5 and DOPE, liposomes could not be prepared from the acetyl derivative 4 and DOPE, even after extending the exposure to the ultrasound bath to 10 minutes. The acetyl derivative, which was well homogenized in buffer, was investigated for transfection properties nevertheless. Lipid 4 is apparently not capable of forming lipid bilayers in conjunction with DOPE. As described earlier, this is a prerequisite for the formation of effective lipoplexes, however, in which these lipid bilayers are a central structural unit [Battersby et al., 1998].

While it is not the intention of the applicant to be bound to any particular theory, nor to affect in any measure the scope of the appended claims, the following discussion on liposome formation is proffered solely for the purpose of illustration and explanation. The various abilities of the two lipids 4 and 5 to form bilayers can be explained by their chemical structure. All phospholipids that can form bilayers carry either a charged or a neutral, zwitterionic head group [Litzinger and Huang, 1992]. Due to the resultant strong hydration of the head groups, these lipids have a balanced relationship between the surface required by their apolar and polar molecular portion and tend to form bilayers in water/buffer. The lipids shown in FIG. 8 carry a tertiary amino group as the head group. Whether and to what extent these amino groups protonate under physiological conditions and, therefore, are charged (degree of protonation) depends on the pHₐ value of the amino group: the lower this value is, the less likely it is that the tertiary amino group is protonated.

Unlike pKₐ values, e.g., for trimethyl- and triethylamine (pKₐ˜10 in aqueous solution), tertiary amino groups, as the hydrophilic head groups of lipids, have a lowered pKₐ value [Bottega and Epand, 1992] due to the localization in the head group region of bilayers. Additionally, the electron density and, therefore, the pKₐ value, is affected by the electron attraction by the spacer. The electron attraction on the tertiary amino group by the spacer can be compared based on the chemical shift of the methylene protons adjacent to the amino group in the ¹H-NMR spectrum.

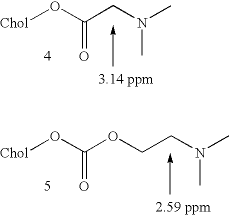

It becomes clear that the acetyl spacer (3.14 ppm) exerts a much stronger electron attraction on the amino function than the carbonate spacer (2.59 ppm). This difference is a decisive indicator of the fact that the tertiary amino function of compound 4 is not (or incompletely) protonated under the buffer conditions (pH 7.4). Based on the resultant unfavorable surface ratios between the lipid anchor and the uncharged and, therefore, only slightly hydrated head group, lipid 4 is apparently not able to form bilayers or liposomes. Additionally, the positive charge for an interaction with the negatively charged DNA is missing. The carbonate spacer (lipid 5), on the other hand, allows sufficient protonation, apparently due to a lower electron attraction compared with the acetyl spacer (successful liposome preparation). In addition to the lower electron attraction on the amino group, the somewhat longer carbonate spacer as compared with the acetyl spacer may also allow the amino group to project further into the aqueous medium, which simplifies protonation.

The preceding discussion of the formation of liposomes was provided solely by way of illustration and explanation, and is not intended to limit the scope of the appended claims.

Acetyl and Carbonate Derivative with a Quaternary Amino Group

Since the acetyl spacer (but not the carbonate spacer, which is similar in terms of polarity) apparently protonation of the tertiary amino groups under physiological pH conditions due to a strong electron attraction, it made sense to create a quaternary, permanently charged amino group as the head group by means of an additional alkylation step. This also made it possible to investigate the basic suitability of the acetyl spacer as a structural unit for transfection lipids.

Quaternary amino groups are often used as the head group of transfection lipids. In addition to quaternization via introduction of an additional methyl group (e.g., DOTMA [Felgner et al., 1987]), lipids have also been described, the amino function of which carries an additional 2-hydroxy ethyl group (e.g., DMRIE [Felgner et al., 1994]).

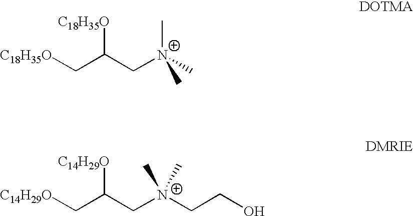

The introduction of the 2-hydroxy ethyl group not only imparts a permanently positive charge as a result of the quaternization of the amino group, but an additional polar hydroxy function as well. This hydroxy function should have numerous positive effects on the efficiency of a transfection lipid [Deshmukh and Huang, 1997; Felgner et al., 1994]. While it is not the intention of the applicant to be bound to any particular theory, nor to affect in any measure the scope of the appended claims, it is presently believed that the positive effects of the polar hydroxy function involve: more efficiently packed lipoplexes due to the additional formation of hydrogen bridge bonds with the DNA; and strengthening the hydration of the head groups of cationic lipids and, therefore, the hydration sheath of the bilayer surface as well. This results in stable bilayers, which should have a positive effect on the formation of stable lipoplexes and, therefore, benefit the transfection efficiency. The preceding discussion of the effects of the polar hydroxy group was provided solely by way of illustration and explanation, and is not intended to limit the scope of the appended claims. To clarify the effect of quaternization of 4 and 5, the transfection properties of both compounds were investigated in quaternary form 10 and 11 (additional methyl group) and in the quaternary form 12 and 13 (additional 2-hydroxy ethyl group)

Quaternary Acetyl and Carbonate Derivative with Additional Methyl Group

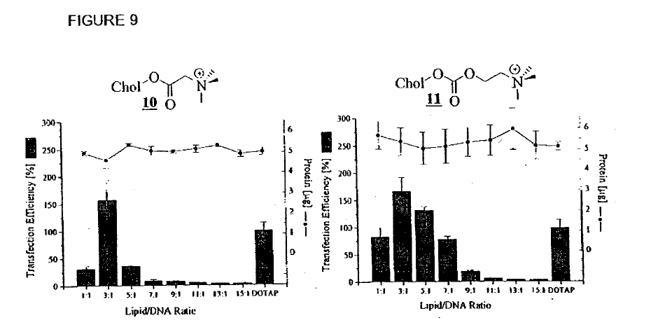

Unlike lipid 4, liposomes could be manufactured with the acetyl derivative 10 in mixture with DOPE. Apparently the introduction of a permanent, positive charge created a favorable relationship between the polar and the apolar portion of the molecule, which made it possible to form bilayers. The fact that liposomes could be prepared successfully confirmed the speculation that the acetyl derivative with a tertiary amino group (4) was unable to form bilayers due to the non-protonated state. Lipid 10 was also used successfully in transfection, unlike 4 (FIG. 9): the lipid exhibited good transfection properties, with a transfection efficiency of 157%.

The cholesterol derivative 11 was used without a problem for liposome preparation in analogous fashion to the homologous linkage with a tertiary, protonatable amino group. The transfection properties of the permethylated compound 11 were similar to those of the non-permethylated compound 5 in terms of the maximum transfection efficiency of 167% (5: 169%) and the transfection profile. Although the additional methyl group does not have a greater effect on the transfection efficiency, it does on the cytotoxicity due on the various lipoplexes, which is practically non-existent in the permethylated compound.

Quaternary Acetyl and Carbonate Derivative with Additional 2-Hydroxy Ethyl Group

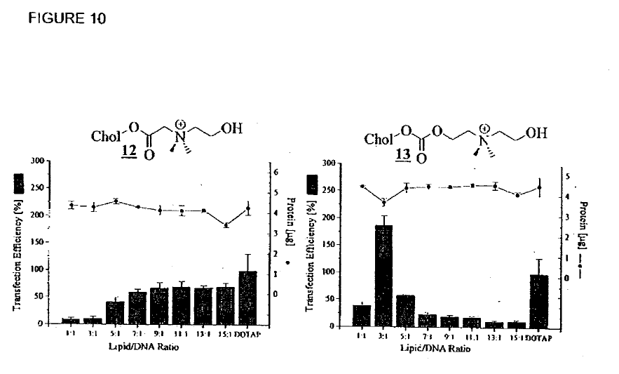

As with the permethylated acetyl derivative 10, liposomes were successfully prepared with the quaternary lipid with an additional 2-hydroxy ethyl group (12) due to the permanently charged head group. Transfection efficiencies (70%) were found as well (FIG. 10) that are similar to those found with DOTAP, the standard lipid. In comparison with the permethylated compound 10 (157%), the additional polar hydroxy function had a negative effect on the transfection properties. This head group variation had a positive effect on the carbonate derivative 13 (FIG. 10); however, lipid 13 exhibited a relative transfection efficiency of 188%. This was 20% higher than that of the homologous lipids with a tertiary or permethylated amino group. Analogous to the permethylated carbonate derivative, a quaternary amino group with an additional 2-hydroxy ethyl group also appears to have positive effects in terms of toxic side effects.

The transfection profiles of lipids 12 and 13 are very different: the acetyl derivative 12 formed a plateau in terms of transfection efficiencies as the proportion of lipid/DNA increased. In conformance with the speculation discussed in the literature that lipids with an additional 2-hydroxy ethyl unit form especially stable lipoplexes [Felgner et al., 1994], an effective complex was produced using 12 at a ratio of just 9:1. The composition of this stable lipoplex would not be changed by adding additional cationic liposomes, which means that the same type of lipoplex is formed at higher ratios (ratio 9:1). The profile of transfection efficiencies of the carbonate derivative 13, on the other hand, formed a very sharp maximum at a ratio of 3:1, and deviations from optimal ratios led to lipoplexes with much poorer transfection efficiencies.

Lipids with Polar Succinyl Spacers

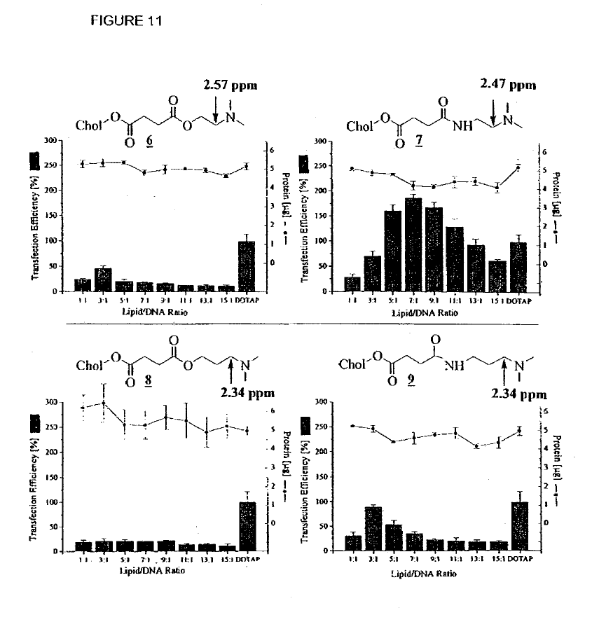

In addition to a tertiary amino group, all simple cationic lipids of this group contain a succinyl unit that is more polar as compared with the acetyl and carbonate spacer. This is extended by means of an ester or amide bond with an additional alkyl chain consisting of 2 or 3 methylene groups, respectively (FIG. 11).

The chemical shifts of the methylene protons adjacent to the tertiary amino groups were in the range of 2.34 to 2.57 ppm and were therefore below the value that was measured with the tertiary amino group (5) in the case of the carbonate derivative (see above). While it is not the intention of the applicant to be bound to any particular theory, nor to affect in any measure the scope of the appended claims, it is presently believed that the tertiary amino groups of the succinyl derivatives are present in protonated form and that all compounds therefore carry a positive charge. This speculation is supported by the fact that liposomes could indeed be manufactured from all lipids in mixture with DOPE. Since it was demonstrated for the group of carbonate derivatives that a permethylated amino group leads to comparable transfection efficiencies as compared with a tertiary but protonated amino group, no quaternary succinyl derivatives were investigated.

In the group of lipids with two methylene groups and in the group of lipids with three methylene groups in the alkyl chain, the derivatives 7 and 9 linked via amide bond were characterized by a much higher transfection efficiency than the ester derivatives 6 and 8, which are homologous to them. While it is not the intention of the applicant to be bound to any particular theory, nor to affect in any measure the scope of the appended claims, it is presently believed that the amide derivatives have a higher polarity than the ester derivatives and that, as a result, the amide spacers can form additional hydrogen bridge bonds and thereby project farther into the aqueous environment of the membrane. The protonated, positively charged amino groups are therefore apparently presented to the negatively charged phosphate backbone of the DNA very effectively for a complexation. On the other hand, the spacer may not be too flexible, because it would then interfere with the desired interaction with the DNA [Deshmukh and Huang, 1997]. Apparently the amide spacer with two methylene groups (7, 187%), as compared with the amide spacer with three methylene units (9, 90%), represented a good compromise between the length required for the bond and the rather destructive flexibility. In comparison with the two ester derivatives as well, transfection efficiency of the spacer with two methylene units (6), at 50%, was twice as high as that of the spacer with three methylene units (8, 25%). The good transfection efficiencies of lipid 7 found here therefore confirm the good transfection properties of this compound, which were described previously [Farhood et al., 1992].

Transfection Results of Bicationic Lipids with One Lipid Anchor

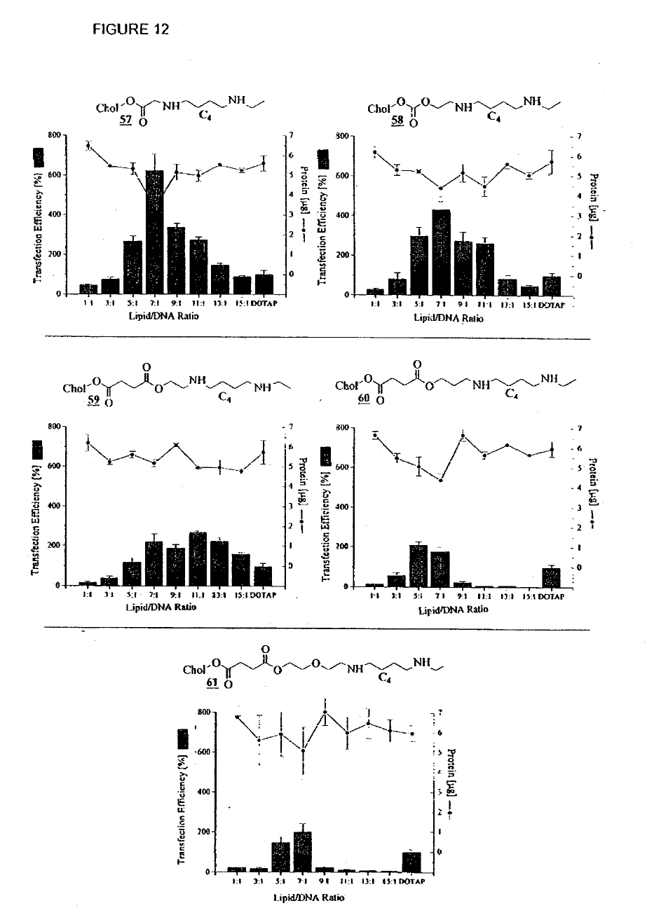

Lipids with Varied Spacers: The transfection results of the bicationic lipids varied in the spacer are presented in FIG. 12. All lipids in this group have the same head group structure with the natural distance of 4 methylene groups between the amino groups (putrescine). Diamines with this distance are known for their ability to interact intensively with DNA [Balasundaram and Tyagi, 1991].

Comparison of Transfection Efficiencies:

The transfection efficiencies of all the bicationic lipids presented here are much higher than those of DOTAP, the standard lipid. The transfection data revealed that the spacer structure has a clear effect on the transfection efficiency: the lipids with the relatively apolar acetyl and carbonate spacers exhibited higher transfection efficiencies (57: 625%, 58: 431%) than the lipids with the polar, longers succinyl spacers (59: 269%, 60: 201%, 61: 203%).

In the group of lipids with succinyl spacers, lipid 59, which has the shortest succinyl spacer compared with the other lipids, exhibited the highest transfection efficiencies. Extending the alkyl chain between the succinyl unit and the first amino group from 2 to 3 methylene groups (60), or also inserting an ethylene glycol unit (61), had a negative effect on the efficiency. While it is not the intention of the applicant to be bound to any particular theory, nor to affect in any measure the scope of the appended claims, it is presently believed that an unfavorable, excessive flexibility is made possible by the longer succinyl spacers, which interferes with the desired interaction with the DNA [Deshmukh and Huang, 1997].

Comparison of Transfection Profiles

A common feature of all the transfection profiles shown in FIG. 12 was the fact that lipoplexes with a lipid/DNA ratio of from about 5:1 to 7:1 achieved the highest transfection efficiencies. This corresponds to a molar ratio of lipid to DNA base of about 3:1, a ratio that was also found in the transfection profiles of the simple cationic lipids. While it is not the intention of the applicant to be bound to any particular theory, nor to affect in any measure the scope of the appended claims, the following discussion on the extent of amine protonation is proffered solely for the purpose of illustration and explanation. It is presently believed that just one amino group per lipid was present in protonated form and was used for the complexation with the DNA. Two possible causes can be considered: first, the acetyl spacer used to prepare bicationic lipids exerts a strong electron attraction on the directly adjacent, secondary amino group, as discussed in context with the simple cationic lipids. In the case of the acetyl derivative, it is therefore suspected that the secondary amino group adjacent to the acetyl spacer is not protonated under the selected pH conditions of 7.4 (see Section 3.2.1.1). Secondly, a simple protonation in the case of the other spacer variations is also feasible: it should be taken into account that the amino group adjacent to the spacer experiences a decrese in alkalinity simply due to the arrangement on a membrane surface [Eastman et al., 1997; Zuidam and Barenholz, 1997; Zuidam and Barenholz, 1998]. The preceding discussion of the extent of amine protonation was provided solely by way of illustration and explanation, and is not intended to limit the scope of the appended claims.

Comparison of Cytotoxicities

In the case of all lipids with varied spacers it was observed that such lipoplexes that lead to increased transfection efficiency were also characterized by an increase in cytotoxic effects (decrease in protein quantity by up to 30%). This effect was especially pronounced with acetyl derivative 57. Apparently an increased uptake of lipoplexes not only led to elevated transfection efficiencies, but to increased cell damage as well. The underlying data did not unequivocally point to the cationic lipids as the sole cause of the toxicity, because equimolar mixtures of cationic lipid and DOPE, the helper lipid, were always used for transfection experiments. The neutral zwitterionic lipid DOPE is known for the fact that it can initiate membrane perturbation processes such as membrane fusions, because it cannot form a bilayer itself [Litzinger and Huang, 1992]. Such processes may also be responsible for the cytotoxicities, because an increased uptake of DOPE in the cells can be correlated with an increased transfection efficiency.

Lipids with Varied Head Groups

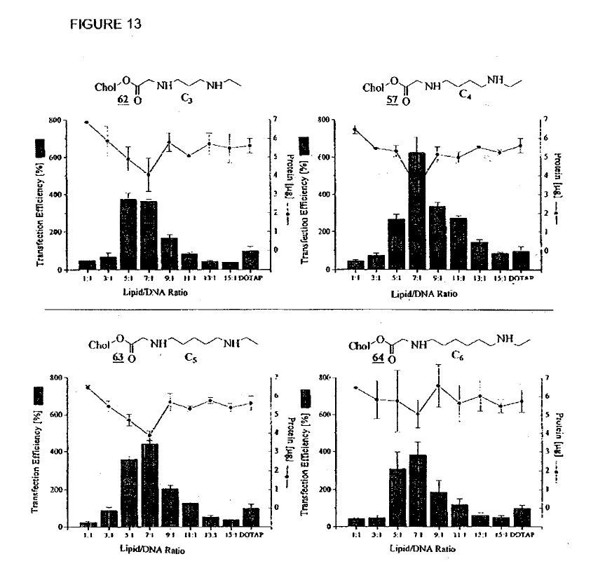

The acetyl spacer was selected based on the transfection results presented previously to synthesize lipids varied in the head group. Although the lipid with the acetyl spacer was characterized by increased cytotoxicity, it proved to be the most effective compound. The transfection diagrams of the bicationic lipids that are systematically varied in terms of the distance between the amino groups (3-6 methylene groups) are shown in FIG. 13.

The acetyl spacer is a very suitable structural unit for synthesizing effective transfection lipids. This was confirmed by the transfection results of all lipids with the various head group variations. Apparently, a distance of 4 methylene groups proved to be the most effective structural unit (57: 625%). Deviating from 4 methylene groups as the distance between the amino groups led to a decrease in the maximum transfection efficiency achieved to 377% (62), 44% (63), and 385% (64), respectively.

While it is not the intention of the applicant to be bound to any particular theory, nor to affect in any measure the scope of the appended claims, it is presently believed that due to the strong electron attraction by the acetyl spacer, the terminal secondary amino group is present in protonated form (see above). The amino group that is adjacent to the acetyl spacer and that is not overly protonated therefore did not interact directly with the DNA. Rather, it must be assigneded to the spacer unit. The amino group therefore increases the polarity of the spacer unit and the lipids are therefore different only in terms of the length of the alkyl chain, on the end of which the protonable amino group is located. These differences must therefore also be responsible for the different transfection efficiencies. The preceding discussion of the extent of amine protonation was provided solely by way of illustration and explanation, and is not intended to limit the scope of the appended claims.

The transfection profiles of the lipids with head group variations varied from each other only slightly and confirm the results of the experiments with lipids with spacer variations: lipoplexes with bicationic lipids were especially effective at a lipid/DNA ratio of from 5:1 to 7:1, although they exhibited elevated levels of cytotoxicity at these ratios.

Transfection Results of Bicationic Lipids with Two Lipid Anchors

The structures of the compounds with two lipid anchors therefore differ from the structures of the bicationic lipids described above, whereby the ethyl group of the terminal amino group is substituted with an additional lipid component, which results in a symmetrical arrangement.

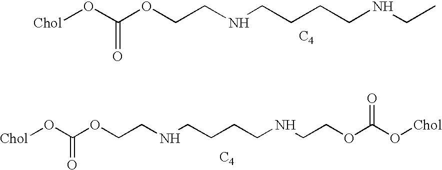

Lipid with One Lipid Anchor

Lipid with Two Lipid Anchors

Lipids of this type of compounds were especially interesting for studies of structure/effect relationships because they contain two amino groups with the same chemical environment. The transfection results should therefore provide important findings about the effect of the amino group adjacent to the spacer. They should be transferrable to the lipids with a lipid anchor described previously, because one of the two amino groups has the same chemical environment.

Lipids with Acetyl Spacers

None of synthesized acetyl derivatives with two lipid anchors formed liposomes in mixtures with DOPE, nor were transfections successful.

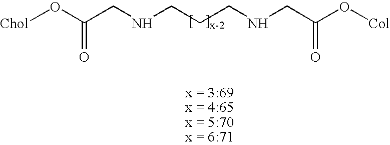

Apparently, varying the number of methylene units between the two amino groups did not affect the protonability of the amino groups. While it is not the intention of the applicant to be bound to any particular theory, nor to affect in any measure the scope of the appended claims, it is presently believed that the acetyl spacer is responsible for affecting the protonability due to its strong electron attraction. This result concurred with the experiences collected in the studies using simple cationic lipids; liposomes could not be prepared with lipid 4 either, because the tertiary amino group is not protonated due to the strong electron attraction by the acetyl spacer. It was therefore demonstrated that the amino group adjacent to the acetyl spacer in the bicationic acetyl derivatives that carry just one lipid anchor is not protonated and therefore cannot enter into an interaction with the DNA. These are therefore lipids with likely just one positive charge per compound.

Lipids with Carbonate Spacers and Succinyl Spacers

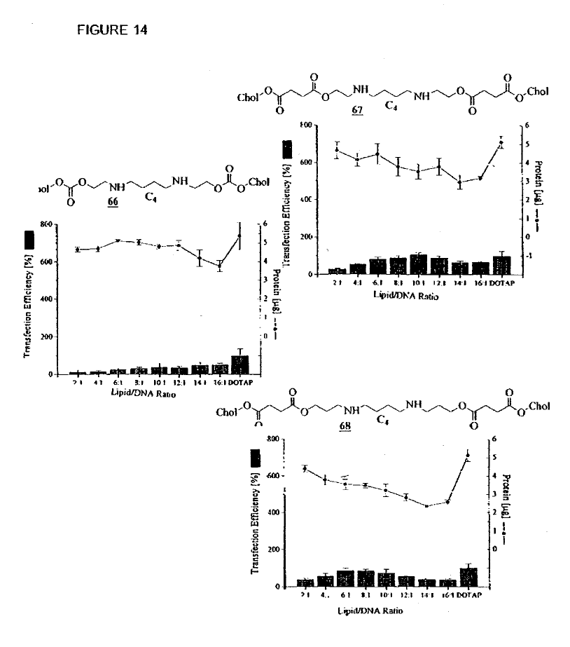

Lipids with succinyl spacers (67) and (68) and with the carbonate spacer (66) were used successfully in mixture with DOPE to prepare liposomes. From this it can be deduced that the succinyl spacer or the carbonate spacer exerts just a slight electron attraction on the adjacent amino group and therefore allow protonation of the amino group adjacent to the spacer. This also conformed the results of the studies carried out using the homologous, simple cationic lipids. Based on the transfection efficiencies of the three lipids that were obtained (FIG. 14), even though they were lower, it also became clear that the protonated head groups enter into interaction with the DNA, which resulted in an uptake of lipoplexes in the cell.

The transfection efficiencies of the three lipids were 52% (66), 87% (68), and 107% (67). The lipids with the succinyl spacers, which were longer and more polar as compared with the carbonate spacer, were somewhat more effective. While it is not the intention of the applicant to be bound to any particular theory, nor to affect in any measure the scope of the appended claims, it is presently believed that a possible explanation for this is the better interaction between the amino groups and the DNA. Toxic side effects occurred with all three lipids as the lipid/DNA ratios of the respective lipoplexes increased. This is indicative of increasing destabilization of the cell membrane due to high lipid concentrations.

The preceding results confirmed the basic protonability of the amino group adjacent to the succinyl and carbonate spacer, even in the bicationic acetyl derivatives with a lipid anchor that should therefore be available for a complexation with the DNA lipids.

Transfection Results of Tricationic Lipids

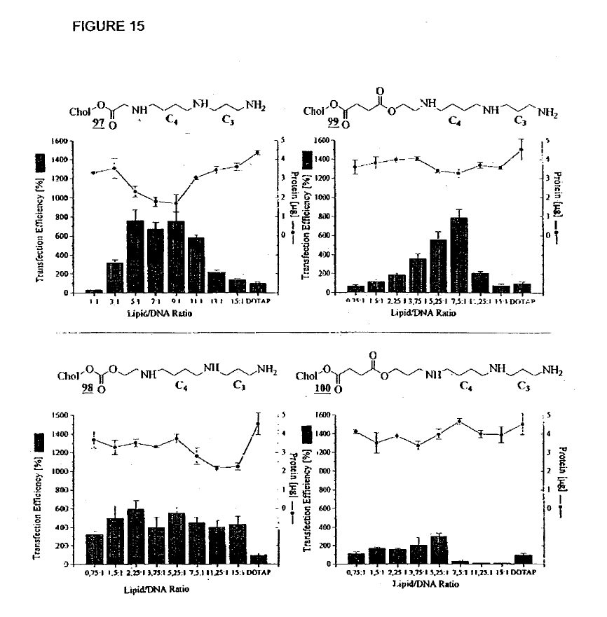

Lipids with Varied Spacers: All lipids in this group carry the natural spermidine unit with distances of between 4 and 3 methylene units as their common structural element. Spermidine, a natural polyamine, carries three positive charges under the pH conditions used (pH 7.4) (pKₐ values of all amino groups are above 8 [Aikens et al., 1983]), and is known for its natural ability to effectively complex DNA due to its strong interaction with it. Additionally, transfection lipids were described previously that contain spermidine as the head group and exhibit good transfection efficiencies [Cooper et al., 1998; Guy-Caffey et al., 1995]. The transfection results of the tricationic lipids varied in the spacer are shown in FIG. 15.

Comparison of Transfection Efficiencies:

All tricationic lipids studies had transfection efficiencies that were much higher than that of DOTAP, the standard lipid. The transfection data revealed that the spacer has a direct effect on the transfection efficiency: the lipids with the apolar acetyl spacer (97) and the succinyl spacer with two methylene uints in the alkyl chain (99) were the most effective compounds in this group, with transfection efficiencies of 763% and 789%, respectively. The fact that the acetyl derivative 97, in contrast to the succinyl derivative 99, likely only allows protonation of two amino groups due to the electron attraction by the spacer had to be taken into account. Apparently this did not decrease the transfection efficiency. With a transfection efficiency of 598%, the carbonate derivative (98), which is similar to the acetyl derivative in terms of the polarity of the spacer, exhibited very high transfection efficiencies. Although the succinyl derivative with three methylene units in the alkyl chain (100) is very similar to the homologous succinyl derivative 99 in terms of chemical structure, the maximum transfection efficiencies, at 303%, were significantly reduced (by more than half.

Comparison of Transfection Profiles

All lipids in this group exhibited maximum transfection efficiencies at lipid/DNA ratios of 7.5:1 (except for 100: 5.25:1). This was described previously in the literature for a lipid with spermidine as the head group [Moradpour et al., 1996]. The transfection profile of the succinyl derivative 99 exhibited a clear maximum transfection efficiency at a lipid/DNA ratio of 7.5:1. Deviations from this optimal ratio of 7.5:1 to 11.25:1, for instance, led to a clear reduction in transfection efficiency, from 789% to about 200%. The transfection profiles of the acetyl derivative (97) and the carbonate derivative (98) were different: both lipids form a clear plateau in terms of transfection efficiencies. This property was especially pronounced with the carbonate derivative 98, however, which exhibited very high transfection efficiencies at all ratios.

Comparison of Cytotoxicities:

On the one hand, the acetyl derivative 97 and the succinyl derivative 99 exhibited similarly high maximum transfection efficiencies. On the other hand, both lipids differ clearly in terms of cytotoxicity due to the various types of lipoplexes. Lipoplexes of the acetyl derivatives that had maximum transfection efficiency led to a reduction in the protein quantity to 50%. The succinyl derivative 99, on the other hand, only exhibited an insignificant reduction in the quantity of total protein. While it is not the intention of the applicant to be bound to any particular theory, nor to affect in any measure the scope of the appended claims, it is presently believed that the low cytotoxicity of the succinyl derivative 100 with the three methylene units in the alkyl chain could be explained by the fact that, due to the lower transfection efficiency of these lipids, less potentially toxic cationic lipid entered the cells. The course of cytotoxicities for the different lipoplexes of the carbonate derivative 98 did not exhibit this close relationship between transfection efficiency and cytotoxicity: while the lipoplexes with low lipid/DNA ratios, which were already very effective, were hardly toxic, the toxicity began to increase at higher ratios. In this case as well, the cell-damaging effect was likely due to the increased quantity of lipid that was taken up by the cell.

Lipids with Varied Head Groups

Based on the transfection results of lipids with a varied spacer structure, the acetyl spacer was selected as a suitable spacer for preparing the lipids that are varied in terms of the head group. Although lipid 97 led to similarly high transfection efficiencies like lipid 99 and also exhibited lower levels of cytotoxicity, it had a very broad plateau with high transfection efficiencies. This was the deciding factor in the selection of the spacer. All lipids in this group therefore contained the acetyl spacer and differed in terms of the structure of their head groups: the number of methylene groups varied from 3 to 6 between the first two amino groups, and amounts to 2 and 3 methylene units between the last two (terminal) amino groups, respectively.

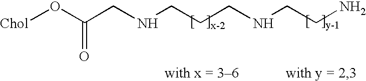

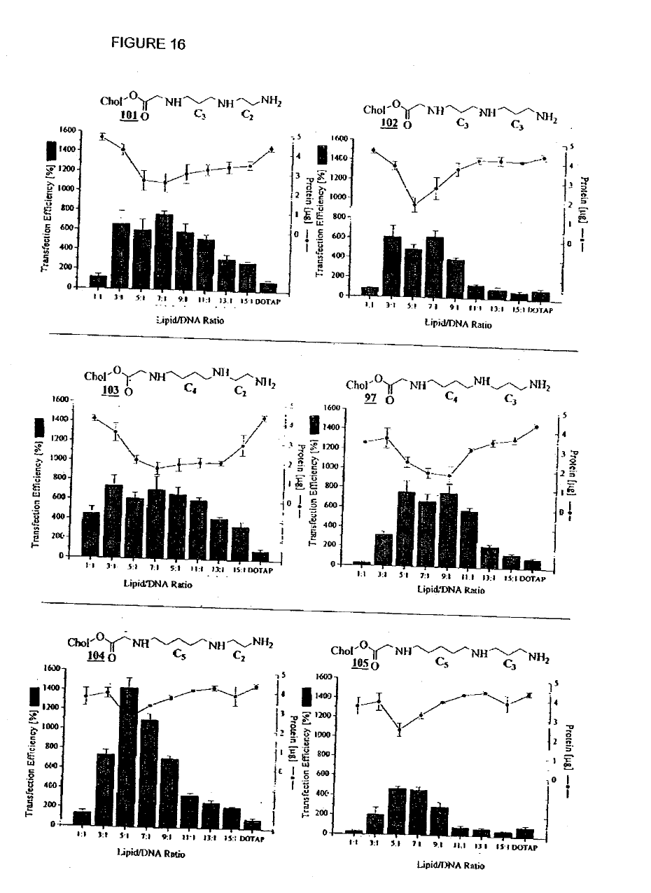

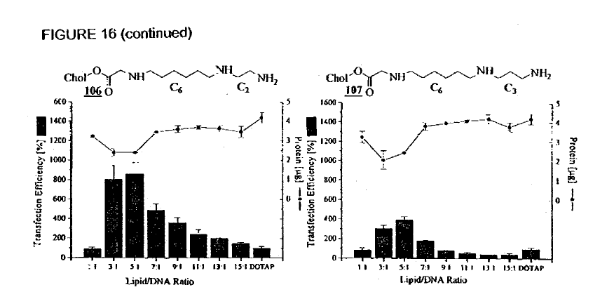

Out of the combination of the two variable distances between the amino groups we arrive at 8 potential structural variations, the transfection properties of which are illustrated in FIG. 16:

All compounds exhibited much, much higher transfection efficiencies than DOTAP, the standard lipid, but they clearly differed in terms of the structure of the head group. To simplify the comparison of the transfection results of the various lipids, lipids with the same distance between the first two amino groups will be compared first, and then the lipids with the same distance between the last two amino groups will be discussed.

Comparison of the Transfection Efficiencies of Lipids with the Same Distance Between the First Two Amino Groups

In comparing these lipids, it became clear that reducing the distance between the last two amino groups from 3 to 2 methylene groups had a positive effect on the maximum transfection efficiency; the lipid pair with 3 (101: 769%, 102: 626%), 5 (104: 1428%, 105: 474%), and 6 (106: 860%, 107: 390%) methylene units between the first two amino groups exhibited this dependency, wherease the structural variations in the lipid pair with 5 methylene units led to a particularly dramatic increase in maximum transfection efficiency (by a factor of 3). Lipids 103 and 97, with 4 methylene groups between the first two amino groups, were the exceptions. They led to similar transfection efficiencies (103: 742%, 97: 763%).

As described, amino groups in polyamines have different pKₐ values, depending on the number of amino groups and the number of methylene groups between the amino groups [Bergeron et al., 1995; Bernardo et al., 1996; Aikens et al., 1983; Takeda et al., 1983]. For instance, the protonation of one of two adjacent amino groups led to a reduction in the pKₐ value of the second amino group and, therefore, to a more difficult complete protonation of all amino groups. While it is not the intention of the applicant to be bound to any particular theory, nor to affect in any measure the scope of the appended claims, the following discussion on the protonation of amino groups and DNA bond strengths is proffered solely for the purpose of illustration and explanation. Based on the studies described in the literature, the last two amino groups, which are separated by an alkyl chain with 3 methylene groups, should have pKₐ values above 8. Under the pH conditions used (pH 7.4), these two amino groups are very likely present in a protonated state. Since the first amino group adjacent to the acetyl spacer is not protonatable due to the strong electron attraction, lipids in this group have a head group with 2 positive charges.

Due to the natural distance of 3 methylene groups between the two charges, these lipids should enter into an interaction with DNA very well. Head groups with two amino groups that are separated by an alkyl chain with 2 methylene groups should carry just one positive charge under physiological pH conditions. This is demonstrated via the determination of the pKₐ values of amino groups with polyethylene imines as the head group [Geall et al., 1998].

Diamines with a distance of 2 methylene groups are characterized by a lower DNA bond strength than the natural diamines with 3 and 4 methylene groups as the distance between the amino groups [Balasundaram and Tyagi, 1991]. As a result, a lower bond strength would correlate with an elevated transfection efficiency for the lipids described here. This relationship may be explained by a more efficient release of DNA from the lipoplexes within the cell. This release of DNA is considered to be a limiting step for successful transfection [Xu and Szoka, 1996]. The preceding discussion on the protonation of amino groups and DNA bond strengths was provided solely by way of illustration and explanation, and is not intended to limit the scope of the appended claims.

Comparison of Transfection Efficiencies of Lipids with the Same Distance between the Last Two Amino Groups

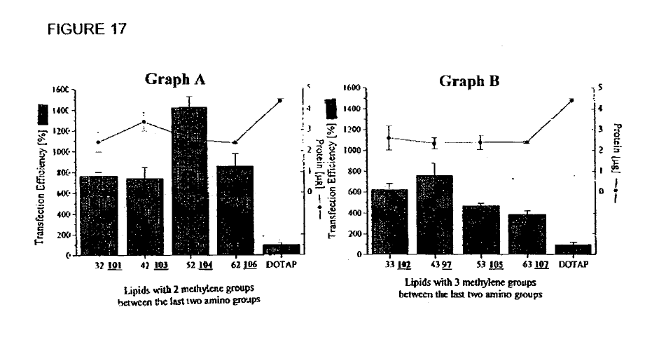

As demonstrated by a comparison of maximum transfection efficiencies within the group of lipids with 2 methylene groups between the last two amino groups, a distance of 5 (104: 1428%) methylene groups led to the highest transfection efficiencies (Graph A in FIG. 17). The lipids with 3 (101: 769%), 4 (103: 742%), and 6 (106: 860%) methylene groups as the distance, on the other hand, exhibited much lower transfection efficiencies that differed from each other only slightly.

In the group of lipids with 3 methylene groups between the last two amino groups, the compound with 4 (97: 763%) methylene groups between the first two amino groups exhibited the highest transfection efficiency (Graph B in FIG. 17). Lipids with 3 (102: 626%), 5 (105: 474%), and 6 (107: 390%) methylene groups led to lower transfection efficiencies. A trend towards a clear reduction in transfection efficiency of a lipid varied in this manner as the deviation from the efficient distance of 4 methylene groups increased was observed.

Transfection Results of DMG Derivatives

The transfection results of the single cationic, bicationic, and tricationic lipids with the 1-(2,3-di-tetradecyloxy)-propanol unit (DMG) as the lipid anchor are described in this section. The head groups are linked with the lipid anchor via the acetyl spacer.

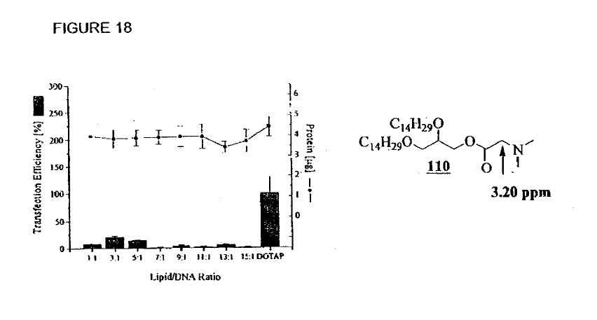

DMG Lipids with a Simple Cationic Head Group: The DMG lipid with a tertiary amino group (110) formed no liposomes in a mixture with the helper lipid DOPE, and also led to only very low transfection efficiencies (FIG. 18).

The chemical shift of the methylene protons adjacent to the amino group, at 3.20 ppm, is higher than the corresponding value of the homologous cholesterol derivative 4 (3.14 ppm). While it is not the intention of the applicant to be bound to any particular theory, nor to affect in any measure the scope of the appended claims, it is presently believed that a protonation of the tertiary amino group can be ruled out due to the strong electron attraction by the acetyl spacer under the pH conditions used. A resultant unfavorable relation of the surface requirement between the lipid anchor and the uncharged amino group apparently does not allow bilayers (liposomes) to form. This explanation was provided solely by way of illustration, and is not intended to limit the scope of the appended claims.

The DMG derivative with a permethylated amino group (111) was used successfully in a mixture with DOPE to prepare liposomes. While it is not the intention of the applicant to be bound to any particular theory, nor to affect in any measure the scope of the appended claims, it is presently believed that the introduction of a permanent, positive charge created a favorable relationship between the polar and the apolar part of the molecule, which makes it possible for lipid bilayers to form. In contrast to lipid 110 with a tertiary amino group, the DMG derivative 111 also led to much higher transfection efficiencies, with 83%.

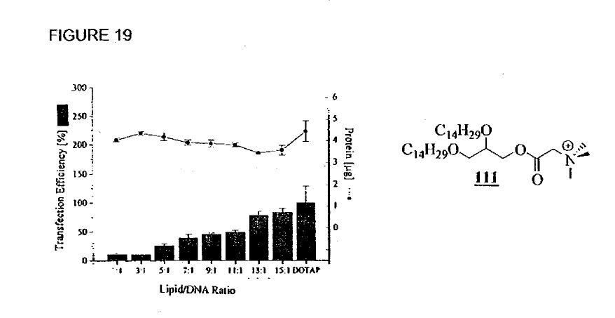

An almost linear increase in transfection efficiency with increasing lipid/DNA ratio was observed. It also correlated with a slight increase in cytotoxicity (FIG. 19).

DMG Lipid with a Bicationic Head Group

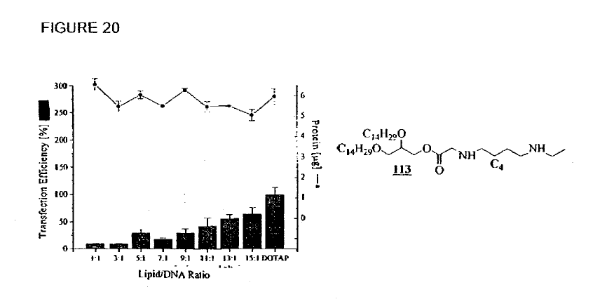

The highest transfection efficiency of DMG lipid 113 with an acetyl spacer and bicationic head group took place with a lipid/DNA ratio of 15:1. It was 65% (FIG. 20).

The transfection profile was similar to that of the simple cationic DMG derivative with a permethylated amino group. While it is not the intention of the applicant to be bound to any particular theory, nor to affect in any measure the scope of the appended claims, it is presently believed that this may be due to the fact that because of the acetyl spacer, only the terminal amino group is protonated under the pH conditions used. Lipid 113, like lipid 111, would then have just one positive charge. An increasing lipid/DNA ratio correlated with an only slight increase in cytotoxicity.

DMG Lipid with Tricationic Head Group

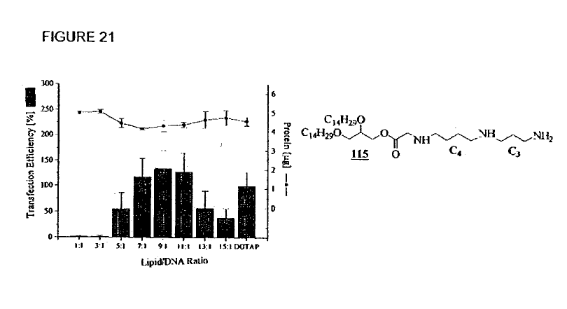

With a transfection efficiency of 135%, the DMG derivative with an acetyl spacer and a tricationic head group (spermidine unit) was more effective than the homologous derivative with a simple cationic or bicationic head group (FIG. 21).

The transfection efficiency of this lipid exhibited a plateau in a range of a lipid/DNA ratio of from 7:1 to 11:1. The transfection profile was therefore similar to the profiles characterized for many tricationic cholesterol derivatives.

The transfection results of the DMG derivatives showed that the 1-(2,3-di-tetradecyloxy)-propanol unit (DMG) can also be used as a lipid anchor to synthesize transfection lipids. In comparison with their homologous cholesterol derivatives, however, the transfection efficiencies were much lower. This must be due to the lipid anchor that was selected. While it is not the intention of the applicant to be bound to any particular theory, nor to affect in any measure the scope of the appended claims, it is presently believed that this is probably due to changes in the biophysics, e.g., the fluidity.

Summary and Outlook

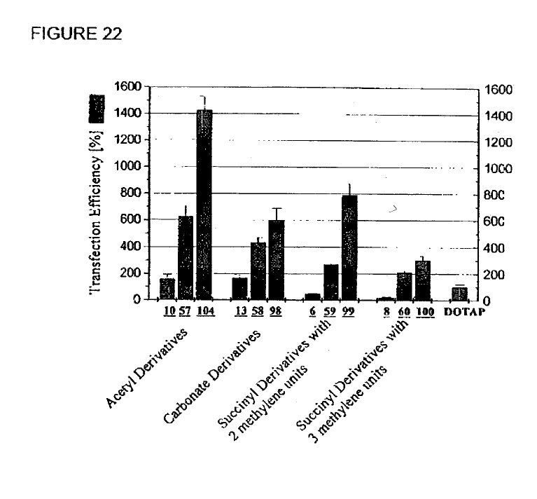

The transfection properties of all lipids were investigated in a standardized transfection assay. A comparison of transfection results revealed the presence of clear relationships between the structures of the spacers and the head groups as well as the transfection properties of the cationic lipids (FIG. 22, transfection efficiencies given in % based on commercially available liquid DOTAP, i.e., 100%):

Lipids with the relatively apolar and short acetyl spacers were therefore the most effective compounds in the class of lipids with one, two, or three amino group(s). The homologous carbonate derivatives—which are also relatively apolar—and, mainly, the polar and longer succinyl derivatives led to lower transfection efficiencies.

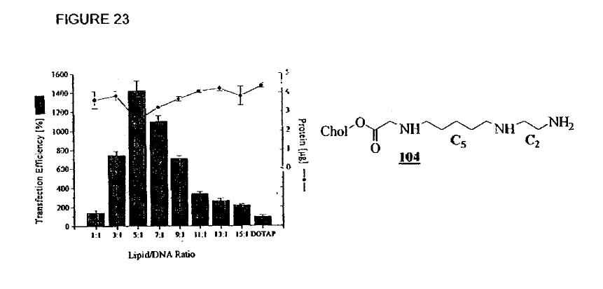

Additionally, the transfection efficiency of lipids correlated with the number of amino groups in the head groups. In all compound groups with the same spacer, lipids with three amino groups have the highest transfection efficiency. The distance between the amino groups also had a strong effect on the transfection properties of the lipids. In the triamino head groups, a distance of two methylene groups between the last two amino groups was optimal. The optimal structural elements are combined in the structure of lipid 104 (FIG. 23), which resulted in high transfection efficiencies. The positive correlation between transfection efficiency and the number of amino groups in the head group is apparently not dependent on the complete protonation of all amino groups. While it is not the intention of the applicant to be bound to any particular theory, nor to affect in any measure the scope of the appended claims, it is presently believed that only one amino group was protonated in lipid 104, for instance. It contains three amino groups and exhibited very good transfection efficiencies.

The achievement of high transfection efficiencies using lipids, the amino acid groups of which are not completely protonated, leads to the speculation that these uncharged amino groups could be substituted with other polar groups. A substitution of this nature could lead to transfection lipids that are less toxic and exhibit higher rates of transfection efficiency. Based on the results of this study, a greater number of amino groups in the head group should also lead to improved transfection properties. Additionally, the systematic variation of the lipid anchor is a promising way to optimize transfection properties. The synthesis strategies developed here can be used to realize the additional preferred embodiments of the present invention.

The manner in which a cationic amphiphile embodying features of the present invention is made, and the process by which it is used to transport a biologically active molecules into a cell, will be abundantly clear to one of ordinary skill in the art based upon joint consideration of both the preceding discussion, and the following representative protocols.

Abbreviations and Glossary

| | |
|---|---|
| AcOH | Acetic acid |
| Assay | Test |
| Bilayer | Lipid bilayer |
| BCA | Bicinchoninic acid |
| Bn | Benzyl protective group |
| Boc | tert-butyloxycarbonyl protective group |
| BSA | Bovine serum albumin |
| CH | Cyclohexane |
| CHCl₃ | Chloroform |
| CH₂Cl₂ | Dichlormethane |
| DC-Chol | 3□-[N-(N',N'-dimethylaminoethyl)carbamoyl]-cholesterol |
| DMAP | 4-Dimethylaminopyridine |
| DMRIE | N-(1,2-dimyristyloxypropyl)-N,N-dimethyl-N- |
| | hydroxyethylammoniumbromide |
| DMSO | Dimethyl sulfoxide |
| DOGS | N,N-dioctodecyl-amidoglycylspermine |
| DOPE | 1,2-dioleoyl-sn-glycero-3-phosphatidylethanolamine |
| DORI | N-(1,2-dioleoyloxypropyl)-N,N-dimethyl-N- |
| | hydroxyethylammoniumbromide |
| DOTAP | N-[1-(2,3-dioleoyloxy)propyl]-N,N, |
| | N-trimethylammoniumchloride |
| DOTMA | N-[1-(2,3-dioleyloxy)propyl]-N,N, |
| | N-trimethylammoniumchloride |
| EE | Ethyl acetate |
| eq. | Molar equivalent |
| HCO₂H | Formic acid |
| HEPES | 4-(2-hydroxyethyl)-piperazine-1-ethane sulphonic acid |
| MeOH | Methanol |
| Mes | Mesylate group (methanesulphonyl group) |
| PBS | Phosphate buffer |
| Pd-C | Palladium-carbon catalyst |
| RF | Reflux |
| PG | Protective group |
| Spacer | Chemical linker between a lipid anchor and a head group |
| SpdC | Spermidine cholesterol |
| THF | Tetrahydrofuran |
| well | Well in a microtiter plate |
| Z | Benzyloxycarbonyl protective group |

Materials and Methods

Instruments, Materials, and Reagents for Synthetic Procedures Thin-Layer Chromatography

Ready-to-use thin-layer chromatography plates silica gel 60 (with and without fluorescence indicator F₂₅₄) from Merck, Darmstadt

Detection via Fluorescence Quenching

Viewed under UV light (254 nm)

Detection via Dipping Solutions

After dipping the thin-layer chromatography plate, warm to 100-600° C. using the hot air blower as necessary.

1. 2% methanolic sulphuric acid

2. Molybdenum blue reagent [Dittmer and Lester, 1964]

Solution A: 40 g ammonium molybdate, 150 ml water, 350 ml 98% sulphuric acid

Solution B: 900 mg molybdenum powder, 250 ml solution A Detection solution:30 ml solution A, 30 ml solution B, 120 ml water

3. Ninhydrin

6 mg ninhydrin in 12 ml ethanol

A red color develops in the presence of heat if primary and secondary amines are present. A yellow color develops if tertiary amines are present.

4. Anisaldehyde

22 ml anisaldehyde, 800 ml ethanol, 14.4 ml concentrated sulphuric acid, 8.8 ml acetic acid

Column Chromatography

Silica gel 60 for column chromatography (particle size 0.063-0.100 mm) from Merck, Darmstadt

¹H-NMR Spectroscopy

250 MHz Bruker AC 250 or 400 MHz Bruker AM 400

Tetramethylsilane (δ=0 ppm) was used as the internal standard. Deuterated chloroform (CDCl₃), methanol (CD₃OD) and water (D₂O) were used as solvents.

Only the signals from cholesterol for compound 1 are presented in order to present the synthesis procedures with the corresponding ¹H-NMR data in compact form. This is permissible because the position of the signals for cholesterol was the same in all compounds.

Solvents and Chemicals

Solvents and chemicals from Merck and Fluka were used in the synthesis procedures.

Triethylamine was dried over and distilled from calcium oxide. Chloroform, dichloromethane, tetrahydrofuran, and toluene were dried over 4 A molecular sieves. Ethyl acetate was dried over sodium sulphate.

Synthetic Procedures for Simple Cationic Lipids

Chloroacetic acid cholesterylester (1):

Slowly add a solution of 9.54 ml (120 mmol) chloroacetylchloride in 50 ml dichloromethane in drops to a solution of 38.67 g (100 mmol) cholesterol and 20.79 ml (150 mmol) triethylamine in 150 ml dichloromethane under refrigeration. Stir the formulation overnight at room temperature and then extract twice with 100 ml 2 N HCl each time. Remove the solvent and stir the residue in 200 ml dichloromethane with 5 g activated charcoal at room temperature. Filter off the activated charcoal, remove the solvent, and purify the residue via column chromatography on 50 g silica gel (dichloromethane). The yield is 45.04 g 1 as a colorless solid.

| | |
|---|---|
| Yield: | 45.04 g(97% of theoretical value) |
| Mᵣ: | 463.14(C₂₉H₄₇ClO₂) |
| R_{f}: | 0.69(cyclohexane/ethyl acetate 4:1) |
| ¹H-NMR | |
| (250 MHz, CDCl₃): | |
| Cholesterol | |
| signals: | |
| δ=0.67 | (s, 3H, CH₃-18) |
| δ=0.86 and 0.88 | (2d, ³J = 6.6 Hz, 6H, diastereotope CH₃-26/27) |
| δ=0.92 | (d, ³J = 6.4 Hz, 3H, CH₃-21) |
| δ=0.94-1.62 | (m, 21H) |
| δ=1.04 | (s, 3H, CH₃-19) |
| δ=1.78-1.89 | (m, 3H) |
| δ=1.93-2.05 | (m, 2H) |
| δ=2.33 and 2.36 | (2d broad, 2H, allyl. CH₂-4) |
| δ=4.62-4.78 | (m, 1H, CH-3) |
| δ=5.34-5.41 | (m, 1H, vinyl. CH-6) |
| Non-cholesterol | |
| signals: | |
| δ=4.04 | (s, 2H, OCOCH₂Cl) |

Cholesterylhemisuccinate (2)

Warm a solution of 38.7 g (100 mmol) cholesterol, 20.0 g (200 mmol) succinic acid anhydride, 0.6 g (5 mmol) DMAP and 51.2 ml (400 mmol) triethylamine in 500 ml ethyl acetate for 12 hours with reflux. Add 100 ml ethyl acetate and 100 ml methanol, then extract with 200 ml 2 N HCl. Add an additional 100 ml ethyl acetate, then extract the organic phase twice with 150 ml each time of a mixture (2:1) of 0.2 N HCl and methanol. Concentrate the organic phase to a small volume and take up the residue in 300 ml methanol. Stir the resultant suspension for 15 minutes at room temperature. Add 300 ml water to completely precipitate the product. Siphon off the raw product, wash twice with 200 ml water each time, vacuum-dry, then recrystallize out of 200 ml diisopropyl ether. The yield is 43.4 g 2 as a colorless powder.

| | |
|---|---|
| Yield: | 43.4 g(89% of theoretical value) as solid |
| Mᵣ: | 486.74(C₃₁H₅₀O₄) |
| R_{f}: | 0.38(ethyl acetate) |
| ¹H-NMR | |
| (CDCl₃, 400 MHz): | |
| Non-cholesterol signals: | |
| δ=2.58-2.70 | (m, 4H, —OCO(CH₂)₂COO—) |

Cholesterylhemisuccinate (3)

Slowly add 12.7 ml (175 mmol) thionylchloride in drops to 34.1 g (70 mmol) cholesterylhemisuccinate (2) in 200 ml toluene under refrigeration. Stir for 2 hours at an oil bath temperature of 90° C. A clear solution is produced that remains clear in cold conditions. Remove excess thionylchloride and any SO₂ and HCl produced using a slight vacuum (caution: delay in boiling!). Remove the toluene completely. Cholesterylhemisuccinoylchloride is obtained as red crystals. Use them to make a 0.5 M stock solution in toluene. To calculate the quantity of solvent to use, assume a 100% reaction for the acid chloride preparation.

| | |
|---|---|
| Mᵣ: | 505.21(C₃₁H₄₉ClO₃) |
| R_{f}: | 0.21(ethyl acetate) |

Synthesis Procedures for Lipids with Tertiary Amino Groups

N-Cholesteryloxycarbonylmethyl-N,N-dimethylamine (4)

Slowly add a solution of 4.63 g (10 mmol) chloroacetic acid cholesterylester (1) in 10 ml toluene in drops to 17.9 ml (100 mmol) of a 5.6 molar solution of dimethylamine in ethanol under refrigeration. Stir overnight at room temperature, then concentrate the formulation to a small volume. Take up the residue in 100 ml dichloromethane and extract twice against 1 N hydrochloric acid. Remove the solvent-and purify the residue via column chromatography on 60 g silica gel. Elute apolar impurities with cyclohexane/ethyl acetate (6:1) and the product with cyclohexane/ethyl acetate (2:1).

| | |
|---|---|
| Yield: | 2.5 g(53% of theoretical value) as a colorless solid |
| Mᵣ: | 471.77(C₃₁H₅₃NO₂) |
| R_{f}: | 0.19(cyclohexane/ethyl acetate 1:1) |
| ¹H-NMR | |
| (250 MHz, CDCl₃): | |
| Non-cholesterol | |
| signals: | |
| δ=2.35 | (s, 6H, —N(CH₃)₂) |
| δ=3.14 | (s, 2H, —OCOCH₂N(CH₃)₂) |

N-(2-Cholesteryloxycarbonyloxy-ethyl)-N,N-dimethylamine (5)

Add a solution of 4.5 g (10 mmol) cholesterylchloroformiate in drops to a solution of 1.3 ml (12 mmol) 2-(dimethylamino)-ethanol and 4.2 ml (30 mmol) triethylamine in 50 ml dichloromethane under refrigeration. Stir for 30 minutes, then extract twice against 1 N hydrochloric acid and then once against 1 N sodium hydroxide solution. Concentrate the organic phase to a small volume, then purify the residue via column chromatography on 60 g silica gel. Elute apolar impurities with cyclohexane/ethyl acetate (1:1), and elute the product with ethyl acetate/methanol (9:1).

| | |
|---|---|
| Yield: | 4.0 g(80% of theoretical value) as a colorless solid |
| Mᵣ: | 501.79(C₃₂H₅₅NO₃) |
| R_{f}: | 0.11(ethyl acetate) |
| ¹H-NMR | |
| (250 MHz, CDCl₃): | |
| Non-cholesterol | |
| signals: | |
| δ=2.29 | (s, 6H, —N(CH₃)₂) |
| δ=2.59 | (t, ³J = 5.8 Hz, 2H, —OCH₂CH₂N(CH₃)₂) |
| δ=4.21 | (t, ³J = 6.0 Hz, 2H, —OCH₂CH₂N(CH₃)₂) |

N-(cholesterylhemisuccinoyloxy-2-ethyl)-N,N-dimethylamine (6)

Add 4 ml (2.0 mmol) of a 0.5 molar stock solution of cholesterylhemisuccinoylchloride (3) in toluene by drops to a solution of 241 pi (2.4 mmol) 2-(dimethylamino)-ethanol and 832 μl (6.0 mmol) triethylamine in 10 ml dichloromethane under refrigeration. Stir for 60 minutes, then extract twice against 1 N hydrochloric acid and then once against 1 N sodium hydroxide solution. Concentrate the organic phase to a small volume. Recrystallize the residue from 10 ml acetonitrile.

| | |
|---|---|
| Yield: | 692 mg(62% of theoretical value) as a |
| | colorless solid |
| Mᵣ: | 557.86(C₃₅H₅₉NO₄) |
| R_{f}: | 0.36(chloroform/methanol/ammonia (25%) 90:10:1) |
| ¹H-NMR | |
| (250 MHz, CDCl₃): | |
| Non-cholesterol | |
| signals: | |
| δ=2.28 | (s, 6H, —N(CH₃)₂) |
| δ=2.57 | (t, ³J = 5.9 Hz, 2H, —OCH₂CH₂N(CH₃)₂) |
| δ=2.57-2.67 | (m, 4H, —OCO(CH₂)₂COO—) |
| δ=4.19 | (t, ³J = 5.8 Hz, 2H, —OCH₂CH₂N(CH₃)₂) |

N-(cholesterylhemisuccinoylamino-2-ethyl)-N,N-dimethylamine (7)

Add 2 ml (1.0 mmol) of a 0.5 molar stock solution of cholesterylhemisuccinoylchloride (3) in toluene in drops to a solution of 131 μl (1.2 mmol) 2-(dimethylamino)-ethylamine and 416 μl (3.0 mmol) triethylamine in 10 ml dichloromethane under refrigeration. Stir for 60 minutes, then extract twice against 1 N hydrochloric acid and then once against 1 N sodium hydroxide solution. Concentrate the organic phase to a small volume, then purify the residue via column chromatography on 15 g silica gel. Elute the product with ethyl acetate/methanol (4:1).

| Yield: | 290 mg(52% of theoretical value) as a |
|---|---|
| | colorless solid |
| Mᵣ: | 556.87(C₃₅H₆₀N₂O₃) |
| R_{f}: | 0.15(ethyl acetate/methanol 4:1) |
| ¹H-NMR | |
| (250 MHz, CDCl₃): | |
| Non-cholesterol | |
| signals: | |
| δ=2.28 | (s, 6H, —N(CH₃)₂) |
| δ=2.47 | (t, ³J = 5.9 Hz, 2H, —NHCH₂CH₂N(CH₃)₂) |
| δ=2.48 | (t, ³J = 6.8 Hz, 2H, —OCOCH₂CH₂CONH—) |
| δ=2.64 | (t, ³J = 6.8 Hz, 2H, —OCOCH₂CH₂CONH—) |
| δ=3.35 | (quart, ³J = 5.6 Hz, 2H, —NHCH₂CH₂N(CH₃)₂) |
| □ = 6.15 | (s broad, 1H, —OCOCH₂CH₂CONH—) |

N-(cholesterylhemisuccinoyloxy-3-propyl)-N,N-dimethylamine (8)

Add 2 ml (1.0 mmol) of a 0.5 molar stock solution of cholesterylhemisuccinoylchloride (3) in toluene in drops to a solution of 139 μl (1.2 mmol) 3-(dimethylamino)-propanol and 416 μl (3.0 mmol) triethylamine in 5 ml dichloromethane under refrigeration. Stir for 60 minutes, then extract twice against 1 N hydrochloric acid and then once against 1 N sodium hydroxide solution. Concentrate the organic phase to a small volume then recrystallize the residue out of 8 ml acetonitrile.

| | |
|---|---|
| Yield: | 240 mg(42% of theoretical value) |
| | as a colorless solid |
| Mᵣ: | 571.88(C₃₆H₆₁NO₄) |
| R_{f}: | 0.34(chloroform/methanol/ammonia (25%) 90:10:1) |
| ¹H-NMR | |
| (250 MHz, CDCl₃): | |
| Non-cholesterol | |
| signals: | |
| δ=1.80 | (quint, ³J = 7.0 Hz, 2H, —OCH₂CH₂CH₂N(CH₃)₂) |
| δ=2.22 | (s, 6H, —N(CH₃)₂) |
| δ=2.34 | (t, ³J = 7.4 Hz, 2H, —O(CH₂)₂CH₂N(CH₃)₂) |
| δ=2.58-2.62 | (m, 4H, —OCO(CH₂)₂COO—) |
| δ=4.14 | (t, ³J = 5.8 Hz, 2H, —OCH₂(CH₂)₂N(CH₃)₂) |

N-(cholesterylhemisuccinoylamino-3-propyl-N,N-dimethylamine (9)

Add 8 ml (4.0 mmol) of a 0.5 molar stock solution of cholesterylhemisuccinoylchloride (3) in toluene in drops to a solution of 604 μl (4.8 mmol) 3-(dimethylamino)-propylamine and 1.66 ml (12.0 mmol) triethylamine in 10 ml dichloromethane under refrigeration. Stir for 60 minutes, then extract twice against 1 N hydrochloric acid and then once against 1 N sodium hydroxide solution. Concentrate the organic phase to a small volume then purify the residue via column chromatography on 40 g silica gel. Elute the product with ethyl acetate/methanol (2:1).

| Yield: | 1.46 g(64% of theoretical value) as a colorless solid |
|---|---|
| Mᵣ: | 570.90(C₃₆H₆₂N₂O₃) |
| R_{f}: | 0.18(ethyl acetate/methanol 1:1) |
| ¹H-NMR | |
| (250 MHz, CDCl₃): | |
| Non-cholesterol | |
| signals: | |
| δ=1.80 | (quint, |
| | ³J = 7.0 Hz, 2H, —NHCH₂CH₂CH₂N(CH₃)₂) |
| δ=2.22 | (s, 6H, —N(CH₃)₂) |
| δ=2.34 | (t, ³J = 7.4 Hz, 2H, —NH(CH₂)₂CH₂N(CH₃)₂) |
| δ=2.58-2.62 | (m, 4H, —OCO(CH₂)₂CONH—) |
| δ=4.14 | (t, ³J = 5.8 Hz, 2H, —NHCH₂(CH₂)₂N(CH₃)₂) |
| □ = 6.15 | (s broad, 1H, —OCOCH₂CH₂CONH—) |

Synthesis Procedures for Lipids with a Quaternary Amino Group

N-Cholesteryloxycarbonylmethyl-N,N,N-trimethylammoniummethylsulphate (10)

Add 2.0 ml (21.0 mmol) dimethyl sulphate in drops to a solution of 2.0 g (4.2 mmol) N-cholesteryloxycarbonylmethyl-N,N-dimethylamine (4) in 50 ml acetone. Stir for 15 minutes, then filter off the product-which precipitates out as a colorless precipitate-and rewash with acetone.

| | |
|---|---|
| Yield: | 1.8 g(72% of theoretical value) as a colorless solid |
| Mᵣ: | 597.89(C₃₃H₅₉NO₆S) |
| R_{f}: | 0.40(chloroform/methanol/formic |
| | acid/water 60:40:6:6) |
| ¹H-NMR | |
| (250 MHz, CDCl₃): | |
| Non-cholesterol | |
| signals: | |
| δ=3.49 | (s, 9H, —N(CH₃)₃) |
| δ=3.72 | (s, 3H, CH₃OSO₃⁻) |
| δ=4.49 | (s, 2H, —OCOCH₂N(CH₃)₃) |

N-(2-Cholesteryloxycarbonyloxy-ethyl)-N,N,N-trimethylammoniummethylsulphate (11)

Add 1.0 g (2.0 mmol) N-(2-cholesteryloxycarbonyloxy-ethyl)-N,N-dimethylamine (5) in 30 ml acetone in drops to 1.0 ml (10.5 mmol) dimethyl sulphate. Stir for 15 minutes, then filter off the product—which precipitates out as a colorless precipitate—and rewash with acetone.

| | |
|---|---|
| Yield: | 1.1 g(88% of theoretical value) as a colorless solid |
| Mᵣ: | 627.92(C₃₄H₆₁NO₇S) |
| R_{f}: | 0.40(chloroform/methanol/formic |
| | acid/water 60:40:6:6) |
| ¹H-NMR | |
| (250 MHz, CDCl₃): | |
| Non-cholesterol | |
| signals: | |
| δ=3.36 | (s, 9H, —N(CH₃)₃) |
| δ=3.70 | (s, 3H, CH₃OSO₃⁻) |
| δ=3.87-3.96 | (m, 2H, —OCH₂CH₂N(CH₃)₃) |
| δ=4.54-4.64 | (m, 2H, —OCH₂CH₂N(CH₃)₃) |

N-Cholesteryloxycarbonylmethyl)-N,N-dimethy-N-hydroxyethylammoniumiodide (12)

Add 469 μl (6.0 mmol) 2-iodoethanol in drops to a solution of 283 mg (0.6 mmol) N-cholesteryloxycarbonylmethyl-N,N-dimethylamine (4) in 3 ml acetone. Stir overnight. Filter off the product, which precipitates out as a colorless precipitate, and rewash with acetone.

| | |
|---|---|
| Yield: | 270 mg(70% of theoretical value) as a |
| | colorless solid |
| Mᵣ: | 643.73(C₃₃H₅₈INO₃) |
| R_{f}: | 0.40(chloroform/methanol/formic acid/water 60:40: |
| | 6:6) |
| ¹H-NMR | |
| (250 MHz, | |
| CDCl₃/CD₃OD 6:2): | |
| Non-cholesterol | |
| signals: | |
| δ=3.48 | (s, 6H, —OCOCH₂N(CH₃)₂(CH₂)₂OH) |
| δ=3.82-3.89 | (m, 2H, —OCOCH₂N(CH₃)₂CH₂CH₂OH) |
| δ=3.99-4.06 | (m, 2H, —OCOCH₂N(CH₃)₂CH₂CH₂OH) |
| δ=4.47 | (s, 2H, —OCOCH₂N(CH₃)₂(CH₂)₂OH) |

N-(2-Cholesteryloxycarbonyloxy-ethyl)-N,N-dimethyl-N-hydroxyethylammoniumiodide (13)

Add 469 μl (6.0 mmol) 2-iodoethanol in drops to a solution of 301 mg (0.6 mmol) N-(2-cholesteryloxycarbonyloxy-ethyl)-N,N-dimethylamine (5) in 3 ml acetone. Stir overnight. Filter off the product, which precipitates out as a colorless precipitate, and rewash with acetone.

| | |
|---|---|
| Yield: | 193 mg(49% of theoretical value) |
| | as a colorless solid |
| Mᵣ: | 657.76(C₃₄H₆₀INO₄) |
| R_{f}: | 0.40(chloroform/methanol/formic acid/water 60:40: |
| | 6:6) |
| ¹H-NMR | |
| (250 MHz, | |
| CDCl₃/CD₃OD 6:2): | |
| Non-cholesterol | |
| signals: | |
| δ=3.35 | (s, 6H, —OCOO(CH₂)₂N(CH₃)₂(CH₂)₂OH) |
| δ=3.69-3.76 | (m, 2H, —OCOO(CH₂)₂N(CH₃)₂CH₂CH₂OH) |
| δ=3.91-3.98 | (m, 2H, —OCOOCH₂CH₂N(CH₃)₂(CH₂)₂OH) |
| δ=4.02-4.11 | (m, 2H, —OCOO(CH₂)₂N(CH₃)₂CH₂CH₂OH) |
| δ=4.55-4.65 | (m, 2H, —OCOOCH₂CH₂N(CH₃)₂CH₂CH₂OH) |

Synthesis Procedures for Bicationic Lipids

2-bromoethyl-cholesterylcarbonate (14)

Add a solution of 4.49 g (10 mmol) cholesterylchloroformiate in 10 ml dichloromethane in drops to a solution of 0.71 ml (10 mmol) 2-bromoethanol and 4.16 ml (30 mmol) triethylamine in 50 ml dichloromethane under refrigeration. Stir the formulation for 2 hours at room temperature then extract twice with 40 ml 1 N HCl each time. Concentrate the organic phase to a small volume, then purify the residue via column chromatography on 100 g silica gel (cyclohexane/dichloromethane 4:1). The yield is 3.22 g 14 as a colorless solid.

| | |
|---|---|
| Yield: | 3.22 g(60% of theoretical value) |
| Mᵣ: | 537.62(C₃₀H₄₉Br₁O₃) |
| R_{f}: | 0.46(cyclohexane/eiisopropyl ether 4:1) |
| ¹H-NMR | |
| (250 MHz, CDCl₃): | |
| Non-cholesterol signals: | |
| δ=3.52 | (t, ³J = 6.4 Hz, 2H, —OCH₂CH₂Br) |
| δ=4.42 | (t, ³J = 6.3 Hz, 2H, —OCH₂CH₂Br) |

2-bromoethyl-cholesterylsuccinate (15)

Slowly add 61.2 ml (29 mmol) a 0.5 M solution of cholesterylhemisuccinoylchloride (3) in toluene in drops to a solution of 2.0 ml (29 mmol) 2-bromoethanol and 11.5 ml (83 mmol) triethylamine in 90 ml dichloromethane under refrigeration. Stir the formulation for 14 hours at room temperature. Take up the formulation in 200 ml dichloromethane and extract with 200 ml 2 N HCl. Add 50 ml methanol to improve phase separation. Remove the solvent, then purify the residue via column chromatography on 100 g silica gel. Elute the apolar impurities with cyclohexane/diisopropyl ether (10:1) and elute the product with cyclohexane/diisopropyl ether (5:1). The yield is 10.61 g 15 as a colorless solid.

| | |
|---|---|
| Yield: | 10.61 g(63% of theoretical value) |
| Mᵣ: | 593.69(C₃₃H₅₃BrO₄) |
| R_{f}: | 0.22(cyclohexane/ethyl acetate 10:1) |
| ¹H-NMR | |
| (250 MHz, CDCl₃): | |
| Non-cholesterol signals: | |
| δ=2.57-2.71 | (m, 4H, —OCO(CH₂)₂COO—) |
| δ=3.50 | (t, ³J = 6.1 Hz, 2H, —OCH₂CH₂Br) |
| δ=4.41 | (t, ³J = 6.3 Hz, 2H, —OCH₂CH₂Br) |

3-bromoethyl-cholesterylsuccinate (16)

Slowly add 70 ml (35 mmol) of a 0.5 solution of cholesterylhemisuccinoylchloride (3) in toluene in drops to a solution of 3.1 ml (35 mmol) 3-bromopropanol and 14.6 ml (105 mmol) triethylamine in 100 ml dichloromethane under refrigeration. Stir for 14 hours. Take up the formulation in 200 ml dichloromethane and extract with 200 ml 2 N HCl. Remove the solvent and purify the residue via column chromatography on 100 g silica gel. Elute the apolar impurities with cyclohexane/diisopropyl ether (10:1) and elute the product with cyclohexane/diisopropyl ether (5:1). The yield is 15.25 g 16 as a colorless solid.

| | |
|---|---|
| Yield: | 15.25 g(72% of theoretical value) |
| Mᵣ: | 607.71(C₃₄H₅₅BrO₄) |
| R_{f}: | 0.32(cyclohexane/ethyl acetate 10:1) |
| ¹H-NMR | |
| (250 MHz, CDCl₃): | |
| Non-cholesterol signals: | |
| δ=2.18 | (quint, 2H, ³J = 6,3, —OCH₂CH₂CH₂Br) |
| δ=2.56-2.69 | (m, 4H, —OCO(CH₂)₂COO—) |
| δ=3.47 | (t, ³J = 6.4 Hz, 2H, —OCH₂CH₂CH₂Br) |
| δ=4.24 | (t, ³J = 6.1 Hz, 2H, —OCH₂CH₂CH₂Br) |

N-(2-bromoethyl)-cholesterylsuccinylamide (17)

Slowly add 23.2 ml (11 mmol) of a 0.5 M solution of cholesterylhemisuccinoylchloride (3) in toluene in drops to a solution of 2.1 g (10 mmol) 2-bromoethylaminehydrobromide and 4.2 ml (30 mmol) triethylamine in 50 ml dichloromethane under refrigeration. Stir for 14 hours at room temperature. Take up the formulation in 100 ml dichloromethane and extract against 100 ml 2 N HCl. Add 50 ml methanol to improve phase separation. Remove the solvent then purify the residue via column chromatography on 70 g silica gel. Elute the apolar impurities with dichloromethane/cyclohexane/ethyl acetate (20:1:1) and elute the product with dichloromethane/ethyl acetate (10:1). The yield is 2.07 g 17 as a colorless solid.

| | |
|---|---|
| Yield: | 2.07 g(35% of theoretical value) |
| Mᵣ: | 592.70(C₃₃H₅₄BrNO₃) |
| R_{f}: | 0.37(cyclohexane/ethyl acetate 1:1) |
| ¹H-NMR | |
| (250 MHz, CDCl₃): | |
| Non-cholesterol signals: | |
| δ=2.48 | (t, ³J = 6.8 Hz, 2H, —OCOCH₂CH₂CONH—) |
| δ=2.64 | (t, ³J = 6.8 Hz, 2H, —OCOCH₂CH₂CONH—) |
| δ=3.53-3.68 | (m, 4H, —NHCH₂CH₂Br) |
| δ=6.15 | (s broad, 1H, —OCOCH₂CH₂CONH—) |

N-(3-bromopropyl)-cholesterylsuccinylamide (18)

Slowly add 23.2 ml (11 mmol) of a 0.5 M solution of cholesterylhemisuccinoylchloride (3) in toluene in drops to a solution of 2.2 g (10 mmol) 3-bromopropylaminehydrobromide and 4.2 ml (30 mmol) triethylamine in 50 ml dichloromethane under refrigeration. Stir for 14 hours at room temperature. Take up the formulation in 100 ml dichloromethane and extract against 100 ml 2 N HCl. Add 50 ml methanol to improve phase separation. Remove the solvent, then recrystallize the residue out of 40 ml methanol. The yield is 3.03 g 18 as a colorless solid.

| | |
|---|---|
| Yield: | 3.03 g(50% of theoretical value) |
| Mᵣ: | 606.73(C₃₄H₅₆BrNO₃) |
| R_{f}: | 0.34(cyclohexane/ethyl acetate 1:1) |
| ¹H-NMR | |
| (250 MHz, CDCl₃): | |
| Non-cholesterol signals: | |
| δ=2.08 | (quint, 2H, ³J = 6,5, —NHCH₂CH₂CH₂Br) |
| δ=2.48 | (t, ³J = 6.9 Hz, 2H, —OCOCH₂CH₂CONH—) |
| δ=2.65 | (t, ³J = 6.9 Hz, 2H, —OCOCH₂CH₂CONH—) |
| δ=3.30-3.48 | (m, 4H, —NHCH₂CH₂CH₂Br) |
| δ=5.88 | (s broad, 1H, —OCOCH₂CH₂CONH—) |

Cholesteryl-(2-(2-hydroxyethyloxy)-ethyl)-succinate (19)

Slowly add 40 ml (20 mmol) of a 0.5 M solution of cholesterylhemisuccinoylchloride (3) in toluene in drops to a solution of 19.1 ml (200 mmol) diethylenglycol and 8.3 ml (60 mmol) triethylamine in 50 ml dichloromethane under refrigeration. Stir for 14 hours at room temperature. Take up the formulation in 200 ml dichloromethane and extract with 200 ml 2 N HCl. Remove the solvent, the purify the residue via column chromatography on 150 g silica gel (diisopropyl ether). The yield is 9, 15 g 19 as a colorless solid.

| | |
|---|---|
| Yield: | 9.15 g(80% of theoretical value) |
| Mᵣ: | 574.84(C₃₅H₅₈O₆) |
| R_{f}: | 0.45(ethyl acetate) |
| ¹H-NMR | |
| (250 MHz, CDCl₃): | |
| Non-cholesterol | |
| signals: | |
| δ=2.57-2.70 | (m, 4H, —OCO(CH₂)₂COO—) |
| δ=3.57-3.63 | (m, 2H, —COOCH₂CH₂OCH₂CH₂OH) |
| δ=3.68-3.78 | (m, 4H, —COOCH₂CH₂OCH₂CH₂OH) |
| δ=4.28 | (t, ³J = 4.7 Hz, 2H, —COOCH₂CH₂OCH₂CH₂OH) |

Cholesteryl-(2-(2-mesyloxyethyloxy)-ethyl)-succinate (20)

Add a solution of 1.17 ml (15 mmol) methane sulfonylchloride in 10 ml dichloromethane in drops to a solution of 5.75 g (10 mmol) cholesteryl-(2-(2-hydroxyethyloxy)-ethyl)-succinate (19) and 4.16 ml (30 mmol) triethylamine in 40 ml dichloromethane under refrigeration. Stir the formulation for 2 hours at room temperatur and then extract twice with 40 ml 2 N HCl and twice with 40 ml water. After the solvent is removed, the yield is 6.23 g of clean 20 as a colorless slime.

| | |
|---|---|
| Yield: | 6.23 g(95% of theoretical value) |
| Mᵣ: | 652.93(C₃₆H₆₀O₈S) |
| R_{f}: | 0.27(cyclohexane/ethyl acetate 1:1) |
| ¹H-NMR(250 | |
| MHz, CDCl₃): | |
| Non-cholesterol | |
| signals: | |
| δ=2.57-2.70 | (m, 4H, —OCO(CH₂)₂COO—) |
| δ=3.07 | (s, 3H, —OCH₂CH₂OSO₂CH₃) |
| δ=3.69-3.80 | (m, 4H, —COOCH₂CH₂OCH₂CH₂O—) |
| δ=4.26 | (t, ³J=4.7Hz, 2H, —COOCH₂CH₂OCH₂CH₂O—) |
| δ=4.38 | (t, ³J=4.4Hz, 2H, —OCH₂CH₂OSO₂CH₃) |

N,N′-dibenzyl-α,ω-diaminoalkane (21-25)

General Synthesis Instructions:

Add a solution of 440 mmol benzaldehyde in 50 ml ethyl acetate in drops to a solution of 200 mmol of the respective α,ω-diaminoalkane, 100 mmol triethylamine and 200 mmol sodium sulphate in 100 ml ethyl acetate under refrigeration and then stir for 14 hours at room temperature. Add 150 ml methanol, then add 800 mmol sodium borohydride in portions under refrigeration. The substance should be added over a period of 6 hours to avoid formation of foam. Siphon off the solid (also use a 3 cm-high silica gel layer as a filtration aid) and rewash four times with 100 ml chloroform each time. Extract the filtrate twice against 200 ml of a 1 N NaOH (solution) and concentrate the organic phase to a small volume. Purify the residue via column chromatography on 200 g silica gel. Elute the apolar impurities with cyclohexane/ethyl acetate (2:1+1 vol % triethylamine). Then elute the product with ethyl acetate/methanol (1:1+1 vol % triethylamine).

N,N′-dibenzyl-1,2-diaminoethane (21)

Quantities Used:

13.4 ml (200 mmol) 1,2-diaminoethane

13.9 ml (100 mmol) triethylamine

28.4 g (200 mmol) sodium sulphate

44.5 ml (440 mmol) benzaldehyde

30.3 g (800 mmol) sodium borohydride

| | |
|---|---|
| Yield: | 33.65 g(140 mmol, 70% of theoretical value) as a slightly |
| | yellow solide |
| Mᵣ: | 240.35(C₁₆H₂₀N₂) |
| R_{f}: | 0.20(ethyl acetate/methanol 1:1 + 1 vol % triethylamine) |
| ¹H-NMR(250MHz, CDCl₃): | |
| δ=2.75 | (s, 4H, —NCH₂CH₂N—) |
| δ=3.77 | (s, 4H, —NHCH₂C₆H₅) |
| δ=7.19-7.37 | (m, 10H, H_{aromat.}) |

N,N′-dibenzyl-1,3-diaminopropane (22)

Quantities Used:

16.7 ml (200 mmol) 1,3-diaminopropane

13.9 ml (100 mmol) triethylamine

28.4 g (200 mmol) sodium sulphate

44.5 ml (440 mmol) benzaldehyde

30.3 g (800 mmol) sodium borohydride

| | |
|---|---|
| Yield: | 26.73 g(105 mmol, 52% of theoretical value) as a |
| | slightly yellow oil |
| Mᵣ: | 254.38(C₁₇H₂₂N₂) |
| R_{f}: | 0.20(ethyl acetate/methanol 1:1 + 1 vol % triethylamine) |
| ¹H-NMR(250 | |
| MHz, CDCl₃): | |
| δ=1.72 | (quint, ³J=6.8Hz, 2H, —NCH₂CH₂CH₂N—) |
| δ=2.70 | (t, ³J=6.7Hz, 4H, —NCH₂CH₂CH₂N—) |
| δ=3.77 | (s, 4H, —NHCH₂C₆H₅) |
| δ=7.20-7.36 | (m, 10H, H_{aromat.}) |

N,N′-dibenzyl-1,2-diaminobutane (23)

Quantities Used:

20.0 ml (200 mmol) 1,4-diaminobutane

13.9 ml (100 mmol) triethylamine

28.4 g (200 mmol) sodium sulphate

44.5 ml (440 mmol) benzaldehyde

30.3 g (800 mmol) sodium borohydride

| | |
|---|---|
| Yield: | 28.16 g(105 mmol, 52% of theoretical |
| | value) as a slightly yellow oil |
| Mᵣ: | 268.40(C₁₈H₂₄N₂) |
| R_{f}: | 0.21(ethyl acetate/methanol 1:1 + 1 vol % |
| | triethylamine) |
| ¹H-NMR(250MHz, CDCl₃): | |
| δ=1.53-1.60 | (m, 4H, —NCH₂(CH₂)₂CH₂N—) |
| δ=2.64 | (m, 4H, —NCH₂(CH₂)₂CH₂N—) |
| δ=3.78 | (s, 4H, —NHCH₂C₆H₅) |
| δ=7.20-7.34 | (m, 10H, H_{aromat.}) |

N,N′-dibenzyl-1,5-diaminopentane (24)

Quantities Used:

23.5 (200 mmol) 1,5-diaminopentane

13.9 ml (100 mmol) triethylamine

28.4 g (200 mmol) sodium sulphate

44.5 ml (440 mmol) benzaldehyde

30.3 g (800 mmol) sodium borohydride

| | |
|---|---|
| Yield: | 40.67 g(144 mmol, 72% of theoretical |
| | value) as a colorless slime |
| Mᵣ: | 282.43(C₁₉H₂₆N₂) |
| R_{f}: | 0.20(ethyl acetate/methanol 1:1 + 1 vol % |
| | triethylamine) |
| ¹H-NMR(250MHz, CDCl₃): | |
| δ=1.43-1.59 | (m, 6H, —NCH₂(CH₂)₃CH₂N—) |
| δ=2.62 | (t, ³J=7.0Hz, 4H, |
| | —NCH₂(CH₂)₃CH₂N—) |
| δ=3.77 | (s, 4H, —NHCH₂C₆H₅) |
| δ=7.20-7.33 | (m, 10H, H_{aromat.}) |

N,N′-dibenzyl-1,6-diaminohexane (25)

Quantities Used:

23.24 g (200 mmol) 1,6-diaminohexane

13.9 ml (100 mmol) triethylamine

28.4 g (200 mmol) sodium sulphate

44.5 ml (440 mmol) benzaldehyde

30.3 g (800 mmol) sodium borohydride

| | |
|---|---|
| Yield: | 29.05 g(49% of theoretical |
| | value) as a colorless solid |
| Mᵣ: | 296.46(C₂₀H₂₈N₂) |
| R_{f}: | 0.20(ethyl acetate/methanol 1:1 + 1 vol % |
| | triethylamine) |
| ¹H-NMR(250MHz, CDCl₃): | |
| δ=1.28-1.40 | (m, 4H, —N(CH₂)₂(CH₂)₂(CH₂)₂N—) |
| δ=1.40-1.58 | (m, 4H, —NCH₂CH₂(CH₂)₂CH₂CH₂N—) |
| δ=2.61 | (t, ³J=7.2Hz, 4H, |
| | —NCH₂(CH₂)₄CH₂N—) |
| δ=3.77 | (s, 4H, —HNCH₂C₆H₅) |
| δ=7.20-7.34 | (m, 10H, H_{aromat.}) |

N-tert-butyloxycarbonyl-N,N′-dibenzyl-α,ω-diaminoalkane (26-30)

General Synthesis Instructions:

Slowly add a solution of 4 mmol di-tert-butyl-dicarbonate in 3 ml dichloromethane in drops to a-solution of 6 mmol of the respective N,N′-dibenzyl-α,ω-diaminoalkane and 12 mmol triethylamine in 20 ml dichloromethane under refrigeration. Stir for 4 hours at room temperature, then remove the solvent. Purify the residue via column chromatography on 40 g silica gel. Elute the apolar impurities with cyclohexane/ethyl acetate (2:1+1 vol % triethylamine), then switch to ethyl acetate (+1 vol % triethylamine) to elute the product.

N-tert-butyloxycarbonyl-N,N′-dibenzyl-1,2-diaminoethane (26)

Quantities Used:

1.44 g (6 mmol) N,N′-dibenzyl-1,2-diaminoethane (21)

0.87 g (4 mmol) di-tert-butyl-dicarbonate

1.66 ml (12 mmol) triethylamine

| | |
|---|---|
| Yield: | 1.03 g(76% of theoretical |
| | value) as a yellow oil |
| Mᵣ: | 340.47(C₂₁H₂₈N₂O₂) |
| R_{f}: | 0.24(ethyl acetate/methanol 9:1 + 1 vol % |
| | triethylamine) |
| ¹H-NMR(250MHz, CDCl₃): | |
| δ=1.32-1.59 | (m, 9H, —OC(CH₃)₃) |
| δ=1.63-1.84 | (m, 2H, —HNCH₂CH₂N—) |
| δ=3.19-3.48 | (m, 2H, —HNCH₂CH₂N—) |
| δ=3.73 | (s, 2H, C₆H₅CH₂—HN(CH₂)₂N—) |
| δ=4.35-4.52 | (m, 2H, —HNCH₂CH₂N—CH₂C₆H₅) |
| δ=7.17-7.34 | (m, 10H, H_{aromat.}) |

N-tert-butyloxycarbonyl-N,N′-dibenzyl-1,3-diaminopropane (27)

Quantities Used:

1.53 g (6 mmol) N,N′-dibenzyl-1,3-diaminopropane (22)

0.87 g (4 mmol) di-tert-butyl-dicarbonate

1.66 ml (12 mmol) triethylamine

| | |
|---|---|
| Yield: | 0.90 g(63% of theoretical |
| | value) as yellow oil |
| Mᵣ: | 354.49(C₂₂H₃₀N₂O₂) |
| R_{f}: | 0.24(ethyl acetate/methanol 9:1 + 1 vol % |
| | triethylamine) |
| ¹H-NMR(250MHz, CDCl₃): | |
| δ=1.37-1.51 | (m, 9H, —OC(CH₃)₃) |
| δ=1.60-1.79 | (m, 2H, —HNCH₂CH₂CH₂N—) |
| δ=2.59 | (t, ³J=7.0Hz, 2H, —HNCH₂(CH₂)₂N—) |
| δ=3.11-3.37 | (m, 2H, —HN(CH₂)₂CH₂N—) |
| δ=3.74 | (s, 2H, C₆H₅CH₂—HN(CH₂)₃N—) |
| δ=4.35-4.48 | (m, 2H, —HNCH₂CH₂CH₂N—CH₂C₆H₅) |
| δ=7.17-7.34 | (m, 10H, H_{aromat.}) |

N-tert-butyloxycarbonyl-N,N′-dibenzyl-1,4-diaminobutane (28)

Quantities Used:

1.61 g (6 mmol) N,N′-dibenzyl-1,4-diaminobutane (23)

0.87 g (4 mmol) di-tert-butyl-dicarbonate

1.66 ml (12 mmol) triethylamine

| | |
|---|---|
| Yield: | 0.91 g(65% of theoretical |
| | value) as yellow oil |
| Mᵣ: | 368.52(C₂₃H₃₂N₂O₂) |
| R_{f}: | 0.27(ethyl acetate/methanol 9:1 + 1 vol % |
| | triethylamine) |
| ¹H-NMR(250MHz, CDCl₃): | |
| δ=1.37-1.61 | (m, 13H, —OC(CH₃)₃ and |
| | —HNCH₂(CH₂)₂CH₂N—) |
| δ=2.60 | (t, ³J=7.0Hz, 2H, —HNCH₂(CH₂)₃N—) |
| δ=3.00-3.32 | (m, 2H, —HN(CH₂)₃CH₂N—) |
| δ=3.76 | (s, 2H, C₆H₅CH₂—HN(CH₂)₄N—) |
| δ=4.30-4.52 | (m, 2H, —HN(CH₂)₄N—CH₂C₆H₅) |
| δ=7.15-7.40 | (m, 10H, H_{aromat.}) |

N-tert-butyloxycarbonyl-N,N′-dibenzyl-1,5-diaminopentane (29)

Quantities Used:

1.70 g (6 mmol) N,N′-dibenzyl-1,5-diaminopentane (24)

0.87 g (4 mmol) di-tert-butyl-dicarbonate

1.66 ml (12 mmol) triethylamine

| | |
|---|---|
| Yield: | 0.96 g(63% of theoretical |
| | value) as yellow oil |
| Mᵣ: | 382.55(C₂₄H₃₄N₂O₂) |
| R_{f}: | 0.26(ethyl acetate/methanol 9:1 + 1 vol % |
| | triethylamine) |
| ¹H-NMR(250MHz, CDCl₃): | |
| δ=1.17-1.33 | (m, 2H, —HN(CH₂)₂CH₂(CH₂)₂N—) |
| δ=1.33-1.61 | (m, 13H, —OC(CH₃)₃ and |
| | —HNCH₂CH₂CH₂CH₂CH₂N—) |
| δ=2.59 | (t, ³J=7.2Hz, 2H, —HNCH₂(CH₂)₄N—) |
| δ=3.02-3.28 | (m, 2H, —HN(CH₂)₄CH₂N—) |
| δ=3.77 | (s, 2H, C₆H₅CH₂—HN(CH₂)₅N—) |
| δ=4.30-4.51 | (m, 2H, —HN(CH₂)₅N—CH₂C₆H₅) |
| δ=7.15-7.40 | (m, 10H, H_{aromat.}) |

N-tert-butyloxycarbonyl-N,N′-dibenzyl-1,6-diaminohexane (30)

Quantities Used:

1.78 g (6 mmol) N,N′-dibenzyl-1,6-diaminohexane (25)

0.87 g (4 mmol) di-tert-butyl-dicarbonate

1.66 ml (12 mmol) triethylamine

| | |
|---|---|
| Yield: | 1.12 g(70% of theoretical |
| | value) as yellow oil |
| Mᵣ: | 396.57(C₂₅H₃₆N₂O₂) |
| R_{f}: | 0.33(ethyl acetate/methanol 9:1 + 1 vol % |
| | triethylamine) |
| ¹H-NMR(250MHz, CDCl₃): | |
| δ=1.15-1.38 | (m, 4H, —HN(CH₂)₂(CH₂)₂(CH₂)₂N—) |
| δ=1.38-1.60 | (m, 13H, —OC(CH₃)₃ and |
| | —HNCH₂CH₂(CH₂)₂CH₂CH₂N—) |
| δ=2.60 | (t, ³J=7.2Hz, 2H, —HNCH₂(CH₂)₅N—) |
| δ=3.00-3.28 | (m, 2H, —HN(CH₂)₅CH₂N—) |
| δ=3.77 | (s, 2H, C₆H₅CH₂—HN(CH₂)₆N—) |
| δ=4.30-4.52 | (m, 2H, —HN(CH₂)₆N—CH₂C₆H₅) |
| δ=7.15-7.42 | (m, 10H, H_{aromat.}) |

N-tert-butyloxycarbonyl-N′-ethyl-N,N′-dibenzyl-α,ω-diaminoalkane (31-35)

General Synthesis Instructions:

Add 0.5 mmol potassium carbonate and 1.5 mmol ethyl iodide to a solution of 1.0 mmol of the respective N-tert-butyloxycarbonyl-N,N′-dibenzyl-α,ω-diaminoalkane in 10 ml acetonitrile and stir the formulation overnight (12 h) at 60° C. gerührt. Remove the solvent and excess ethyl iodide. Purify the residue via column chromatography on 30 g silica gel. First elute the apolar impurities with cyclohexane/diisopropyl ether (1:1+1 vol % triethylamine) and then switch to cyclohexane/ethyl acetate (4:1+1 vol % triethylamine) to elute the product.

N-tert-butyloxycarbonyl-N′-ethyl-N,N′-dibenzyl-1,2-diaminoethane (31)

Quantities Used:

340 mg (1.0 mmol) N-tert-butyloxycarbonyl-N,N′-dibenzyl-1,2-diaminoethane (26)

121 μl (1.5 mmol) ethyl iodide

69 mg (0.5 mmol) potassium carbonate

| | |
|---|---|
| Yield: | 287 mg(78% of theoretical |
| | value) as a yellow oil |
| Mᵣ: | 368.52(C₂₃H₃₂N₂O₂) |
| R_{f}: | 0.65(ethyl acetate) |
| ¹H-NMR(250MHz, CDCl₃): | |
| δ=1.00 | (t, ³J=7.2Hz, 3H, —NCH₂CH₃) |
| δ=1.37-1.49 | (m, 9H, —NCOOC(CH₃)₃) |
| δ=2.40-2.65 | (m, 4H, —NCH₂CH₂NCH₂CH₃) |
| δ=3.10-3.36 | (m, 2H, —NCH₂CH₂NCH₂CH₃) |
| δ=3.51 | (s, 2H, C₆H₅CH₂—NCH₂CH₃) |
| δ=4.29-4.44 | (m, 2H, C₆H₅CH₂—NCOOC(CH₃)₃) |
| δ=7.10-7.36 | (m, 10H, H_{aromat.}) |

N-tert-butyloxycarbonyl-N′-ethyl-N,N′-dibenzyl-1,3-diaminopropane (32)

Quantities Used:

354 mg (1.0 mmol) N-tert-butyloxycarbonyl-N,N′-dibenzyl-1,3-diaminopropane (27)

121 μl (1.5 mmol) ethyl iodide

69 mg (0.5 mmol) potassium carbonate

| | |
|---|---|
| Yield: | 331 mg(86% of theoretical |
| | value) as a yellow oil |
| Mᵣ: | 382.55(C₂₄H₃₄N₂O₂) |
| R_{f}: | 0.65(ethyl acetate) |
| ¹H-NMR(250MHz, CDCl₃): | |
| δ=0.99 | (t, ³J=7.2Hz, 3H, —NCH₂CH₃) |
| δ=1.37-1.49 | (m, 9H, —NCOOC(CH₃)₃) |
| δ=1.58-1.79 | (m, 2H, —NCH₂CH₂CH₂N—) |
| δ=2.31-2.47 | (m, 2H, —N(CH₂)₂CH₂NCH₂CH₃) |
| δ=2.45 | (quart, ³J=7.1Hz, 2H, —NCH₂CH₃) |
| δ=3.03-3.32 | (m, 2H, —NCH₂(CH₂)₂NCH₂CH₃) |
| δ=3.51 | (s, 2H, C₆H₅CH₂—NCH₂CH₃) |
| δ=4.26-4.50 | (m, 2H, C₆H₅CH₂—NCOOC(CH₃)₃) |
| δ=7.17-7.35 | (m, 10H, H_{aromat.}) |

N-tert-butyloxycarbonyl-N′-ethyl-N,N′-dibenzyl-1,4-diaminobutane (33)

Quantities Used:

369 mg (1.0 mmol) N-tert-butyloxycarbonyl-N,N′-dibenzyl-1,4-diaminobutane (28)

121 μl (1.5 mmol) ethyl iodide

69 mg (0.5 mmol) potassium carbonate

| | |
|---|---|
| Yield: | 268 mg(68% of theoretical |
| | value) as a yellow oil |
| Mᵣ: | 396.57(C₂₅H₃₆N₂O₂) |
| R_{f}: | 0.65(ethyl acetate) |
| ¹H-NMR(250MHz, CDCl₃): | |
| δ=1.00 | (t, ³J=7.0Hz, 3H, —NCH₂CH₃) |
| δ=1.37-1.55 | (m, 13H, —NCOOC(CH₃)₃ |
| | and —NCH₂(CH₂)₂CH₂N—) |
| δ=2.33-2.45 | (m, 2H, —N(CH₂)₃CH₂NCH₂CH₃) |
| δ=2.47 | (quart, ³J=7.1Hz, 2H, —NCH₂CH₃) |
| δ=3.00-3.30 | (m, 2H, —NCH₂(CH₂)₃NCH₂CH₃) |
| δ=3.52 | (s, 2H, C₆H₅CH₂—NCH₂CH₃) |
| δ=4.25-4.50 | (m, 2H, C₆H₅CH₂—NCOOC(CH₃)₃) |
| δ=7.17-7.36 | (m, 10H, H_{aromat.}) |

N-tert-butyloxycarbonyl-N′-ethyl-N,N′-dibenzyl-1,5-diaminopentane (34)

Quantities Used:

383 mg (1.0 mmol) N-tert-butyloxycarbonyl-N,N′-dibenzyl-1,5-diaminopentane (29)

121 μl (1.5 mmol) ethyl iodide

69 mg (0.5 mmol) potassium carbonate

| | |
|---|---|
| Yield: | 263 mg(64% of theoretical |
| | value) as a yellow oil |
| Mᵣ: | 410.60(C₂₆H₃₈N₂O₂) |
| R_{f}: | 0.69(ethyl acetate) |
| ¹H-NMR(250MHz, CDCl₃): | |
| δ=1.01 | (t, ³J=7.2Hz, 3H, —NCH₂CH₃) |
| δ=1.14-1.31 | (m, 2H, |
| | —N(CH₂)₂CH₂(CH₂)₂N—) |
| δ=1.33-1.60 | (m, 13H, —NCOOC(CH₃)₃ |
| | and —NCH₂CH₂CH₂CH₂CH₂N—) |
| δ=2.38 | (t, ³J=7.3Hz, |
| | 2H, —N(CH₂)₄CH₂NCH₂CH₃) |
| δ=2.48 | (quart, ³J=7.1Hz, 2H, —NCH₂CH₃) |
| δ=3.00-3.28 | (m, 2H, —NCH₂(CH₂)₄NCH₂CH₃) |
| δ=3.54 | (s, 2H, C₆H₅CH₂—NCH₂CH₃) |
| δ=4.30-4.50 | (m, 2H, C₆H₅CH₂—NCOOC(CH₃)₃) |
| δ=7.10-7.37 | (m, 10H, H_{aromat.}) |

N-tert-butyloxycarbonyl-N′-ethyl-N,N′-dibenzyl-1,6-diaminohexane (35)

Quantities Used:

397 mg (1.0 mmol) N-tert-butyloxycarbonyl-N,N′-dibenzyl-1,6-diaminohexane (30)

121 μl (1.5 mmol) ethyl iodide

69 mg (0.5 mmol) potassium carbonate

| | |
|---|---|
| Yield: | 280 mg(66% of theoretical |
| | value) as a yellow oil |
| Mᵣ: | 424.63(C₂₇H₄₀N₂O₂) |
| R_{f}: | 0.66(ethyl acetate) |
| ¹H-NMR(250MHz, CDCl₃): | |
| δ=1.02 | (t, ³J=7.2Hz, 3H, —NCH₂CH₃) |
| δ=1.15-1.34 | (m, 4H, —N(CH₂)₂(CH₂)₂(CH₂)₂N—) |
| δ=1.34-1.55 | (m, 13H, —NCOOC(CH₃)₃ and |
| | —NCH₂CH₂(CH₂)₂CH₂CH₂N—) |
| δ=2.38 | (t, ³J=7.3Hz, 2H, |
| | —N(CH₂)₅CH₂NCH₂CH₃) |
| δ=2.49 | (quart, ³J=7.1Hz, 2H, —NCH₂CH₃) |
| δ=3.00-3.28 | (m, 2H, —NCH₂(CH₂)₅NCH₂CH₃) |
| δ=3.54 | (s, 2H, C₆H₅CH₂—NCH₂CH₃) |
| δ=4.30-4.50 | (m, 2H, C₆H₅CH₂—NCOOC(CH₃)₃) |
| δ=7.10-7.37 | (m, 10H, H_{aromat.}) |

N-ethyl-N,N′-dibenzyl-α,ω-diaminoalkane (36-40)

General Synthesis Instructions:

Add 5 ml trifluoroacetic acid in drops to a solution of 3.0 mmol of the respective N-tert-butyloxycarbonyl-N′-ethyl-N,N′-dibenzyl-α,ω-diaminoalkane in 10 ml dichloromethane and stir the formulation overnight at room temperature. Siphon off the trifluoroacetic acid and dichloromethane in a vacuum. Take up the residue in approx. 20 ml dichloromethane and extract twice against 1 N NaOH. After the organic phase is concentrated to a small volume, the clean product remains as an oil.

N-ethyl-N,N′-dibenzyl-1,2-diaminoethane (36)

Quantities Used:

1.11 g (3.0 mmol) N-tert-butyloxycarbonyl-N′-ethyl-N,N′-dibenzyl-1,2-diaminoethane (31)

| | |
|---|---|
| Yield: | 749 mg(93% of theoretical |
| | value) as a yellow oil |
| Mᵣ: | 268.40(C₁₈H₂₄N₂) |
| R_{f}: | 0.14(ethyl acetate/methanol 4:1 + 1 vol % |
| | triethylamine) |
| ¹H-NMR(250MHz, CDCl₃): | |
| δ=1.01 | (t, ³J=6.7Hz, 3H, —NCH₂CH₃) |
| δ=2.40-2.65 | (m, 4H, —NCH₂CH₂NCH₂CH₃) |
| δ=1.63-1.84 | (m, 2H, —HNCH₂CH₂NCH₂CH₃) |
| δ=3.51 | (s, 2H, C₆H₅CH₂—NCH₂CH₃) |
| δ=3.73 | (s, 2H, C₆H₅CH₂—HN(CH₂)₂N—) |
| δ=7.22-7.35 | (m, 10H, H_{aromat.}) |

N-ethyl-N,N′-dibenzyl-1,3-diaminopropane (37)

Quantities Used:

1.15 g (3.0 mmol) N-tert-butyloxycarbonyl-N′-ethyl-N,N′-dibenzyl-1,3-diaminopropane (32)

| | |
|---|---|
| Yield: | 796 mg(94% of theoretical |
| | value) as a yellow oil |
| Mᵣ: | 282.43(C₁₉H₂₆N₂) |
| R_{f}: | 0.14(ethyl acetate/methanol 4:1 + 1 vol % |
| | triethylamine) |
| ¹H-NMR(250MHz, CDCl₃): | |
| δ=1.02 | (t, ³J=6.7Hz, 3H, —NCH₂CH₃) |
| δ=1.70 | (quint, ³J=6.9Hz, 2H, |
| | —HNCH₂CH₂CH₂N—) |
| δ=2.47 | (t, ³J=7.1Hz, 2H, |
| | —HN(CH₂)₂CH₂NCH₂CH₃) |
| δ=2.49 | (quart, ³J=7.1Hz, 2H, —NCH₂CH₃) |
| δ=2.65 | (t, ³J=6.7Hz, |
| | 2H, —HNCH₂(CH₂)₂NCH₂CH₃) |
| δ=3.53 | (s, 2H, C₆H₅CH₂—NCH₂CH₃) |
| δ=3.75 | (s, 2H, C₆H₅CH₂—HN(CH₂)₃N—) |
| δ=7.22-7.35 | (m, 10H, H_{aromat.}) |

N-ethyl-N,N′-dibenzyl-1,4-diaminobutane (38)

Quantities Used:

1.19 g (3.0 mmol) N-tert-butyloxycarbonyl-N′-ethyl-N,N′-dibenzyl-1,4-diaminobutane (33)

| | |
|---|---|
| Yield: | 854 mg(96% of theoretical |
| | value) as a yellow oil |
| Mᵣ: | 296.46(C₂₀H₂₈N₂) |
| R_{f}: | 0.16(ethyl acetate/methanol 4:1 + 1 vol % |
| | triethylamine) |
| ¹H-NMR(250MHz, CDCl₃): | |
| δ=1.01 | (t, ³J=7.2Hz, |
| | 3H, —NCH₂CH₃) |
| δ=1.47-1.57 | (m, 4H, |
| | —HNCH₂(CH₂)₂CH₂N—) |
| δ=2.43 | (t, ³J=6.9Hz, 2H, |
| | —HN(CH₂)₃CH₂NCH₂CH₃) |
| δ=2.49 | (quart, ³J=7.1Hz, 2H, |
| | —NCH₂CH₃) |
| δ=2.61 | (t, ³J=6.7Hz, 2H, |
| | —HNCH₂(CH₂)₃NCH₂CH₃) |
| δ=3.54 | (s, 2H, C₆H₅CH₂—NCH₂CH₃) |
| δ=3.77 | (s, 2H, C₆H₅CH₂—HN(CH₂)₄N—) |
| δ=7.18-7.37 | (m, 10H, H_{aromat.}) |

N-ethyl-N,N′-dibenzyl-1,5-diaminopentane (39)

Quantities Used:

1.23 g (3.0 mmol) N-tert-butyloxycarbonyl-N′-ethyl-N,N′-dibenzyl-1,5-diaminopentane (34)

| | |
|---|---|
| Yield: | 913 mg(98% of theoretical |
| | value) as a yellow oil |
| Mᵣ: | 310.48(C₂₁H₃₀N₂) |
| R_{f}: | 0.17(ethyl acetate/methanol 4:1 + 1 vol % |
| | triethylamine) |
| ¹H-NMR(250MHz, CDCl₃): | |
| δ=1.02 | (t, ³J=7.0Hz, |
| | 3H, —NCH₂CH₃) |
| δ=1.23-1.37 | (m, 2H, —HN(CH₂)₂CH₂(CH₂)₂N—) |
| δ=1.38-1.57 | (m, 4H, —HNCH₂CH₂CH₂CH₂CH₂N—) |
| δ=2.41 | (t, ³J=7.3Hz, 2H, |
| | —HN(CH₂)₄CH₂NCH₂CH₃) |
| δ=2.49 | (quart, ³J=7.1Hz, 2H, |
| | —NCH₂CH₃) |
| δ=2.60 | (t, ³J=7.0Hz, 2H, |
| | —HNCH₂(CH₂)₄NCH₂CH₃) |
| δ=3.54 | (s, 2H, C₆H₅CH₂—NCH₂CH₃) |
| δ=3.77 | (s, 2H, C₆H₅CH₂—HN(CH₂)₅N—) |
| δ=7.17-7.37 | (m, 10H, H_{aromat.}) |

N-ethyl-N,N′-dibenzyl-1,6-diaminohexane (40)

Quantities Used:

1.27 g (3,0 mmol) N-tert-butyloxycarbonyl-N′-ethyl-N,N′-dibenzyl-1,6-diaminohexane (35)

| | |
|---|---|
| Yield: | 944mg(97% of theoretical value)as a yellow oil |
| Mᵣ: | 324.51(C₂₂H₃₂N₂) |
| R_{f}: | 0.20(ethyl acetate/methanol 4:1 + 1 |
| | vol % triethylamine) |
| ¹H-NMR(250MHz, | |
| CDCl₃): | |
| δ=1.02 | (t, ³J=7.0Hz, 3H, —NCH₂CH₃) |
| δ=1.19-1.37 | (m, 4H, —HN(CH₂)₂(CH₂)₂(CH₂)₂N—) |
| δ=1.38-1.59 | (m, 4H, —HNCH₂CH₂(CH₂)₂CH₂ |
| | CH₂N—) |
| δ=2.39 | (t, ³J=7.3Hz, 2H, —HN(CH₂)₅CH₂NCH₂CH₃) |
| δ=2.49 | (quart, ³J=7.1Hz, 2H, —NCH₂CH₃) |
| δ=2.60 | (t, ³J=7.2Hz, 2H, —HNCH₂(CH₂)₅NCH₂CH₃) |
| δ=3.54 | (s, 2H, C₆H₅CH₂—NCH₂CH₃) |
| δ=3.76 | (s, 2H, C₆H₅CH₂—HN(CH₂)₆N—) |
| δ=7.17-7.38 | (m, 10H, H_{aromat.}) |

Synthesis Procedures for Coupling Lipid Components and Protected Head Groups and Bicationic Lipids with Two Anchors

General Synthesis Instructions for Lipids with a Lipid Anchor:

Stir a mixture of 1.0 mmol of the respective N-ethyl-N,N′-dibenzyl-α,ω-diaminoalkane (36-40), 1.4 mmol of the respective lipid component and 0.5 mmol potassium carbonate in 10 ml acetonitrile/toluene (8:1) overnight with reflux. Remove the solvent completely and purify the residue via column chromatography.

1-(cholesteryloxycarbonylmethyl)-1,6-dibenzyl-1,6-diazaoctane (41)

Quantities Used:

296 mg (1.0 mmol) N-ethyl-N,N′-dibenzyl-1,4-diaminobutane (38)

648 mg (1.4 mmol) chloroacetic acid cholesterylester (1)

69 mg (0.5 mmol) potassium carbonate

Perform purification via column chromatography on 20 g silica gel, then elute the apolar impurities with cyclohexane/diisopropyl ether (1:1), and elute the product with cyclohexane/ethyl acetate (10:1). The yield is 427 mg 41 as a colorless oil.

| | |
|---|---|
| Yield: | 427mg(59% of theoretical value)as a colorless oil |
| Mᵣ: | 723.14(C₄₉H₇₄N₂O₂) |
| R_{f}: | 0.22(cyclohexane/ethyl acetate 6:1) |
| ¹H-NMR(250MHz, | |
| CDCl₃): | |
| Non-cholesterol | |
| signals: | |
| δ=1.01 | (t, ³J=7.0Hz, 3H, —NCH₂CH₃) |
| δ=1.47-1.57 | (m, 4H, —NCH₂(CH₂)₂CH₂N—) |
| δ=2.41 | (t, ³J=6.7Hz, 2H, —N(CH₂)₃CH₂NCH₂CH₃) |
| δ=2.50 | (quart, ³J=7.1Hz, 2H, —NCH₂CH₃) |
| δ=2.60 | (t, ³J=6.6Hz, 2H, —NCH₂(CH₂)₃NCH₂CH₃) |
| δ=3.25 | (s, 2H, OCOCH₂N(CH₂)₄N—) |
| δ=3.55 | (s, 2H, C₆H₅CH₂—NCH₂CH₃) |
| δ=3.75 | (s, 2H, C₆H₅CH₂—N(CH₂)₄NCH₂CH₃) |
| δ=7.18-7.42 | (m, 10H, H_{aromat.}) |

1-(2(cholesteryloxycarbonyloxy)-ethyl)-1,6-dibenzyl-1,6-diazaoctane (42)

Quantities Used:

296 mg (1.0 mmol) N-ethyl-N,N′-dibenzyl-1,4-diaminobutane (38)

753 mg (1.4 mmol) 2-bromoethyl-cholesterylcarbonate (14)

69 mg (0.5 mmol) potassium carbonate

Perform purification via column chromatography on 20 g silica gel, then elute the apolar impurities with cyclohexane/diisopropyl ether (2: 1), and elute the product with diisopropyl ether. The yield is 377 mg 42 as a colorless oil.

| | |
|---|---|
| Yield: | 377mg(50% of theoretical value)as a colorless oil |
| Mᵣ: | 753.17(C₅₀H₇₆N₂O₃) |
| R_{f}: | 0.25(cyclohexane/ethyl acetate 2:1) |
| ¹H-NMR(250MHz, | |
| CDCl₃): | |
| Non-cholesterol | |
| signals: | |
| δ=1.01 | (t, ³J=7.0Hz, 3H, —NCH₂CH₃) |
| δ=1.47-1.57 | (m, 4H, —NCH₂(CH₂)₂CH₂N—) |
| δ=2.33-2.45 | (m, 2H, —N(CH₂)₃CH₂NCH₂CH₃) |
| δ=2.46 | (t, ³J=7.1Hz, 2H, —NCH₂(CH₂)₃NCH₂CH₃) |
| δ=2.48 | (quart, ³J=7.1Hz, 2H, —NCH₂CH₃) |
| δ=2.72 | (t, ³J=6.3Hz, 2H, —OCOOCH₂CH₂N—) |
| δ=3.53 | (s, 2H, C₆H₅CH₂—NCH₂CH₃) |
| δ=3.61 | (s, 2H, C₆H₅CH₂—N(CH₂)₄NCH₂CH₃) |
| δ=4.15 | (t, ³J=6.3Hz, 2H, —OCOOCH₂CH₂N—) |
| δ=7.17-7.35 | (m, 10H, H_{aromat.}) |

1-(cholesterylhemisuccinoyloxy-2-ethyl)-1,6-dibenzyl-1,6-diazaoctane (43)

Quantities Used:

296 mg (1.0 mmol) N-ethyl-N,N′-dibenzyl-1,4-diaminobutane (38)

831 mg (1.4 mmol) 2-bromoethyl-cholesterylsuccinate (15)

69 mg (0.5 mmol) potassium carbonate

Perform purification via column chromatography on 20 g silica gel, then elute the apolar impurities with cyclohexane/diisopropyl ether (1:1), and elute the product with cyclohexane/ethyl acetate (4:1). The yield is 518 mg 43 as a colorless oil.

| | |
|---|---|
| Yield: | 518mg(64% of theoretical value)as a yellow oil |
| Mᵣ: | 809.23(C₅₃H₈₀N₂O₄) |
| R_{f}: | 0.42(ethyl acetate) |
| ¹H-NMR(250MHz, | |
| CDCl₃): | |
| Non-cholesterol | |
| signals: | |
| δ=1.01 | (t, ³J=7.0Hz, 3H, —NCH₂CH₃) |
| δ=1.47-1.57 | (m, 4H, —NCH₂(CH₂)₂CH₂N—) |
| δ=2.33-2.45 | (m, 4H, —NCH₂(CH₂)₂CH₂NCH₂CH₃) |
| δ=2.46 | (quart, ³J=7.1Hz, 2H, —NCH₂CH₃) |
| δ=2.54-2.61 | (m, 4H, —OCO(CH₂)₂COO—) |
| δ=2.68 | (t, ³J=6.1Hz, 2H, —OCOCH₂CH₂N—) |
| δ=3.53 | (s, 2H, C₆H₅CH₂—NCH₂CH₃) |
| δ=3.59 | (s, 2H, C₆H₅CH₂—N(CH₂)₄NCH₂CH₃) |
| δ=4.13 | (t, ³J=6.3Hz, 2H, —OCOCH₂CH₂N—) |
| δ=7.17-7.37 | (m, 10H, H_{aromat.}) |

1-(cholesterylhemisuccinoyloxy-3-propyl)-1,6-dibenzyl-1,6-diazaoctane (44)

Quantities Used:

296 mg (1.0 mmol) N-ethyl-N,N′-dibenzyl-1,4-diaminobutane (38)

851 mg (1.4 mmol) 3-bromopropyl-cholesterylsuccinate (16)

69 mg (0.5 mmol) potassium carbonate

Perform purification via column chromatography on 20 g silica gel, then elute the apolar impurities with cyclohexane/diisopropyl ether (1:1), and elute the product with cyclohexane/ethyl acetate (4:1). The yield is 543 mg 44 as a colorless oil

| | |
|---|---|
| Yield: | 543mg(66% of theoretical value)as a yellow oil |
| Mᵣ: | 823.26(C₅₄H₈₂N₂O₄) |
| R_{f}: | 0.36(ethyl acetate) |
| ¹H-NMR(250MHz, | |
| CDCl₃): | |
| Non-cholesterol | |
| signals: | |
| δ=1.01 | (t, ³J=7.2Hz, 3H, —NCH₂CH₃) |
| δ=1.47-1.57 | (m, 4H, —NCH₂(CH₂)₂CH₂N—) |
| δ=1.68-1.82 | (m, 2H, —COOCH₂CH₂CH₂N) |
| δ=2.32-2.56 | (m, 12H, |
| | —OCO(CH₂)₂COO(CH₂)₂CH₂NCH₂ |
| | (CH₂)₂CH₂NCH₂CH₃ |
| δ=3.51 | (s, 2H, C₆H₅CH₂—N(CH₂)₄NCH₂CH₃) |
| δ=3.53 | (s, 2H, C₆H₅CH₂—NCH₂CH₃) |
| δ=4.10 | (t, ³J=6.6Hz, 2H, —COOCH₂(CH₂)₂N—) |
| δ=7.17-7.35 | (m, 10H, H_{aromat.}) |

1-((cholesterylhemisuccinoyloxy-2-ethyloxy)-2-ethyl)-1,6-dibenzyl-1,6-diazaoctane (45)

Quantities Used:

296 mg (1.0 mmol) N-ethyl-N,N′-dibenzyl-1,4-diaminobutane (38)

916 mg (1.4 mmol) cholesteryl-(2-(2-mesyloxyethyloxy)-ethyl)-succinate (20)

69 mg (0.5 mmol) potassium carbonate

Perform purification via column chromatography on 30 g silica gel, then elute the apolar impurities with diisopropyl ether, and elute the product with cyclohexane/ethyl acetate (2:1). The yield is 580 mg 45 as a colorless oil.

| | |
|---|---|
| Yield: | 580mg(68% of theoretical value)as a yellow oil |
| Mᵣ: | 853.28(C₅₅H₈₄N₂O₅) |
| R_{f}: | 0.22(ethyl acetate) |
| ¹H-NMR(250MHz, | |
| CDCl₃): | |
| Non-cholesterol | |
| signals: | |
| δ=1.01 | (t, ³J=7.2Hz, 3H, —NCH₂CH₃) |
| δ=1.47-1.57 | (m, 4H, —NCH₂(CH₂)₂CH₂N—) |
| δ=2.35-2.52 | (m, 4H, —NCH₂(CH₂)₂CH₂NCH₂CH₃) |
| δ=2.48 | (quart, ³J=7.1Hz, 2H, —NCH₂CH₃) |
| δ=2.55-2.65 | (m, 4H, —OCO(CH₂)₂COO—) |
| δ=2.65 | (t, ³J=6.4Hz, 2H, —OCH₂CH₂OCH₂CH₂N—) |
| δ=3.49-3.78 | (m, 4H, —OCH₂CH₂OCH₂CH₂N—) |
| δ=3.53 | (s, 2H, C₆H₅CH₂—NCH₂CH₃) |
| δ=3.60 | (s, 2H, C₆H₅CH₂—N(CH₂)₄NCH₂CH₃) |
| δ=4.17-4.30 | (m, 2H, —OCH₂CH₂O—CH₂CH₂N—) |
| δ=7.16-7.35 | (m, 10H, H_{aromat.}) |

1-(cholesteryloxycarbonylmethyl)-1,4-dibenzyl-1,4-diazahexane (46)

Quantities Used:

268 mg (1.0 mmol) N-ethyl-N,N′-dibenzyl-1,2-diaminoethane (36)

648 mg (1.4 mmol) chloroacetic acid cholesterylester (1)

69 mg (0.5 mmol) potassium carbonate

Perform purification via column chromatography on 20 g silica gel, elute the apolar impurities with cyclohexane/diisopropyl ether (1:1), and elute the product with cyclohexane/ethyl acetate (10:1). The yield is 445 mg 46 as a colorless oil.

| | |
|---|---|
| Yield: | 445mg(64% of theoretical value)as a yellow oil |
| Mᵣ: | 695.08(C₄₇H₇₀N₂O₂) |
| R_{f}: | 0.22(cyclohexane/ethyl acetate 6:1) |
| ¹H-NMR(250MHz, | |
| CDCl₃): | |
| Non-cholesterol | |
| signals: | |
| δ=1.00 | (t, ³J=7.2Hz, 3H, —NCH₂CH₃) |
| δ=2.40-2.73 | (m, 6 H, —NCH₂CH₂NCH₂CH₃) |
| δ=3.27 | (s, 2H, OCOCH₂N(CH₂)₂N—) |
| δ=3.51 | (s, 2H, C₆H₅CH₂—NCH₂CH₃) |
| δ=3.79 | (s, 2H, C₆H₅CH₂—N(cH₂)₂NCH₂CH₃) |
| δ=7.10-7.36 | (m, 10H, H_{aromat.}) |

1-(cholesteryloxycarbonylmethyl)-1,5-dibenzyl-1,5-diazaheptane (47)

Quantities Used:

282 mg (1.0 mmol) N-ethyl-N,N′-dibenzyl-1,3-diaminopropane (37)

648 mg (1.4 mmol) chloroacetic acid cholesterylester (1)

69 mg (0.5 mmol) potassium carbonate

Perform purification via column chromatography on 20 g silica gel, elute the apolar impurities with cyclohexane/diisopropyl ether (1:1), and elute the product with cyclohexane/ethyl acetate (10:1). The yield is 468 mg 47 as a colorless oil.

| | |
|---|---|
| Yield: | 468mg(66% of theoretical value)as a yellow oil |
| Mᵣ: | 709.11(C₄₈H₇₂N₂O₂) |
| R_{f}: | 0.23(cyclohexane/ethyl acetate 6:1) |
| ¹H-NMR(250MHz, | |
| CDCl₃): | |
| Non-cholesterol | |
| signals: | |
| δ=1.01 | (t, ³J=7.0Hz, 3H, —NCH₂CH₃) |
| δ=1.59-1.76 | (m, 2H, —NCH₂CH₂CH₂N—) |
| δ=2.46 | (t, ³J=7.3Hz, 2H, —N(CH₂)₂CH₂NCH₂CH₃) |
| δ=2.48 | (quart, ³J=7.1Hz, 2H, —NCH₂CH₃) |
| δ=2.65 | (t, ³J=7.2Hz, 2H, —NCH₂(CH₂)₂NCH₂CH₃) |
| δ=3.24 | (s, 2H, OCOCH₂N(CH₂)₃N—) |
| δ=3.53 | (s, 2H, C₆H₅CH₂—NCH₂CH₃) |
| δ=3.75 | (s, 2H, C₆H₅CH₂—N(CH₂)₃NCH₂CH₃) |
| δ=7.17-7.35 | (m, 10H, H_{aromat.}) |

1-(cholesteryloxycarbonylmethyl)-1,7-dibenzyl-1,7-diazaononane (48)

Quantities Used:

310 mg (1.0 mmol) N-ethyl-N,N′-dibenzyl-1,5-diaminopentane (39)

648 mg (1.4 mmol) chloroacetic acid cholesterylester (1)

69 mg (0.5 mmol) potassium carbonate

Perform purification via column chromatography on 20 g silica gel, elute the apolar impurities with cyclohexane/diisopropyl ether (1:1), and elute the product with cyclohexane/ethyl acetate (10:1). The yield is 553 mg 48 as a colorless oil.

| | |
|---|---|
| Yield: | 553mg(75% of theoretical value)as a yellow oil |
| Mᵣ: | 737.17(C₅₀H₇₆N₂O₂) |
| R_{f}: | 0.26(cyclohexane/ethyl acetate 6:1) |
| ¹H-NMR(250MHz, | |
| CDCl₃): | |
| Non-cholesterol | |
| signals: | |
| δ=1.01 | (t, ³J=7.0Hz, 3H, —NCH₂CH₃) |
| δ=1.23-1.57 | (m, 6 H, —NCH₂(CH₂)₃CH₂N—) |
| δ=2.40 | (t, ³J=7.3Hz, 2H, —N(CH₂)₄CH₂NCH₂CH₃) |
| δ=2.48 | (quart, ³J=7.1Hz, 2H, —NCH₂CH₃) |
| δ=2.60 | (t, ³J=7.3Hz, 2H, —NCH₂(CH₂)₄NCH₂CH₃) |
| δ=3.26 | (s, 2H, OCOCH₂N(CH₂)₃N—) |
| δ=3.53 | (s, 2H, C₆H₅CH₂—NCH₂CH₃) |
| δ=3.76 | (s, 2H, C₆H₅CH₂—N(CH₂)₅NCH₂CH₃) |
| δ=7.17-7.37 | (m, 10H, H_{aromat.}) |

1-(cholesteryloxycarbonylmethyl)-1,8-dibenzyl-1,8-diazadekan (49)

Quantities Used:

325 mg (1.0 mmol) N-ethyl-N,N′-dibenzyl-1,6-diaminohexane (40)

648 mg (1.4 mmol) chloroacetic acid cholesterylester (1)

69 mg (0.5 mmol) potassium carbonate

Perform purification via column chromatography on 20 g silica gel, elute the apolar impurities with cyclohexane/diisopropyl ether (1:1), and elute the product with cyclohexane/ethyl acetate (10:1). The yield is 518 mg 49 as a colorless oil.

| | |
|---|---|
| Yield: | 518mg(69% of theoretical value)as a yellow oil |
| Mᵣ: | 751.19(C₅₁H₇₈N₂O₂) |
| R_{f}: | 0.25(cyclohexane/ethyl acetate 6:1) |
| ¹H-NMR(250MHz, | |
| CDCl₃): | |
| Non-cholesterol | |
| signals: | |
| δ=1.02 | (t, ³J=7.0Hz, 3H, —NCH₂CH₃) |
| δ=1.20-1.34 | (m, 4H, —N(CH₂)₂(CH₂)₂(CH₂)₂N—) |
| δ=1.38-1.55 | (m, 4H, —NCH₂CH₂(CH₂)₂CH₂CH₂N—) |
| δ=2.39 | (t, ³J=7.3Hz, 2H, —N(CH₂)₅CH₂NCH₂CH₃) |
| δ=2.49 | (quart, ³J=7.1Hz, 2H, —NCH₂CH₃) |
| δ=2.60 | (t, ³J=7.2Hz, 2H, —NCH₂(CH₂)₅NCH₂CH₃) |
| δ=3.26 | (s, 2H, OCOCH₂N(CH₂)₆N—) |
| δ=3.54 | (s, 2H, C₆H₅CH₂—NCH₂CH₃) |
| δ=3.76 | (s, 2H, C₆H₅CH₂—N(CH₂)₆NCH₂CH₃) |
| δ=7.17-7.38 | (m, 10H, H_{aromat.}) |

General Synthesis Instructions for Lipids with Two Lipid Anchors:

Stir a mixture of 1.0 mmol of the respective N,N′-dibenzyl-α,ω-diaminoalkane (22-25), 2.6 mmol of the respective lipid component, and 0.5 mmol potassium carbonate in 10 ml acetonitrile/toluene (8:1) overnight with reflux. Remove the solvent completely and purify the residue via column chromatography.

N,N′-to-(cholesteryloxycarbonylmethyl)-N,N′-1,4-dibenzyl-1,4-diaminobutane (50)

Quantities Used:

268 mg (1.0 mmol) N,N′-dibenzyl-1,4-diaminobutane (3)

1204 mg (2.6 mmol) chloroacetic acid cholesterylester (1)

69 mg (0.5 mmol) potassium carbonate

Perform purification via column chromatography on 30 g silica gel, elute the lipid component with cyclohexane/diisopropyl ether (6:1), and elute the product with cyclohexane/diisopropyl ether (4:1). The yield is 830 mg 50 as a colorless oil.

| | |
|---|---|
| Yield: | 830mg(74% of theoretical value)as a yellow oil |
| Mᵣ: | 1121.77(C₇₆H₁₁₆N₂O₄) |
| R_{f}: | 0.53(cyclohexane/ethyl acetate 4:1) |
| ¹H-NMR(250MHz, | |
| CDCl₃): | |
| Non-cholesterol | |
| signals: | |
| δ=1.50-1.62 | (m, 4H, —NCH₂(CH₂)₂CH₂N—) |
| δ=2.54-2.70 | (m, 4H, —NCH₂(CH₂)₂CH₂N—) |
| δ=3.25 | (s, 4H, —OCOCH₂N(CH₂)₄NCH₂COO—) |
| δ=3.75 | (s, 4H, C₆H₅CH₂—N |
| | (CH₂)₄N—CH₂C₆H₅) |
| δ=7.18-7.37 | (m, 10H, H_{aromat.}) |

N,N′-to-(cholesteryloxycarbonyloxy)-ethyl)-N,N′-1,4-dibenzyl-1,4- diaminobutane (51)

Quantities Used:

268 mg (1.0 mmol) N,N′-dibenzyl-1,4-diaminobutane (23)

1398 mg (2.6 mmol) 2-bromoethyl-cholesterylcarbonate (14)

69 mg (0.5 mmol) potassium carbonate

Perform purification via column chromatography on 30 g silica gel, elute the lipid component with cyclohexane/diisopropyl ether (6:1), and elute the product with cyclohexane/diisopropyl ether (4:1). The yield is 721 mg 51 as a colorless oil.

| | |
|---|---|
| Yield: | 721mg(61% of theoretical value)as a yellow oil |
| Mᵣ: | 1181.82(C₇₈H₁₂₀N₂O₆) |
| R_{f}: | 0.41(cyclohexane/diisopropyl ether 1:1) |
| ¹H-NMR(250MHz, | |
| CDCl₃): | |
| Non-cholesterol | |
| signals: | |
| δ=1.50-1.62 | (m, 4H, —NCH₂(CH₂)₂CH₂N—) |
| δ=2.39-2.51 | (m, 4H, —NCH₂(CH₂)₂CH₂N—) |
| δ=2.71 | (t, ³J=6.4Hz, 4H, —OCH₂CH₂N |
| | (CH₂)₄NCH₂CH₂O—) |
| δ=3.60 | (s, 4H, C₆H₅CH₂—N(CH₂)₄ |
| | N—CH₂C₆H₅) |
| δ=4.14 | (t, ³J=6.3Hz, 4H, —OCH₂CH₂N |
| | (CH₂)₄NCH₂CH₂O—) |
| δ=7.15-7.33 | (m, 10H, H_{aromat.}) |

N,N′-to-(cholesterylhemisuccinoyloxy-2-ethyl)-N,N′-1,4-dibenzyl-1,4- diaminobutane (52)

Quantities Used:

268 mg (1.0 mmol) N,N′-dibenzyl-1,4-diaminobutane (23)

1544 mg (2.6 mmol) 2-bromoethyl-cholesterylsuccinate (15)

69 mg (0.5 mmol) potassium carbonate

Perform purification via column chromatography on 30 g silica gel, elute the excess lipid component with cyclohexane/diisopropyl ether (2:1), and elute the product with diisopropyl ether. The yield is 686 mg 52 as a colorless oil.

| | |
|---|---|
| Yield: | 686mg(53% of theoretical value)as a yellow oil |
| Mᵣ: | 1293.95(C₈₄H₁₂₈N₂O₈) |
| R_{f}: | 0.59(cyclohexane/ethyl acetate 2:1) |
| ¹H-NMR(250MHz, | |
| CDCl₃): | |
| Non-cholesterol | |
| signals: | |
| δ=1.50-1.62 | (m, 4H, —NCH₂(CH₂)₂CH₂N—) |
| δ=2.40-2.50 | (m, 4H, —NCH₂(CH₂)₂CH₂N—) |
| δ=2.53-2.61 | (m, 8 H, —OCO(CH₂)₂COO(CH₂)₂N(CH₂)₄ |
| | N(CH₂)₂OCO(CH₂)₂COO—) |
| δ=2.68 | (t, ³J=6.3Hz, 4H, —OCH₂CH₂N |
| | (CH₂)₄NCH₂CH₂O—) |
| δ=3.59 | (s, 4H, C₆H₅CH₂—N(CH₂)₄ |
| | N—CH₂C₆H₅) |
| δ=4.12 | (t, ³J=6.1Hz, 4H, —OCH₂CH₂N |
| | (CH₂)₄NCH₂CH₂O—) |
| δ=7.15-7.32 | (m, 10H, H_{aromat.}) |

N,N′-to-(cholesterylhemisuccinoyloxy-3-propyl)-N,N′-1,4-dibenzyl-1,4- diaminobutane (53)

Quantities Used:

268 mg (1.0 mmol) N,N′-dibenzyl-1,4-diaminobutane (23)

1580 mg (2.6 mmol) 3-bromopropyl-cholesterylsuccinate (16)

69 mg (0.5 mmol) potassium carbonate

Perform purification via column chromatography on 30 g silica gel, elute the excess lipid component with cyclohexane/diisopropyl ether (1:1), and elute the product with diisopropyl ether. The yield is 595 mg 53 as a colorless oil.

| | |
|---|---|
| Yield: | 595mg(45% of theoretical value)as a yellow oil |
| Mᵣ: | 1322.00(C₈₆H₁₃₂N₂O₈) |
| R_{f}: | 0.42(cyclohexane/ethyl acetate 2:1) |
| ¹H-NMR(250MHz, | |
| CDCl₃): | |
| Non-cholesterol | |
| signals: | |
| δ=1.50-1.62 | (m, 4H, —NCH₂(CH₂)₂CH₂N—) |
| δ=2.26-2.50 | (m, 8 H, —OCH₂CH₂CH₂NCH₂ |
| | (CH₂)₂CH₂NCH₂CH₂CH₂O—) |
| δ=2.44 | (t, ³J=6.9Hz, 4H, —O(CH₂)₂CH₂ |
| | N(CH₂)₄NCH₂(CH₂)₂O—) |
| δ=2.48-2.58 | (m, 8 H, |
| | —O(CH₂)₂COO(CH₂)₃N(CH₂)₄ |
| | N(CH₂)₃OCO(CH₂)₂COO—) |
| δ=3.50 | (s, 4H, C₆H₅CH₂—N(CH₂)₄ |
| | N—CH₂C₆H₅) |
| δ=4.09 | (t, ³J=6.6Hz, 4H, —OCH₂(CH₂)₂ |
| | N(CH₂)₄N(CH₂)₂CH₂O—) |
| δ=7.15-7.40 | (m, 10H, H_{aromat.}) |

N,N′-to-(cholesteryloxycarbonylmethyl)-N,N′-1,3-dibenzyl-1,3-diaminopropane (54)

Quantities Used:

254 mg (1.0 mmol) N,N′-dibenzyl-1,3-diaminopropane (22)

1204 mg (2.6 mmol) chloroacetic acid cholesterylester (1)

69 mg (0.5 mmol) potassium carbonate

Perform purification via column chromatography on 30 g silica gel, elute the excess lipid component with cyclohexane/diisopropyl ether (6:1), and elute the product with cyclohexane/diisopropyl ether (4:1). The yield is 709 mg 54 as a colorless oil.

| | |
|---|---|
| Yield: | 709mg(64% of theoretical value)as a yellow oil |
| Mᵣ: | 1107.74(C₇₅H₁₁₄N₂O₄) |
| R_{f}: | 0.54(cyclohexane/ethyl acetate 4:1) |
| ¹H-NMR(250MHz, | |
| CDCl₃): | |
| Non-cholesterol | |
| signals: | |
| δ=1.60-1.74 | (m, 2H, —NCH₂CH₂CH₂N—) |
| δ=2.67 | (t, ³J=7.0Hz, 4H, —NCH₂CH₂CH₂N—) |
| δ=3.24 | (s, 4H, —OCOCH₂ |
| | N(CH₂)₃NCH₂COO—) |
| δ=3.75 | (s, 4H, C₆H₅CH₂—N(CH₂)₃ |
| | N—CH₂C₆H₅) |
| δ=7.17-7.33 | (m, 10H, H_{aromat.}) |

N,N′-to-(cholesteryloxycarbonylmethyl)-N,N′-1,5-dibenzyl-1,5-diaminopentane (55)

Quantities Used:

282 mg (1.0 mmol) N,N′-dibenzyl-1,5-diaminopentane (24)

1204 mg (2.6 mmol) chloroacetic acid cholesterylester (1)

69 mg (0.5 mmol) potassium carbonate

Perform purification via column chromatography on 30 g silica gel, elute the excess lipid component with cyclohexane/diisopropyl ether (6:1), and elute the product with cyclohexane/diisopropyl ether (4:1). The yield is 863 mg 55 as a colorless oil.

| | |
|---|---|
| Yield: | 863mg(76% of theoretical value)as a yellow oil |
| Mᵣ: | 1135.79(C₇₇H₁₁₈N₂O₄) |
| R_{f}: | 0.59(cyclohexane/ethyl acetate 4:1) |
| ¹H-NMR(250MHz, | |
| CDCl₃): | |
| Non-cholesterol | |
| signals: | |
| δ=1.25-1.57 | (m, 6 H, —NCH₂(CH₂)₃CH₂N—) |
| δ=2.60 | (t, 4H, ³J=7.2Hz, —NCH₂ |
| | (CH₂)₃CH₂N—) |
| δ=3.23 | (s, 4H, —OCOCH₂N(CH₂)₅ |
| | NCH₂COO—) |
| δ=3.76 | (s, 4H, C₆H₅CH₂—N |
| | (CH₂)₅N—CH₂C₆H₅) |
| δ=7.15-7.37 | (m, 10H, H_{aromat.}) |

N,N′-to-(cholesteryloxycarbonylmethyl)-N,N′-1,6-dibenzyl-1,6-diaminohexane (56)

Quantities Used:

296 mg (1.0 mmol) N,N′-dibenzyl-1,6-diaminohexane (25)

1204 mg (2.6 mmol) chloroacetic acid cholesterylester (1)

69 mg (0.5 mmol) potassium carbonate

Perform purification via column chromatography on 30 g silica gel, elute the excess lipid component with cyclohexane/diisopropyl ether (6:1), and elute the product with cyclohexane/diisopropyl ether (4:1). The yield is 793 mg 56 as a colorless oil.

| | |
|---|---|
| Yield: | 793mg(69% of theoretical value)as a yellow oil |
| Mᵣ: | 1149.82(C₇₈H₁₂₀N₂O₄) |
| R_{f}: | 0.62(cyclohexane/ethyl acetate 4:1) |
| ¹H-NMR(250MHz, | |
| CDCl₃): | |
| Non-cholesterol | |
| signals: | |
| δ=1.20-1.34 | (m, 4H, —N(CH₂)₂(CH₂)₂(CH₂)₂N—) |
| δ=1.38-1.55 | (m, 4H, —NCH₂CH₂(CH₂)₂CH₂CH₂N—) |
| δ=2.60 | (t, 4H, ³J=7.3Hz, —NCH₂ |
| | (CH₂)₄CH₂N—) |
| δ=3.26 | (s, 4H, —OCOCH₂N(CH₂)₆ |
| | NCH₂COO—) |
| δ=3.76 | (s, 4H, C₆H₅CH₂—N(CH₂)₆ |
| | N—CH₂C₆H₅) |
| δ=7.20-7.37 | (m, 10H, H_{aromat.}) |

General Synthesis Instructions for Lipids with One Lipid Anchor:

Add 0.1 mmol palladium/activated charcoal (10%) to a solution of 1.0 mmol of the respective benzyl-protected, bicationic lipid with two lipid anchors (57-64) in 4 ml of a solvent mixture of dichloromethane/methanol/acetic acid (2:1:1). Stir overnight in a hydrogen atmosphere. Concentrate the formulation to a small volume until dry and purify the residue via column chromatography.

1-(cholesteryloxycarbonylmethyl)-1,6-diazaoctane acetic acid salt (57)

Quantities Used:

723 mg (1.0 mmol) 1-(cholesteryloxycarbonylmethyl)-1,6-dibenzyl-1,6-diazaoctane (41)

106 mg (0.1 mmol) palladium/activated charcoal

Perform purification via column chromatography on 20 g silica gel, elute the apolar impurities with chloroform/methanol/acetic acid (80:20:2), and elute the product with chloroform/methanol/acetic acid (80:20:6). After the solvent is removed, precipitate the product out of an acetone/diisopropyl ether mixture. The yield is 451 mg 57 as a colorless solid.

| | |
|---|---|
| Yield: | 451mg(68% of theoretical value)as a colorless solid |
| Mᵣ: | 662.99(C₃₉H₇₀N₂O₆) |
| R_{f}: | 0.12(chloroform/methanol/acetic acid 80:20:4) |
| ¹H-NMR(250MHz, | |
| CDCl₃/CD₃OD/ | |
| D₂O 20:10:1): | |
| Non-cholesterol | |
| signals: | |
| δ=1.29 | (t, ³J=7.3Hz, 3H, —NCH₂CH₃) |
| δ=1.79-1.91 | (m, 4H, —NCH₂(CH₂)₂CH₂N—) |
| δ=1.96 | (s, 6 H, 2 CH₃COO⁻) |
| δ=2.67 | (t, ³J=6.3Hz, 2H, —N(CH₂)₃CH₂NCH₂CH₃) |
| δ=2.92 | (t, ³J=6.6Hz, 2H, —NCH₂(CH₂)₃NCH₂CH₃) |
| δ=2.99 | (quart, ³J=7.3Hz, 2H, —NCH₂CH₃) |
| δ=3.44 | (s, 2H, —OCOCH₂N(CH₂)₄N—) |

1-(2-(cholesteryloxycarbonyloxy)-ethyl)-1,6-diazaoctane acetic acid salt (58)

Quantities Used:

753 mg (1.0 mmol) 1-(2-(cholesteryloxycarbonyloxy)-ethyl)-1,6-dibenzyl-1,6-diazaoctane (42)

106 mg (0.1 mmol) palladium/activated charcoal

Perform purification via column chromatography on 15 g silica gel, elute the apolar impurities with chloroform/methanol (60:40), and elute the product with chloroform/methanol/acetic acid (60:40:6). After the solvent is removed, precipitate the product out of an acetone/diisopropyl ether mixture. The yield is 318 mg 58 as a colorless solid.

| | |
|---|---|
| Yield: | 318mg(45% of theoretical value)as a colorless solid |
| Mᵣ: | 707.00(C₄₀H₇₀N₂O₈) |
| R_{f}: | 0.14(chloroform/methanol/acetic acid 80:20:4) |
| ¹H-NMR(250MHz, | |
| CDCl₃/CD₃OD/ | |
| D₂O 20:10:1): | |
| Non-cholesterol | |
| signals: | |
| δ=1.31 | (t, ³J=7.3Hz, 3H, —NCH₂CH₃) |
| δ=1.70-1.82 | (m, 4H, —NCH₂(CH₂)₂CH₂N—) |
| δ=2.00 | (s, 6 H, 2 CH₃COO⁻) |
| δ=2.93-3.10 | (m, 4H, —NCH₂(CH₂)₂CH₂NCH₂CH₃) |
| δ=3.03 | (quart, ³J=7.3Hz, 2H, —NCH₂CH₃) |
| δ=3.31 | (t, ³J=5.0Hz, 2H, —OCOOCH₂CH₂N—) |
| δ=4.32-4.47 | (m, 2H, —OCOOCH₂CH₂N—) |

1-(cholesterylhemisuccinoyloxy-2-ethyl)-1,6-diazaoctane acetic acid salt (59)

Quantities Used:

809 mg (1.0 mmol) 1-(cholesterylhemisuccinoyloxy-2-ethyl)-1,6-dibenzyl-1,6-diazaoctane (43)

106 mg (0.1 mmol) palladium/activated charcoal

Perform purification via column chromatography on 15 g silica gel, elute the apolar impurities with chloroform/methanol/acetic acid (60:40:6), and elute the product with chloroform/methanol/acetic acid/water (60:40:6:2). After the solvent is removed, precipitate the product out of an acetone/diisopropyl ether mixture. The yield is 374 mg 59 as a colorless solid.

| | |
|---|---|
| Yield: | 374mg(50% of theoretical value)as a colorless solid |
| Mᵣ: | 747.07(C₄₃H₇₄N₂O₈) |
| R_{f}: | 0.52(chloroform/methanol/acetic acid/ |
| | water 60:40:6:2) |
| ¹H-NMR(250MHz, | |
| CDCl₃/CD₃OD/ | |
| D₂O 20:10:1): | |
| Non-cholesterol | |
| signals: | |
| δ=1.31 | (t, ³J=7.3Hz, 3H, —NCH₂CH₃) |
| δ=1.68-1.81 | (m, 4H, —NCH₂(CH₂)₂CH₂N—) |
| δ=1.96 | (s, 6H, 2CH₃COO⁻) |
| δ=2.62-2.69 | (m, 4H, —OCO(CH₂)₂COO—) |
| δ=2.85 | (t, ³J=6.9Hz, 2H, —N(CH₂)₃CH₂NCH₂CH₃—) |
| δ=2.94 | (t, ³J=7.0Hz, 2H, |
| | —OCH₂CH₂NCH₂(CH₂)₃NCH₂CH₃) |
| δ=3.00 | (quart, ³J=7.3Hz, 2H —NCH₂CH₃) |
| δ=3.09 | (t, ³J=5.3Hz, 2H, —OCH₂CH₂N(CH₂)₄NCH₂CH₃) |
| δ=4.32 | (t, ³J=5.3Hz, 2H, —OCH₂CH₂N(CH₂)₄NCH₂CH₃) |

1-(cholesterylhemisuccinoyloxy-3-propyl)-1,6-diazaoctane acetic acid salt (60)

Quantities Used:

823 mg (1.0 mmol) 1-(cholesterylhemisuccinoyloxy-3-propyl)-1,6-dibenzyl-1,6-diazaoctane (44)

106 mg (0.1 mmol) palladium/activated charcoal

Perform purification via column chromatography on 15 g silica gel, elute the apolar impurities with chloroform/methanol/acetic acid (60:40:6), and elute the product with chloroform/methanol/acetic acid/water (60:40:6:2). After the solvent is removed, precipitate the product out of an acetone/diisopropyl ether mixture. The yield is 441 mg 60 as a colorless solid.

| | |
|---|---|
| Yield: | 441mg(58% of theoretical value)as a colorless solid |
| Mᵣ: | 761.10(C₄₄H₇₆N₂O₈) |
| R_{f}: | 0.41(chloroform/methanol/acetic |
| | acid/water 60:40:6:2) |
| ¹H-NMR(250MHz, | |
| CDCl₃/ | |
| CD₃OD/ | |
| D₂O 20:10:1): | |
| Non-cholesterol | |
| signals: | |
| δ=1.30 | (t, ³J=7.3Hz, 3H, —NCH₂CH₃) |
| δ=1.68-1.81 | (m, 6H, —OCH₂CH₂CH₂NCH₂(CH₂)₂CH₂N—) |
| δ=1.95 | (s, 6H, 2CH₃COO⁻) |
| δ=2.60-2.67 | (m, 4H, —OCO(CH₂)₂COO—) |
| δ=2.88-3.05 | (m, H, —O(CH₂)₂CH₂NCH₂(CH₂)₂CH₂NCH₂CH₃) |
| δ=2.98 | (quart, ³J=7.3Hz, 2H —NCH₂CH₃) |
| δ=4.19 | (t, ³J=6.1Hz, 2H, —COOCH₂(CH₂)₂N—) |

1-((cholesterylhemisuccinoyloxy-2-ethyloxy)-2-ethyl)-1,6-diazaoctane acetic acid salt (61)

Quantities Used:

853 mg (1.0 mmol) 1-((cholesterylhemisuccinoyloxy-2-ethyloxy)-2-ethyl)-1,6-dibenzyl-1,6-diazaoctane (45)

106 mg (0.1 mmol) palladium/activated charcoal

Perform purification via column chromatography on 15 g silica gel, elute the apolar impurities with chloroform/methanol (90:10), and elute the product with chloroform/methanol/ammonia (25%) (90:10:1). After the solvent is removed and one drop of acetic acid is added, precipitate the product out of an acetone/diisopropyl ether mixture. The yield is 374 mg 61 as a colorless solid.

| | |
|---|---|
| Yield: | 348mg(44% of theoretical value)as a colorless solid |
| Mᵣ: | 793.14(C₄₅H₈₀N₂O₉) |
| R_{f}: | 0.09(chloroform/methanol/ammonia |
| | (25%) 90:10:1) |
| ¹H-NMR(250MHz, | |
| CDCl₃/ | |
| CD₃OD/ | |
| D₂O 20:10:1): | |
| Non-cholesterol | |
| signals: | |
| δ=1.31 | (t, ³J=7.3Hz, 3H, —NCH₂CH₃) |
| δ=1.68-1.81 | (m, 4H, —NCH₂(CH₂)₂CH₂N—) |
| δ=2.00 | (s, 6H, 2CH₃COO⁻) |
| δ=2.62-2.68 | (m, 4H, —OCO(CH₂)₂COO—) |
| δ=2.90-3.01 | (m, 4H, —OCH₂CH₂NCH₂(CH₂)₂CH₂NCH₂CH₃) |
| δ=3.02 | (quart, ³J=7.3Hz, 2H, —NCH₂CH₃) |
| δ=3.11 | (t, ³J=5.2Hz, 2H, —OCH₂CH₂N(CH₂)₄NCH₂CH₃) |
| δ=3.70-3.81 | (m, 4H, —OCH₂CH₂OCH₂CH₂N—) |
| δ=4.29 | (t, ³J=4.7Hz, 2H, —OCH₂CH₂OCH₂CH₂N—) |

1-(cholesteryloxycarbonylmethyl)-1,5-diazaheptane acetic acid salt (62)

Quantities Used:

709 mg (1.0 mmol) 1-(cholesteryloxycarbonylmethyl)-1,5-dibenzyl-1,5-diazaheptane (47)

106 mg (0.1 mmol) palladium/activated charcoal

Perform purification via column chromatography on 20 g silica gel, elute the apolar impurities with chloroform/methanol/acetic acid (80:20:2), and elute the product with chloroform/methanol/acetic acid (80:20:6). After the solvent is removed, precipitate the product out of an acetone/diisopropyl ether mixture. The yield is 350 mg 62 as a colorless solid.

| | |
|---|---|
| Yield: | 350mg(54% of theoretical value)as a colorless solid |
| Mᵣ: | 648.97(C₃₈H₆₈N₂O₆) |
| R_{f}: | 0.17(chloroform/methanol/acetic acid 80:20:4) |
| ¹H-NMR(250MHz, | |
| CDCl₃/ | |
| CD₃OD/ | |
| D₂O 20:10:1): | |
| Non-cholesterol | |
| signals: | |
| δ=1.33 | (t, ³J=7.2Hz, 3H, —NCH₂CH₃) |
| δ=1.80-1.91 | (m, 2H, —NCH₂CH₂CH₂N—) |
| δ=1.95 | (s, 6H, 2CH₃COO⁻) |
| δ=2.79 | (t, ³J=6.1Hz, 2H, —N(CH₂)₂CH₂NCH₂CH₃) |
| δ=3.07 | (quart, ³J=7.3Hz, 2H —NCH₂CH₃) |
| δ=3.11 | (t, ³J=6.7Hz, 2H, —NCH₂(CH₂)₂NCH₂CH₃) |
| δ=3.46 | (s, 2H, —OCOCH₂N(CH₂)₃N—) |

1-(cholesteryloxycarbonylmethyl)-1,7-diazanonane acetic acid salt (63)

Quantities Used:

737 mg (1.0 mmol) 1-(cholesteryloxycarbonylmethyl)-1,7-dibenzyl-1,7-diazanonane (48)

106 mg (0.1 mmol) palladium/activated charcoal

Perform purification via column chromatography on 20 g silica gel, elute the apolar impurities with chloroform/methanol/acetic acid (80:20:2), and elute the product with chloroform/methanol/acetic acid (80:20:6). After the solvent is removed, precipitate the product out of an acetone/diisopropyl ether mixture. The yield is 596 mg 63 as a colorless solid.

| | |
|---|---|
| Yield: | 596mg(88% of theoretical value)as a colorless solid |
| Mᵣ: | 677.02(C₄₀H₇₂N₂O₆) |
| R_{f}: | 0.15(chloroform/methanol/acetic acid 80:20:4) |
| ¹H-NMR(250MHz, | |
| CDCl₃/ | |
| CD₃OD/ | |
| D₂O 20:10:1): | |
| Non-cholesterol | |
| signals: | |
| δ=1.32 | (t, ³J=7.2Hz, 3H, —NCH₂CH₃) |
| δ=1.23-1.37 | (m, 2H, —N(CH₂)₂CH₂(CH₂)₂N—) |
| δ=1.38-1.57 | (m, 4H, —HNCH₂CH₂CH₂CH₂CH₂N—) |
| δ=2.00 | (s, 6H, 2CH₃COO⁻) |
| δ=2.84 | (t, ³J=7.3Hz, 2H, —N(CH₂)₄CH₂NCH₂CH₃) |
| δ=2.93 | (t, ³J=7.9Hz, 2H, —NCH₂(CH₂)₄NCH₂CH₃) |
| δ=3.01 | (quart, ³J=7.4Hz, 2H —NCH₂CH₃) |
| δ=3.64 | (s, 2H, —OCOCH₂N(CH₂)₅N—) |

1-(cholesteryloxycarbonylmethyl)-1,8-diazadekan acetic acid salt (64)

Quantities Used:

751 mg (1.0 mmol) 1-(cholesteryloxycarbonylmethyl)-1,8-dibenzyl-1,8-diazadekan (49)

106 mg (0.1 mmol) palladium/activated charcoal

Perform purification via column chromatography on 20 g silica gel, elute the apolar impurities with chloroform/methanol/acetic acid (80:20:2), and elute the product with chloroform/methanol/acetic acid (80:20:6). After the solvent is removed, precipitate the product out of an acetone/diisopropyl ether mixture. The yield is 580 mg 64 as a colorless solid.

| | |
|---|---|
| Yield: | 580mg(84% of theoretical value)as a colorless solid |
| Mᵣ: | 691.05(C₄₁H₇₄N₂O₆) |
| R_{f}: | 0.10(chloroform/methanol/acetic acid 80:20:4) |
| ¹H-NMR(250MHz, | |
| CDCl₃/ | |
| CD₃OD/ | |
| D₂O 20:10:1): | |
| Non-cholesterol | |
| signals: | |
| δ=1.33 | (t, ³J=7.3Hz, 3H, —NCH₂CH₃) |
| δ=1.20-1.34 | (m, 4H, —N(CH₂)₂(CH₂)₂(CH₂)₂N—) |
| δ=1.38-1.55 | (m, 4H, —NCH₂CH₂(CH₂)₂CH₂CH₂N—) |
| δ=2.01 | (s, 6H, 2CH₃COO⁻⁾ |
| δ=2.80-3.03 | (m, 4H, —NCH₂(CH₂)₄CH₂N—) |
| δ=3.03 | (quart, ³J=7.3Hz, 2H, —NCH₂CH₃) |
| δ=3.69 | (s, 2H, —OCOCH₂N(CH₂)₆N—) |

General Synthesis Instructions for Lipids with Two Lipid Anchors:

Add 0.1 mmol palladium/activated charcoal (10%) to a solution of 1.0 mmol of the respective benzyl-protected bicationic lipid with two-lipid anchors (65-71) in 4 ml of a solvent mixture of dichloromethane/methanol/acetic acid (2:1:1). Stir overnight in a hydrogen atmosphere. Concentrate the formulation to a small volume to dry it and purify the residue via column chromatography.

N,N′-to-(cholesteryloxycarbonylmethyl)-1,4-diaminobutane acetic acid salt (65)

Quantities Used:

1122 mg (1.0 mmol) N,N′-to-(cholesteryloxycarbonylmethyl)-N,N′-1,4-dibenzyl-1,4-diaminobutane (50)

106 mg (0.1 mmol) palladium/activated charcoal

Perform purification via column chromatography on 25 g silica gel with chloroform/2-propanol/acetic acid (60:40:1). After the solvent is removed, precipitate the product out of an acetone/diisopropyl ether mixture. The yield is 499 mg 65 as a colorless solid.

| | |
|---|---|
| Yield: | 499mg(47% of theoretical value)as a colorless solid |
| Mᵣ: | 1061.62(C₆₆H₁₁₂N₂O₈) |
| R_{f}: | 0.27(chloroform/methanol/2-propanol/acetic |
| | acid 80:20:20:1) |
| ¹H-NMR(250MHz, | |
| CDCl₃/ | |
| CD₃OD/ | |
| D₂O 20:10:1): | |
| Non-cholesterol | |
| signals: | |
| δ=1.68-1.82 | (m, 4H, —NCH₂(CH₂)₂CH₂N—) |
| δ=1.97 | (s, 6H, 2CH₃COO⁻) |
| δ=2.70-2.83 | (m, 4H, —NCH₂(CH₂)₂CH₂N—) |
| δ=3.56 | (s, 4H, —OCOCH₂N(CH₂)₄NCH₂COO—) |

N,N′-to-(2-(cholesteryloxycarbonyloxy)-ethyl)-1,4-diaminobutane (66)

Quantities Used:

1182 mg (1.0 mmol) N,N′-to-(2-(cholesteryloxycarbonyloxy)-ethyl)-N,N′-1,4-dibenzyl-1,4-diaminobutane (51)

106 mg (0.1 mmol) palladium/activated charcoal

Perform purification via column chromatography on 25 g silica gel with chloroform/2-propanol/acetic acid (80:20:2). After the solvent is removed, precipitate the product out of an acetone/diisopropyl ether mixture. The yield is 740 mg 66 as a colorless solid.

| | |
|---|---|
| Yield: | 740mg(66% of theoretical value)as a colorless solid |
| Mᵣ: | 1121.68(C₆₈H₁₁₆N₂O₁₀) |
| R_{f}: | 0.12(chloroform/methanol/2-propanol/acetic |
| | acid 80:20:20:1) |
| ¹H-NMR(250MHz, | |
| CDCl₃/ | |
| CD₃OD/ | |
| D₂O 20:10:1): | |
| Non-cholesterol | |
| signals: | |
| δ=1.68-1.82 | (m, 4H, —NCH₂(CH₂)₂CH₂N—) |
| δ=2.02 | (s, 6H, 2CH₃COO⁻⁾ |
| δ=2.91-3.03 | (m, 4H, —NCH₂(CH₂)₂CH₂N—) |
| δ=3.18-3.27 | (m, 4H, —OCH₂CH₂N(CH₂)₄NCH₂CH₂O—) |
| δ=4.35-4.50 | (m, 4H, —OCH₂CH₂N(CH₂)₄NCH₂CH₂O—) |

N,N′-to-(cholesterylhemisuccinoyloxy-2-ethyl)-1,4-diaminobutane (67)

Quantities Used:

1294 mg (1.0 mmol) N,N′-to-(cholesterylhemisuccinoyloxy-2-ethyl)-N,N′-1,4-dibenzyl-1,4-diaminobutane (52)

106 mg (0.1 mmol) palladium/activated charcoal

Perform purification via column chromatography on 25 g silica gel, elute the apolar impurities with chloroform/methanol/acetic acid (90:10:2), and elute the product with chloroform/methanol/acetic acid (80:20:2). Precipitate the product out of an acetone/diisopropyl ether mixture. The yield is 901 mg 67 as a colorless solid.

| | |
|---|---|
| Yield: | 901mg(73% of theoretical value)as a colorless solid |
| Mᵣ: | 1233.80(C₇₄H₁₂₄N₂O₁₂) |
| R_{f}: | 0.23(chloroform/methanol/acetic acid 80:20:2) |
| ¹H-NMR(250MHz, | |
| CDCl₃/ | |
| CD₃OD/ | |
| D₂O 20:10:1): | |
| Non-cholesterol | |
| signals: | |
| δ=1.68-1.82 | (m, 4H, —NCH₂(CH₂)₂CH₂N—) |
| δ=2.02 | (s, 6H, 2CH₃COO⁻) |
| δ=2.60-2.66 | (m, 8H, —OCO(CH₂)₂COO(CH₂)₂ |
| | N(CH₂)₄N(CH₂)₂OCO(CH₂)₂COO—) |
| δ=2.72-2.84 | (m, 4H, —NCH₂(CH₂)₂CH₂N—) |
| δ=2.97-3.07 | (m, 4H, —OCH₂CH₂N(CH₂)₄NCH₂CH₂O—) |
| δ=4.27-4.36 | (m, 4H, —OCH₂CH₂N(CH₂)₄NCH₂CH₂O—) |

N,N′-to-(cholesterylhemisuccinoyloxy-3-propyl)-1,4-diaminobutane (68)

Quantities Used:

1322 mg (1.0 mmol) N,N′-to-(cholesterylhemisuccinoyloxy-3-propyl)-N,N′-1,4-dibenzyl-1,4-diaminobutane (53)

106 mg (0.1 mmol) palladium/activated charcoal

Perform purification via column chromatography on 25 g silica gel, elute the apolar impurities with chloroform/methanol/acetic acid (90:10:2), and elute the product with chloroform/methanol/acetic acid (80:20:2). Precipitate the product out of an acetone/diisopropyl ether mixture. The yield is 1161 mg 68 as a colorless solid.

| | |
|---|---|
| Yield: | 1161mg(92% of theoretical value)as a colorless |
| | solid |
| Mᵣ: | 1261.86(C₇₆H₁₂₈N₂O₁₂) |
| R_{f}: | 0.29(chloroform/methanol/acetic acid 80:20:2) |
| ¹H-NMR(250MHz, | |
| CDCl₃/ | |
| CD₃OD/ | |
| D₂O 20:10:1): | |
| Non-cholesterol | |
| signals: | |
| δ=1.68-1.82 | (m, 4H, —NCH₂(CH₂)₂CH₂N—) |
| δ=1.98 | (s, 6H, 2CH₃COO⁻) |
| δ=2.02-2.17 | (m, 4H, —OCH₂CH₂CH₂N(CH₂)₄ |
| | NCH₂CH₂CH₂O—) |
| δ=2.60-2.67 | (m, 8H, —OCO(CH₂)₂COO(CH₂)₃N(CH₂)₄ |
| | N(CH₂)₃OCO(CH₂)₂COO—) |
| δ=2.93-3.09 | (m, 8H, —O(CH₂)₂CH₂NCH₂(CH₂)₂CH₂ |
| | NCH₂(CH₂)₂O—) |
| δ=4.22 | (t, ³J=6.1Hz, 4H, —OCH₂(CH₂)₂ |
| | N(CH₂)₄N(CH₂)₂CH₂O—) |

N,N′-to-(cholesteryloxycarbonylmethyl)-1,3-diaminopropaneacetic acid salt (69)

Quantities Used:

1108 mg (1.0 mmol) N,N′-to-(cholesteryloxycarbonylmethyl)-N,N′-1,3-dibenzyl-1,3-diaminopropane (54)

106 mg (0.1 mmol) palladium/activated charcoal

Perform purification via column chromatography on 25 g silica gel with chloroform/2-propanol/acetic acid (60:40:1). After the solvent is removed, precipitate the product out of an acetone/diisopropyl ether mixture. The yield is 513 mg 69 as a colorless solid.

| | |
|---|---|
| Yield: | 513mg(49% of theoretical value) as |
| | a colorless solid |
| Mᵣ: | 1047.60(C₆₅H₁₁₀N₂O₈) |
| R_{f}: | 0.30(chloroform/methanol/2-propanol/ |
| | acetic acid 80:20:20:1) |
| ¹H-NMR (250MHz, | |
| CDCl₃/CD₃OD/ | |
| D₂O 20:10:1): | |
| Non-cholesterol signals: | |
| δ=1.80-1.95 | (m, 2H, —NCH₂CH₂CH₂N—) |
| δ=2.02 | (s, 6H, 2CH₃COO⁻) |
| δ=2.92 | (t, ³J=6.1Hz, 4H, —NCH₂CH₂CH₂N—) |
| δ=3.46 | (s, 4H, |
| | —OCOCH₂N(CH₂)₃NCH₂COO—) |

N,N′-to-(cholesteryloxycarbonylmethyl)-1,5-diaminopentane acetic acid salt (70)

Quantities Used:

1136 mg (1.0 mmol) N,N′-to-(cholesteryloxycarbonylmethyl)-N,N′-1,5-dibenzyl-1,5-diaminopentane (55)

106 mg (0.1 mmol) palladium/activated charcoal

Perform purification via column chromatography on 25 g silica gel with chloroform/2-propanol/acetic acid (60:40:1). After the solvent is removed, precipitate the product out of an acetone/diisopropyl ether mixture. The yield is 699 mg 70 as a colorless solid.

| | |
|---|---|
| Yield: | 699mg(65% of theoretical value) as |
| | a colorless solid |
| Mᵣ: | 1075.65(C₆₇H₁₁₄N₂O₈) |
| R_{f}: | 0.22(chloroform/methanol/2-propanol/ |
| | acetic acid 80:20:20:1) |
| ¹H-NMR(250MHz, | |
| CDCl₃/CD₃OD/ | |
| D₂O 20:10:1): | |
| Non-cholesterol signals: | |
| δ=1.32-1.57 | (m, 6H, —NCH₂(CH₂)₃CH₂N—) |
| δ=1.96 | (s, 6H, 2CH₃COO⁻) |
| δ=2.66-2.79 | (m, 4H, —NCH₂(CH₂)₃CH₂N—) |
| δ=3.51 | (s, 4H, |
| | —OCOCH₂N(CH₂)₄NCH₂COO—) |

N,N′-to-(cholesteryloxycarbonylmethyl)-1,6-diaminohexane acetic acid salt (71)

Quantities Used:

1150 mg (1.0 mmol) N,N′-to-(cholesteryloxycarbonylmethyl)-N,N′-1,6-dibenzyl-1,6-diaminohexane (56)

106 mg (0.1 mmol) palladium/activated charcoal

Perform purification via column chromatography on 25 g silica gel with chloroform/2-propanol/acetic acid (60:40:1). After the solvent is removed, precipitate the product out of an acetone/diisopropyl ether mixture. The yield is 774 mg 71 as a colorless solid.

| | |
|---|---|
| Yield: | 774mg(71% of theoretical value) as |
| | a colorless solid |
| Mᵣ: | 1089.68(C₆₈H₁₁₆N₂O₈) |
| R_{f}: | 0.22(chloroform/methanol/2-propanol/acetic |
| | acid 80:20:20:1) |
| ¹H-NMR(250MHz, | |
| CDCl₃/CD₃OD/ | |
| D₂O 20:10:1): | |
| Non-cholesterol signals: | |
| δ=1.20-1.34 | (m, 4H, —N(CH₂)₂(CH₂)₂(CH₂)₂N—) |
| δ=1.38-1.55 | (m, 4H, —NCH₂CH₂(CH₂)₂CH₂CH₂N—) |
| δ=1.98 | (s, 6H, 2CH₃COO⁻) |
| δ=2.86 | (t, ³J=7.5Hz, 4H, —NCH₂(CH₂)₄CH₂N—) |
| δ=3.67 | (s, 4H, —OCOCH₂N(CH₂)₆NCH₂COO—) |

Synthesis Procedures for Tricationic Lipids

General Synthesis Instructions for N-Z-2-bromoethylamine/N-Z-3-bromopropylamine:

Add a solution of 1.5 equivalents of benzylchloroformiate in 50 ml Rotisol in drops to a solution of one equivalent of 2-bromoethylamine-hydrobromide or 3-bromopropylamine-hydrobromide and three equivalents of triethylamine in 150 ml Rotisol under refrigeration. Stir for 20 hours at room temperature, then concentrate the formulation to a small volume, take it up in 200 ml ethyl acetate, and extract twice against 200 ml 2 N hydrochloric acid each time. Remove the solvent and purify the residue via column chromatography on 100 g silica gel. Elute the apolar impurities with cyclohexane/diisopropyl ether (10:1), and elute the product with diisopropyl ether.

N-Z-2-bromoethylamine (72)

Quantities Used:

34.8 g (170 mmol) 2-bromoethylamine-hydrobromide

72.0 ml (255 mmol) 50% solution of benzylchloroformiate in toluene

70.6 ml (510 mmol) triethylamine

| | |
|---|---|
| Yield: | 38.2g(87% of theoretical value) |
| | as a yellow oil |
| Mᵣ: | 258.11(C₁₀H₁₂BrNO₂) |
| R_{f}: | 0.39(cyclohexane/ethyl acetate 4:1) |
| ¹H-NMR(250MHz, | |
| CDCl₃): | |
| δ=3.47 | (t, ³J=5.6Hz, 2H, BrCH₂CH₂NHZ) |
| δ=3.56-3.66 | (m, 2H, BrCH₂CH₂NHZ) |
| δ=5.12 | (s, 2H, —OCH₂C₆H₅) |
| δ=5.15-5.26 | (m, 1H, —NHCOO—) |
| δ=7.26-7.39 | (m, 5H, H_{aromat.}) |

N-Z-3-bromopropylamine (73)

Quantities Used:

36.9 g (168 mmol) 3-bromopropylamine-hydrobromide

71.0 ml (252 mmol) 50% solution of benzylchloroformiate in toluene

69.8 ml (504 mmol) triethylamine

| | |
|---|---|
| Yield: | 32.5g(71% of theoretical value) |
| | as a yellow oil |
| Mᵣ: | 272.14(C₁₁H₁₄BrNO₂) |
| R_{f}: | 0.24(cyclohexane/diisopropyl ether 10:1) |
| ¹H-NMR(250MHz, | |
| CDCl₃): | |
| δ=2.07 | (quint, ³J=6.5Hz, 2H, BrCH₂CH₂CH₂NHZ) |
| δ=3.35 | (quart, ³J=6.4Hz, 2H, BrCH₂CH₂CH₂NHZ) |
| δ=3.43 | (t, ³J=6.4Hz, 2H, BrCH₂CH₂CH₂NHZ) |
| δ=4.90-5.04 | (m, 1H, —NHCOO—) |
| δ=5.09 | (s, 2H, —OCH₂C₆H₅) |
| δ=7.26-7.38 | (m, 5H, H_{aromat.}) |

General Synthesis Instructions for Protected Tricationic Head Groups:

Dissolve three equivalents of the respective N,N′-dibenzyl-α,ω-diaminoalkane (21-25) and one equivalent of N-Z-2-bromoethylamine (72) or N-Z-3-bromopropylamine (73) in 60 ml acetonitrile. Stir with one equiavlent of potassium carbonate for approx. 4 hours with reflux. When no traces of N-Z-3-bromopropylamine or N-Z-2-bromoethylamine can be detected via thin-layer chromatography, add an additional equivalent of this adduct to the formulation and stir overnight with reflux. Remove the solvent, then purify residue on 100 g silica gel via column chromatography. Elute the apolar impurities with ethyl acetate/cyclohexane (2:1). Slowly switch to ethyl acetate and then ethyl acetate/methanol (6:1) to elute the product completely. Elute the unreacted N,N′-dibenzyl-α,ω-diaminoalkane with ethyl acetate/methanol 1:1.

1-Z-4,7-dibenzyl-1,4,7-triazaheptane (74)

Quantities Used:

2.16 g (9.0 mmol) N,N′-dibenzyl-1,2-diaminoethane (21)

2.58 g (6.0 mmol) N-Z-2-bromoethylamine (72)

0.41 g (3.0 mmol) potassium carbonate

| | |
|---|---|
| Yield: | 1.45g(58% of theoretical value) |
| | as a yellow oil |
| Mᵣ: | 417.55(C₂₆H₃₁N₃O₂) |
| R_{f}: | 0.28(ethyl acetate/methanol 9:1) |
| ¹H-NMR(250MHz, | |
| CDCl₃): | |
| δ=2.59 | (t, ³J=6.1Hz, 2 H, —NCH₂CH₂NHZ) |
| δ=2.65 | (s, 4H, C₆H₅CH₂NHCH₂CH₂N—) |
| δ=3.23 | (t, ³J=5.9Hz, 2H, —CH₂NHZ) |
| δ=3.57 | (s, 2H, tert-NCH₂C₆H₅) |
| δ=3.64 | (s, 2H, sek-NHCH₂C₆H₅) |
| δ=5.05 | (s, 2H, —NHCOOCH₂C₆H₅) |
| δ=7.20-7.37 | (m, 15H, H_{aromat.}) |

1-Z-5,8-dibenzyl-1,5,8-triazaoctane (75)

Quantities Used:

2.16 g (9 mmol) N,N′-dibenzyl-1,2-diaminoethane (21)

1.63 g (6 mmol) N-Z-3-bromopropylamine (73)

0.41 g (3 mmol) potassium carbonate

| | |
|---|---|
| Yield: | 1.29g(50% of theoretical value) |
| | as a yellow oil |
| Mᵣ: | 431.58(C₂₇H₃₃N₃O₂) |
| R_{f}: | 0.36(ethyl acetate/methanol 9:1) |
| ¹H-NMR(250MHz, | |
| CDCl₃): | |
| δ=1.66 | (quint, ³J=6.3Hz, 2H, —NCH₂CH₂CH₂NHZ) |
| δ=2.48 | (t, ³J=6.3Hz, 2H, C₆H₅CH₂NHCH₂CH₂N—) |
| δ=2.54 | (t, ³J=5.8Hz, 2H, C₆H₅CH₂NHCH₂CH₂NCH₂—) |
| δ=2.68 | (t, ³J=5.8Hz, 2H, C₆H₅CH₂NHCH₂—) |
| δ=3.23 | (quart, ³J=6.1Hz, 2H, —CH₂NHZ) |
| δ=3.49 | (s, 2H, tert-NCH₂C₆H₅) |
| δ=3.67 | (s, 2H, sek-NHCH₂C₆H₅) |
| δ=5.06 | (s, 2H, —NHCOOCH₂C₆H₅) |
| δ=6.15-6.19 | (m, 1H —NHCOO—) |
| δ=7.21-7.32 | (m, 15H, H_{aromat.}) |

1-Z-4,8-dibenzyl-1,4,8-triazaoctane (76)

Quantities Used:

3.82 g (15 mmol) N,N′-dibenzyl-1,3-diaminopropane (22)

2.58 g (10 mmol) N-Z-2-bromoethylamine (72)

0.69 mg (5 mmol) potassium carbonate

| | |
|---|---|
| Yield: | 2.76g(64% of theoretical value) |
| | as a slightly orange-colored oil |
| Mᵣ: | 431.58(C₂₇H₃₃N₃O₂) |
| R_{f}: | 0.25(ethyl acetate/methanol 9:1) |
| ¹H-NMR(250MHz, | |
| CDCl₃): | |
| δ=1.66 | (quint, ³J=6.8Hz, 2H; —NCH₂CH₂CH₂N—) |
| δ=2.46-2.57 | (m, 4H, C₆H₆CH₂NHCH₂CH₂CH₂N—) |
| δ=2.62 | (t, ³J=6.7Hz, 2H, —NCH₂CH₂NHZ) |
| δ=3.25 | (quart, ³J=5.6Hz, 2H, —CH₂NHZ) |
| δ=3.53 | (s, 2H, tert-NCH₂C₆H₅) |
| δ=3.71 | (s, 2H, sek-NHCH₂C₆H₅) |
| δ=5.08 | (s, 2H, —NHCOOCH₂C₆H₅) |
| δ=5.63-5.68 | (m, 1H, —NHCOO—) |
| δ=7.19-7.37 | (m, 15H, H_{aromat.}) |

1-Z-5,9-dibenzyl-1,5,9-triazanonane (77)

Quantities Used:

5.34 g (21 mmol) N,N′-dibenzyl-1,3-diaminopropane (22)

3.81 g (14 mmol) N-Z-3-bromopropylamine (73)

0.97 g (7 mmol) potassium carbonate

| | |
|---|---|
| Yield: | 3.07g(57% of theoretical value) |
| | as a slightly orange-colored oil |
| Mᵣ: | 445.60(C₂₈H₃₅N₃O₂) |
| R_{f}: | 0.22(ethyl acetate/methanol 9:1) |
| ¹H-NMR | |
| (250MHz, | |
| CDCl₃): | |
| δ=1.69 | (quint, ³J=6.7 Hz, 2H, C₆H₅CH₂NHCH₂CH₂CH₂N—) |
| δ=1.88-2.02 | (m, 2H, —NCH₂CH₂CH₂NHZ) |
| δ=2.39-2.49 | (m, 4H, C₆H₅CH₂NHCH₂CH₂CH₂N—) |
| δ=2.61 | (t, ³J=6.9Hz, 2H, —NCH₂(CH₂)₂NHZ) |
| δ=3.19 | (quart, ³J=5.9Hz, 2H, —CH₂NHZ) |
| δ=3.47 | (S, 2H, tert-NCH₂C₆H₅) |
| δ=3.70 | (s, 2H, sec-NHCH₂C₆H₅) |
| δ=5.08 | (s, 2H, NHCOOCH₂C₆H₅) |
| δ=5.88-5.93 | (m, 1H, —NHCOO—) |
| δ=7.20-7.38 | (m, 15H, H_{aromat.}) |

1-Z-4,9-dibenzyl-1,4,9-triazanonane (78)

Quantities Used:

4.03 g (15 mmol) N,N′-dibenzyl-1,4-diaminobutane (3)

2.58 g (10 mmol) N-Z-2-bromoethylamine (72)

0.69 g (5 mmol) potassium carbonate

| | |
|---|---|
| Yield: | 2.23g(50% of theoretical value) |
| | as a yellow oil |
| Mᵣ: | 445.60(C₂₈H₃₅N₃O₂) |
| R_{f}: | 0.25(ethyl acetate/methanol 9:1) |
| ¹H-NMR | |
| (250MHz, | |
| CDCl₃): | |
| δ=1.43-1.52 | (m, 4H, C₆H₅CH₂HNCH₂(CH₂)₂CH₂N—) |
| δ=2.38-2.48 | (m, 2H, C₆H₅CH₂HN(CH₂)₃CH₂N—) |
| δ=2.48-2.62 | (m, 4H, —NCH₂(CH₂)₂CH₂NCH₂CH₂NHZ) |
| δ=3.22 | (quart, ³J=5.7Hz, 2H, —CH₂NHZ) |
| δ=3.54 | (s, 2H, tert-NCH₂C₆H₅) |
| δ=3.75 | (s, 2H, sec-NHCH₂C₆H₅) |
| δ=5.07 | (s, 2H, —NHCOOCH₂C₆H₅) |
| δ=5.19-5.28 | (m, 1H —NHCOO—) |
| δ=7.19-7.38 | (m, 15H, H_{aromat.}) |

1-Z-5,10-dibenzyl-1,5,10-triazadekan (79)

Quantities Used:

5.64 g (21 mmol) N,N′-dibenzyl-1,4-diaminobutane (23)

3.81 g (14 mmol) N-Z-3-bromopropylamine (73)

0.97 g (7 mmol) potassium carbonate

| | |
|---|---|
| Yield: | 3.28g(51% of theoretical value) as a yellow oil |
| Mᵣ: | 459.63(C₂₉H₃₇N₃O₂) |
| R_{f}: | 0.28(ethyl acetate/methanol 9:1) |
| ¹H-NMR | |
| (250MHz, CDCl₃): | |
| δ=1.45-1.56 | (m, 4H, C₆H₅CH₂HNCH₂(CH₂)₂CH₂N—) |
| δ=1.63 | (quint, ³J=6.3Hz, 2H, —NCH₂CH₂CH₂NHZ) |
| δ=2.38 | (t, ³J=6.7Hz, 2H, —CH₂N(CH₂)₃NHZ) |
| δ=2.45 | (t, ³J=6.3Hz, 2H, C₆H₅CH₂NHCH₂(CH₂)₃N—) |
| δ=2.56 | (t, ³J=6.6Hz, 2H, —NCH₂(CH₂)₂NHZ) |
| δ=3.20 | (quart, ³J=5.9Hz, 2H, —CH₂NHZ) |
| δ=3.49 | (s, 2H, tert—NCH₂C₆H₅) |
| δ=3.73 | (s, 2H, sec—NHCH₂C₆H₅) |
| δ=5.07 | (s, 2H, —NHCOOCH₂C₆H₅) |
| δ=5.78-5.89 | (m, 1H, —NHCOO—) |
| δ=7.19-7.36 | (m, 15H, H_{aromat.}) |

1-Z-4,10--dibenzyl-1,4,10-triazadekan (80)

Quantities Used:

5.93 g (21 mmol) N,N′-dibenzyl-1,5-diaminopentane (24)

3.61 g (14 mmol) N-Z-2-bromoethylamine (72)

0.97 g (7 mmol) potassium carbonate

| | |
|---|---|
| Yield: | 3.41g(53% of theoretical value) as an orange-colored oil |
| Mᵣ: | 459.63(C₂₉H₃₇N₃O₂) |
| R_{f}: | 0.24(ethyl acetate/methanol 9:1) |
| ¹H-NMR | |
| (250MHz, | |
| CDCl₃): | |
| δ=1.20-1.36 | (m, 2H, —HN(CH₂)₂CH₂(CH₂)₂N—) |
| δ=1.37-1.60 | (m, 4H, —HNCH₂CH₂CH₂CH₂CH₂N—) |
| δ=2.42 | (t, ³J=7.0Hz, 2H, CH₂N(CH₂)₂NHZ) |
| δ=2.47-2.63 | (m, 4H, C₆H₅CH₂HNCH₂(CH₂)₄NCH₂—) |
| δ=3.15-3.28 | (m, 2H, —CH₂NHZ) |
| δ=3.53 | (s, 2H, tert—NCH₂C₆H₅) |
| δ=3.76 | (s, 2H, sec—NHCH₂C₆H₅) |
| δ=5.07 | (s, 2H, —NHCOOCH₂C₆H₅) |
| δ=5.14-5.24 | (m, 1H, —NHCOO—) |
| δ=7.20-7.37 | (m, 15H, H_{aromat.}) |

1-Z-5,11-dibenzyl-1,5,11-triazaundekan (81)

Quantities Used:

5.93 g (21 mmol) N,N′-dibenzyl-1,5-diaminopentane (24)

3.81 g (14 mmol) N-Z-3-bromopropylamine (73)

0.97 g (7 mmol) potassium carbonate

| | |
|---|---|
| Yield: | 3.28g(50% of theoretical value) as a yellow oil |
| Mᵣ: | 473.66(C₃₀H₃₉N₃O₂) |
| R_{f}: | 0.29(ethyl acetate/methanol 9:1) |
| ¹H-NMR | |
| (250MHz, CDCl₃): | |
| δ=1.19-1.35 | (m, 2H, —HN(CH₂)₂CH₂(CH₂)₂N—) |
| δ=1.39-1.55 | (m, 4H, —HNCH₂CH₂CH₂CH₂CH₂N—) |
| δ=1.58-1.69 | (m, 2H, —NCH₂CH₂CH₂NHZ) |
| δ=2.37 | (t, ³J=7.3Hz, 2H, —CH₂N(CH₂)₃NHZ) |
| δ=2.45 | (t, ³J=6.1Hz, 2H, C₆H₅CH₂HNCH₂(CH₂)₄N—) |
| δ=2.58 | (t, ³J=7.0Hz, 2H, —NCH₂(CH₂)₂NHZ) |
| δ=3.21 | (quart, ³J=5.9Hz, 2H, —N(CH₂)₂CH₂NHZ) |
| δ=3.49 | (s, 2H, tert—NCH₂C₆H₅) |
| δ=3.75 | (s, 2H, sec—NHCH₂C₆H₅) |
| δ=5.07 | (s, 2H, —NHCOOCH₂C₆H₅) |
| δ=5.84-5.93 | (m, 1H, —NHCOO—) |
| δ=7.19-7.37 | (m, 15H, H_{aromat.}) |

1-Z-4,11-dibenzyl-1,4,11-triazaundekan (82)

Quantities Used:

4.45 g (15 mmol) N,N′-dibenzyl-1,6-diaminohexane (25)

2.58 g (10 mmol) N-Z-2-bromoethylamine (72)

0.69 g (5 mmol) potassium carbonate

| | |
|---|---|
| Yield: | 2.37g(50% of theoretical value) as a yellow oil |
| Mᵣ: | 473.66(C₃₀H₃₉N₃O₂) |
| R_{f}: | 0.25(ethyl acetate/methanol 9:1) |
| ¹H-NMR | |
| (250MHz, CDCl₃): | |
| δ=1.20-1.32 | (m, 4H, —HN(CH₂)₂(CH₂)₂(CH₂)₂N—) |
| δ=1.37-1.54 | (m, 4H, —HNCH₂CH₂(CH₂)₂CH₂CH₂N—) |
| δ=2.41 | (t, ³J=7.2Hz, 2H, —CH₂N(CH₂)₂NHZ) |
| δ=2.49-2.63 | (m, 4H, —NHCH₂(CH₂)₅NCH₂CH₂NHZ) |
| δ=3.22 | (t, ³J=7.2Hz, 2H, —CH₂NHZ) |
| δ=3.54 | (s, 2H, tert—NCH₂C₆H₅) |
| δ=3.77 | (s, 2H, sec—NHCH₂C₆H₅) |
| δ=5.07 | (s, 2H, —NHCOOCH₂C₆H₅) |
| δ=5.13-5.20 | (m, 1H, —NHCOO—) |
| δ=7.20-7.37 | (m, 15H, H_{aromat.}) |

1-Z-5,12-dibenzyl-1,5,12-triazadodekan (83)

Quantities Used:

6.23 g (21 mmol) N,N′-dibenzyl-1,6-diaminohexane (25)

3.81 g (14 mmol) N-Z-3-bromopropylamine (73)

0.97 g (7 mmol) potassium carbonate

| | |
|---|---|
| Yield: | 3.32g(49% of theoretical value) as a slightly |
| | orange-colored oil |
| Mᵣ: | 487.69(C₃₁H₄₁N₃O₂) |
| R_{f}: | 0.27(ethyl acetate/methanol 9:1) |
| ¹H-NMR | |
| (250MHz, CDCl₃): | |
| δ=1.20-1.32 | (m, 4H, —HN(CH₂)₂(CH₂)₂(CH₂)₂N—) |
| δ=1.37-1.54 | (m, 4H, —HNCH₂CH₂(CH₂)₂CH₂CH₂N—) |
| δ=1.57-1.70 | (m, 2H, —NCH₂CH₂CH₂NHZ) |
| δ=2.36 | (t, ³J=7.3Hz, 2H, —CH₂N(CH₂)₃NHZ) |
| δ=2.46 | (t, ³J=6.1Hz, 2H, C₆H₅CH₂HNCH₂—) |
| δ=2.58 | (t, ³J=7.0Hz, 2H, —NCH₂(CH₂)₂NHZ) |
| δ=3.21 | (quart, ³J=5.9Hz, 2H, —CH₂NHZ) |
| δ=3.49 | (s, 2H, tert—NCH₂C₆H₅) |
| δ=3.77 | (s, 2H, sek—NHCH₂C₆H₅) |
| δ=5.08 | (s, 2H, —NHCOOCH₂C₆H₅) |
| δ=5.85-5.95 | (m, 1H, —NHCOO—) |
| δ=7.19-7.37 | (m, 15H, H_{aromat.}) |

Synthesis Procedures for Coupling of Lipid Components and Head Group

General Synthesis Instructions:

Add 1.0 mmol potassium carbonate to a solution of 1.0 mmol of the respective protected tricationic head group (74-83) and 1.8 mmol of the respective lipid component in acetonitrile/toluene (8:1). Stir overnight with reflux. Remove the solvent and purify the residue via column chromatography on 25 g silica gel. Eluate excess quantities of lipid component with cyclohexane/diisopropyl ether (2:1), and eluate the product with cyclohexane/ethyl acetate (4:1 to 2:1).

10-(cholesteryloxycarbonyl-methyl)-1-Z-5,10-dibenzyl-1,5,10-triazadekan (84)

Quantities Used:

460 mg (1.0 mmol) 1-Z-5,10-dibenzyl-1,5,10-triazadekan (9)

834 mg (1.8 mmol) chloroacetic acid cholesterylester (1)

138 mg (1.0 mmol) potassium carbonate

| | |
|---|---|
| Yield: | 780 mg(88% of theoretical value) as a yellow oil |
| Mᵣ: | 886.31(C₅₈H₈₃N₃O₄) |
| R_{f}: | 0.27(cyclohexane/ethyl acetate 2:1) |
| ¹H-NMR | |
| (250MHz, CDCl₃): | |
| Non-cholesterol | |
| signals: | |
| δ=1.43-1.68 | (m, 6H, —NCH₂(CH₂)₂CH₂NCH₂CH₂CH₂NHZ) |
| δ=2.36 | (t, ³J=6.1Hz, 2H, —N(CH₂)₃CH₂N(CH₂)₃NHZ) |
| δ=2.44 | (t, ³J=6.1Hz, 2H, —N(CH₂)₄NCH₂(CH₂)₂NHZ) |
| δ=2.58 | (t, ³J=6.3Hz, 2H, —NCH₂(CH₂)₃N(CH₂)₃NHZ) |
| δ=3.15-3.27 | (m, 2H, —N(CH₂)₂CH₂NHZ) |
| δ=3.23 | (s, 2H, —OCOCH₂N(CH₂)₄N—) |
| δ=3.48 | (s, 2H, C₆H₅CH₂—N(CH₂)₃NHZ) |
| δ=3.73 | (s, 2H, C₆H₅CH₂—N(CH₂)₄N(CH₂)₃NHZ) |
| δ=5.07 | (s, 2H, —NHCOOCH₂C₆H₅) |
| δ=5.73-5.82 | (m, 1H, —NHZ) |
| δ=7.17-7.37 | (m, 15H, H_{aromat.}) |

10-(2-(cholesteryloxycarbonyloxy)-ethyl)-1-Z-5,10-dibenzyl-1,5,10-triazadekan (85)

Quantities Used:

460 mg (1.0 mmol) 1-Z-5,10-dibenzyl-1,5,10-triazadekan (79)

968 mg (1.8 mmol) 2-bromoethyl-cholesterylcarbonate (14)

138 mg (1.0 mmol) potassium carbonate

| | |
|---|---|
| Yield: | 568 mg(62% of theoretical value) as a yellow oil |
| Mᵣ: | 916.34(C₅₉H₈₅N₃O₅) |
| R_{f}: | 0.23(cyclohexane/ethyl acetate 2:1) |
| ¹H-NMR | |
| (250MHz, CDCl₃): | |
| Non-cholesterol | |
| signals: | |
| δ=1.45-1.63 | (m, 6H, —NCH₂(CH₂)₂CH₂NCH₂CH₂CH₂NHZ) |
| δ=2.28-2.49 | (m, 6H, —NCH₂(CH₂)₂CH₂NCH₂(CH₂)₂NHZ) |
| δ=2.69 | (t, ³J=6.1Hz, 2H, —OCH₂CH₂N—) |
| δ=3.14-3.28 | (m, 2H, —N(CH₂)₂CH₂NHZ) |
| δ=3.48 | (s, 2H, C₆H₅CH₂—N(CH₂)₃NHZ) |
| δ=3.58 | (s, 2H, C₆H₅CH₂—N(CH₂)₄N(CH₂)₃NHZ) |
| δ=4.09-4.19 | (m, 2H, —OCH₂CH₂N—) |
| δ=5.07 | (s, 2H, —NHCOOCH₂C₆H₅) |
| δ=5.77-5.81 | (m, 1H, —NHZ) |
| δ=7.20-7.37 | (m, 15H, H_{aromat.}) |

10-(cholesterylhemisuccinoyloxy-2-ethyl)-1-Z-5,10-dibenzyl-1,5,10- triazadekan (86)

Quantities Used:

460 mg (1.0 mmol) 1-Z-5,10-dibenzyl-1,5,10-triazadekan (9)

1069 mg (1.8 mmol) 2-bromoethyl-cholesterylsuccinate (15)

138 mg (1.0 mmol) potassium carbonate

| | |
|---|---|
| Yield: | 574 mg(59% of theoretical value) as a yellow oil |
| Mᵣ: | 972.41(C₆₂H₈₉N₃O₆) |
| R_{f}: | 0.27(cyclohexane/ethyl acetate 2:1) |
| ¹H-NMR | |
| (250MHz, CDCl₃): | |
| Non-cholesterol | |
| signals: | |
| δ=1.45-1.63 | (m, 6H, —NCH₂(CH₂)₂CH₂NCH₂CH₂CH₂NHZ) |
| δ=2.28-2.49 | (m, 6H, —NCH₂(CH₂)₂CH₂NCH₂(CH₂)₂NHZ) |
| δ=2.52-2.61 | (m, 4H, —OCO(CH₂)₂COO—) |
| δ=2.61-2.70 | (m, 2H, —OCH₂CH₂N—) |
| δ=3.20 | (quart, ³J=5.9Hz, 2H, —N(CH₂)₂CH₂NHZ) |
| δ=3.49 | (s, 2H, C₆H₅CH₂—N(CH₂)₃NHZ) |
| δ=3.58 | (s, 2H, C₆H₅CH₂—N(CH₂)₄N(CH₂)₃NHZ) |
| δ=4.12 | (t, ³J=6.1Hz, 2H, —OCH₂CH₂N—) |
| δ=5.08 | (s, 2H, —NHCOOCH₂C₆H₅) |
| δ=5.75-5.85 | (m, 1H, —NHZ) |
| δ=7.17-7.37 | (m, 15H, H_{aromat.}) |

10-(cholesterylhemisuccinoyloxy-3-propyl)-1-Z-5,10-dibenzyl-1,5,10- triazadekan (87)

Quantities Used:

460 mg (1.0 mmol) 1-Z-5,10-dibenzyl-1,5,10-triazadekan (79)

1094 mg (1.8 mmol) 3-bromopropyl-cholesterylsuccinate (16)

138 mg (1.0 mmol) potassium carbonate

| | |
|---|---|
| Yield: | 572 mg(58% of theoretical value) as a yellow oil |
| Mᵣ: | 986.43(C₆₃H₉₁N₃O₆) |
| R_{f}: | 0.21(cyclohexane/ethyl acetate 2:1) |
| ¹H-NMR | |
| (250MHz, CDCl₃): | |
| Non-cholesterol | |
| signals: | |
| δ=1.45-1.63 | (m, 6H, —NCH₂(cH₂)₂CH₂NCH₂CH₂CH₂NHZ) |
| δ=2.26-2.48 | (m, 10H, —OCH₂(CH₂)₂NCH₂(CH₂)₂CH₂NCH₂ |
| | (CH₂)₂NHZ) |
| δ=2.50-2.58 | (m, 4H, —OCO(CH₂)₂COO—) |
| δ=3.20 | (quart, ³J=6.0Hz, 2H, —N(CH₂)₂CH₂NHZ) |
| δ=3.48 | (s, 4H, 2 —NCH₂C₆H₅) |
| δ=4.08 | (t, ³J=6.6Hz, 2H, —OCH₂CH₂CH₂N—) |
| δ=5.07 | (s, 2H, —NHCOOCH₂C₆H₅) |
| δ=5.75-5.85 | (m, 1H, —NHZ) |
| δ=7.17-7.37 | (m, 15H, H_{aromat.}) |

7-(cholesteryloxycarbonyl-methyl)-1-Z-4,7-dibenzyl-1,4,7-triazaheptane (88)

Quantities Used:

418 mg (1.0 mmol) 1-Z-4,7-dibenzyl-1,4,7-triazaheptane (74)

834 mg (1.8 mmol) chloroacetic acid cholesterylester (1)

138 mg (1.0 mmol) potassium carbonate

| | |
|---|---|
| Yield: | 549 mg(65% of theoretical value) as a yellow oil |
| Mᵣ: | 844.23(C₅₅H₇₇N₃O₄) |
| R_{f}: | 0.43(cyclohexane/ethyl acetate 2:1) |
| ¹H-NMR\| | |
| (250MHz, CDCl₃): | |
| Non-cholesterol | |
| signals: | |
| δ=2.50-2.62 | (m, 4H, —NCH₂CH₂NCH₂CH₂NHZ) |
| δ=2.75 | (t, ³J=6.4Hz, 2H, —NCH₂CH₂N(CH₂)₂NHZ) |
| δ=3.23 | (s, 2H, —OCOCH₂N(CH₂)₂N—) |
| δ=3.13-3.29 | (m, 2H, —NCH₂CH₂NHZ) |
| δ=3.55 | (s, 2H, C₆H₅CH₂—N(CH₂)₂NHZ) |
| δ=3.70 | (s, 2H, C₆H₅CH₂—N(CH₂)₂N(CH₂)₂NHZ) |
| δ=5.07 | (s, 2H, —NHCOOCH₂C₆H₅) |
| δ=5.80-5.91 | (m, 1H, —NHZ) |
| δ=7.15-7.40 | (m, 15H, H_{aromat.}) |

8-(cholesteryloxycarbonyl-methyl)-1-Z-5,8-dibenzyl-1,5,8-triazaoctane (89)

Quantities Used:

432 mg (1.0 mmol) 1-Z-5,8-dibenzyl-1,5,8-triazaoctane (75)

834 mg (1.8 mmol) chloroacetic acid cholesterylester (1)

138 mg (1.0 mmol) potassium carbonate

| | |
|---|---|
| Yield: | 687 mg(80% of theoretical |
| | value) as a yellow oil |
| Mᵣ: | 858.26(C₅₆H₇₉N₃O₄) |
| R_{f}: | 0.42(cyclohexane/ethyl acetate 2:1) |
| ¹H-NMR(250MHz, CDCl₃): | |
| Non-cholesterol signals: | |
| δ=1.60-1.75 | (m, 2H, —NCH₂CH₂CH₂NHZ) |
| δ=2.41-2.57 | (m, 4H, —NCH₂CH₂NCH₂(CH₂)₂NHZ) |
| δ=2.76 | (t, ³J=6.4Hz, 2H, |
| | —NCH₂CH₂N(CH₂)₃NHZ) |
| δ=3.17-3.31 | (m, 2H, —N(CH₂)₂CH₂NHZ) |
| δ=3.21 | (s, 2H, —OCOCH₂N(CH₂)₂N—) |
| δ=3.48 | (s, 2H, |
| | C₆H₅CH₂—N(CH₂)₃NHZ) |
| δ=3.70 | (s, 2H, |
| | C₆H₅CH₂—N(CH₂)₂N(CH₂)₃NHZ) |
| δ=5.06 | (s, 2H, —NHCOOCH₂C₆H₅) |
| δ=6.03-6.15 | (m, 1H, —NHZ) |
| δ=7.18-7.38 | (m, 15H, H_{aromat.}) |

8-(cholesteryloxycarbonyl-methyl)-1-Z-4,8-dibenzyl-1,4,8-triazaoctane (90)

Quantities Used:

432 mg (1.0 mmol) 1-Z-4,8-dibenzyl-1,4,8-triazaoctane (76)

834 mg (1.8 mmol) chloroacetic acid cholesterylester (1)

138 mg (1.0 mmol) potassium carbonate

| | |
|---|---|
| Yield: | 549 mg(64% of theoretical |
| | value) as a yellow oil |
| Mᵣ: | 858.26(C₅₆H₇₉N₃O₄) |
| R_{f}: | 0.47(cyclohexane/ethyl acetate 2:1) |
| ¹H-NMR(250MHz, CDCl₃): | |
| Non-cholesterol signals: | |
| δ=1.59-1.76 | (m, 2H, —NCH₂CH₂CH₂N(CH₂)₂NHZ) |
| δ=2.42-2.53 | (m, 2H, —N(CH₂)₂CH₂N(CH₂)₂NHZ) |
| δ=2.51 | (t, ³J=6.4Hz, 2H, |
| | —N(CH₂)₃NCH₂CH₂NHZ) |
| δ=2.63 | (t, ³J=7.0Hz, 2H, |
| | —NCH₂(CH₂)₂N(CH₂)₂NHZ) |
| δ=3.15-3.27 | (m, 2H, —NCH₂CH₂NHZ) |
| δ=3.22 | (s, 2H, —OCOCH₂N(CH₂)₃N—) |
| δ=3.53 | (s, 2H, |
| | C₆H₅CH₂—N(CH₂)₂NHZ) |
| δ=3.73 | (s, 2H, |
| | C₆H₅CH₂—N(CH₂)₃N(CH₂)₂NHZ) |
| δ=5.06 | (s, 2H, —NHCOOCH₂C₆H₅) |
| δ=5.15-5.25 | (m, 1H, —NHZ) |
| δ=7.17-7.37 | (m, 15H, H_{aromat.}) |

9-(cholesteryloxycarbonyl-methyl)-1-Z-5,9-dibenzyl-1,5,9-triazanonane (91)

Quantities Used:

446 mg (1.0 mmol) 1-Z-5,9-dibenzyl-1,5,9-triazanonane (77)

834 mg (1.8 mmol) chloroacetic acid cholesterylester (1)

138 mg (1.0 mmol) potassium carbonate

| | |
|---|---|
| Yield: | 672 mg(77% of theoretical |
| | value) as a yellow oil |
| Mᵣ: | 872.29(C₅₇H₈₁N₃O₄) |
| R_{f}: | 0.39(cyclohexane/ethyl acetate 2:1) |
| ¹H-NMR(250MHz, CDCl₃): | |
| Non-cholesterol signals: | |
| δ=1.59-1.76 | (m, 4H, |
| | —NCH₂CH₂CH₂NCH₂CH₂CH₂NHZ) |
| δ=2.37-2.56 | (m, 4H, |
| | —N(CH₂)₂CH₂NCH₂(CH₂)₂NHZ) |
| δ=2.62 | (t, ³J=7.2Hz, 2H, |
| | —NCH₂(CH₂)₂N(CH₂)₃NHZ) |
| δ=3.13-3.26 | (m, 2H, —N(CH₂)₂CH₂NHZ) |
| δ=3.21 | (s, 2H, —OCOCH₂N(CH₂)₃N—) |
| δ=3.48 | (s, 2H, C₆H₅CH₂—N(CH₂)₃NHZ) |
| δ=3.72 | (s, 2H, |
| | C₆H₅CH₂—N(CH₂)₃N(CH₂)₃NHZ) |
| δ=5.06 | (s, 2H, —NHCOOCH₂C₆H₅) |
| δ=5.62-5.73 | (m, 1H, —NHZ) |
| δ=7.17-7.37 | (m, 15H, H_{aromat.}) |

9-(cholesteryloxycarbonyl-methyl)-1-Z-4,9-dibenzyl-1,4,9-triazanonane (92)

Quantities Used:

446 mg (1.0 mmol) 1-Z-4,9-dibenzyl-1,4,9-triazanonane (78)

834 mg (1.8 mmol) chloroacetic acid cholesterylester (1)

138 mg (1.0 mmol) potassium carbonate

| | |
|---|---|
| Yield: | 663 mg(76% of theoretical |
| | value) as a yellow oil |
| Mᵣ: | 872.29(C₅₇H₈₁N₃O₄) |
| R_{f}: | 0.41(cyclohexane/ethyl acetate 2:1) |
| ¹H-NMR(250MHz, CDCl₃): | |
| Non-cholesterol signals: | |
| δ=1.43-1.56 | (m, 4H, |
| | —NCH₂(CH₂)₂CH₂N(CH₂)₂NHZ) |
| δ=2.37-2.56 | (m, 2H, |
| | —N(CH₂)₃CH₂N(CH₂)₂NHZ) |
| δ=2.52 | (t, ³J=5.8Hz, 2H, |
| | —N(CH₂)₄NCH₂CH₂NHZ) |
| δ=2.59 | (t, ³J=6.4Hz, 2H, |
| | —NCH₂(CH₂)₃N(CH₂)₂NHZ) |
| δ=3.15-3.26 | (m, 2H, —NCH₂CH₂NHZ) |
| δ=3.24 | (s, 2H, —OCOCH₂N(CH₂)₄N—) |
| δ=3.53 | (s, 2H, |
| | C₆H₅CH₂—N(CH₂)₂NHZ) |
| δ=3.74 | (s, 2H, |
| | C₆H₅CH₂—N(CH₂)₄N(CH₂)₂NHZ) |
| δ=5.07 | (s, 2H, —NHCOOCH₂C₆H₅) |
| δ=5.03-5.20 | (m, 1H, —NHZ) |
| δ=7.16-7.40 | (m, 15H, H_{aromat.}) |

10-(cholesteryloxycarbonyl-methyl)-1-Z-4,10-dibenzyl-1,4,10-triazadekan (93)

Quantities Used:

460 mg (1.0 mmol) 1-Z-4,10-dibenzyl-1,4,10-triazadekan (80)

834 mg (1.8 mmol) chloroacetic acid cholesterylester (1)

138 mg (1.0 mmol) potassium carbonate

| | |
|---|---|
| Yield: | 700 mg(79% of theoretical |
| | value) as a yellow oil |
| Mᵣ: | 886.31(C₅₈H₈₃N₃O₄) |
| R_{f}: | 0.35(cyclohexane/ethyl acetate 2:1) |
| ¹H-NMR(250MHz, CDCl₃): | |
| Non-cholesterol signals: | |
| δ=1.25-1.57 | (m, 6H, |
| | —NCH₂(CH₂)₃CH₂N(CH₂)₂NHZ) |
| δ=2.41 | (t, ³J=7.3Hz, |
| | 2H, —N(CH₂)₄CH₂N(CH₂)₂NHZ) |
| δ=2.52 | (t, ³J=6.0Hz, 2H, |
| | —N(CH₂)₅NCH₂CH₂NHZ) |
| δ=2.59 | (t, ³J=7.3Hz, 2H, |
| | —NCH₂(CH₂)₄N(CH₂)₂NHZ) |
| δ=3.16-3.28 | (m, 2H, —NCH₂CH₂NHZ) |
| δ=3.24 | (s, 2H, —OCOCH₂N(CH₂)₅N—) |
| δ=3.53 | (s, 2H, C₆H₅CH₂—N(CH₂)₂NHZ) |
| δ=3.74 | (s, 2H, |
| | C₆H₅CH₂—N(CH₂)₅N(CH₂)₂NHZ) |
| δ=5.07 | (s, 2H, —NHCOOCH₂C₆H₅) |
| δ=5.03-5.20 | (m, 1H, —NHZ) |
| δ=7.17-7.37 | (m, 15H, H_{aromat.}) |

11-(cholesteryloxycarbonyl-methyl)-1-Z-5,11-dibenzyl-1,5,11-triazaundekan (94)

Quantities Used:

474 mg (1.0 mmol) 1-Z-5,11-dibenzyl-1,5, 11-triazaundekan (81)

834 mg (1.8 mmol) chloroacetic acid cholesterylester (1)

138 mg (1.0 mmol) potassium carbonate

| | |
|---|---|
| Yield: | 585 mg(65% of theoretical |
| | value) as a yellow oil |
| Mᵣ: | 900.34(C₅₉H₈₅N₃O₄) |
| R_{f}: | 0.37(cyclohexane/ethyl acetate 2:1) |
| ¹H-NMR(250MHz, | |
| CDCl₃): | |
| Non-cholesterol signals: | |
| δ=1.25-1.68 | (m, 8H, |
| | —NCH₂(CH₂)₃CH₂NCH₂CH₂CH₂NHZ) |
| δ=2.37 | (t, ³J=7.0Hz, 2H, |
| | —N(CH₂)₄CH₂N(CH₂)₃NHZ) |
| δ=2.45 | (t, ³J=6.1Hz, 2H, |
| | —N(CH₂)₅NCH₂(CH₂)₂NHZ) |
| δ=2.58 | (t, ³J=7.0Hz, 2H, |
| | —NCH₂(CH₂)₄N(CH₂)₃NHZ) |
| δ=3.13-3.27 | (m, 2H, —N(CH₂)₂CH₂NHZ) |
| δ=3.24 | (s, 2H, —OCOCH₂N(CH₂)₅N—) |
| δ=3.49 | (s, 2H, C₆H₅CH₂—N(CH₂)₃NHZ) |
| δ=3.74 | (s, 2H, C₆H₅CH₂—N(CH₂)₅N(CH₂)₃NHZ) |
| δ=5.07 | (s, 2H, —NHCOOCH₂C₆H₅) |
| δ=5.73-5.82 | (m, 1H, —NHZ) |
| δ=7.17-7.37 | (m, 15H, H_{aromat.}) |

11-(cholesteryloxycarbonyl-methyl)-1-Z-4,11-dibenzyl-1,4,11-triazaundekan (95)

Quantities Used:

474 mg (1.0 mmol) 1-1,4,11-dibenzyl-1,4,11-triazaundekan (82)

834 mg (1.8 mmol) chloroacetic acid cholesterylester (1)

138 mg (1.0 mmol) potassium carbonate

| | |
|---|---|
| Yield: | 720 mg(80% of theoretical value) as a yellow oil |
| Mᵣ: | 900.34(C₅₉H₈₅N₃O₄) |
| R_{f}: | 0.34(cyclohexane/ethyl acetate 2:1) |
| ¹H-NMR(250MHz, | |
| CDCl₃): | |
| Non-cholesterol | |
| signals: | |
| δ=1.20-1.32 | (m, 4H, —N(CH₂)₂(CH₂)2(CH₂)₂N(CH₂)₂NHZ) |
| δ=1.37-1.54 | (m, 4H, —NCH₂CH₂(CH₂)₂CH₂CH₂N(CH₂)₂NHZ) |
| δ=2.40 | (t, ³J=7.3Hz, 2H, —N(CH₂)₅CH₂N(CH₂)₂NHZ) |
| δ=2.53 | (t, ³J=5.9Hz, 2H, —N(CH₂)₆NCH₂CH₂NHZ) |
| δ=2.59 | (t, ³J=7.0Hz, 2H, —NCH₂(CH₂)₅N(CH₂)₂NHZ) |
| δ=3.15-3.28 | (m, 2H, —NCH₂CH₂NHZ) |
| δ=3.26 | (s, 2H, —OCOCH₂N(CH₂)₆N—) |
| δ=3.54 | (s, 2H, C₆H₅CH₂—N(CH₂)₂NHZ) |
| δ=3.75 | (s, 2H, C₆H₅CH₂—N(CH₂)₆N(CH₂)₂NHZ) |
| δ=5.07 | (s, 2H, —NHCOOCH₂C₆H₅) |
| δ=5.10-5.20 | (m, 1H, —NHZ) |
| δ=7.17-7.37 | (m, 15H, H_{aromat.}) |

12-(cholesteryloxycarbonyl-methyl)-1-Z-5,12-dibenzyl-1,5,12-triazadodekan (96)

Quantities Used:

488 mg (1.0 mmol) 1-Z-5,12-dibenzyl-1,5,12-triazadodekan (83)

834 mg (1.8 mmol) chloroacetic acid cholesterylester (1)

138 mg (1.0 mmol) potassium carbonate

| | |
|---|---|
| Yield: | 658 mg(72% of theoretical value) as a yellow oil |
| Mᵣ: | 914.37(C₆₀H₈₇N₃O₄) |
| R_{f}: | 0.37(cyclohexane/ethyl acetate 2:1) |
| ¹H-NMR(250MHz, | |
| CDCl₃): | |
| Non-cholesterol | |
| signals: | |
| δ=1.20-1.32 | (m, 4H, —N(CH₂)₂(CH₂)₂(CH₂)₂N(CH₂)₃NHZ) |
| δ=1.37-1.68 | (m, 6H, |
| | —NCH₂CH₂(CH₂)₂CH₂CH₂NCH₂CH₂CH₂NHZ) |
| δ=2.36 | (t, ³J=7.3Hz, 2H, —N(CH₂)₅CH₂N(CH₂)₃NHZ) |
| δ=2.45 | (t, ³J=6.1Hz, 2H, —N(CH₂)₆NCH₂(CH₂)₂NHZ) |
| δ=2.58 | (t, ³J=7.3Hz, 2H, —NCH₂(CH₂)₅N(CH₂)₃NHZ) |
| δ=3.13-3.28 | (m, 2H, —N(CH₂)₂CH₂NHZ) |
| δ=3.25 | (s, 2H, —OCOCH₂N(CH₂)₆N—) |
| δ=3.49 | (s, 2H, C₆H₅CH₂—N(CH₂)₃NHZ) |
| δ=3.75 | (s, 2H, C₆H₅CH₂—N(CH₂)₆N(CH₂)₃NHZ) |
| δ=5.08 | (s, 2H, —NHCOOCH₂C₆H₅) |
| δ=5.76-5.87 | (m, 1H, —NHZ) |
| δ=7.17-7.38 | (m, 15H, H_{aromat.}) |

General Synthesis Instructions:

Add 0.1 mmol palladium/activated charcoal (10%) to a solution of 1.0 mmol of the respective benzyl-protected tricationic lipid (84-96) in 4 ml of a solvent mixture of dichloromethane/methanol (1:1). Stir overnight in a hydrogen atmosphere. Concentrate the formulation to a small volume to dry it and purify the residue via column chromatography on 30-40 g silica gel. Elute apolar impurities chloroform/methanol (9:1) or with chloroform/methanol/ammonia (25%) (90:10:1), and elute the product with chloroform/methanol/ammonia (25%) (60:40:1). Combine the fractions containing product, remove the solvent, and dry the residue well in a high vacuum. Take up the product in 1-2 ml dichloromethane/acetone (1:1), then add 1 ml acetic acid. A majority of the project will then precipitate out as acetic acid salt. Remove the solvent and acetic acid. The product is obtained as a solid or a sticky mass.

10-(cholesteryloxycarbonyl-methyl)-1,5,10-triazadekan acetic acid salt (97)

Quantities Used:

886 mg (1.0 mmol) 10-(cholesteryloxycarbonyl-methyl)-1-Z-5,10-dibenzyl-1,5,10-triazadekan (84)

106 mg (0.1 mmol) palladium/activated charcoal

| | |
|---|---|
| Yield: | 309 mg(41% of theoretical value) |
| Mᵣ: | 752.09(C₄₂H₇₇N₃O₈) |
| R_{f}: | 0.13(chloroform/methanol/ammonia(25%) 60:40:2) |
| ¹H-NMR(250MHz, | |
| CDCl₃/CD₃OD/ | |
| D₂O 20:01:1): | |
| Non-cholesterol | |
| signals: | |
| δ=1.75-1.96 | (m, 4H, —NCH₂(CH₂)₂CH₂N(CH₂)₃N—) |
| δ=1.99-2.09 | (m, 2H, —N(CH₂)₄NCH₂CH₂CH₂N—) |
| δ=2.01 | (s, 9H, 3 CH₃COO⁻) |
| δ=2.71 | (t, ³J=6.1Hz, 2H, |
| | —OCOCH₂N(CH₂)₃CH₂N(CH₂)₃N—) |
| δ=2.95 | (t, ³J=6.4Hz, 2H, |
| | —OCOCH₂NCH₂(CH₂)₃N(CH₂)₃N—) |
| δ=3.02 | (t, ³J=7.3Hz, 2H, |
| | —OCOCH₂N(CH₂)₄NCH₂(CH₂)₂N—) |
| δ=3.05 | (t, ³J=7.6Hz, 2H, |
| | —OCOCH₂N(CH₂)₄N(CH₂)₂CH₂N—) |
| δ=3.49 | (s, 2H, —OCOCH₂N(CH₂)₄N—) |

10-(2-(cholesteryloxycarbonyloxy)-ethyl)-1,5,10-triazadekan acetic acid salt (98)

Quantities Used:

916 mg (1.0 mmol) 10-(2-(cholesteryloxycarbonyloxy)-ethyl)-1-Z-5,10-dibenzyl-1,5,10-triazadekan (85)

106 mg (0.1 mmol) palladium/activated charcoal

| | |
|---|---|
| Yield: | 399 mg(51% of theoretical value) |
| Mᵣ: | 782.11(C₄₃H₇₉N₃O₉) |
| R_{f}: | 0.14(chloroform/methanol/ammonia(25%) 60:40:2) |
| ¹H-NMR(250MHz, | |
| CDCl₃/CD₃OD/ | |
| D₂O 20:01:1): | |
| Non-cholesterol | |
| signals: | |
| δ=1.62-1.78 | (m, 4H, —NCH₂(CH₂)₂CH₂N(CH₂)₃N—) |
| δ=1.89-2.10 | (m, 2H, —N(CH₂)₄NCH₂CH₂CH₂N—) |
| δ=1.96 | (s, 9H, 3 CH₃COO⁻) |
| δ=2.85 | (t, ³J=6.6Hz, 2H, —N(CH₂)₃CH₂N(CH₂)₃N—) |
| δ=2.87-3.06 | (m, 6H, —NCH₂(CH₂)₃NCH₂CH₂CH₂N—) |
| δ=3.11 | (t, ³J=5.2Hz, 2H, —OCH₂CH₂N(CH₂)₄N—) |
| δ=4.33 | (t, ³J=5.2Hz, 2H, —OCH₂CH₂N(CH₂)₄N—) |

10-(cholesterylhemisuccinoyloxy-2-ethyl)-1,5,10-triazadekan acetic acid salt (99)

Quantities Used:

972 mg (1.0 mmol) 10-(cholesterylhemisuccinoyloxy-2-ethyl)-1-Z-5,10-dibenzyl-1,5, 10-triazadekan (86)

106 mg (0.1 mmol) palladium/activated charcoal

| | |
|---|---|
| Yield: | 444mg(53% of theoretical value) |
| Mᵣ: | 838.18(C₄₆H₈₃N₃O₁₀) |
| R_{f}: | 0.16(chloroform/methanol/ammonia(25%)60:40:2) |
| ¹H-NMR | |
| (250MHz, | |
| CDCl₃/CD₃OD/ | |
| D₂O 20:01:1): | |
| Non-cholesterol | |
| signals: | |
| δ=1.62-1.78 | (m, 4H, —NCH₂(CH₂)₂CH₂N(CH₂)₃N—) |
| δ=1.89-2.10 | (m, 2H, —N(CH₂)₄NCH₂CH₂CH₂N—) |
| δ=1.98 | (s, 9H, 3CH₃COO⁻) |
| δ=2.61-2.68 | (m, 4H, —OCO(CH₂)₂COO—) |
| δ=2.76-3.07 | (m, 8H, —NCH₂(CH₂)₂CH₂NCH₂CH₂CH₂N—) |
| δ=3.66-3.83 | (m, 2H, —OCH₂CH₂N(CH₂)₄N—) |
| δ=4.30 | (t, ³J=5.2Hz, 2H, —OCH₂CH₂N(CH₂)₄N—) |

10-(cholesterylhemisuccinoyloxy-3-propyl)1,5,10-triazadekan acetic acid salt (100)

Quantities Used:

1094 mg (1.0 mmol) 10-(cholesterylhemisuccinoyloxy-3-propyl)-1-Z-5,10-dibenzyl-1,5,10-triazadekan (87)

106 mg (0.1 mmol) palladium/activated charcoal

| | |
|---|---|
| Yield: | 443mg(52% of theoretical value) |
| Mᵣ: | 852.21(C₄₇H₈₅N₃O₁₀) |
| R_{f}: | 0.12(chloroform/methanol/ammonia(25%)60:40:2) |
| ¹H-NMR(250MHz, | |
| CDCl₃/CD₃OD/ | |
| D₂O 20:01:1): | |
| Non-cholesterol | |
| signals: | |
| δ=1.64-1.82 | (m, 4H, —NCH₂(CH₂)₂CH₂N(CH₂)₃N—) |
| δ=1.89-2.11 | (m, 4H, —OCH₂CH₂CH₂N(CH₂)₄NCH₂ |
| | CH₂CH₂N—) |
| δ=1.98 | (s, 9H, 3CH₃COO⁻) |
| δ=2.59-2.68 | (m, 4H, —OCO(CH₂)₂COO—) |
| δ=2.85-3.07 | (m, 10H, —O(CH₂)₂CH₂NCH₂(CH₂)₂CH₂NCH₂ |
| | CH₂CH₂N—) |
| δ=4.21 | (t, ³J=6.0Hz, 2H, —OCH₂(CH₂)₂N(CH₂)₄N—) |

8-(cholesteryloxycarbonyl-methyl)-1,4,8-triazaoctane acetic acid salt (101)

Quantities Used:

858 mg (1.0 mmol) 8-(cholesteryloxycarbonyl-methyl)-1-Z4,8-dibenzyl-1,4,8-triazaoctane (90)

106 mg (0.1 mmol) palladium/activated charcoal

| | |
|---|---|
| Yield: | 507mg(70% of theoretical value) |
| Mᵣ: | 724.03(C₄₀H₇₃N₃O₈) |
| R_{f}: | 0.12(chloroform/methanol/ammonia(25%)60:40:2) |
| ¹H-NMR(250MHz, | |
| CDCl₃/CD₃OD/ | |
| D₂O 20:01:1): | |
| Non-cholesterol | |
| signals: | |
| δ=1.89-2.10 | (m, 2H, —NCH₂CH₂CH₂N(CH₂)₂N—) |
| δ=1.96 | (s, 9H, 3CH₃COO⁻) |
| δ=2.85 | (t, ³J=6.1Hz, 2H, —OCOCH₂N(CH₂)₂CH₂ |
| | N(CH₂)₂N—) |
| δ=2.99 | (t, ³J=6.3Hz, 2H, —OCOCH₂NCH₂(CH₂)₂ |
| | N(CH₂)₂N—) |
| δ=3.03-3.13 | (m, 4H, —N(CH₂)₃N(CH₂)₂N—) |
| δ=3.48 | (s, 2H, —OCOCH₂N(CH₂)₃N—) |

9-(cholesteryloxycarbonyl-methyl)-1,5,9-triazanonane acetic acid salt (102)

Quantities Used:

872 mg (1.0 mmol) 9-(cholesteryloxycarbonyl-methyl)-1-Z-5,9-dibenzyl-1,5,9-triazanonane (91)

106 mg (0.1 mmol) palladium/activated charcoal

| | |
|---|---|
| Yield: | 362mg(49% of theoretical value) |
| Mᵣ: | 738.06(C₄₁H₇₅N₃O₈) |
| R_{f}: | 0.16(chloroform/methanol/ammonia(25%)60:40:2) |
| ¹H-NMR(250MHz, | |
| CDCl₃/CD₃OD/ | |
| D₂O 20:01:1): | |
| Non-cholesterol | |
| signals: | |
| δ=1.89-2.10 | (m, 4H, —NCH₂CH₂CH₂NCH₂CH₂CH₂N—) |
| δ=1.99 | (s, 9H, 3CH₃COO⁻) |
| δ=2.78 | (t, ³J=5.9Hz, 2H, —OCOCH₂N(CH₂)₂CH₂N |
| | (CH₂)₃N—) |
| δ=2.98 | (t, ³J=6.7Hz, 2H, —OCOCH₂NCH₂(CH₂)₂N |
| | (CH₂)₃N—) |
| δ=3.04 | (t, ³J=7.0Hz, 2H, —OCOCH₂N(CH₂)₃NCH₂ |
| | (CH₂)₂N—) |
| δ=3.06 | (t, ³J=6.1Hz, 2H, —OCOCH₂N(CH₂)₃N(CH₂)₂ |
| | CH₂N—) |
| δ=3.42 | (s, 2H, —OCOCH₂N(CH₂)₃N—) |

9-(cholesteryloxycarbonyl-methyl)-1,4,9-triazanonane acetic acid salt (103)

Quantities Used:

872 mg (1.0 mmol) 9-(cholesteryloxycarbonyl-methyl)-1-Z4,9-dibenzyl-1,4,9-triazanonane (92)

106 mg (0.1 mmol) palladium/activated charcoal

| | |
|---|---|
| Yield: | 524mg(71% of theoretical value) |
| Mᵣ: | 738.06(C₄₁H₇₅N₃O₈) |
| R_{f}: | 0.21(chloroform/methanol/ammonia(25%)60:40:2) |
| ¹H-NMR | |
| (250MHz, CDCl₃/ | |
| CD₃OD/ | |
| D₂O 20:01:1): | |
| Non-cholesterol | |
| signals: | |
| δ=1.75-1.99 | (m, 4H, —NCH₂(CH₂)₂CH₂N(CH₂)₂N—) |
| δ=2.02 | (s, 9H, 3CH₃COO⁻) |
| δ=2.98-3.21 | (m, 4H, —OCOCH₂NCH₂(CH₂)₂CH₂N(CH₂)₂N—) |
| δ=3.38-3.48 | (m, 4H, —OCOCH₂N(CH₂)₄N(CH₂)₂N—) |
| δ=3.86 | (s, 2H, —OCOCH₂N(CH₂)₄N—) |

10-(cholesteryloxycarbonyl-methyl)-1,4,10-triazadekan acetic acid salt (104)

Quantities Used:

886 mg (1.0 mmol) 10-(cholesteryloxycarbonyl-methyl)-1-Z-4,10-dibenzyl-1,4,10-triazadekan (93)

106 mg (0.1 mmol) palladium/activated charcoal

| | |
|---|---|
| Yield: | 587mg(78% of theoretical value) |
| Mᵣ: | 752.09(C₄₂H₇₇N₃O₈) |
| R_{f}: | 0.12(chloroform/methanol/ammonia(25%)60:40:2) |
| ¹H-NMR(250MHz, | |
| CDCl₃/ | |
| CD₃OD/ | |
| D₂O 20:01:1): | |
| Non-cholesterol | |
| signals: | |
| δ=1.30-1.57 | (m, 6H, —NCH₂(CH₂)₃CH₂N(CH₂)₂N—) |
| δ=2.02 | (s, 9H, 3CH₃COO⁻) |
| δ=3.00-3.17 | (m, 4H, —OCOCH₂NCH₂(CH₂)₃CH₂N(CH₂)₃N—) |
| δ=3.38-3.46 | (m, 4H, —OCOCH₂N(CH₂)₅N(CH₂)₂N—) |
| δ=3.84 | (s, 2H, —OCOCH₂N(CH₂)₅N—) |

11-(cholesteryloxycarbonyl-methyl)-1,5,11-triazaundekan acetic acid salt (105)

Quantities Used:

900 mg (1.0 mmol) 11-(cholesteryloxycarbonyl-methyl)-1-Z-5,11-dibenzyl-1,5,11-triaza-undekan (94)

106 mg (0.1 mmol) palladium/activated charcoal

| | |
|---|---|
| Yield: | 398mg(52% of theoretical value) |
| Mᵣ: | 766.12(C₄₃H₇₉N₃O₈) |
| R_{f}: | 0.14(chloroform/methanol/ammonia(25%)60:40:2) |
| ¹H-NMR(250MHz, | |
| CDCl₃/CD₃OD/ | |
| D₂O 20:01:1): | |
| Non-cholesterol | |
| signals: | |
| δ=1.30-1.57 | (m, 6H, —NCH₂(CH₂)₃CH₂N(CH₂)₂N—) |
| δ=1.99-2.10 | (m, 2H, —N(CH₂)₅NCH₂CH₂CH₂N—) |
| δ=2.00 | (s, 9H, 3CH₃COO⁻) |
| δ=2.67 | (t, ³J=7.2Hz, 2H, —OCOCH₂N(CH₂)₄CH₂N |
| | (CH₂)₃N—) |
| δ=2.88 | (t, ³J=7.6Hz, 2H, —OCOCH₂NCH₂(CH₂)₄N |
| | (CH₂)₃N—) |
| δ=2.93-3.03 | (m, 4H, —OCOCH₂N(CH₂)₅NCH₂CH₂CH₂N—) |
| δ=3.44 | (s, 2H, —OCOCH₂N(CH₂)₅N—) |

11-(cholesteryloxycarbonyl-methyl)-1,4,11-triazaundekan acetic acid salt (106)

Quantities Used:

900 mg (1.0 mmol) 11-(cholesteryloxycarbonyl-methyl)-1-Z-4,11-dibenzyl-1,4,11-triaza-undekan (95)

106 mg (0.1 mmol) palladium/activated charcoal

| | |
|---|---|
| Yield: | 529mg(69% of theoretical value) |
| Mᵣ: | 766.12(C₄₃H₇₉N₃O₈) |
| R_{f}: | 0.13(chloroform/methanol/ammonia(25%)60:40:2) |
| ¹H-NMR(250MHz, | |
| CDCl₃/ | |
| CD₃OD/ | |
| D₂O 20:01:1): | |
| Non-cholesterol | |
| signals: | |
| δ=1.30-1.76 | (m, 8H, —NCH₂(CH₂)₄CH₂N(CH₂)₂N—) |
| δ=2.01 | (s, 9H, 3CH₃COO⁻) |
| δ=2.86 | (t, ³J=7.5Hz, 2H, —OCOCH₂N(CH₂)₅CH₂N |
| | (CH₂)₂N—) |
| δ=2.94 | (t, ³J=7.5Hz, 2H, —OCOCH₂NCH₂(CH₂)₅N |
| | (CH₂)₂N—) |
| δ=3.19-3.26 | (m, 4H, —OCOCH₂N(CH₂)₆N(CH₂)₂N—) |
| δ=3.64 | (s, 2H, —OCOCH₂N(CH₂)₆N—) |

12-(cholesteryloxycarbonyl-methyl)-1,5,12-triazadodekan acetic acid salt (107)

Quantities Used:

914 mg (1.0 mmol) 12-(cholesteryloxycarbonyl-methyl)-1-Z-5,12-dibenzyl-1,5,12-triaza-dodekan (96)

106 mg (0.1 mmol) palladium/activated charcoal

| | |
|---|---|
| Yield: | 515mg(66% of theoretical value) |
| Mᵣ: | 780.14(C₄₄H₈₁N₃O₈) |
| R_{f}: | 0.09(chloroform/methanol/ammonia(25%)60:40:2) |
| ¹H-NMR(250MHz, | |
| CDCl₃/CD₃OD/ | |
| D₂O 20:01:1): | |
| Non-cholesterol | |
| signals: | |
| δ=1.30-1.76 | (m, 8H, —NCH₂(CH₂)₄CH₂N(CH₂)₃N—) |
| δ=1.99-2.10 | (m, 2H, —N(CH₂)₆NCH₂CH₂CH₂N—) |
| δ=1.97 | (s, 9H, 3CH₃COO⁻) |
| δ=2.63 | (t, ³J=7.2Hz, 2H, —OCOCH₂N(CH₂)₅CH₂N |
| | (CH₂)₃N—) |
| δ=2.80-2.90 | (m, 2H, —OCOCH₂NCH₂(CH₂)₅N(CH₂)₃N—) |
| δ=2.96 | (t, ³J=7.0Hz, 2H, —OCOCH₂N(CH₂)₆NCH₂ |
| | (CH₂)₂N—) |
| δ=2.97 | (t, ³J=7.2Hz, 2H, —OCOCH₂N(CH₂)₆N(CH₂)₂ |
| | CH₂N—) |
| δ=3.40 | (s, 2H, —OCOCH₂N(CH₂)₆N—) |

Synthesis Procedures for DMG derivatives

2,3-di-tetradecyloxy-propanol (108)

Starting with 2,3-diisopropyliden-glycerin, the product 2,3-di-tetradecyloxy-propanol was synthesized in a four-step procedure:

I) 24.7 g (0.22 mol) potassium-tert-butylate is added in portions to a solution of 27.2 ml (0.22 mol) 2,3-diisopropyliden-glycerin in 200 ml tetrahydrofuran under refrigeration. Stir for 15 min at 0° C., then add a solution of 23.0 ml (0.20 mol) benzylchloride in 100 ml tetrahydrofuran in drops within 30 minutes. Stir for another 2 hours at room temperature.

II) Add 100 ml 2 N hydrochloric acid in drops and stir overnight. Remove the solvent, then extract the residue three times with 100 ml ethyl acetate each time. Concentrate the organic phase to a small volume and dry in a high vacuum to obtain sufficiently clean 3-benzyloxy-propane-1,2-diole as an oil.

III) Dissolve the product (approx. 0.2 mol) in 400 ml toluene. Add 67.3 g (0.6 mol) potassium-tert-butylate and 163.8 ml (0.6 mol) tetradecylbromide, then warm for 4 hours with reflux. Extract the formulation twice against 100 ml 2 N hydrochloric acid each time. Concentrate the organic phase to a small volume. To separate off the polar impurities, purify the residue via column chromatography on 300 g silica gel with cyclohexane/diisopropyl ether (10:1) as the flow agent. The product 1-(2,3-di-tetradecyloxy)-propyl-benzylether contains very few apolar impurities.

IV) To debenzylate the product, dissolve a solution of the triether product in 200 ml tetrahydrofuran/methanol/acetic acid/water (10:10:2:2), add 2.1 g (0.002 mol) palladium/activated charcoal (10%), then stir vigorously overnight in a hydrogen atmosphere. Suction off the catalyst in a filter (also filled with a 1-cm thick layer of silica gel). Concentrate the organic phase to a small volume. Dissolve the solid residue in 200 ml cyclohexane in the boiling heat. A majority (42 g, 43%) of the product 2,3-di-tetradecyl-propanol precipitates out as a colorless crystal during cooling.

Concentrate the filtrate that still contains product to a small volume, purify the residue via column chromatography on 200 g silica gel (eluent is first cyclohexane, then cyclohexane/ethyl acetate 10:1 to 10:1). This produces another 25 g of product.

| | |
|---|---|
| Yield: | 67 g(69% in all 4 synthesis steps combined) |
| Mᵣ: | 484.85(C₃₁H₆₄O₃) |
| R_{f}: | 0.43(diisopropyl ether) |
| ¹H-NMR(250MHz, | |
| CDCl₃): | |
| δ=0.88 | (t, ³J=6.6Hz, 6H, 2 -O(CH₂)₁₃CH₃) |
| δ=1.16-1.40 | (m, 44H, 2 -O(CH₂)₂(CH₂)₁₁CH₃) |
| δ=1.48-1.64 | (m, 4H, 2 -OCH₂CH₂(CH₂)₁₁CH₃) |
| δ=2.15-2.27 | (m, 1H, —OH) |
| δ=3.43 | (t, ³J=6.5Hz, 2H, —CH₂OCH₂(CH₂)₁₂CH₃) |
| δ=3.45-3.67 | (m, 7H, HOCH₂CH(OCH₂(CH₂)₁₂CH₃)CH₂O—) |

Chloroacetic acid-(2,3-di-tetradecyloxy)-propylester (109)

Slowly add a solution of 1.6 g (9.1 mmol) chloroacetic acidanhydrid in 5 ml dichloromethane in drops to a solution of 3.7 g (7.6 mmol) 2,3-di-tetradecyloxy-propanol (108) and 2.1 ml (15.2 mmol) triethylamine in 20 ml dichloromethane under refrigeration. Stir for one hour at room temperature, then remove the solvent. Take up the residue in 50 ml ethyl acetate and extract twice against 2 N hydrochloric acid. Concentrate the organic phase to a small volume and purify on 50 g silica gel via column chromatography. Elute the apolar impurities with cyclohexane, and elute the product with cyclohexane/ethyl acetate (50:1 to 20:1). The yield is 4.1 g 109 as a colorless oil.

| | |
|---|---|
| Yield: | 4.1 g (96% of theoretical value) |
| Mᵣ: | 561.33 (C₃₃H₆₅ClO₄) |
| R_{f}: | 0.31 (cyclohexane/ethyl acetate 20:1) |

¹H-NMR (250 MHz, CDCl₃):

| | |
|---|---|
| δ = 0.88 | (t, ³J = 6.7 Hz, 6 H, 2 -O(CH₂)₁₃CH₃) |
| δ = 1.15 − 1.40 | (m, 44 H, 2 -O(CH₂)₂(CH₂)₁₁CH₃) |
| δ = 1.47 − 1.62 | (m, 4 H, 2 -OCH₂CH₂(CH₂)₁₁CH₃) |
| δ = 3.43 | (t, ³J = 6.7 Hz, 2 H, —CH₂OCH₂(CH₂)₁₂CH₃) |
| δ = 3.41 − 3.55 | (m, 2 H, —OCH₂CH(O—)CH₂OCOCH₂Cl) |
| δ = 3.55 | (t, ³J = 6.6 Hz, 2 H, |
| | —OCH₂CH(OCH₂(CH₂)₁₂CH₃)CH₂O—) |
| δ = 3.60 − 3.70 | (m, 1 H, —OCH₂CH(O—)CH₂O—) |
| δ = 4.09 | (s, 2 H, —OCH₂CH(O—)CH₂OCOCH₂Cl) |
| δ = 4.22 | (dd, ³J = 5.8 Hz and ²J = 11.6 Hz, 1 H, |
| | —CHHOCOCH₂Cl) |
| δ = 4.37 | (dd, ³J = 4.0 Hz and ²J = 11.6 Hz, 1 H, |
| | —CHHOCOCH₂Cl) |

### Synthesis Procedures for Simple Cationic DMG Derivatives

### N-(1-(2,3-di-tetradecyloxy)-propyloxycarbonylmethyl)-N,N-dimethylamine (110)

Slowly add a solution of 2.25 g (4 mmol) chloroacetic acid-1-(2,3-di-tetradecyloxy)-propylester (109) in 5 ml toluene in drops to 7.14 ml (40 mmol) of a 5.6 molar solution of dimethylamine in ethanol under refrigeration. Stir overnight at room temperature. Concentrate the formulation to a small volume and purify the residue via column chromatography on 30 g silica gel. Elute the apolar impurities with cyclohexane/ethyl acetate (10:1), and elute the product with cyclohexane/ethyl acetate (6:1).

| | |
|---|---|
| Yield: | 0.73 g (32% of theoretical value) as a milky slime |
| Mᵣ: | 569.95 (C₃₅H₇₁NO₄) |
| R_{f}: | 0.11 (cyclohexane/ethyl acetate 6:1) |

¹H-NMR (250 MHz, CDCl₃):

| | |
|---|---|
| δ = 0.88 | (t, ³J = 6.7 Hz, 6 H, 2 -O(CH₂)₁₃CH₃) |
| δ = 1.20 − 1.37 | (m, 44 H, 2 -O(CH₂)₂(cH₂)₁₁CH₃) |
| δ = 1.48 − 1.61 | (m, 4 H, 2 -OCH₂CH₂(CH₂)₁₁CH₃) |
| δ = 2.36 | (s, 6 H, —N(CH₃)₂) |
| δ = 3.20 | (s, 2 H, —OCOCH₂N(CH₃)₂) |
| δ = 3.43 | (t, ³J = 6.7 Hz, 2 H, —CH₂OCH₂(CH₂)₁₂CH₃) |
| δ = 3.44 − 3.50 | (m, 2 H, —OCH₂CH(O—)CH₂OCOCH₂N—) |
| δ = 3.55 | (t, ³J = 6.6 Hz, 2 H, |
| | —OCH₂CH(OCH₂(cH₂)₁₂CH₃)CH₂O—) |
| δ = 3.59 − 3.69 | (m, 1 H, —OCH₂CH(O—)CH₂O—) |
| δ = 4.15 | (dd, ³J = 5.8 Hz and ²J = 11.6 Hz, 1 H, |
| | —CHHOCOCH₂N—) |
| δ = 4.30 | (dd, ³J = 4.0 Hz and ²J = 11.6 Hz, 1 H, |
| | —CHHOCOCH₂N—) |

### N-(1-(2,3-di-tetradecyloxy)-propyloxycarbonylmethyl)-N,N,N-trimethylammoniummethylsulphate (111)

Add 474 μl (5.0 mmol) dimethyl sulphate in drops to a solution of 285 mg (0.5 mmol) N-(1-(2,3-di-tetradecyloxy)-propyloxycarbonylmethyl)-N,N-dimethylamine (110) in 5 ml acetone. Stir for 2 hours, the filter off the product—which precipitates out as a colorless precipitate—and rewash with a small quantity of acetone.

| | |
|---|---|
| Yield: | 139 mg (40% of theoretical value) as a colorless solid |
| Mᵣ: | 696.08 (C₃₇H₇₇NO₈S) |
| R_{f}: | 0.17 (chloroform/methanol/acetic acid 80:20:2) |

¹H-NMR (250 MHz, CDCl₃):

| | |
|---|---|
| δ = 0.88 | (t, ³J = 6.7 Hz, 6 H, 2 -O(CH₂)₁₃CH₃) |
| δ = | (m, 44 H, 2 -O(CH₂)₂(CH₂)₁₁CH₃) |
| 1.18 − 1.38 | |
| δ = | (m, 4 H, 2 -OCH₂CH₂(CH₂)₁₁CH₃) |
| 1.45 − 1.62 | |
| δ = | (m, 6 H, |
| 3.40 − 3.57 | —OCH₂CH(OCH₂(CH₂)₁₂CH₃)CH₂OCH₂(CH₂)₁₂CH₃) |
| δ = 3.50 | (s, 9 H, —N(CH₃)₃) |
| δ = | (m, 1 H, —OCH₂CH(O—)CH₂O—) |
| 3.57 − 3.67 | |
| δ = 3.71 | (s, 3 H, CH₃OSO₃) |
| δ = 4.26 | (dd, ³J = 5.5 Hz and ²J = 11.3 Hz, 1 H, |
| | —CHHOCOCH₂N—) |
| δ = 4.38 | (dd, ³J = 4.0 Hz and ²J = 11.3 Hz, 1 H, |
| | —CHHOCOCH₂N—) |
| δ = 4.54 | (s, 2 H, —OCOCH₂N(CH₃)₃) |

### Synthesis Procedures for Bicationic DMG Derivatives

### 1-((2,3-di-tetradecyloxy)-propyloxycarbonylmethyl)-1,6-dibenzyl-1,6-diazaoctane (112)

Stir a mixture of 296 mg (1.0 mmol) N-ethyl-N,N′-dibenzyl-1,4-diaminobutane (38), 786 mg (1.4 mmol) chloroacetic acid-(2,3-di-tetradecyloxy)-propylester (109) and 69 mg (0.5 mmol) potassium carbonate in 10 ml acetonitrile/toluene (8:1) overnight with reflux. Remove the solvent completely and purify the residue via column chromatography on 20 g silica gel. Elute the apolar impurities with cyclohexane/diisopropyl ether (4:1), and elute the product with cyclohexane/diisopropyl ether (1:1).

| | |
|---|---|
| Yield: | 542 mg (66% of theoretical value) as a yellow oil |
| Mᵣ: | 821.32 (C₅₃H₉₂N₂O₄) |
| R_{f}: | 0.28 (cyclohexane/ethyl acetate 6:1) |

¹H-NMR (250 MHz, CDCl₃):

| | |
|---|---|
| δ = 0.88 | (t, ³J = 6.6 Hz, 6 H, 2 -O(CH₂)₁₃CH₃) |
| δ = 1.01 | (t, ³J = 7.0 Hz, 3 H, −NCH₂CH₃) |
| δ = | (m, 44 H, 2 -O(CH₂)₂(CH₂)₁₁CH₃) |
| 1.20 − 1.37 | |
| δ = | (m, 8 H, 2 -OCH₂CH₂(CH₂)₁₁CH₃ and |
| 1.40 − 1.65 | —NCH₂(CH₂)₂CH₂N—) |
| δ = | (m, 2 H, —N(CH₂)₃CH₂NCH₂CH₃) |
| 2.34 − 2.44 | |
| δ = 2.47 | (quart, ³J = 7.1 Hz, 2 H, —NCH₂CH₃) |
| δ = | (m, 2 H, —NCH₂(CH₂)₃NCH₂CH₃) |
| 2.57 − 2.67 | |
| δ = 3.31 | (s, 2 H, —OCOCH₂N(CH₂)₄N—) |
| δ = 3.42 | (t, ³J = 6.7 Hz, 2 H, —CH₂OCH₂(CH₂)₁₂CH₃) |
| δ = | (m, 2 H, —OCH₂CH(O—)CH₂OCOCH₂N) |
| 3.41 − 3.55 | |
| δ 3.53 | (s, 2 H, C₆H₅CH₂—NCH₂CH₃) |
| δ 3.53 | (t, ³J = 6.4 Hz, 2 H, |
| | —OCH₂CH(OCH₂(CH₂)₁₂CH₃)CH₂O—) |
| δ = | (m, 1 H, —OCH₂CH(O—)CH₂O—) |
| 3.57 − 3.66 | |
| δ = 3.76 | (s, 2 H, C₆H₅CH₂—N(CH₂)₄NCH₂CH₃) |
| δ = 4.11 | (dd, ³J = 5.2 Hz and ²J = 10.8 Hz, 1 H, |
| | —CHHOCOCH₂N—) |
| δ = 4.26 | (dd, ³J = 4.0 Hz and ²J = 11.6 Hz, 1 H, |
| | —CHHOCOCH₂N—) |
| δ = | (m, 10 H, H_{aromat.}) |
| 7.16 − 7.37 | |

### 1-((2,3-di-tetradecyloxy)-propyloxycarbonylmethyl)-1,6-diazaoctane formic acid salt (113)

Add 106 mg (0.1 mmol) palladium/activated charcoal (10%) to a solution of 821 mg (1.0 mmol) 1-((2,3-di-tetradecyloxy)-propyloxcarbonylmethyl)-1,6-dibenzyl-1,6-diazaoctane (112) in 4 ml of a solvent mixture of dichloromethane/methanol/formic acid (2:1:1). Stir overnight in a hydrogen atmosphere. Concentrate the formulation to a small volume to dry it and purify the residue via column chromatography on 25 g silica gel. Elute the apolar impurities with chloroform/methanol/formic acid (90:10:1), and elute the product with chloroform/methanol/formic acid (80:20:2). After the solvent is removed, precipitate the product out of an acetone/diisopropyl ether mixture.

| | |
|---|---|
| Yield: | 484 mg (66% of theoretical value) as a colorless solid |
| Mᵣ: | 733.13 (C₄₁H₈₄N₂O₈) |
| R_{f}: | 0.55 (chloroform/methanol/formic acid/water 60:40:6:2) |

¹H-NMR (250 MHz, CDCl₃/ CD₃OD/D₂O 20:10:1):

| | |
|---|---|
| δ = 0.89 | (t, ³J = 6.7 Hz, 6 H, 2 -O(CH₂)₁₃CH₃) |
| δ = | (m, 44 H, 2 -O(CH₂)₂(CH₂)₁₁CH₃) |
| 1.15 − 1.43 | |
| δ = 1.35 | (t, ³J = 7.3 Hz, 3 H, —NCH₂CH₃) |
| δ = | (m, 4 H, 2 -OCH₂CH₂(CH₂)₁₁CH₃) |
| 1.48 − 1.66 | |
| δ = | (m, 4 H, —NCH₂(CH₂)₂CH₂N—) |
| 1.70 − 1.92 | |
| δ = 2.90 | (t, ³J = 6.3 Hz, 2 H, —N(CH₂)₃CH₂NCH₂CH₃) |
| δ = 2.97 | (t, ³J = 6.6 Hz, 2 H, —NCH₂(CH₂)₃NCH₂CH₃) |
| δ = 3.02 | (quart, ³J = 7.3 Hz, 2 H, —NCH₂CH₃) |
| δ = 3.47 | (t, ³J = 6.6 Hz, 2 H, —CH₂OCH₂(CH₂)₁₂CH₃) |
| δ = 3.52 | (t, ³J = 5.5 Hz, 2 H, —OCH₂CH(O—)CH₂OCOCH₂N—) |
| δ = 3.59 | (t, ³J = 6.6 Hz, 2 H, |
| | —OCH₂CH(OCH₂(CH₂)₁₂CH₃)CH₂O—) |
| δ = | (m, 1 H, —OCH₂CH(O—)CH₂O—) |
| 3.62 − 3.75 | |
| δ = 3.73 | (s, 2 H, —OCOCH₂N(CH₂)₄N—) |
| δ = 4.23 | (dd, ³J = 5.7 Hz and ²J = 11.4 Hz, 1 H, |
| | —CHHOCOCH₂N—) |
| δ = 4.37 | (dd, ³J = 3.8 Hz and ²J = 11.4 Hz, 1 H, |
| | —CHHOCOCH₂N—) |
| δ = 8.25 | (s broad, 2 H, 2 HCOO) |

### Synthesis Procedures for Bicationic DMG Derivatives

### 10-((2,3-di-tetradecyloxy)-propyloxcarbonylmethyl)-1-Z-5,10-dibenzyl-1,5,10-triazadekan (114)

Add 69 mg (0.5 mmol) potassium carbonate to a solution of 460 mg (1.0 mmol) 1-Z-5,10-dibenzyl-1,5,10-triazadekan (79) and 1010 mg (1.8 mmol) chloroacetic acid-(2,3-di-tetradecyloxy)-propylester (109) in acetonitrile/toluene (8:1). Stir overnight with reflux. Remove the solvent and purify the residue via column chromatography on 25 g silica gel. Elute excess quantities of lipid component with cyclohexane/diisopropyl ether (2:1), and elute the product with cyclohexane/ethyl acetate (4:1 to 2:1).

| | |
|---|---|
| Yield: | 709 mg (72% of theoretical value) as a yellow oil |
| Mᵣ: | 984.50 (C₆₂H₁₀₁N₃O₆) |
| R_{f}: | 0.31 (cyclohexane/ethyl acetate 2:1) |

¹H-NMR (250 MHz, CDCl₃):

| | |
|---|---|
| δ = 0.88 | (t, ³J = 6.7 Hz, 6 H, 2 -O(CH₂)₁₃CH₃) |
| δ = | (m, 44 H, 2 -O(CH₂)₂(CH₂)₁₁CH₃) |
| 1.12 − 1.38 | |
| δ = | (m, 10 H, 2 -OCH₂CH₂C₁₂H₂₅and |
| 1.39 − 1.69 | —NCH₂(cH₂)₂CH₂NCH₂CH₂CH₂N—) |
| δ = 2.36 | (t, ³J = 6.6 Hz, 2 H, —N(CH₂)₃CH₂N(CH₂)₃NHZ) |
| δ = 2.44 | (t, ³J = 6.1 Hz, 2 H, —N(CH₂)₄NCH₂(CH₂)₂NHZ) |
| δ = 2.59 | (t, ³J = 6.6 Hz, 2 H, —NCH₂(CH₂)₃N(CH₂)₃NHZ) |
| δ = | (m, 2 H, —N(CH₂)₂CH₂NHZ) |
| 3.14 − 3.27 | |
| δ = 3.29 | (s, 2 H, —OCOCH₂N(CH₂)₄N—) |
| δ = 3.41 | (t, ³J = 6.6 Hz, 2 H, —CH₂OCH₂(CH₂)₁₂CH₃) |
| δ = | (m, 2 H, —OCH₂CH(O—)CH₂OCOCH₂N) |
| 3.43 − 3.51 | |
| δ = 3.48 | (s, 2 H, C₆H₅CH₂—N(CH₂)₃NHZ) |
| δ = 3.52 | (t, ³J = 6.7 Hz, 2 H, |
| | —OCH₂CH(OCH₂(CH₂)₁₂CH₃)CH₂O—) |
| δ = | (m, 1 H, —OCH₂CH(O—)CH₂O—) |
| 3.55 − 3.65 | |
| δ = 3.73 | (s, 2 H, C₆H₅CH₂—N(CH₂)₄N(CH₂)₃NHZ) |
| δ = 4.10 | (dd, ³J = 5.8 Hz and ²J = 11.6 Hz, 1 H, |
| | —CHHOCOCH₂N—) |
| δ = 4.25 | (dd, ³J = 4.0 Hz and ²J = 11.6 Hz, 1 H, |
| | —CHHOCOCH₂N—) |
| δ = 5.07 | (s, 2 H, —NHCOOCH₂C₆H₅) |
| δ = | (m, 1 H, —NHZ) |
| 5.70 − 5.81 | |
| δ = | (m, 15 H, H_{aromat.}) |
| 7.15 − 7.37 | |

### 10-((2,3-di-tetradecyloxy)-propyloxcarbonylmethyl)-1,5,10-triazadekan formic acid salt (1150

Add 106 mg (0.1 mmol) palladium/activated charcoal (10%) to a solution of 1010 mg (1.0 mmol) 10-((2,3-di-tetradecyloxy)-propyloxcarbonylmethyl)-1-Z-5,10-dibenzyl-1,5,10-triazadekan (114) in 4 ml of a solvent mixture of dichloromethane/methanol (1:1). Stir overnight in a hydrogen atmosphere. Concentrate the formulation to a small volume to dry it and purify the residue via column chromatography on 30-40 g silica gel. Elute the apolar impurities with chloroform/methanol (9:1) or with chloroform/methanol/ammonia (25%) (90:10:1), and elute the product with chloroform/methanol/ammonia (25%) (60:40:1). Combine the fractions that contain product, remove the solvent, and dry the residue well in a high vacuum. Take up the product in 1-2 ml dichloromethane/acetone (1:1) and add 1 ml formic acid. This causes a majority of the product to precipitate out as formic acid salt. Remove the solvent and acetic acid. The product 115 is obtained as a solid or a sticky mass.

| | |
|---|---|
| Yield: | 442 mg (52% of theoretical value) |
| Mᵣ: | 850.27 (C₄₆H₉₅N₃O₁₀) |
| R_{f}: | 0.14 (chloroform/methanol/ammonia (25%) 60:40:2) |

¹H-NMR (250 MHz, CDCl₃/ CD₃OD/D₂O 20:10:1):

| | |
|---|---|
| δ = 0.89 | (t, ³J = 6.6 Hz, 6 H, 2 -O(CH₂)₁₃CH₃) |
| δ = | (m, 44 H, 2 -O(CH₂)₂(CH₂)₁₁CH₃) |
| 1.15 − 1.42 | |
| δ = | (m, 4 H, 2 -OCH₂CH₂(CH₂)₁₁CH₃) |
| 1.50 − 1.66 | |
| δ = | (m, 4 H, —NCH₂(CH₂)₂CH₂N(CH₂)₃N—) |
| 1.75 − 1.91 | |
| δ = 2.13 | (quint, ³J = 7.6 Hz, 2 H, —N(CH₂)₄NCH₂CH₂CH₂N—) |
| δ = | (m, 8 H, |
| 3.01 − 3.18 | —NCH₂(CH₂)₂CH₂NCH₂CH₂CH₂N—) |
| δ = 3.48 | (t, ³J = 6.7 Hz, 2 H, —CH₂OCH₂(CH₂)₁₂CH₃) |
| δ = | (m, 2 H, —OCH₂CH(O—)CH₂OCOCH₂N—) |
| 3.49 − 3.58 | |
| δ = 3.59 | (t, ³J = 6.9 Hz, 2 H, |
| | —OCH₂CH(OCH₂(CH₂)₁₂CH₃)CH₂O—) |
| δ = | (m, 1 H, —OCH₂CH(O—)CH₂O—) |
| 3.64 − 3.75 | |
| δ = 3.91 | (s, 2 H, —OCOCH₂N(CH₂)₄N—) |
| δ = 4.25 | (dd, ³J = 5.5 Hz and ²J = 11.6 Hz, 1 H, |
| | —CHHOCOCH₂N—) |
| δ = 4.39 | (dd, ³J = 4,1 Hz and ²J = 11.5 Hz, 1 H, |
| | —CHHOCOCH₂N—) |
| δ = 8.34 | (s broad, 3 H, 2 HCOO) |

Materials and Methods for Transfection Results

Liposome Preparations

To make the liposome preparation, dissolve 1.29 μmol of the simple cationic lipid to be tested (bicationic: 0.645 μmol, tricationic: 0.43 μmol) in chloroform/methanol (2:1 v/v) in a test tube with an equimolar quantity of DOPE. Use the simple cationic lipid DOTAP without the addition of DOPE.

Slowly blow off the organic solvent with a TCS sample preparation system (Vapotherm, BARLEY, Bielefeld, Germany) using nitrogen (30-60 minutes) at room temperature. The dried lipid films can be stored for many months at −20° C. To prepare the liposomes, hydrate the lipid films with 1 ml HBS buffer at room temperature for 20 minutes, then treat in an ultrasound bath (BANDELIN Sonopuls GM 200; Berlin, Germany) for 2 minutes at 37° C.

Composition of the HBS buffer:

20 mM HEPES

130 mM NaCl

pH 7.4

Characterize the liposomes by determining the size distribution using a Submicron Particle Sizer (Autodilute Modell 370, NICOMP, Santa Barbara, Calif., USA).

Making the Lipoplexes

Dilute the liposome dispersions 10-fold. To accomplish this, add 90 μl of the respective liposome dispersion to each 810 μl HBS buffer. Make the following liposome dilutions to prepare 8 different lipoplexes with different lipid/DNA ratios:

| Lipid/DNA | | | | | | | | |
|---|---|---|---|---|---|---|---|---|
| Ratio | 1:1 | 3:1 | 5:1 | 7:1 | 9:1 | 11:1 | 13:1 | 15:1 |
| Liposomen | 12 | 36 | 60 | 84 | 108 | 132 | 156 | 180 |
| Dispersion [μl] | | | | | | | | |
| HBS | 228 | 204 | 180 | 156 | 132 | 108 | 84 | 60 |
| Buffer [μl] | | | | | | | | |

Prepare a 1:240 dilution in HBS from a stock solution of the plasmid (pCMXluc 8600 bp, 1 mg/ml). Add 120 μl of the plasmid solution to the 240 μl of liposome disperson and mix carefully. Allow the mixtures to stand at room temperature for 60 minutes to form the lipoplexes before they are applied to cells. Manufacture the lipoplexes containing the lipid DOTAP using a lipid/DNA ratio of 2.5:1 recommended by ROCHE. To accomplish this, dilute 75 μl of the 1:10 diluted liposome dispersion with 525 μl HBS buffer, then add 300 μl plasmid solution. (The quantities are calculated for 8-fold determinations). Allow the mixture to stand at room temperature for 60 minutes to form the lipoplexes.

Cell Transfection

Cultivate the COS-7 cells in 250 ml cell culture bottles (GREINER) in EMEM medium in an incubator (model 600 HERAEUS INSTRUMENTS) at 37° C. and 5% CO₂ in a saturated steam atmosphere.

Composition of the Culture Medium:

EMEM medium (BIO WHITTAKER, Verviers, Belgium)

10% FCS (fetal calf serum, SERVA, Heidelberg, Germany heat-inactivated for 30 min at 56° C.)

1% penicillin (10000 U/ml, BIO WHITTAKER)

1% streptomycin (10 g/ml, BIO WHITTAKER)

24 hours before transfection, sow 5000 cells in 200 μl medium per well in 96-well microtiter plates. The cells should exhibit a confluence of 40-50% one hour before transfection. Carefully remove 100 μl medium and replace it with 90 μl lipoplex solution. Transfect 3 wells each per lipid/DNA ratio, and transfect 8 wells for DOTAP. On a 96-well microtiter plate, therefore, 3 different lipids-in addition to DOTAP, the standard-can be tested in 8 different lipid/DNA ratios. Centrifuge the 96-well microtiter plate at 280×g for 2 minutes, then incubate the cells in the incubator for 4 hours. Remove the medium completely and replace it with 200 μl of fresh, pre-warmed medium. Cultivate the cells for another 44 hours in the incubator.

Determination of Luciferase Activity and the Total Protein Content

Remove the supernatant medium completely, wash the cells once with a 0.9% NaCl solution, then add 50 μl lysis buffer (ROCHE, Germany) per well. Incubate at room temperature for 20 minutes, then add another 30 μl NaCl solution per well and mix thoroughly. Transfer 20 μl of the lysate from each cell to a new 96-well microtiter plate for determination of the total protein content. Transfer another 20 μl of the lysate to a white 96-well microtiter plate (cOSTAR-CORNING, Germany) for determination of the luciferase activity. Use a BCA test (PIERCE, Rockford, USA) to determine the quantity of total protein per well. First, plot a calibration curve using a BSA (bovine serum albumin) dilution series. Add 200 μl BCA reagent per well to each 20 μl lysate and incubate for 2 hours at room temperature and protected from light. Determine the protein concentration using the calibration curve in the spectral photometer (Spectra, TECAN, Germany) by quantifying the absorption at λ=550 nm.

To determine luciferase activity, transfer 80 μl of a luciferin substrate solution to each well in a luminometer (Lumistar, BMG LABTECHNOLOGIES, Offenburg, Gemany) and measure the light emission over a period of 10 seconds.

Composition of the Luciferin Substrate Solution:

25 mM glycylglycin (FLUKA)

5 mM ATP (ROCHE)

0.2 mM luciferin (PROMEGA)

pH 7.8

The luciferase activity is expressed in relative light units (RLU) per well and is then based on the total protein quantity per well (RLU/μg protein). The corresponding values for the various lipids are based on DOTAP and are therefore expressed in relative percent. The relative transfection efficiency is defined as follows:

$$\text{relative transfection efficiency [\%]} = 100 \times \frac{tr \cdot eff \cdot lipid [ RLU \text{/} µg protein ]}{tr \cdot eff \cdot DOTAP [ RLU \text{/} µg protein ]}$$

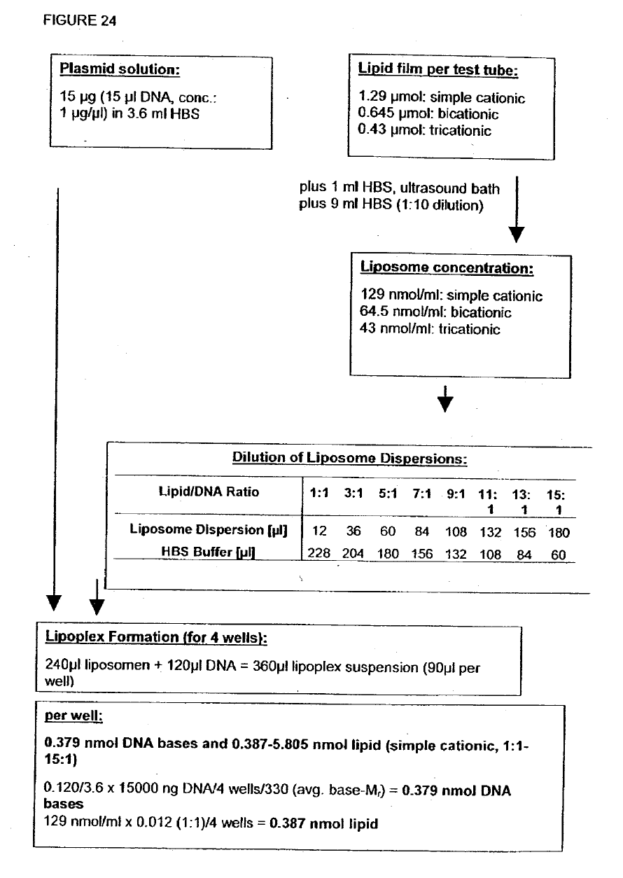

FIG. 24 shows an example of calculating lipid/DNA ratios (illustrating with a lipid in 8 different lipid/DNA ratios).

### REFERENCES

A. M. Aberle, F. Tablin, J. Zhu, N. J. Walker, D. C. Gruenert and M. H. Nantz (1998) Biochemistry 37, 6533

D. Aikens, S. Bunce, F. Onasch, R. Parker, C. Hurwitz and S. Clemans (1983) Biophys. J. 17, 67

D. Balasundaram and A. K. Tyagi (1991) Mol. Cell. Biochem. 100, 129

B. J. Battersby, R. Grimm, S. Huebner and G. Cevc (1998) Biochim. Biophys. Acta 1372, 379

J.-P. Behr, B. Demeneix, J.-P. Loeffler and J. Perez-Mutul (1989) Proc. Natl. Acad. Sci. USA 86, 6982

J.-P. Behr (1993) Acc. Chem. Res. 26, 274

J.-P. Behr (1994) Pure & Appl. Chem. 66(4), 827

M. J. Bennett, R. W. Malone and M. H. Nantz (1995) Tetrahedron Lett. 36, 2207

G. Benz (1984) Liebigs Ann. Chem., 1424

R. J. Bergeron (1986) Acc. Chem. Res. 19, 105

R. J. Bergeron, J. S. McManis, W. R. Weimar, K. M. Schreier, F. Gao, Q. Wu, J. Ortiz-Ocasio, G. R. Luchetta, C. Porter and J. R. T. Vinson (1995) J. Med. Chem 38, 2278

M. A. Bernardo, J. A. Guerrero, E. Garcia-Espana, S. V. Luis, J. M. Llinares, F. Pina, J. A. Ramirez and C. Soriano (1996) J. Chem. Soc., Perkin Trans. 2, 2335

W. Bertling, K. Hunger-Bertling and M. J. Cline (1987) J. Biochem. Biophys. Methods 14, 223

S. Bhaftacharya and S. S. Mandal (1998) Biochemistry 37, 7764

R. M. Blaese (1997) Spektrum der Wissenschaft 12, 48

I. S. Blagbrough, E. Moya and S. P. Walfort (1996) Tetrahedron Lett. 37, 551

I. S. Blagbrough and A. J. Geall (1998) Tetrahedron Lett 39, 439

R. Bottega and R. M. Epand (1992) Biochemistry 31, 9025

G. Byk, C. Dubertret, V. Escriou, M. Frederic, G. Jaslin, R. Rangara, B. Pitard, J. Crouzet, P. Wils, B. Schwartz and D. Scherman (1998) J. Med. Chem. 41, 224

M. R. Capecchi (1980) Cell 22, 478

N. J. Caplen, E. W. Alton, P. G. Middleton, J. R. Dorin, B. J. Stevenson, X. Gao, S. R. Durham, P. K. Jeffery, M. E. Hodson, C. Coutelle, L. Huang, D. J. Porteous, R. Williamson and D. M. Geddes (1995) Nature Med. 1, 39

L. Chen, P. R. Ziegelhoffer and N. S. Yang (1993) Proc. Natl. Acad. Sci. USA 90,

R. G. Cooper, C. J. Etheridge, L. Stewart, J. Marshall, S. Rudginsky, S. H. Cheng and A. D. Miller (1998) Chem. Eur. J. 4(1),137

K. Crook, B. J. Stevenson, M. Dubouchet and D. J. Porteous (1999) Gene Ther. 5, 137

R. G. Crystal (1995) Science 270, 404

N. Dan (1998) Biochim. Biophys. Acta 1369, 34

H. M. Deshmukh and L. Huang (1997) New J. Chem. 21, 113

J. C. Dittmer and R. L. Lester (1964) J. Lipid Res. 5, 126

S. J. Eastman, C. Siegel, J. Tousignant, A. E. Smith, S. H. Cheng and R. K. Scheule (1997) Biochim. Biophys. Acta 1325, 41

H. Eibl and P. Woolley (1986) Chem. Phys. Lipids 33, 53

P. W. Erhardt (1983) Synth. Commun. 13(2),103

V. Escriou, C. Ciolina, A. Helbling-Leclerc, P. Wils and D. Scherman (1998) Cell Biol. Toxicology 14, 95

H. Farhood, R. Bottega, R. M. Epand and L. Huang (1992) Biochim. Biophys. Acta 1111, 239

H. Farhood, N. Serbina and L. Huang (1995) Biochim. Biophys. Acta 1235, 289

P. L. Felgner, T. R. Gadek, M. Holm, R. Roman, H. W. Chan, M. Wenz, J. P. Northrop, G. M. Ringold and M. Danielsen (1987) Proc. Natl. Acad. Sci. USA 84, 7413

J. H. Felgner, R. Kumar, C. N. Sridhar, C. J. Wheeler, Y. J. Tsai, R. Border, P. Ramsey, M. Martin and P. L. Felgner (1994) J. Biol. Chem. 269, 2550

T. Ferkol, J. C. Perales, F. Mularo and R. W. Hanson (1996) Proc. Natl. Acad. Sci. USA 93, 101

W. J. Fiedler and M. Hesse (1993) Helv. Chim. Acta 76, 1511

Th. Fichert (1996) Diplomarbeit, Albert-Ludwigs-Universität, Freiburg im Breisgau

Th. Fichert and U. Massing (1998) Tetrahedron Lett. 39, 5017

T. Friedmann (1997) Spektrum der Wissenschaft 10, 50

T. Fukuyama, C.-K. Jow and M. Cheung (1995) Tetrahedron Lett. 36, 6373

X. Gao and L. Huang (1991) Biochem. Biophys. Res. Commun. 179, 280

X. Gao and L. Huang (1993) J. Liposome Res. 3, 17

X. Gao and L. Huang (1995) Gene Ther. 2, 710

F. Garro-Helion, A. Merzouk and F. Guibé (1993) J. Org. Chem. 58, 6109

A. J. Geall and I. S. Blagbrough (1998) Tetrahedron Lett. 39, 443

A. J. Geall, R. J. Taylor, M. E. Earl, M. A. Eaton uns I. S. Blagbrough (1998) Chem. Commun. 1403

H. Geshon, R. Ghirlando, S. B. Guttman and A. Minsky (1993) Biochemistry 32,

C. Goulaouic-Dubois, A. Guggisberg and M. Hesse (1995) Tetrahedron 51, 12573

Th. W. Greene and P. G. Wuts, 1991, in: Protective Groups in Organic Synthesis, pp. 364-368, John Wiley & Sons, Inc., New York

J. Gustasson, G. Arvidson, G. Karlsom and M. Almgren (1995) Biochim. Biophys. Acta 1235, 305

J. K. Guy-Caffey, V. Bodepudi, J. S. Bishop, K. Jayaraman and N. Chaudhary (1995) J. Biol. Chem. 270, 31391

J. B. Hendrickson and R. Bergeron (1973) Tetrahedron Lett. 14, 3839

Y. Hidai, T Kann and T. Fukuyama (1999) Tetrahedron Lett. 40, 4711

C.-Y. Huang, T. Uno, J. E. Murphy, S. Lee, J. D. Hamer, J. A. Escobedo, F. E. Cohen, R. Radhakrishnan, V. Dwarki and R. N. Zuckermann (1998) Chem. Biol. 5, 345

P. A. Jacobi, M. J. Martinelli and S. J. Polanc (1984) J. Am. Chem. Soc. 106, 5594

W. A. Keown, C. R. Campbell and R. S. Kucherlapati (1990) In: Methods in Enzymology (Vol. 185) pp. 527-537, Academic Press, New York

H. A. Khan and D. J. Robins (1985) J. Chem. Soc., Perkin Trans. 1, 101

G. v. Kiedrowski and F. Z. Dörwald (1988) Liebigs Ann. Chem., 787

T. M. Klein, E. D. Wolf, R. Wu and J. C. Sanford (1987) Nature 327, 70

J. T. Kley, Th. Fichert and U. Massing (1998) Monatsh. Chem. 129, 319

M. R. Knowles, K. W. Hohneker, Z. Zhou, J. C. Olsen, T. L. Noah, P. C. Hu, M. W. Leigh, J. F. Engelhardt, L. J. Edwards, K. R. Jones, M. Grossmann, J. M. Wilson, L. G. Johnson and R. C. Boucher (1995) N. Engl. J. Med. 333, 823

Ph. J. Kocienski, 1994, in: Protecting Groups, (D. Enders, R. Noyori, B. Trost, eds.) pp. 220-227, Georg Thieme Verlag, Stuttgart

D. D. Lasic (1994) Angew. Chem. Int. Ed. 33, 1479

D. D. Lasic and D. Papahadjopoulos (1995) Science 267, 1275

D. D. Lasic, H. Strey, M. C. Stuart, R. Podornik and P. M. Frederik (1997) J. Am. Chem. Soc. 119, 832

E. R. Lee, J. Marshall, C. S. Siegel, C. Jiang, N. S. Yew, M. R. Nichols, J. B. Nietupsky, R. J. Ziegler, M. B. Lane, K. X. Wang, N. C. Wan, R. K. Schuele, D. J. Harris, A. E. Smith and S. H. Cheng (1996) Hum. Gene Ther. 7, 1701

R. Leventis and J. R. Silvius (1990) Biochim. Biophys. Acta 1023, 124

D. C. Litzinger and L. Huang (1992) Biochim. Biophys. Acta 1113, 201

M. Lochner, H. Geneste and M. Hesse (1998) Helv. Chim. Acta 81, 2270

A. D. Miller (1998) Angew. Chem. Int. Ed. 37, 1768

R. Moog (1999) Doktorarbeit, Albert-Ludwigs-Universität, Freiburg im Breisgau

D. Moradpour, J. I. Schauer, V. R. Zurawski, Jr., J. R. Wands and R. H. Boutin (1996) Biochem. Biophys. Res. Commun. 221(1), 82

R. A. Morgan and W. F. Anderson (1993) Annu. Rev. Biochem. 62, 191

I. Mortimer, P. Tom, I. MacLachlan, R. W. Graham, E. G. Saravolac and P. B. Joshi (1999) Gene Ther. 6, 403

R. C. Mulligan (1993) Science 260, 926

M. Niitsu and K. Samejima (1986) Chem. Pharm. Bull. 34, 1032

J. E. Nordlander, D. B. Catalane, T. H. Eberlein, L. V. Farkas, R. S. Howe, R. M. Stevens and N. A. Tripoulas (1078) Tetrahedron Lett. 19, 4987

S. Obika, W. Yu, A. Shimoyama, T. Uneda, K. Miyashita, T. Doi and T. Imanishi (1997) Bioorg. Med. Chem. Lett. 7(14), 1817

S. Obika, W. Yu, A. Shimoyama, T. Uneda, T. Minami, K. Miyashita, T. Doi and T. Imanishi (1999) Biol. Pharm. Bull. 22(2), 187

R. Okayama, M. Noji and M. Nakanishi (1997) FEBS Lett. 408, 232

L. E. Overman, L. T. Mendelson and E. J. Jacobsen (1983) J. Am. Chem. Soc. 105, 6629

J. K. Pak and M. Hesse (1998) J. Org. Chem. 63, 8200

J. O. Rädler, I. Koltover, T. Salditt and C. R. Safinya (1997) Science 275, 810

A. Regelin (2000) Doktorarbeit, Albert-Ludwigs-Universität, Freiburg im Breisgau

F. Reifers and J. Kreuzer (1995) J. Mol. Med. 73, 595

T. Ren and D. Liu (1999) Bioorg. Med. Chem. Lett. 9, 1247

K. Samejima, Y. Takeda, M. Kawase, M. Okada and Y. Kyoguku (1984) Chem. Pharm. Bull. 32, 3428

J. G. Smith, R. L. Walzem and J. B. German (1993) Biochim. Biophys, Acta 1154, 327

I. Solodin, C. S. Brown and T. D. Heath (1996) Synlett. 5, 457

B. Sternberg, F. L. Sorgi and L. Huang (1994) FEBS Lett. 256, 361

Y. Takeda, K. Samejima, K. Nagano, M. Watanabe, H. Sugeta and Y. Kyogoku (1983) Eur. J. Biochem. 130, 383

K. Takeuchi, M. Ishihara, C. Kawaura, M. Noji, T. Furuno and M. Nakanishi (1996) FEBS Lett. 397, 207

L. Velluz, G. Amiard and R. Heymes (1954) Bull. Soc. Chim. Fr., 1012

J. Wang, X. Guo, Y. Xu, L. Barron and C. Szoka (1998) J. Med. Chem. 41, 2207

J.-M. Weibel, A. Kichler, J.-S. Remy, C. Gaiddon, J.-P. Loeffler, G. Duportail and D. Heissler (1995) Chem. Lett., 473

M. J. Welsh and J. Zabner (1999) Hum Gene Ther. 10, 1559

T.-K. Wong, C. Nicolau and P. H. Hofschneider (1980) Gene 10, 87

I. Wrobel and D. Collins (1995) Biochim. Biophys. Acta 1235, 296

Y. Xu and F. C. Szoka, Jr. (1996) Biochemistry 35, 5616

J.-P. Yang and L. Huang (1998) Gene Ther 5, 380

Y. Yoshikawa, N. Emi, T. Kanbe, K. Yoshikawa and H. Saito (1996) FEBS Lett. 396, 71

J. You, M. Mamihira and S. Iijima (1999) J. Biochem. 125, 1160

W. Yu, A. Shimoyama, T. Uneda, S. Obika, K. Miyashita, T. Doi and T. Imanishi (1999) J. Biochem. 125, 1034

J. Zabner, A. J. Fasbender, T. Moninger, K. A. Poellinger and M. J. Welsh (1995) J. Biol. Chem. 270, 18997

N. J. Zuidam and Y. Barenholz (1997) Biochim. Biophys. Acta 1329, 211

N. J. Zuidam and Y. Barenholz (1998) Biochim. Biophys. Acta 1368, 115

1. A cationic amphiphile having the structure A-F-D, wherein:
A is a lipid anchor;
F is a spacer group having the structure
O—C(O)-G¹-[C(R¹)(R²)]ₘ-G²-{C(O)-E-[C(R³)(R⁴)]ₙ}ₚ; and
D is a head group; and wherein:
G¹ and G² are the same or different, and are independently either oxygen or a bond;
R¹, R², R³ and R⁴ are the same or different, and are independently selected from the group consisting of hydrogen and alkyl radicals;
m, n and p are the same or different, and are independently either 0, 1, 2, 3, 4, 5, or 6; and
E is oxygen or N(R⁵), wherein R⁵ is hydrogen or an alkyl radical, provided that E does not contain nitrogen when D is N(CH₃)₂ and when A is cholesterol, and when R⁵ is hydrogen, and when both G¹ and G² are bonds, and when each of R¹, R², R³ and R⁴ is hydrogen, and when both m and n are 2, and when p is 1.

2. The amphiphile of claim 1, wherein R¹, R², R³ and R⁴ are all hydrogen.

3. The amphiphile of claim 1, wherein R¹, R², R³ and R⁴ are alkyl radicals, either unsaturated, straight, branched or any combination thereof.

4. The amphiphile of claim 1, wherein said lipid anchor is a steroid.

5. The amphiphile of claim 4, wherein said steroid is cholesterol.

6. The amphiphile of claim 1, wherein said lipid anchor is a lipophilic lipid comprising two alkyl chains, said alkyl chains containing at least eight contiguous methylene units.

7. The amphiphile of claim 1, wherein said lipid anchor is a lipophilic lipid comprising two alkyl chains, wherein the length of said alkyl chains is from eight to twenty-four carbon atoms.

8. The amphiphile of claim 1, wherein said lipid anchor is a lipophilic lipid comprising two alkyl chains, wherein the length of said alkyl chains is from eight to twenty-four carbon atoms, and wherein said alkyl chains may be saturated, unsaturated, straight, branched or any combination thereof.

9. The amphiphile of claim 1, wherein said lipid anchor is selected from the group consisting of cholesterol, dierucylglycerol, diacylglycerol, and 1,2-dimyristyloxypropan-3-ol.

10. The amphiphile of claim 1, wherein said lipid anchor is 1,2-dimyristyloxypropan-3-ol.

11. The amphiphile of claim 1, wherein said spacer group F is selected from the group consisting of O—C(O)—CH₂, O—C(O)—(CH₂)₂, O—C(O)—(CH₂)₃, O—C(O)—O—(CH₂)₂, O—C(O)—(CH₂)₂—C(O)—O—(CH₂)₂, O—C(O)—(CH₂)₂—C(O)—O—(CH₂)₃, and O—C(O)—(CH₂)₂—C(O)—NH—(CH₂)₃.

12. The amphiphile of claim 1, wherein said spacer group F is selected from the group consisting of O—C(O)—CH₂, O—C(O)—O—(CH₂)₂, O—C(O)—(CH₂)₂—C(O)—O—(CH₂)₂, O—C(O)—(CH₂)₂—C(O)—O—(CH₂)₃, and O—C(O)—(CH₂)₂—C(O)—NH—(CH₂)₃.

13. The amphiphile of claim 12, wherein A is cholesterol.

14. The amphiphile of claim 1 or 13, wherein D is selected from the group consisting of NH—(CH₂)₄—NH—CH₂CH₃, NH—(CH₂)₆—NH—CH₂CH₃, NH—(CH₂)₄—NH—(CH₂)₃—N H₂, N H—(CH₂)₄—N H—(CH₂)₃—N H₂, NH—(CH₂)₃—N H—(CH₂)₂—NH₂, NH—(CH₂)₃—NH—(CH₂)₃—NH₂, NH—(CH₂)₄—NH—(CH₂)₂—NH₂, NH—(CH₂)₅—NH—(CH₂)₂—NH₂, and NH—(CH₂)₆—NH—(CH₂)₂—NH₂.

15. The amphiphile of claim 12, wherein A is 1,2-dimyristyloxypropan-3-ol.

16. The amphiphile of claim 1 or 15, wherein D is selected from the group consisting of N(CH₃)₃, and NH—(CH₂)₄—NH—(CH₂)₃—NH₂.

17. The amphiphile of claim 1, wherein said head group is an amino group.

18. The amphiphile of claim 17, wherein said amino group is selected from the group consisting of primary amines, secondary amines, tertiary amines and quaternary amines.

19. The amphiphile of claim 18, wherein said secondary amines, said tertiary amines, and said quaternary amines are alkylated with at least one radical selected from the group consisting of methyl, ethyl, propyl, isopropyl, hydroxymethyl, hydroxyethyl, hydroxypropyl, glycerol, and mannitol.

20. The amphiphile of claim 1, wherein said head group comprises two amino groups.

21. The amphiphile of claim 20, wherein said amino groups are selected from the group consisting of primary amines, secondary amines, tertiary amines and quaternary amines.

22. The amphiphile of claim 21, wherein said secondary amines, said tertiary amines, and said quaternary amines are alkylated with at least one radical selected from the group consisting of methyl, ethyl, propyl, isopropyl, hydroxymethyl, hydroxyethyl, hydroxypropyl, glycerol, and mannitol.

23. The amphiphile of claim 1, wherein said head group comprises three amino groups.

24. The amphiphile of claim 23, wherein said amino groups are selected from the group consisting of primary amines, secondary amines, tertiary amines and quaternary amines.

25. The amphiphile of claim 24, wherein said secondary amines, said tertiary amines, and said quaternary amines are alkylated with at least one radical selected from the group consisting of methyl, ethyl, propyl, isopropyl, hydroxymethyl, hydroxyethyl, hydroxypropyl, glycerol, and mannitol.

26. The amphiphile of claim 1, 17, 18 or 19, wherein said head group is selected from the group consisting of N(CH₃)₂, N(CH₃)₃, and N(CH₃)₂CH₂CH₂OH.

27. The amphiphile of claim 1, 20, 21 or 22, wherein said head group is a diamine having the structure N(L¹)(L²)—(CH₂)ⱼ—N(L³)(L⁴), wherein:
j=2, 3, 4, 5 or 6; and
L¹, L², L³ and L⁴ are the same or different, and are independently selected from the group consisting of hydrogen, methyl, ethyl, propyl, isopropyl, hydroxymethyl, hydroxyethyl, hydroxypropyl, glycerol, and mannitol.

28. The amphiphile of claim 1, 23, 24 or 25, wherein said head group is a triamine having the structure N(L¹)(L²)—(CH₂)ⱼ—N(L³)(L⁴)—(CH₂)ₖ—N(L⁵)(L⁶), wherein:
j=2, 3, 4, 5 or 6;
L¹, L², L³ and L⁴ are the same or different, and are independently selected from the group consisting of hydrogen, methyl, ethyl, propyl, isopropyl, hydroxymethyl, hydroxyethyl, hydroxypropyl, glycerol, and mannitol;
L⁵ and L⁶ are the same or different and are independently selected from the group consisting of hydrogen, methyl, ethyl, propyl, isopropyl, hydroxymethyl, hydroxyethyl, hydroxypropyl, glycerol, and mannitol; and
k is 2, 3, 4, 5 or 6.

29. The amphiphile of claim 1, wherein said head group is a polyamine having a repeating structure [N(L¹)(L²)—(CH₂)ⱼ]_{q}—N(L³)(L⁴), wherein:
j=2, 3, 4, 5 or 6;
L¹, L², L³ and L⁴ are the same or different, and are independently selected from the group consisting of hydrogen, methyl, ethyl, propyl, isopropyl, hydroxymethyl, hydroxyethyl, hydroxypropyl, glycerol, and mannitol; and
q is greater than 3.

30. The amphiphile of claim 1, wherein said head group is spermidine.

31. The amphiphile of claim 1, wherein said head group is spermine.

32. A cationic amphiphile having the structure A-F-D, wherein:
A is a lipid anchor;
F is a spacer group having the structure O—C(O)-Q-(CH₂)ₘ; and
D is a head group; and wherein:
m=0, 1, 2, 3, 4, 5 or 6; and
Q is oxygen or has the structure (CH₂)ₓ—[C(O)E]ᵢ, wherein:
x=1 or 2;
i=0 or 1;
E is oxygen or N(R⁵), wherein R⁵ is hydrogen or an alkyl radical, provided that E is not nitrogen when D is N(CH₃)₂ and A is cholesterol, and when R⁵ is hydrogen, and when m is 2, and when x=2, and when i=1.

33. A lipid mixture comprising:
a cationic amphiphile having a structure as recited in claim 1 or 12; and
at least one helper lipid.

34. A lipid mixture comprising:
a cationic amphiphile having a structure as recited in claim 14; and
at least one helper lipid.

35. A lipid mixture comprising:
a cationic amphiphile having a structure as recited in claim 16; and
at least one helper lipid.

36. The lipid mixture of claim 33, wherein said helper lipid is selected from the group consisting of DOPE, cholesterol and lecithins.

37. The lipid mixture of claim 33, wherein said helper lipid is DOPE.

38. A liposome comprising:
a cationic amphiphile having a structure as recited in claim 1 or 12; and
at least one helper lipid.

39. A liposome comprising:
a cationic amphiphile having a structure as recited in claim 14; and
at least one helper lipid.

40. A liposome comprising:
a cationic amphiphile having a structure as recited in claim 16; and
at least one helper lipid.

41. The liposome of claim 38, wherein said helper lipid is selected from the group consisting of DOPE, cholesterol and lecithins.

42. The liposome of claim 38, wherein said helper lipid is DOPE.

43. The lipid mixture of claim 33, 34, 35, 36 or 37, wherein said cationic amphiphile and said helper lipid are present in a molar mixing ratio of from about five to one to about one to five.

44. The liposome of claim 38, 39, 40, 41 or 42, wherein said cationic amphiphile and said helper lipid are present in a molar mixing ratio of from about five to one to about one to five.

45. A method for facilitating transport of a biologically active molecule into a cell, said method comprising:
preparing a lipid mixture comprising a cationic amphiphile having the structure A-F-D, wherein:
A is a lipid anchor;
F is a spacer group having the structure
O—C(O)-G¹-[C(R¹)(R²)]ₘ-G²-{C(O)-E-[C(R³)(R⁴)]ₙ}ₚ; and
D is a head group; and wherein:
G¹ and G² are the same or different, and are independently either oxygen or a bond;
R¹, R², R³ and R⁴ are the same or different, and are independently selected from the group consisting of hydrogen and alkyl radicals;
m, n and p are the same or different, and are independently either 0, 1, 2, 3, 4, 5, or 6; and
E is oxygen or N(R⁵), wherein R⁵ is hydgrogen or an alkyl radical, provided that E does not contain nitrogen when D is N(CH₃)₂ and A is cholesterol, and when R⁵ is hydrogen, and when both G¹ and G² are bonds, and when each of R¹, R², R³ and R⁴ is hydrogen, and when both m and n are 2, and when p is 1;
preparing a lipoplex by contacting said lipid mixture with a biologically active molecule; and
contacting said lipoplex with a cell, thereby facilitating transport of said biologically active molecule into said cell.

46. The method of claim 45, wherein said lipid mixture is in the form of liposome.

47. The method of claim 46, wherein said liposome is in a dispersion.

48. The method of claim 47, wherein the average size of said liposomes is between about 20 and about 1000 nanometers.

49. The method of claim 47, wherein the average particle size of said dispersion is between about 50 and about 200 nanometers.

50. The method of claim 45, wherein said cationic amphiphile has a structure A-F-D, wherein:
A is a lipid anchor;
F is a spacer group selected from the group consisting of O—C(O)—CH₂, O—C(O)—O—(CH₂)₂, O—C(O)—(CH₂)₂—C(O)—O—(CH₂)₂, O—C(O)—(CH₂)₂—C(O)—O—(CH₂)₃, and O—C(O)—(CH₂)₂—C(O)—NH—(CH₂)₃; and
D is a head group.

51. The method of claim 45, wherein said cationic amphiphile has a structure A-F-D, wherein:
A is cholesterol;
F is a spacer group selected from the group consisting of O—C(O)—CH₂, O—C(O)—O—(CH₂)₂, O—C(O)—(CH₂)₂—C(O)—O—(CH₂)₂, O—C(O)—(CH₂)₂—C(O)—O—(CH₂)₃, and O—C(O)—(CH₂)₂—C(O)—NH—(CH₂)₃; and
D is a head group selected from the group consisting of NH—(CH₂)₄—N H—CH₂CH₃, NH—(CH₂)₆—N H—CH₂CH₃, NH—(CH₂)₄—N H—(CH₂)₃—NH₂, NH—(CH₂)₄—NH—(CH₂)₃—NH₂, NH—(CH₂)₃—NH—(CH₂)₂—NH₂, NH—(CH₂)₃—NH—(CH₂)₃—NH₂, NH—(CH₂)₄—NH—(CH₂)₂—NH₂, NH—(CH₂)₅—NH—(CH₂)₂—NH₂, and NH—(CH₂)₆—NH—(CH₂)₂—NH₂.

52. The method of claim 45, wherein said cationic amphiphile has a structure A-F-D, wherein:
A is 1,2-dimyristyloxypropan-3-ol;
F is a spacer group selected from the group consisting of O—C(O)—CH₂, O—C(O)—O—(CH₂)₂, O—C(O)—(CH₂)₂—C(O)—O—(CH₂)₂, O—C(O)—(CH₂)₂—C(O)—O—(CH₂)₃, and O—C(O)—(CH₂)₂—C(O)—NH—(CH₂)₃; and
D is a head group selected from the group consisting of N(CH₃)₃, and NH—(CH₂)₄—NH—(CH₂)₃—NH₂.

53. The method of claim 45, 50, 51, or 52 wherein said biologically active molecule is a polyanion.

54. The method of claim 53 wherein the charge ratio of said cationic amphiphile to said polyanion is selected so to provide said lipoplex with a maximum degree of transfection efficiency.

55. The method of claim 53, wherein the charge ratio of said cationic amphiphile to said polyanion ranges from about 1 to 1 to about 15 to 1.

56. The method of claim 45, 50, 51, or 52 wherein said biologically active molecule is selected from the group consisting of DNA, RNA, synthetic polynucleotides, antisense polynucleotides, missense polyncletides, nonsense polynucleotides, ribozymes, proteins, biogically active polypeptides, small molecular weight drugs, antibiotics and hormones.

57. A method for treating a patient suffering from cancer, said method comprising:
preparing a lipid mixture comprising a cationic amphiphile having the structure A-F-D, wherein:
A is a lipid anchor;
F is a spacer group having the structure
O—C(O)-G¹-[C(R¹)(R²)]ₘ-G²-{C(O)-E-[C(R³)(R⁴)]ₙ}ₚ; and
D is a head group; and wherein:
G¹ and G² are the same or different, and are independently either oxygen or a bond;
R¹, R², R³ and R⁴ are the same or different, and are independently selected from the group consisting of hydrogen and alkyl radicals;
m, n and p are the same or different, and are independently either 0, 1, 2, 3, 4, 5, or 6; and
E is oxygen or N(R⁵), wherein R⁵ is hydgrogen or an alkyl radical, provided that E does not contain nitrogen when D is N(CH₃)₂ and A is cholesterol, and when R⁵ is hydrogen, and when both G¹ and G² are bonds, and when each of R¹, R², R³ and R⁴ is hydrogen, and when both m and n are 2, and when p is 1;
preparing a lipoplex by contacting said lipid mixture with a polyanion; and
providing said lipoplex in a therapeutically effective amount for contacting at least some of the cells involved in said cancer.

58. The method of claim 57, wherein said cells are tumor cells.

59. The method of claim 57, wherein said cells are tumor vasculature cells.

60. A method for treating a patient suffering from cancer, said method comprising:
preparing a lipid mixture comprising a cationic amphiphile having the structure A-F-D, wherein:
A is a lipid anchor;
F is a spacer group having the structure
O—C(O)-G¹-[C(R¹)(R²)]ₘ-G²-{C(O)-E-[C(R³)(R⁴)]ₙ}ₚ; and
D is a head group; and wherein:
G¹ and G² are the same or different, and are independently either oxygen or a bond;
R¹, R², R³ and R⁴ are the same or different, and are independently selected from the group consisting of hydrogen and alkyl radicals;
m, n and p are the same or different, and are independently either 0, 1, 2, 3, 4, 5, or 6; and
E is oxygen or N(R⁵), wherein R⁵ is hydgrogen or an alkyl radical, provided that E does not contain nitrogen when D is N(CH₃)₂ and A is cholesterol, and when R⁵ is hydrogen, and when both G¹ and G² are bonds, and when each of R¹, R², R³ and R⁴ is hydrogen, and when both m and n are 2, and when p is 1;
preparing a lipoplex by contacting said lipid mixture with an anti-tumor agent; and
providing said lipoplex in a therapeutically effective amount for contacting at least some of the cells involved in said cancer.

61. The method of claim 60, wherein said cells are tumor cells.

62. The method of claim 60, wherein said cells are tumor vasculature cells.

63. A method for treating a patient suffering from an inflammatory disease, said method comprising:
preparing a lipid mixture comprising a cationic amphiphile having the structure A-F-D, wherein:
A is a lipid anchor;
F is a spacer group having the structure
O—C(O)-G¹-[C(R¹)(R²)]ₘ-G²-{C(O)-E-[C(R³)(R⁴)]ₙ}ₚ; and
D is a head group; and wherein:
G¹ and G² are the same or different, and are independently either oxygen or a bond;
R¹, R², R³ and R⁴ are the same or different, and are independently selected from the group consisting of hydrogen and alkyl radicals;
m, n and p are the same or different, and are independently either 0, 1, 2, 3, 4, 5, or 6; and
E is oxygen or N(R⁵), wherein R⁵ is hydgrogen or an alkyl radical, provided that E does not contain nitrogen when D is N(CH₃)₂ and A is cholesterol, and when R⁵ is hydrogen, and when both G¹ and G² are bonds, and when each of R¹, R², R³ and R⁴ is hydrogen, and when both m and n are 2, and when p is 1;
preparing a lipoplex by contacting said lipid mixture with an anti-anti-inflammatory agent; and
providing said lipoplex in a therapeutically effective amount for contacting at least some of the cells involved in said inflammatory disesase.
